(19) United States
(12) Patent Application Publication
(10) Pub. No.: US 2009/0163708 A1
(43) Pub. Date: Jun. 25, 2009

(54) PROCESS FOR PREPARING MESYLATE SALTS OF IL-12 INHIBITORY COMPOUNDS

(75) Inventors: Elena Kostik, Arlington, MA (US); Lijun Sun, Harvard, MA (US); Keizo Koya, Chestnut Hill, MA (US); Pierre L. Boulas, Andover, MA (US)

Correspondence Address: EDWARDS ANGELL PALMER & DODGE LLP, P.O. BOX 55874, BOSTON, MA, 02205, US

(73) Assignee: Synta Pharmaceuticals Corp., Lexington, MA (US)

(21) Appl. No.: 12/110,317

(22) Filed: Apr. 26, 2008

Related U.S. Application Data
(63) Continuation of application No. PCT/US06/42211, filed on Oct. 27, 2006.
(60) Provisional application No. 60/731,038, filed on Oct. 27, 2005.

(51) Int. Cl. C07D 417/14; C07D 413/14; C07D 403/14

(52) U.S. Cl. 544/60; 544/113; 544/122; 544/124; 544/82; 544/121; 544/209

(57) ABSTRACT

This invention relates to a method of preparing mesylate salts of compounds that inhibit IL-12, IL-23 and/or IL-27 production.

### CROSS-REFERENCE TO RELATED APPLICATIONS

This application is a continuation of PCT Application PCT/US2006/042211, filed Oct. 27, 2006, which claims the benefit of U.S. Provisional Patent Application Ser. No. 60/731,038, filed Oct. 27, 2005. The contents of these applications are incorporated herein by reference in their entirety.

### BACKGROUND

Interleukin-12 (IL-112) is a heterodimeric cytokine (p70) which plays key roles in immune responses by bridging innate resistance and antigen-specific adaptive immunity. Trinchieri (1993) Immunol Today 14: 335. For example, it promotes type 1 T helper cell (T_{H}1) responses and, hence, cell-mediated immunity. Chan et al. (1991) J Exp Med 173: 869; Seder et al. (1993) Proc Natl Acad Sci USA 90: 10188; Manetti et al. (1993) J Exp Med 177: 1199; and Hsieh et al. (1993) Science 260: 547. IL-12 is composed of two, disulfide linked, independently regulated subunits, p35 and p40. IL-12 is produced by phagocytic cells and antigen presenting cells, in particular, macrophages and dendritic cells, upon stimulation with bacteria, bacterial products such as lipopolysaccharide (LPS), and intracellular parasites. The well-documented biological functions of IL-12 are induction of interferon-γ expression from T and NK cells and differentiation toward the T_{H}1 T lymphocyte type. IFN-γ, expression of which is induced by IL-12, is a strong and selective enhancer of IL-12 production from monocytes and macrophages. The cytokine IL-23 is a heterodimer composed of a p19 subunit and the same p40 subunit of IL-12. IL-23, similarly to IL-12, is involved in type 1 immune defenses and induces IFN-γ secretion from T cells. IL-27 is formed by the association of EBI3, a polypeptide related to the p40 subunit of IL-12, and p28, a protein related to the p35 subunit of IL-12. IL-27 promotes the growth of T cells and is thought to play a role in the differentiation of T_{H}1 cells. Pflanz et al., Immunity (2002), 16:779-790.

It has been suggested that, particularly in chronic diseases in which there is ongoing production of IFN-γ, IL-12 production is augmented by IFN-γ. It is presumed that after an infective or inflammatory stimulus that provokes IL-12 production, the powerful feedback loop promotes IL-12- and IL-23-induced IFN-γ to further augment IL-12 production, leading to consequent excessive production of pro-inflammatory cytokines. Furthermore, it has been suggested that IL-27 induces the expression of T-bet, a major T_{H}1-specific transcription factor, and its downstream target IL-12R β2, independently of IFN-γ. In addition, IL-27 suppresses the expression of GATA-3. GATA-3 inhibits T_{H}1 development and causes loss of IL-12 signaling through suppression of IL-12R β2 and Stat4 expression. Lucas et al., PNAS (2003), 100:15047-15052.

IL-12, as well as IL-23 and IL-27, play a critical role in multiple-T_{H}1 dominant autoimmune diseases including, but not limited to, multiple sclerosis, sepsis, myasthenia gravis, autoimmune neuropathies, Guillain-Barré syndrome, autoimmune uveitis, autoimmune hemolytic anemia, pernicious anemia, autoimmune thrombocytopenia, temporal arteritis, anti-phospholipid syndrome, vasculitides, Wegener's granulomatosis, Behcet's disease, psoriasis, psoriatic arthritis, dermatitis herpetiformis, pemphigus vulgaris, vitiligo, Crohn's disease, ulcerative colitis, interstitial pulmonary fibrosis, myelofibrosis, hepatic fibrosis, myocarditis, thyroditis, primary biliary cirrhosis, autoimmune hepatitis, Type 1 or immune-mediated diabetes mellitus, Grave's disease, Hashimoto's thyroiditis, autoimmune oophoritis and orchitis, autoimmune disease of the adrenal gland; rheumatoid arthritis, juvenile rheumatoid arthritis, systemic lupus erythematosus, scleroderma, polymyositis, dermatomyositis, spondyloarthropathies, ankylosing spondylitis, Sjogren's syndrome, and graft-versus-host disease. See, for example, Gately et al. (1998) Annu Rev Immunol. 16: 495; and Abbas et al. (1996) Nature 383: 787.

Inhibiting IL-12 overproduction, or inhibiting the production of cytokines such as IL-23 and IL-27 which promote IL-12 production and/or T_{H}1 development is an approach to treating the just-mentioned diseases. Trembleau et al. (1995) Immunol. Today 16: 383; and Adorini et al. (1997) Chem. Immunol. 68: 175. For example, overproduction of IL-12 and the resultant excessive T_{H}1 type responses can be suppressed by modulating IL-12, IL-23 and/or IL-27 production. Therefore, compounds that down-regulate IL-12, IL-23 and/or IL-27 production can be used for treating inflammatory diseases. Ma et al. (1998) Eur Cytokine Netw 9: 54.

IL-12 also plays a role in bone loss diseases, particularly those involving osteoclasts. Osteoclasts are unique multinucleated cells within bone that are responsible for bone degradation and resorption. These are the only cells in the body known to be capable of this function. Osteoclasts have a high capacity for the synthesis and storage of enzymes, including acid hydrolases and carbonic anhydrase isoenzyme II. Osteoclasts share phenotypic characteristics with circulating monocytes and tissue macrophages (N. Kurihara et al., Endocrinology 126: 2733-41 (1990); G. Hattersley et al, Endocrinology 128: 259-62 (1991)). These cells are derived from mononuclear precursors that are the progeny of stem-cell populations located in the bone marrow, spleen, and liver. Proliferation of these stem-cell populations produces osteoclastic precursors, which migrate via vascular routes to skeletal sites. These cells then differentiate and fuse with each other to form osteoclasts, or alternatively, fuse with existing osteoclasts.

The regulation of osteoclastic formation and activity is only partly understood but it is known that excessive bone resorption by osteoclasts contributes to the pathology of many human diseases associated with excessive bone loss, including periodontal disease, non-malignant bone disorders (such as osteoporosis, Paget's disease of bone, osteogenesis imperfecta, fibrous dysplasia, and primary hyperparathyroidism) estrogen deficiency, inflammatory bone loss, bone malignancy, arthritis, osteopetrosis, and certain cancer-related disorders (such as hypercalcemia of malignancy (HCM), osteolytic bone lesions of multiple myeloma and osteolytic bone metastases of breast cancer and other metastatic cancers).

U.S. patent application Ser. No. 11/105,818, filed on Apr. 13, 2005, the entire teachings of which are incorporated herein by reference, discloses salt forms of compounds that inhibit IL-12, IL-23 and/or IL-27 production, including mesylate disalt forms which had the advantage of being more soluble in aqueous formulations than the parent compounds. The method of making these salt forms involves dissolving the compounds in a heated solution of absolute ethanol or in a mixture of ethanol and toluene, adding an acid to the heated solution, and allowing the solution to cool to room temperature. The salt form of the compounds precipitates out of the solution during cooling. This method was found to produce some degradation of the IL-12, IL-23 and/or IL-27 inhibitory compounds during the heating process and, in particular, when the acid used to prepare the salt was methanesulfonic acid, this method produced methanesulfonic acid ethyl ester which is a genotoxic impurity. Therefore, it is desirable to develop a new process for preparing mesylate salt forms of these compounds that does not does not generate methanesulfonic acid esters and reduces the degradation of the IL-12, Il-23, and/or IL-27 inhibitory compounds.

### SUMMARY OF THE INVENTION

This invention relates to a method of preparing mesylate salts of nitrogen-heteroaryl inhibitors of IL-12, IL-23 and/or IL-27 production.

In a first aspect, the invention relates to a method of preparing a methanesulfonic acid salt represented by formula (I):

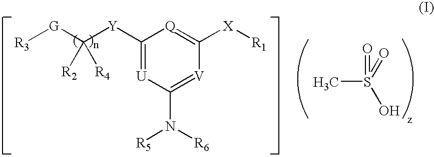

or a pharmaceutically acceptable solvate, clathrate, hydrate, polymorph or prodrug thereof, wherein:

R₁ is optionally substituted aryl, optionally substituted heteroaryl, or a group represented by the following formula:

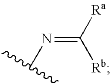

R₂ and R₄, for each occurrence, are independently, H, an optionally substituted alkyl, an optionally substituted alkylcarbonyl, —OR^{k}, —SR^{k}, —NR^{h}R^{j}, hydroxylalkyl, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, haloalkyl, aminoalkyl, mercaptoalkyl, cyano, nitro, nitroso, azide, an optionally substituted alkylcarbonylalkyl, an optionally substituted cyclyl, an optionally substituted cycloalkyl, an optionally substituted heterocyclyl, an optionally substituted heterocycloalkyl, an optionally substituted aryl, an optionally substituted aralkyl, an optionally substituted heteroaryl, an optionally substituted heteroaralkyl, or isothionitro; or R₂ and R₄ taken together are ═O, ═S, or ═NR;

R₃ is R_{g};

R₅ and R₆ are each, independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R₅ and R₆ taken together with the N to which they are attached is an optionally substituted heterocyclyl, an optionally substituted heterocycloalkyl, or an optionally substituted heteroaryl;

X is O, S, S(O), S(O)₂, or NR^{k};

Y is (CH(R^{g}))ₘ, C(O), C(NR), O, S, S(O), S(O)₂, N(R^{k}), or absent;

G is a bond, —C(O)NR^{k}NR^{k}—, —NR^{k}NR^{k}C(O)—, —NR^{k}N═CR^{k}—, —CR═NNR^{k}—, —NR^{k}NR^{k}—, —N(OH)—, —NR^{k}—, —ONR^{k}—, —C(O)—, —C(NR)—, —NR^{k}C(O)—, —C(O)NR^{k}—, —OC(O)—, —C(O)O—, —OC(O)O—, —NR^{k}C(O)O, —OC(O)NR^{k}—, —NR^{k}C(S)O—, —OC(S)NR^{k}—, —NR^{k}—C(NR)—NR^{k}—, —NR^{k}—C(O)—NR^{k}—, —NR^{k}—C(S)—NR^{k}—, —NR^{k}—S(O)₂—NR^{k}—, —P(O)(R^{c})—, —P(O)(R^{c})O—, —OP(O)(R^{c})—, —OP(O)(R^{c})O—, an optionally substituted cycloalkylene, an optionally substituted cyclylene, an optionally substituted heterocycloalkylene, an optionally substituted heterocyclylene, an optionally substituted arylene, an optionally substituted aralkylene, an optionally substituted heteroarylene, an optionally substituted heteroaralkylene, an optionally substituted heteroarylene-NR^{k}—, an optionally substituted heteroarylene-S—, an optionally substituted heteroaralkylene-O—, —Si(OR^{k})₂—, —B(OR^{k}), —C(NR)—NR^{k}—, —NR^{k}—CR^{g}R^{g}—C(O)—, —C(O)—ONR^{k}—, —C(O)—NR^{k}O—, —C(S)—ONR^{k}—, —C(S)—NR^{k}O—, —C(NR)—ONR^{k}—, —C(NR)—NR^{k}O—, —OS(O)₂—NR^{k}NR^{k}—, —OC(O)—NR^{k}NR^{k}—, —OC(S)—NR^{k}NR^{k}, —OC(NR)—NR^{k}NR^{k}—, —NR^{k}NR^{k}S(O)₂O—, —NR^{k}NR^{k}C(S)O—, —NR^{k}NR^{k}C(NR)O—, —OP(O)(R^{c})O—, —NR^{k}P(O)(R^{c})O—, —OP(O)(R^{c}C)NR^{k}, —NR^{k}P(O)(R^{c})NR^{k}—, —P(O)(R^{c})NR^{k}—, —NR^{k}P(O)(R^{c})—, —O-alkylene-heterocycloalkylene-NR^{k}—, —NR^{k}—CHR^{g}—C(O)—NR^{k}—CHR^{g}—C(O)—, —NR^{k}—CHR^{g}—C(O)—, —NR^{k}—C(O)—CHR^{g}—, or —C(O)—NR^{k}—CHR^{g}—C(O)—; and

each of Q, U, and V are independently N or CR^{g}, wherein at least one of Q, U, or V is N; and each CR^{g} may be the same or different;

R, for each occurrence, is independently H, an optionally substituted alkyl, an optionally substituted cycloalkyl, an optionally substituted cyclyl, an optionally substituted heterocycloalkyl, an optionally substituted heterocyclyl, an optionally substituted heteroaryl, an optionally substituted aralkyl, an optionally substituted heteroaralkyl, —C(O)R^{c}, —OR^{k}, —SR^{k}, —NR^{h}R^{j}, hydroxylalkyl, nitro, cyano, haloalkyl, aminoalkyl, or —S(O)₂R^{c};

each of R^{a} and R^{b}, independently, is H, optionally substituted alkyl, an optionally substituted cycloalkyl, an optionally substituted cyclyl, an optionally substituted heterocycloalkyl, an optionally substituted heterocyclyl, optionally substituted aryl, or optionally substituted heteroaryl;

R^{c}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, or thioalkoxy;

R^{g}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, thioalkoxy, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, aminoalkyl, mercaptoalkyl, cyano, nitro, nitroso, or azide;

R^{h} and R^{j}, for each occurrence, are independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R^{h} and R^{j} taken together with the N to which they are attached is an optionally substituted heterocyclyl, an optionally substituted heterocycloalkyl, or an optionally substituted heteroaryl;

R^{k}, for each occurrence, is independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, or an optionally substituted heteroaryl;

n is 0, 1, 2, 3, 4, 5, 6 or 7;

m is 0, 1, 2, 3, or 4; and

z is 1 or 2;

said method comprising the steps of:
a) providing a solution of a compound represented by formula (II):

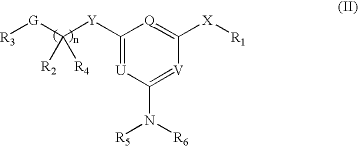

in a water miscible organic solvent, provided that the water miscible solvent is not an alcohol; and
b) adding to the solution provided in step a) a solution of methanesulfonic acid in water;
c) allowing the salt represented by formula (I) to precipitate out of solution; and
d) collecting the precipitate formed in step c), thereby preparing a methanesulfonic acid salt represented by formula (I).

In a second aspect, the invention relates to a method of preparing a methanesulfonic acid salt represented by formula (III):

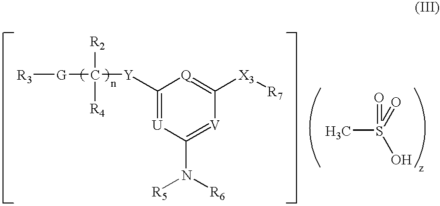

or a pharmaceutically acceptable solvate, clathrate, hydrate, prodrug or polymorph thereof, wherein:

X₃ is —C(R^{g})═N-A-;

A is O, S, S(O), S(O)₂, C(CR^{g})₂, or NR^{k};

R₇ is an optionally substituted aryl or an optionally substituted heteroaryl; and

R₂, R₃, R⁴, R₅, R₆, Y, G, Q, U, V, R^{g}, R^{k}, n and z are defined as above; said method comprising the steps of:
a) providing a solution of a compound represented by formula (IV):

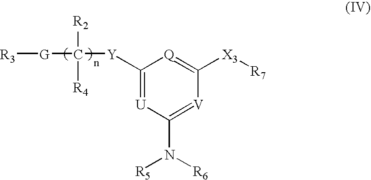

in a water miscible organic solvent, provided that the water miscible solvent is not an alcohol; and
b) adding to the solution provided in step a) a solution of methanesulfonic acid in water;
c) allowing the salt represented by formula (III) to precipitate out of solution; and
d) collecting the precipitate formed in step c), thereby preparing a methanesulfonic acid salt represented by formula (III).

In a third aspect, the invention relates to a method of preparing a methanesulfonic acid salt represented by formula (V):

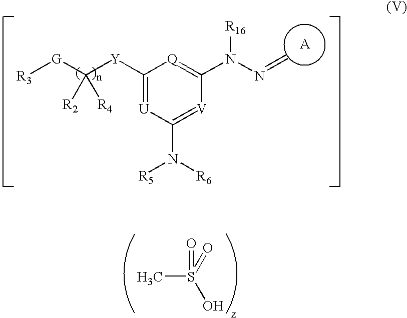

or a pharmaceutically acceptable solvate, clathrate, hydrate, polymorph, or prodrug thereof, wherein:

ring A is an optionally substituted cycloalkyl, an optionally substituted cyclyl, an optionally substituted heterocycloalkyl, or an optionally substituted heterocyclyl, wherein the cycloalkyl, cyclyl, heterocycloalkyl, and heterocyclyl are optionally fused to an optionally substituted cycloalkyl, an optionally substituted cyclyl, an optionally substituted heterocycloalkyl, an optionally substituted heterocyclyl, an optionally substituted aryl, or an optionally substituted heteroaryl;

R₁₆, for each occurrence, is independently, H or a lower alkyl;

R₂, R₃, R₄, R₅, R₆, Y, G, Q, U, V, n and z are defined as above;

said method comprising the steps of:
a) providing a solution of a compound represented by formula (VI):

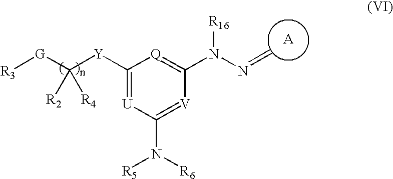

in a water miscible organic solvent, provided that the water miscible solvent is not an alcohol; and
b) adding to the solution provided in step a) a solution of methanesulfonic acid in water;
c) allowing the salt represented by formula (V) to precipitate out of solution; and
d) collecting the precipitate formed in step c), thereby preparing a methanesulfonic acid salt represented by formula (V).

In a fourth aspect, the invention relates to a method of preparing a methanesulfonic acid salt represented by formula (X):

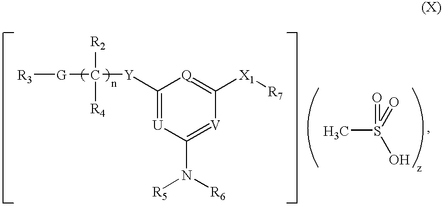

or a pharmaceutically acceptable solvate, clathrate, hydrate or polymorph thereof, wherein:

X₁ is represented by a formula selected from the group consisting of:

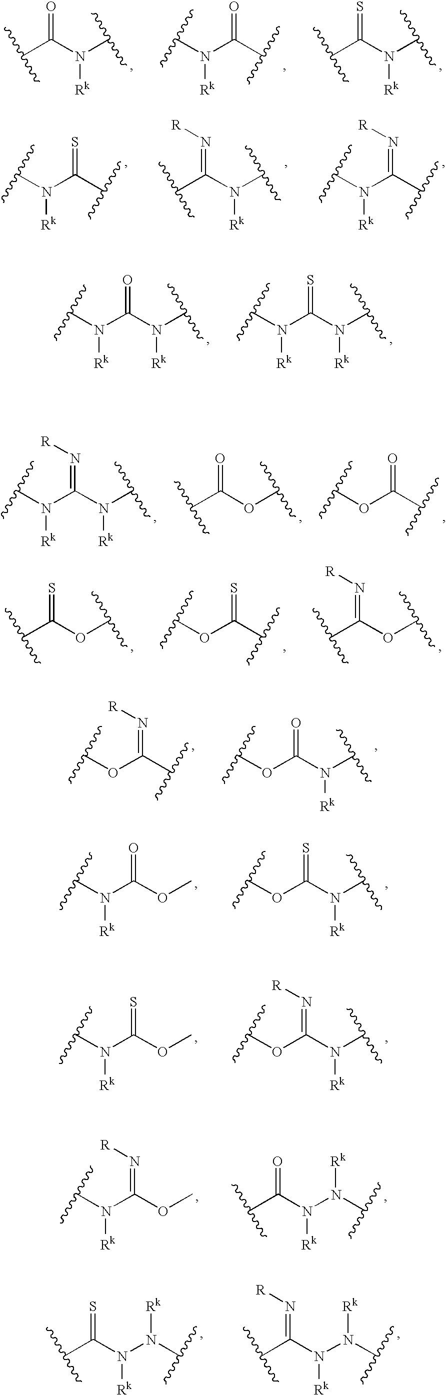

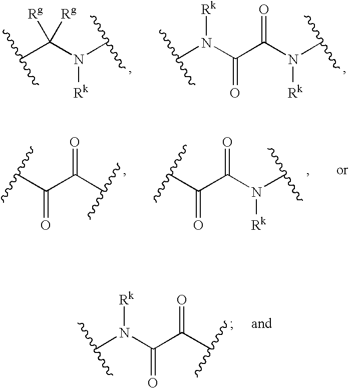

R₂, R₃, R₄, R₅, R₆, R₇, Y, G, Q, U, V, R, R^{g}, R^{k}, n and z are defined as above;

said method comprising the steps of:
a) providing a solution of a compound represented by formula (XI):

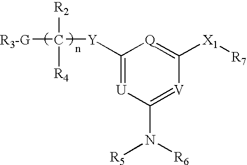

in a water miscible organic solvent, provided that the water miscible solvent is not an alcohol; and
b) adding to the solution provided in step a) a solution of methanesulfonic acid in water;
c) allowing the salt represented by formula (X) to precipitate out of solution; and
d) collecting the precipitate formed in step c), thereby preparing a methanesulfonic acid salt represented by formula (X).

In a fifth aspect, the invention relates to a method of preparing a methanesulfonic acid salt represented by formula (I), or a pharmaceutically acceptable solvate, clathrate, hydrate, polymorph or prodrug thereof. This method comprising the steps of:
a) providing a solution of a compound represented by formula (II) in an organic solvent, provided that the organic solvent is not an alcohol;
b) adding to the solution provided in step a) methanesulfonic acid;
c) allowing the methanesulfonic acid salt represented by formula (I) to precipitate out of solution; and
d) collecting the precipitate formed in step c), thereby preparing a methanesulfonic acid salt represented by formula (I).

In a sixth aspect, the invention relates to a method of preparing a methanesulfonic acid salt represented by formula (III), or a pharmaceutically acceptable solvate, clathrate, hydrate, prodrug or polymorph thereof. This method comprising the steps of:
a) providing a solution of a compound represented by formula (IV) in an organic solvent, provided that the organic solvent is not an alcohol;
b) adding to the solution provided in step a) methanesulfonic acid;
c) allowing the methanesulfonic acid salt represented by formula (III) to precipitate out of solution; and
d) collecting the precipitate formed in step c), thereby preparing a methanesulfonic acid salt represented by formula (III).

In a seventh aspect, the invention relates to a method of preparing a methanesulfonic acid salt represented by formula (V), or a pharmaceutically acceptable solvate, clathrate, hydrate, polymorph, or prodrug thereof. This method comprising the steps of:
a) providing a solution of a compound represented by formula (VI), in an organic solvent, provided that the organic solvent is not an alcohol; and
b) adding to the solution provided in step a) methanesulfonic acid;
c) allowing the methanesulfonic acid salt represented by formula (V) to precipitate out of solution; and
d) collecting the precipitate formed in step c), thereby preparing a methanesulfonic acid salt represented by formula (V).

In an eighth aspect, the invention relates to a method of preparing a methanesulfonic acid salt represented by formula (X), or a pharmaceutically acceptable solvate, clathrate, hydrate or polymorph thereof. This method comprising the steps of:
a) providing a solution of a compound represented by formula (XI) in an organic solvent, provided that the organic solvent is not an alcohol;
b) adding to the solution provided in step a) methanesulfonic acid;
c) allowing the methanesulfonic acid salt represented by formula (X) to precipitate out of solution; and
d) collecting the precipitate formed in step c), thereby preparing a methanesulfonic acid salt represented by formula (X).

The method of the invention produces less degradation products than prior art methods. In addition, since alcohols are not used as a solvent, no methanesulfonic acid alkyl esters are formed. The increased purity and lack of methanesulfonic acid alkyl ester impurities in methanesulfonic acid salts of IL-12 inhibitory compounds prepared by the method of the invention, reduces the cost and time needed for their manufacture.

### DETAILED DESCRIPTION OF THE INVENTION

### Definition of Terms

As used herein, the term “alkyl” refers to a straight-chained or branched hydrocarbon group containing 1 to 12 carbon atoms. The term “lower alkyl” refers to a C₁-C₆ alkyl chain. Examples of alkyl groups include methyl, ethyl, n-propyl, isopropyl, tert-butyl, and n-pentyl. Alkyl groups may be optionally substituted with one or more substituents.

The term “alkenyl” refers to an unsaturated hydrocarbon chain that may be a straight chain or branched chain, containing 2 to 12 carbon atoms and at least one carbon-carbon double bond. Alkenyl groups may be optionally substituted with one or more substituents.

The term “alkynyl” refers to an unsaturated hydrocarbon chain that may be a straight chain or branched chain, containing the 2 to 12 carbon atoms and at least one carbon-carbon triple bond. Alkynyl groups may be optionally substituted with one or more substituents.

The sp² or sp carbons of an alkenyl group and an alkynyl group, respectively, may optionally be the point of attachment of the alkenyl or alkynyl groups.

The term “alkoxy,” as used herein, refers to an alkyl or a cycloalkyl group which is linked to another moiety though an oxygen atom. Alkoxy groups can be optionally substituted with one or more substituents.

The term “mercapto” refers to a —SH group.

The term “alkyl sulfanyl,” as used herein, refers to an alkyl or a cycloalkyl group which is linked to another moiety though a divalent sulfer atom. Alkyl sulfanyl groups can be optionally substituted with one or more substituents.

As used herein, the term “halogen” or “halo” means —F, —Cl, —Br or —I.

As used herein, the term “haloalkyl” means and alkyl group in which one or more (including all) the hydrogen radicals are replaced by a halo group, wherein each halo group is independently selected from —F, —Cl, —Br, and —I. The term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like.

The term “cycloalkyl” refers to a hydrocarbon 3-8 membered monocyclic or 7-14 membered bicyclic ring system which is completely saturated ring. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclooctyl, cyclononyl, cyclodecyl, and bicyclo[2.1.1]hexyl.

The term “cyclyl” refers to a hydrocarbon 3-8 membered monocyclic or 7-14 membered bicyclic ring system having at least one non-aromatic ring, wherein the non-aromatic ring has some degree of unsaturation. Cyclyl groups may be optionally substituted with one or more substituents. In one embodiment, 0, 1, 2, 3, or 4 atoms of each ring of a cyclyl group may be substituted by a substituent. Examples of cyclyl groups include cyclohexenyl, bicyclo[2.2.1]hept-2-enyl, dihydronaphthalenyl, benzocyclopentyl, cyclopentenyl, cyclopentadienyl, cyclohexenyl, cyclohexadienyl, cycloheptenyl, cycloheptadienyl, cycloheptatrienyl, cyclooctenyl, cyclooctadienyl, cyclooctatrienyl, cyclooctatetraenyl, cyclononenyl, cyclononadienyl, cyclodecenyl, cyclodecadienyl and the like.

The term “aryl” refers to a hydrocarbon monocyclic, bicyclic or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

As used herein, the term “aralkyl” means an aryl group that is attached to another group by a (C₁-C₆)alkylene group. Aralkyl groups may be optionally substituted, either on the aryl portion of the aralkyl group or on the alkylene portion of the aralkyl group, with one or more substituent. Representative aralkyl groups include benzyl, 2-phenyl-ethyl, naphth-3-yl-methyl and the like.

As used herein, the term “alkylene” refers to an alkyl group that has two points of attachment. The term “(C₁-C₆)alkylene” refers to an alkylene group that has from one to six carbon atoms. Non-limiting examples of alkylene groups include methylene (—CH₂—), ethylene (—CH₂CH₂—), n-propylene (—CH₂CH₂CH₂—), isopropylene (—CH₂CH(CH₃)—), and the like. Alkylene groups may be optionally substituted.

As used herein, the term “cycloalkylene” refers to a cycloalkyl group that has two points of attachment. Cycloalkylene groups may be optionally substituted.

As used herein, the term “cyclylene” refers to a cyclyl group that has two points of attachment. Cyclylene groups may be optionally substituted.

As used herein, the term “arylene” refers to an aryl group that has two points of attachment. Arylene groups may be optionally substituted.

As used herein, the term “aralkylene” refers to an aralkyl group that has two points of attachment. Each point of attachment may be, independently, on the aryl portion of the aralkyl group or on the alkyl portion. Aralkylene groups may be optionally substituted.

The term “arylalkoxy” refers to an alkoxy substituted with an aryl.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon. Heteroaryl groups may be optionally substituted with one or more substituents. In one embodiment, 0, 1, 2, 3, or 4 atoms of each ring of a heteroaryl group may be substituted by a substituent. Examples of heteroaryl groups include pyridyl, 1-oxo-pyridyl, furanyl, benzo[1,3]dioxolyl, benzo[1,4]dioxinyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, and benzo(b)thienyl, 3H-thiazolo[2,3-c][1,2,4]thiadiazolyl, imidazo[1,2-d]-1,2,4-thiadiazolyl, imidazo[2,1-b]-1,3,4-thiadiazolyl, 1H,2H-furo[3,4-d]-1,2,3-thiadiazolyl, 1H-pyrazolo[5,1-c]-1,2,4-triazolyl, pyrrolo[3,4-d]-1,2,3-triazolyl, cyclopentatriazolyl, 3H-pyrrolo[3,4-c]isoxazolyl, 1H,3H-pyrrolo[1,2-c]oxazolyl, pyrrolo[2,1b]oxazolyl, and the like.

As used herein, the term “heteroaralkyl” or “heteroarylalkyl” means a heteroaryl group that is attached to another group by a (C₁-C₆)alkylene. Heteroaralkyl groups may be optionally substituted, either on the heteroaryl portion of the heteroaralkyl group or on the alkylene portion of the heteroaralkyl group, with one or more substituent. Representative heteroaralkyl groups include 2-(pyridin-4-yl)-propyl, 2-(thien-3-yl)-ethyl, imidazol-4-yl-methyl and the like.

As used herein, the term “heteroarylene” refers to a heteroaryl group that has two points of attachment. Heteroarylene groups may be optionally substituted.

As used herein, the term “heteroaralkylene” refers to a heteroaralkyl group that has two points of attachment. Each point of attachment may be, independently, on the heteroaryl portion of the heteroaralkylene or on the alkyl portion. Heteroaralkylene groups may be optionally substituted.

The term “heterocycloalkyl” refers to a nonaromatic, completely saturated 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si. Preferably, heteroatoms are selected from O, N, and S. Heterocycloalkyl groups may be optionally substituted with one or more substituents. In one embodiment, 0, 1, 2, 3, or 4 atoms of each ring of a heterocycloalkyl group may be substituted by a substituent. Representative heterocycloalkyl groups include piperidinyl, piperazinyl, 2-oxopiperazinyl, 2-oxopiperidinyl, 2-oxopyrrolidinyl, 4-piperidonyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolane, tetrahydrofuranyl, tetrahydrothienyl, an thiirene.

The term “heterocyclyl” refers to a nonaromatic 5-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system has some degree of unsaturation. Heterocyclyl groups may be optionally substituted with one or more substituents. In one embodiment, 0, 1, 2, 3, or 4 atoms of each ring of a heterocyclyl group may be substituted by a substituent. Examples of these groups include thiirenyl, thiadiazirinyl, dioxazolyl, 1,3-oxathiolyl, 1,3-dioxolyl, 1,3-dithiolyl, oxathiazinyl, dioxazinyl, dithiazinyl, oxadiazinyl, thiadiazinyl, oxazinyl, thiazinyl, 1,4-oxathiin, 1,4-dioxin, 1,4-dithiin, 1H-pyranyl, oxathiepinyl, 5H-1,4-dioxepinyl, 5H-1,4-dithiepinyl, 6H-isoxazolo[2,3-d]1,2,4-oxadiazolyl, 7H-oxazolo[3,2-d]1,2,4-oxadiazolyl, and the like.

As used herein, the term “heterocycloalkylene” refers to a heterocycloalkyl group that has two points of attachment. Heterocycloalkylene groups may be optionally substituted.

As used herein, the term “heterocyclylene” refers to a heterocyclyl group that has two points of attachment. Heterocyclylene groups may be optionally substituted.

When a cycloalkyl, cyclyl, heterocycloalkyl, or heterocyclyl is fused to another ring (e.g., a cycloalkyl, cyclyl, heterocycloalkyl, heterocyclyl, aryl, heteroaryl), it shares two or more ring atoms, preferably two to four ring atoms, with the other ring.

The term “amino” refers to —NH₂. The term “alkylamino” refers to an amino in which one hydrogen is replaced by an alkyl group. The term “dialkylamino” refers to an amino in which each of the two hydrogens are replaced by an independently selected alkyl group. The term “aminoalkyl” refers to an alkyl substituent which is further substituted with one or more amino groups.

The term “mercaptoalkyl” refers to an alkyl substituent which is further substituted with one or more mercapto groups.

The term “hydroxyalkyl” or “hydroxylalkyl” refers to an alkyl substituent which is further substituted with one or more hydroxy groups.

The term “sulfonylalkyl” refers to an alkyl substituent which is further substituted with one or more sulfonyl groups.

The term “sulfonylaryl” refers to an aryl substituent which is further substituted with one or more sulfonyl groups.

The term alkylcarbonyl refers to an —C(O)-alkyl.

The term “mercaptoalkoxy” refers to an alkoxy substituent which is further substituted with one or more mercapto groups.

The term “alkylcarbonylalkyl” refers to an alkyl substituent which is further substituted with —C(O)-alkyl.

The alkyl or aryl portion of alkylamino, aminoalkyl, mercaptoalkyl, hydroxyalkyl, mercaptoalkoxy, sulfonylalkyl, sulfonylaryl, alkylcarbonyl, and alkylcarbonylalkyl may be optionally substituted with one or more substituents.

Suitable substituents for an alkyl, alkoxy, alkyl sulfanyl, alkylamino, dialkylamino, alkylene, alkenyl, alkynyl, cycloalkyl, cyclyl, heterocycloalkyl, heterocyclyl, aryl, aralkyl, heteroaryl, heteroaralkyl, cycloalkylene, cyclylene, heterocycloalkylene, heterocyclylene, arylene, aralkylene, heteroalkylene and heteroaryalkylene groups include any substituent which will form a stable compound of the invention. Examples of substituents for an alkyl, alkoxy, alkylsulfanyl, alkylamino, dialkylamino, alkylene, alkenyl, alkynyl, cycloalkyl, cyclyl, heterocycloalkyl, heterocyclyl, aryl, aralkyl, heteroaryl, heteroaralkyl, cycloalkylene, cyclylene, heterocycloalkylene, heterocyclylene, arylene, aralkylene, heteroalkylene and heteroaryalkylene include an optionally substituted alkyl, an optionally substituted alkoxy, an optionally substituted alkyl sulfanyl, an optionally substituted alkenyl, an optionally substituted alkynyl, an optionally substituted cycloalkyl, an optionally substituted cyclyl, an optionally substituted heterocyclyl, an optionally substituted heterocycloalkyl, an optionally substituted aryl, an optionally substituted heteroaryl, an optionally substituted aralkyl, an optionally substituted heteraralkyl, a haloalkyl, halo, cyano, nitro, haloalkoxy, ═O, ═S, ═NR^{k}, OR^{k}, —NR^{h}R^{j}, —SR^{k}, —C(O)R^{k}, —C(O)NR^{h}R^{j}, —NR^{k}C(O)R^{k}, —C(O)OR^{k}, —OC(O)R^{k}, —NR^{k}C(O)NR^{h}R^{j}, —OC(O)NR^{h}R^{j}, —NR^{k}C(O)OR^{k}, —C(NR)R^{k}, —C(NR)NR^{h}R^{j}, —NR^{k}C(NR)R^{k}, —C(NR)OR^{k}, —OC(NR)R^{k}, —NR^{k}C(NR)NR^{h}R^{j}, —OC(NR)NR^{h}R^{j}, —NR^{k}C(NR)OR^{k}, —C(S)R^{k}, —C(S)NR^{h}R^{j}, —NR^{k}C(S)R^{k}, —C(S)OR^{k}, —OC(S)R^{k}, —NR^{k}C(S)NR^{h}R^{j}, —OC(S)NR^{h}R^{j}, —NR^{k}C(S)OR^{k}, —C(O)SR^{k}, —SC(O)R^{k}, —S(O)ₕR^{k}, —S(O)ₕNR^{h}R^{j}, —OS(O)ₕR^{k}, —S(O)ₕOR^{k}, —OS(O)ₕOR^{k}, —P(O)(OR^{k})₂, —OP(O)(OR^{k})₂, —P(S)(OR^{k})₂, —SP(O)(OR^{k})₂, —P(O)(SR^{k})(OR^{k}), —OP(O)(SR^{k})(OR^{k}), —P(O)(SR^{k})₂, or —OP(O)(SR^{k})₂, wherein h is 1 or 2.

In addition, alkyl, cycloalkyl, alkylene, a heterocycloalkyl, and any saturated portion of an alkenyl, cyclyl, alkynyl, heterocyclyl, aralkyl, and heteroaralkyl groups, may also be substituted with ═O, ═S, or ═NR.

When a heterocyclyl, heteroaryl, or heteroaralkyl group contains a nitrogen atom, it may be substituted or unsubstituted. When a nitrogen atom in the aromatic ring of a heteroaryl group has a substituent the nitrogen may be a quaternary nitrogen.

Choices and combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject). Typically, such compounds are stable at a temperature of 30° C. or less, in the absence of excessive moisture, for at least one week. Such choices and combinations will be apparent to those of ordinary skill in the art and may be determined without undue experimentation.

As used herein, the term “lower” refers to a group having up to six atoms. For example, a “lower alkyl” refers to an alkyl radical having from 1 to 6 carbon atoms, and a “lower alkenyl” or “lower alkynyl” refers to an alkenyl or alkynyl radical having from 2 to 6 carbon atoms. A “lower alkoxy” or “lower alkyl sulfanyl” group refers to an alkoxy or alkyl sulfanyl group that has from 1 to 6 carbon atoms.

As used herein, the term “water miscible organic solvent,” includes any carbon containing solvent that is miscible with water. Examples, of water miscible organic solvents include, but are not limited to, acetonitrile, acetone, alcohols, dimethyl formamide, tetrahydrofuran, dioxane, and dimethyl sulfoxide. However, in some embodiments of the method of the invention, alcohols are not used because they can react to form methylsulfonic acid alkyl esters which are undesirable impurities. Preferred water mixable organic solvents include acetonitrile and acetone; more preferred is acetone.

The compounds of the invention are defined herein by their chemical structures and/or chemical names. Where a compound is referred to by both a chemical structure and a chemical name, and the chemical structure and chemical name conflict, the chemical structure is determinative of the compound's identity.

The compounds of this invention include the mesylate salts represented by formulas (I), (III), (V) and (X); the mesylates salts of compounds (II), (IV), (VI), (VII), (VIII), (IX), (XI); and the mesylate salts of the compounds shown in Table 1, as well as solvate, clathrate, hydrate, polymorph, or prodrugs, if applicable.

As used herein, the term “polymorph” means solid crystalline forms of a compound of the present invention or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “pharmaceutically acceptable solvate,” is a solvate formed from the association of one or more solvent molecules to a compounds of any the invention. The term solvate includes hydrates (e.g., hemi-hydrate, mono-hydrate, dihydrate, trihydrate, tetrahydrate, and the like).

As used herein, the term “hydrate” means a compound of the present invention or a salt thereof, which further includes a stoichiometric or non-stoichiometric amount of water bound by non-covalent intermolecular forces.

As used herein, the term “clathrate” means a compound of the present invention or a salt thereof in the form of a crystal lattice that contains spaces (e.g., channels) that have a guest molecule (e.g., a solvent or water) trapped within.

As used herein and unless otherwise indicated, the term “prodrug” means a derivative of a compound that can hydrolyze, oxidize, or otherwise react under biological conditions (in vitro or in vivo) to provide a compound of this invention. Prodrugs may only become active upon such reaction under biological conditions, or they may have activity in their unreacted forms. Examples of prodrugs contemplated in this invention include, but are not limited to, analogs or derivatives of compounds of any one of the formulae disclosed herein that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of any one of the formulae disclosed herein that comprise —NO, —NO₂, —ONO, or —ONO₂ moieties. Prodrugs can typically be prepared using well-known methods, such as those described by 1 BURGER'S MEDICINAL CHEMISTRY AND DRUG DISCOVERY (1995) 172-178, 949-982 (Manfred E. Wolff ed., 5^{th} ed).

As used herein and unless otherwise indicated, the terms “biohydrolyzable amide”, “biohydrolyzable ester”, “biohydrolyzable carbamate”, “biohydrolyzable carbonate”, “biohydrolyzable ureide” and “biohydrolyzable phosphate analogue” mean an amide, ester, carbamate, carbonate, ureide, or phosphate analogue, respectively, that either: 1) does not destroy the biological activity of the compound and confers upon that compound advantageous properties in vivo, such as uptake, duration of action, or onset of action; or 2) is itself biologically inactive but is converted in vivo to a biologically active compound. Examples of biohydrolyzable amides include, but are not limited to, lower alkyl amides, α-amino acid amides, alkoxyacyl amides, and alkylaminoalkylcarbonyl amides. Examples of biohydrolyzable esters include, but are not limited to, lower alkyl esters, alkoxyacyloxy esters, alkyl acylamino alkyl esters, and choline esters. Examples of biohydrolyzable carbamates include, but are not limited to, lower alkylamines, substituted ethylenediamines, aminoacids, hydroxyalkylamines, heterocyclic and heteroaromatic amines, and polyether amines.

In addition, some of the compounds of this invention have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms. All such isomeric forms of these compounds are expressly included in the present invention. The compounds of this invention may also be represented in multiple tautomeric forms, in such instances, the invention expressly includes all tautomeric forms of the compounds described herein (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

Further, the aforementioned compounds also include their N-oxides. The term “N-oxides” refers to one or more nitrogen atoms, when present in a heterocyclic or heteroaryl compound, are in N-oxide form, i.e., N→O. For example, in compounds of any one of formula (I) through (XI) or Table 1 when one of Q, U, or V is N, also included are compounds in which Q, U, or V, respectively, is N→O.

Methods of Preparing Mesylate Salts of Compounds that Inhibit IL-12, IL-23 and/or IL-27

In a first aspect, the invention relates to a method of preparing a methanesulfonic acid salt represented by formula (I):

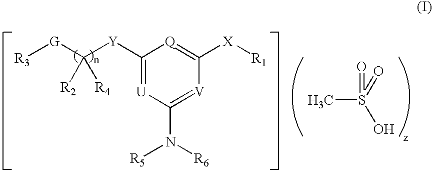

or a pharmaceutically acceptable solvate, clathrate, hydrate, polymorph or prodrug thereof, wherein:

R₁ is optionally substituted aryl, optionally substituted heteroaryl, or a group represented by the following formula:

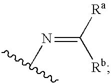

R₂ and R₄, for each occurrence, are independently, H, an optionally substituted alkyl, an optionally substituted alkylcarbonyl, —OR^{k}, —SR^{k}, —NR^{h}R^{j}, hydroxylalkyl, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, haloalkyl, aminoalkyl, mercaptoalkyl, cyano, nitro, nitroso, azide, an optionally substituted alkylcarbonylalkyl, an optionally substituted cyclyl, an optionally substituted cycloalkyl, an optionally substituted heterocyclyl, an optionally substituted heterocycloalkyl, an optionally substituted aryl, an optionally substituted aralkyl, an optionally substituted heteroaryl, an optionally substituted heteroaralkyl, or isothionitro; or R₂ and R₄ taken together are ═O, ═S, or ═NR;

R₃ is R^{g};

R₅ and R₆ are each, independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R₅ and R₆ taken together with the N to which they are attached is an optionally substituted heterocyclyl, an optionally substituted heterocycloalkyl, or an optionally substituted heteroaryl;

X is O, S, S(O), S(O)₂, or NR^{k};

Y is (CH(R^{g}))ₘ, C(O), C(NR), O, S, S(O), S(O)₂, N(R^{k}), or absent;

G is a bond, —C(O)NR^{k}NR^{k}—, —NR^{k}NR^{k}C(O)—, —NR^{k}N═CR^{k}—, —CR═NNR^{k}—, —NR^{k}NR^{k}—, —N(OH)—, —NR^{k}—, —ONR^{k}—, —C(O)—, —C(NR)—, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k}—C(NR)—NR^{k}—, —NR^{k}—C(O)—NR^{k}—, —NR^{k}—C(S)—NR^{k}—, —NR^{k}—S(O)₂—NR^{k}—, —P(O)(R^{c})—, —P(O)(R^{c})O—, —OP(O)(R^{c})—, —OP(O)(R^{c})O—, an optionally substituted cycloalkylene, an optionally substituted cyclylene, an optionally substituted heterocycloalkylene, an optionally substituted heterocyclylene, an optionally substituted arylene, an optionally substituted aralkylene, an optionally substituted heteroarylene, an optionally substituted heteroaralkylene, an optionally substituted heteroarylene-NR^{k}—, an optionally substituted heteroarylene-S—, an optionally substituted heteroaralkylene-O—, —Si(OR^{k})₂—, —B(OR^{k})—, —C(NR)—NR^{k}—, —NR^{k}—CR^{g}R^{g}—C(O)—, —C(O)—ONR^{k}, —C(O)—NR^{k}O—, —C(S)—ONR^{k}—, —C(S)—NR^{k}O—, —C(NR)—ONR^{k}—, —C(NR)—NR^{k}O—, —OS(O)₂—NR^{k}NR^{k}—, —OC(O)—NR^{k}NR^{k}—, —OC(S)—NR^{k}NR^{k}—, —OC(NR)—NR^{k}NR^{k}—, —NR^{k}NR^{k}S(O)₂O—, —NR^{k}NR^{k}C(S)O—, —NR^{k}NR^{k}C(NR)O—, —OP(O)(R^{c})O—, —NR^{k}P(O)(R^{c})O—, —OP(O)(R^{c})NR^{k}—, —NR^{k}P(O)(R^{c})NR^{k}—, —P(O)(R^{c})NR^{k}—, —NR^{k}P(O)(R^{c})—, —O-alkylene-heterocycloalkylene-NR^{k}—, —NR^{k}—CHR^{g}—C(O)—NR^{k}—CHR^{g}—C(O)—, —NR^{k}—CHR^{g}—C(O)—, —NR^{k}—C(O)—CHR^{g}—, or —C(O)—NR^{k}—CHR^{g}—C(O)—; and

each of Q, U, and V are independently N or CR^{g}, wherein at least one of Q, U, or V is N; and each CR^{g} may be the same or different;

R, for each occurrence, is independently H, an optionally substituted alkyl, an optionally substituted cycloalkyl, an optionally substituted cyclyl, an optionally substituted heterocycloalkyl, an optionally substituted heterocyclyl, an optionally substituted heteroaryl, an optionally substituted aralkyl, an optionally substituted heteroaralkyl, —C(O)R^{c}, —OR^{k}, —SR^{k}, —NR^{h}R^{j}, hydroxylalkyl, nitro, cyano, haloalkyl, aminoalkyl, or —S(O)₂R^{c};

each of R^{a} and R^{b}, independently, is H, optionally substituted alkyl, an optionally substituted cycloalkyl, an optionally substituted cyclyl, an optionally substituted heterocycloalkyl, an optionally substituted heterocyclyl, optionally substituted aryl, or optionally substituted heteroaryl;

R^{c}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, or thioalkoxy;

R^{g}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, thioalkoxy, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, aminoalkyl, mercaptoalkyl, cyano, nitro, nitroso, or azide;

R^{h} and R^{j}, for each occurrence, are independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R^{h} and R^{j} taken together with the N to which they are attached is an optionally substituted heterocyclyl, an optionally substituted heterocycloalkyl, or an optionally substituted heteroaryl;

R^{k}, for each occurrence, is independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, or an optionally substituted heteroaryl;

n is 0, 1, 2, 3, 4, 5, 6 or 7;

m is 0, 1, 2, 3, or 4; and

z is 1 or 2;

said method comprising the steps of:
a) providing a solution of a compound represented by formula (II):

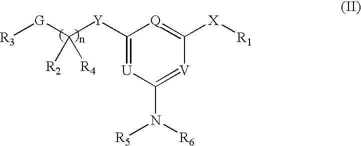

in a water miscible organic solvent, provided that the water miscible solvent is not an alcohol; and
b) adding to the solution provided in step a) a solution of methanesulfonic acid in water;
c) allowing the salt represented by formula (I) to precipitate out of solution; and
d) collecting the precipitate formed in step c), thereby preparing a methanesulfonic acid salt represented by formula (I).

In a second aspect, the invention relates to a method of preparing a methanesulfonic acid salt represented by formula (III):

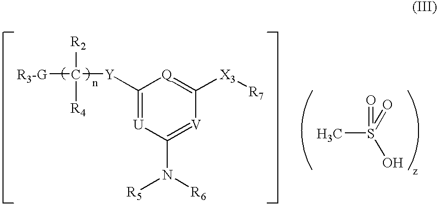

or a pharmaceutically acceptable solvate, clathrate, hydrate, prodrug or polymorph thereof, wherein:

X₃ is —C(R^{g})═N-A-;

A is O, S, S(O), S(O)₂, C(CR^{g})₂, or NR^{k};

R₇ is an optionally substituted aryl or an optionally substituted heteroaryl; and

R₂, R₃, R⁴, R₅, R₆, Y, G, Q, U, V, R^{g}, R^{k}, n and z are defined as above; said method comprising the steps of:
a) providing a solution of a compound represented by formula (IV):

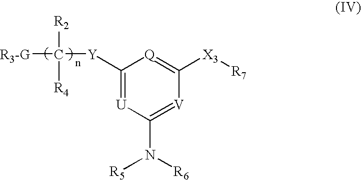

in a water miscible organic solvent, provided that the water miscible solvent is not an alcohol; and
b) adding to the solution provided in step a) a solution of methanesulfonic acid in water;
c) allowing the salt represented by formula (III) to precipitate out of solution; and
d) collecting the precipitate formed in step c), thereby preparing a methanesulfonic acid salt represented by formula (III).

In a third aspect, the invention relates to a method of preparing a methanesulfonic acid salt represented by formula (V):

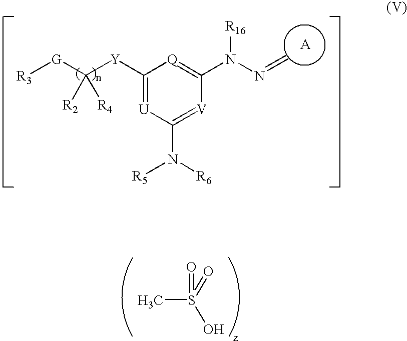

or a pharmaceutically acceptable solvate, clathrate, hydrate, polymorph, or prodrug thereof, wherein:

ring A is an optionally substituted cycloalkyl, an optionally substituted cyclyl, an optionally substituted heterocycloalkyl, or an optionally substituted heterocyclyl, wherein the cycloalkyl, cyclyl, heterocycloalkyl, and heterocyclycl are optionally fused to an optionally substituted cycloalkyl, an optionally substituted cyclyl, an optionally substituted heterocycloalkyl, an optionally substituted heterocyclyl, an optionally substituted aryl, or an optionally substituted heteroaryl;

R₁₆, for each occurrence, is independently, H or a lower alkyl;

R₂, R₃, R⁴, R₅, R₆, Y, G, Q, U, V, n and z are defined as above;

said method comprising the steps of:
a) providing a solution of a compound represented by formula (VI):

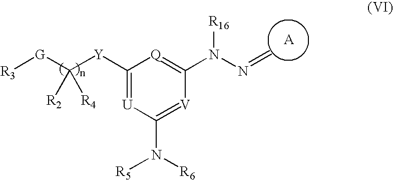

in a water miscible organic solvent, provided that the water miscible solvent is not an alcohol; and
b) adding to the solution provided in step a) a solution of methanesulfonic acid in water;
c) allowing the salt represented by formula (V) to precipitate out of solution; and
d) collecting the precipitate formed in step c), thereby preparing a methanesulfonic acid salt represented by formula (V).

In a fourth aspect, the invention relates to a method of preparing a methanesulfonic acid salt represented by formula (X):

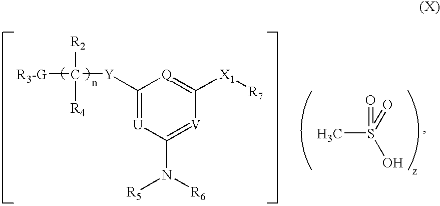

or a pharmaceutically acceptable solvate, clathrate, hydrate or polymorph thereof, wherein:

X₁ is represented by a formula selected from the group consisting of:

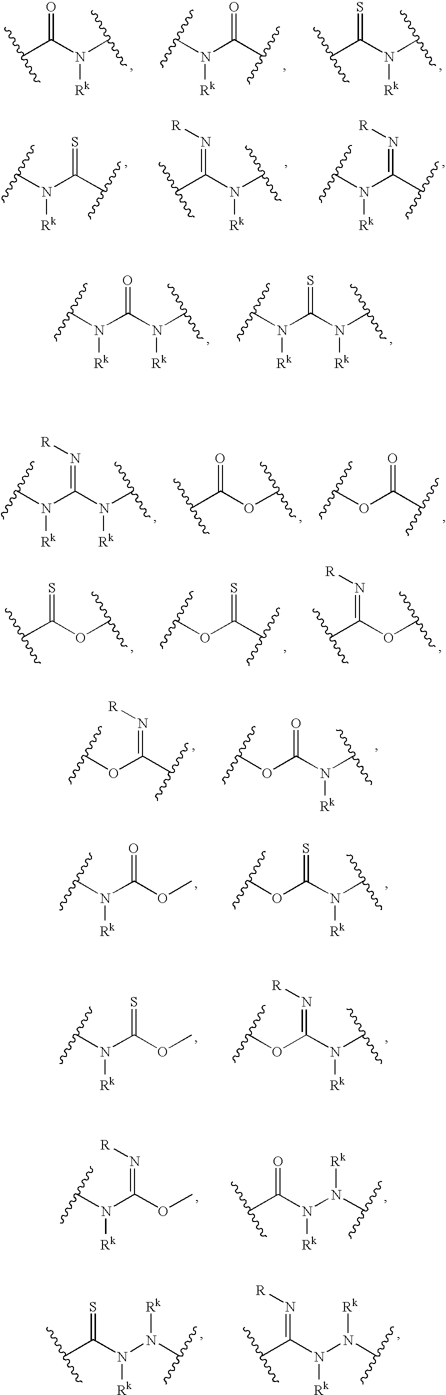

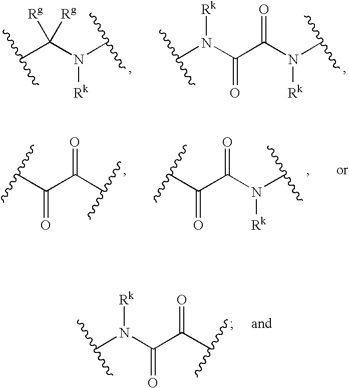

R₂, R₃, R⁴, R₅, R₆, R₇, Y, G, Q, U, V, R, R^{g}, R^{k}, n and z are defined as above;

said method comprising the steps of:
a) providing a solution of a compound represented by formula (XI):

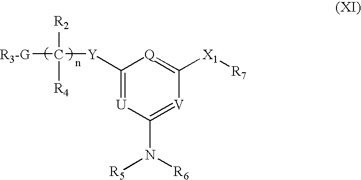

in a water miscible organic solvent, provided that the water miscible solvent is not an alcohol; and
b) adding to the solution provided in step a) a solution of methanesulfonic acid in water;
c) allowing the salt represented by formula (X) to precipitate out of solution; and
d) collecting the precipitate formed in step c), thereby preparing a methanesulfonic acid salt represented by formula (X).

In a fifth aspect, the invention relates to a method of preparing a methanesulfonic acid salt represented by formula (I):

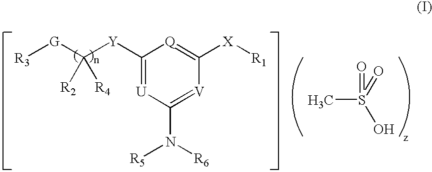

or a pharmaceutically acceptable solvate, clathrate, hydrate, polymorph or prodrug thereof;

wherein R₁, R₂, R₃, R₄, R₅, R₆, X, Y, G, Q, U, V, n and z are defined as above; said method comprising the steps of:
a) providing a solution of a compound represented by formula (II):

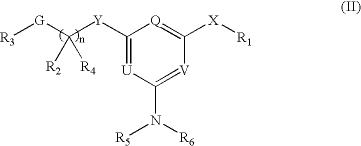

in an organic solvent, provided that the organic solvent is not an alcohol;
b) adding to the solution provided in step a) methanesulfonic acid;
c) allowing the methanesulfonic acid salt represented by formula (I) to precipitate out of solution; and
d) collecting the precipitate formed in step c), thereby preparing a methanesulfonic acid salt represented by formula (I).

In a sixth aspect, the invention relates to a method of preparing a methanesulfonic acid salt represented by formula (III):

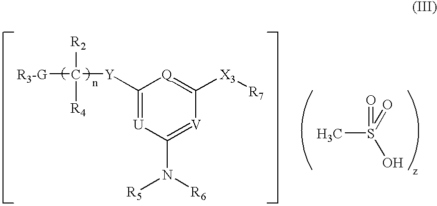

or a pharmaceutically acceptable solvate, clathrate, hydrate, prodrug or polymorph thereof; wherein R₂, R₃, R₄, R₅, R₆, R₇, X₃, Y, G, Q, U, V, n and z are defined as above; said method comprising the steps of:
a) providing a solution of a compound represented by formula (IV):

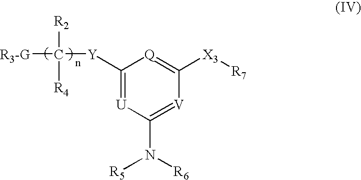

in an organic solvent, provided that the organic solvent is not an alcohol;
b) adding to the solution provided in step a) methanesulfonic acid;
c) allowing the methanesulfonic acid salt represented by formula (III) to precipitate out of solution; and
d) collecting the precipitate formed in step c), thereby preparing a methanesulfonic acid salt represented by formula (III).

In a seventh aspect, the invention relates to a method of preparing a methanesulfonic acid salt represented by formula (V):

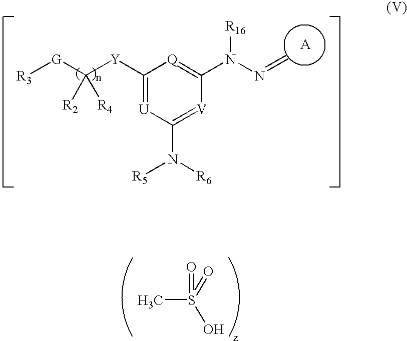

or a pharmaceutically acceptable solvate, clathrate, hydrate, polymorph, or prodrug thereof; wherein ring A, R₂, R₃, R₄, R₅, R₆, R₁₆, Y, G, Q, U, V, n and z are defined as above; said method comprising the steps of:
a) providing a solution of a compound represented by formula (VI):

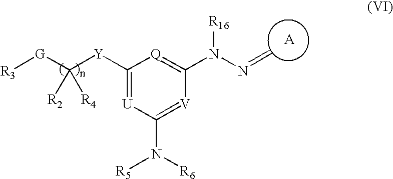

in an organic solvent, provided that the organic solvent is not an alcohol; and
b) adding to the solution provided in step a) methanesulfonic acid;
c) allowing the methanesulfonic acid salt represented by formula (V) to precipitate out of solution; and
d) collecting the precipitate formed in step c), thereby preparing a methanesulfonic acid salt represented by formula (V).

In a eighth aspect, the invention relates to a method of preparing a methanesulfonic acid salt represented by formula (X):

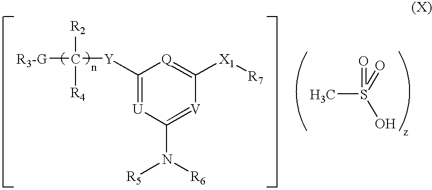

or a pharmaceutically acceptable solvate, clathrate, hydrate or polymorph thereof; wherein R₂, R₃, R₄, R₅, R₆, R₇, X₁, Y, G, Q, U, V, n and z are defined as above; said method comprising the steps of:
a) providing a solution of a compound represented by formula (XI):

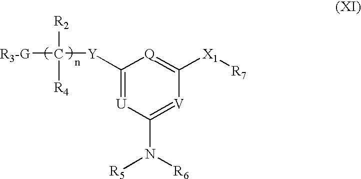

in an organic solvent, provided that the organic solvent is not an alcohol;
b) adding to the solution provided in step a) methanesulfonic acid;
c) allowing the methanesulfonic acid salt represented by formula (X) to precipitate out of solution; and
d) collecting the precipitate formed in step c), thereby preparing a methanesulfonic acid salt represented by formula (X).

In some embodiments of the invention, Q, U, and V are all N.

In some embodiments of the invention, one of Q, U, or V is CR^{g}, and the other two are N. For example, V is CR^{g}, Q and U are N; Q is CR^{g}, V and U are N; or U is CR^{g}, V and Q are N.

In some embodiments of the invention, one of Q, U, or V is N, and the other two are CR^{g}. For example, V is N, and Q and U are CR^{g}; Q is N, and V and U are CR^{g}; or U is N and Q, and V are CR^{g}.

In some embodiments of the invention, —NR₅R₆ is an optionally substituted morpholino, an optionally substituted thiomorpholino, an optionally substituted 1-oxo-thiomorpholino, an optionally substituted 1,1-dioxo-thiomorpholino, an optionally substituted piperidinyl, or an optionally substituted piperazinyl.

In some embodiments of the invention, X is —NR^{k}—. Preferably, R^{k} of group X is —H or a lower alkyl.

In some embodiments of the invention, R₁ is an optionally substituted aryl or an optionally substituted heteroaryl. For example, R₁ is an optionally substituted phenyl, an optionally substituted naphthyl, an optionally substituted anthracenyl, an optionally substituted fluorenyl, an optionally substituted indenyl, an optionally substituted azulenyl, an optionally substituted pyridyl, an optionally substituted 1-oxo-pyridyl, an optionally substituted furanyl, an optionally substituted benzo[1,3]dioxolyl, an optionally substituted benzo[1,4]dioxinyl, an optionally substituted thienyl, an optionally substituted pyrrolyl, an optionally substituted oxazolyl, an optionally substituted imidazolyl, an optionally substituted thiazolyl, an optionally substituted isoxazolyl, an optionally substituted quinolinyl, an optionally substituted pyrazolyl, an optionally substituted isothiazolyl, an optionally substituted pyridazinyl, an optionally substituted pyrimidinyl, an optionally substituted pyrazinyl, an optionally substituted triazinyl, an optionally substituted triazolyl, an optionally substituted thiadiazolyl, an optionally substituted isoquinolinyl, an optionally substituted indazolyl, an optionally substituted benzoxazolyl, an optionally substituted benzofuryl, an optionally substituted indolizinyl, an optionally substituted imidazopyridyl, an optionally substituted tetrazolyl, an optionally substituted benzimidazolyl, an optionally substituted benzothiazolyl, an optionally substituted benzothiadiazolyl, an optionally substituted benzoxadiazolyl, an optionally substituted indolyl, an optionally substituted carbazolyl, an optionally substituted 1,2,3,4-tetrahydro-carbazolyl, an optionally substituted tetrahydroindolyl, an optionally substituted azaindolyl, an optionally substituted indazolyl, an optionally substituted imidazopyridyl, an optionally substituted quinazolinyl, an optionally substituted purinyl, an optionally substituted pyrrolo[2,3]pyrimidinyl, an optionally substituted pyrazolo[3,4]pyrimidinyl, or an optionally substituted benzo(b)thienyl. Preferably, R₁ is an optionally substituted phenyl, an optionally substituted indolyl, an optionally substituted indanyl, an optionally substituted carbazolyl, or an optionally substituted 1,2,3,4-tetrahydro-carbazolyl.
In some embodiments of the invention, R₁ is a group represented by the following formula:

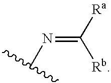

In some embodiments of the invention, one of R^{a} or R^{b} is —H or a lower alkyl, and the other is an optionally substituted aryl or an optionally substituted heteroaryl. In some embodiments of the invention, one of R^{a} or R^{b} is —H or a lower alkyl, and the other is an optionally substituted phenyl, an optionally substituted naphthyl, an optionally substituted anthracenyl, an optionally substituted fluorenyl, an optionally substituted indenyl, an optionally substituted azulenyl, an optionally substituted pyridyl, an optionally substituted 1-oxo-pyridyl, an optionally substituted furanyl, an optionally substituted benzo[1,3]dioxolyl, an optionally substituted benzo[1,4]dioxinyl, an optionally substituted thienyl, an optionally substituted pyrrolyl, an optionally substituted oxazolyl, an optionally substituted imidazolyl, an optionally substituted thiazolyl, an optionally substituted isoxazolyl, an optionally substituted quinolinyl, an optionally substituted pyrazolyl, an optionally substituted isothiazolyl, an optionally substituted pyridazinyl, an optionally substituted pyrimidinyl, an optionally substituted pyrazinyl, an optionally substituted triazinyl, an optionally substituted triazolyl, an optionally substituted thiadiazolyl, an optionally substituted isoquinolinyl, an optionally substituted indazolyl, an optionally substituted benzoxazolyl, an optionally substituted benzofuryl, an optionally substituted indolizinyl, an optionally substituted imidazopyridyl, an optionally substituted tetrazolyl, an optionally substituted benzimidazolyl, an optionally substituted benzothiazolyl, an optionally substituted benzothiadiazolyl, an optionally substituted benzoxadiazolyl, an optionally substituted indolyl, an optionally substituted carbazolyl, an optionally substituted 1,2,3,4-tetrahydro-carbazolyl, an optionally substituted tetrahydroindolyl, an optionally substituted azaindolyl, an optionally substituted indazolyl, an optionally substituted imidazopyridyl, an optionally substituted quinazolinyl, an optionally substituted purinyl, an optionally substituted pyrrolo[2,3]pyrimidinyl, an optionally substituted pyrazolo[3,4]pyrimidinyl, or an optionally substituted benzo(b)thienyl. Preferably, one of R^{a} or R^{b} is —H or a lower alkyl, and the other is an optionally substituted phenyl, an optionally substituted indolyl, an optionally substituted indanyl, an optionally substituted carbazolyl, or an optionally substituted 1,2,3,4-tetrahydro-carbazolyl.

In some embodiments of the invention, Y is O. Alternatively, In some embodiments of the invention, Y is a covalent bond.

In some embodiments of the invention, R₃ is H.

In some embodiments, R₃ is an optionally substituted aryl or an optionally substituted heteroaryl. For example, R₃ is an optionally substituted phenyl, an optionally substituted naphthyl, an optionally substituted anthracenyl, an optionally substituted fluorenyl, an optionally substituted indenyl, an optionally substituted azulenyl, an optionally substituted pyridyl, an optionally substituted 1-oxo-pyridyl, an optionally substituted furanyl, an optionally substituted benzo[1,3]dioxolyl, an optionally substituted benzo[1,4]dioxinyl, an optionally substituted thienyl, an optionally substituted pyrrolyl, an optionally substituted oxazolyl, an optionally substituted imidazolyl, an optionally substituted thiazolyl, an optionally substituted isoxazolyl, an optionally substituted quinolinyl, an optionally substituted pyrazolyl, an optionally substituted isothiazolyl, an optionally substituted pyridazinyl, an optionally substituted pyrimidinyl, an optionally substituted pyrazinyl, an optionally substituted triazinyl, an optionally substituted triazolyl, an optionally substituted thiadiazolyl, an optionally substituted isoquinolinyl, an optionally substituted indazolyl, an optionally substituted benzoxazolyl, an optionally substituted benzofuryl, an optionally substituted indolizinyl, an optionally substituted imidazopyridyl, an optionally substituted tetrazolyl, an optionally substituted benzimidazolyl, an optionally substituted benzothiazolyl, an optionally substituted benzothiadiazolyl, an optionally substituted benzoxadiazolyl, an optionally substituted indolyl, an optionally substituted tetrahydroindolyl, an optionally substituted azaindolyl, an optionally substituted indazolyl, an optionally substituted imidazopyridyl, an optionally substituted quinazolinyl, an optionally substituted purinyl, an optionally substituted pyrrolo[2,3]pyrimidinyl, an optionally substituted pyrazolo[3,4]pyrimidinyl, or an optionally substituted benzo(b)thienyl.

In some embodiments of the invention, R₃ is a hydroxy, an optionally substituted heterocycloalkyl, an optionally substituted heterocyclyl, or an optionally substituted heteroaryl. In some embodiments of the invention, R₃ is a hydroxy, an optionally substituted heterocycloalkyl, or an optionally substituted heterocyclyl.

In some embodiments of the invention, R₃ is a hydroxy, an optionally substituted pyridinyl, an optionally substituted morpholino, or an optionally substituted oxazolidin-2-one.

In some embodiments of the invention, each of R₂ and R₄ is, independently, H, an optionally substituted alkyl, an optionally substituted alkylcarbonyl, an optionally substituted aryl, an optionally substituted heteroaryl, an optionally substituted cycloalkyl, an optionally substituted cyclyl, an optionally substituted heterocycloalkyl, or an optionally substituted heterocyclyl.

In some embodiments of the invention, n is 1, 2, or 3, and R₂ and R₄, for each occurrence are, independently, H or a lower alkyl.

In some embodiments of the invention, G is absent.

In some embodiments of the invention, G is an optionally substituted heteroaryl or an optionally substituted heterocyclyl.

In some embodiments of the invention, G is —C(O)NHNH—, —NHNHC(O)—, —CH═N—NH—, —NH—N═CH—, —NHNH—, —NHO—, —O—NH—, —NR^{k}—O—, —CH═N—O—, —O—N═CH—, —O—C(S)—NH—, or —NH—C(S)—O—.

In some embodiments of the invention, G is —O—C(O)—NH—, —NH—C(NH)—NH—, —NR^{k}—C(NH)—NH—, —NR^{k}—C(NR^{k})—NH—, —NH—C(N(CN))—NH—, —NH—C(NSO₂R^{c})—NH—, —NR^{k}—C(NSO₂R^{c})—NH—, —NH—C(NNO₂)—NH—, NH—C(NC(O)R^{c})—NH—, —NH—C(O)—NH—, or —NH—C(S)—NH—.

In some embodiments of the invention, G is —NH—S(O)₂—NH—, —NR^{k}—S(O)₂—O—, —P(O)(R^{c})—, —P(O)(R^{c})—O—, or —P(O)(R^{c})—NR^{k}—.

In some embodiments of the invention, G is an optionally substituted cyclyl, an optionally substituted cycloalkyl, an optionally substituted heterocycloalkyl or an optionally substituted heterocyclyl.

In some embodiments of the invention, G is an optionally substituted cyclopropyl, an optionally substituted cyclobutyl, an optionally substituted cyclopentyl, an optionally substituted cyclohexyl, an optionally substituted cycloheptyl, an optionally substituted aziridinyl, an optionally substituted oxiranyl, an optionally substituted azetidinyl, an optionally substituted oxetanyl, an optionally substituted morpholinyl, an optionally substituted piperazinyl or an optionally substituted piperidinyl.

In some embodiments of the invention, G is an optionally substituted aryl, an optionally substituted heteroaryl, an optionally substituted heteroaralkyl, —C(N—CN)—NH—, —Si(OH)₂—, —C(NH)—NR^{k}—, or —NR^{k}—CH₂—C(O)—.

In some embodiments of the invention, G is an optionally substituted imidazolyl, an optionally substituted imidazolidinone, an optionally substituted imidazolidineamine, an optionally substituted pyrrolidinyl, an optionally substituted pyrrolyl, an optionally substituted furanyl, an optionally substituted thienyl, an optionally substituted thiazolyl, an optionally substituted triazolyl, an optionally substituted oxadiazolyl, an optionally substituted thiadiazolyl, an optionally substituted pyrazolyl, an optionally substituted tetrazolyl, an optionally substituted oxazolyl, an optionally substituted isoxazolyl, an optionally substituted phenyl, an optionally substituted pyridyl, an optionally substituted pyrimidyl, an optionally substituted indolyl, or an optionally substituted benzothiazolyl.

In some embodiments of the invention, Y is O or CH₂; G is absent; and n is 0, 1, 2, 3 or 4.

In some embodiments of the invention, the compound of formula (II) is represented by the following structural formula:

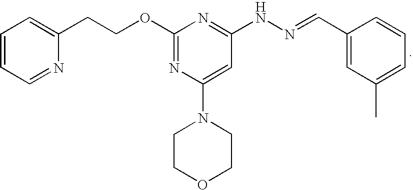

In some embodiments of the invention, X₃ is —C(R^{g})═N—NR^{k}—, wherein R^{g} and R^{k} of X₃ are each, independently, —H or a lower alkyl.

In some embodiments of the invention, R₇ is an optionally substituted phenyl, an optionally substituted naphthyl, an optionally substituted anthracenyl, an optionally substituted fluorenyl, an optionally substituted indenyl, an optionally substituted azulenyl, an optionally substituted pyridyl, an optionally substituted 1-oxo-pyridyl, an optionally substituted furanyl, an optionally substituted benzo[1,3]dioxolyl, an optionally substituted benzo[1,4]dioxinyl, an optionally substituted thienyl, an optionally substituted pyrrolyl, an optionally substituted oxazolyl, an optionally substituted imidazolyl, an optionally substituted thiazolyl, an optionally substituted isoxazolyl, an optionally substituted quinolinyl, an optionally substituted pyrazolyl, an optionally substituted isothiazolyl, an optionally substituted pyridazinyl, an optionally substituted pyrimidinyl, an optionally substituted pyrazinyl, an optionally substituted triazinyl, an optionally substituted triazolyl, an optionally substituted thiadiazolyl, an optionally substituted isoquinolinyl, an optionally substituted indazolyl, an optionally substituted benzoxazolyl, an optionally substituted benzofuryl, an optionally substituted indolizinyl, an optionally substituted imidazopyridyl, an optionally substituted tetrazolyl, an optionally substituted benzimidazolyl, an optionally substituted benzothiazolyl, an optionally substituted benzothiadiazolyl, an optionally substituted benzoxadiazolyl, an optionally substituted indolyl, an optionally substituted carbazolyl, an optionally substituted 1,2,3,4-tetrahydro-carbazolyl, an optionally substituted tetrahydroindolyl, an optionally substituted azaindolyl, an optionally substituted indazolyl, an optionally substituted imidazopyridyl, an optionally substituted quinazolinyl, an optionally substituted purinyl, an optionally substituted pyrrolo[2,3]pyrimidinyl, an optionally substituted pyrazolo[3,4]pyrimidinyl, or an optionally substituted benzo(b)thienyl.

In some embodiments of the invention, R₇ is an optionally substituted phenyl, an optionally substituted indolyl, an optionally substituted indanyl, an optionally substituted carbazolyl, or an optionally substituted 1,2,3,4-tetrahydro-carbazolyl.

In some embodiments of the invention, R₁ or R₇ is a group represented by the following formula:

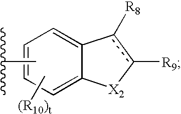

wherein:

the dashed line indicates a double or a single bond;

X₂ is —O—, —S(O)ₚ—, —N(R^{k})—, or —C(R^{g})(R^{g})—;

R₈ and R^{g} are each, independently, R^{g}, —C(O)R^{c}, —C(S)R^{c}, —C(NR)R^{c}, —NR^{k}C(O)R^{c}, —OC(O)R^{c}, —SC(O)R^{c}, —NR^{k}C(S)R^{c}, —OC(S)R^{c}, —SC(S)R^{c}, —NR^{k}C(NR)R^{c}, —OC(NR)R^{c}, or —SC(NR)R^{c}; or R₈ and R^{g}, taken together with the carbons to which they are attached, form a 5- to 7-membered optionally substituted cycloalkyl, a 5- to 7-membered optionally substituted cyclyl, a 5- to 7-membered optionally substituted aryl, a 5- to 7-membered optionally substituted heterocycloalkyl, a 5- to 7-membered optionally substituted heterocyclyl, a 5- to 7-membered optionally substituted heteroaryl;

R₁₀, for each occurrence, is, independently, R^{g}, —C(O)R^{c}, —C(S)R^{c}, —C(NR)R^{c}, —NR^{k}C(O)R^{c}, —OC(O)R^{c}, —SC(O)R^{c}, —NR^{k}C(S)R^{c}, —OC(S)R^{c}, —SC(S)R^{c}, —NR^{k}C(NR)R^{c}, —OC(NR)R^{c}, or —SC(NR)R^{c};

p is 0, 1, or 2; and

t is 0, 1, 2, or, 3.

In some embodiments of the invention, R₇ is (2,3-dimethyl-1H-indol-5-yl), (1H-indol-5-yl), or (6,7,8,9-tetrahydro-5H-carbazol-3-yl).

In some embodiments of the invention, R₁ or R₇ is a group represented by the following formula:

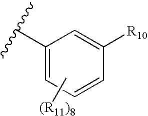

wherein:

R₁₀ is defined as above;

R₁₁, for each occurrence, is, independently, R^{g}, —C(O)R^{c}, —C(S)R^{c}, —C(NR)R^{c}, —NR^{k}C(O)R^{c}, —OC(O)R^{c}, —SC(O)R^{c}, —NR C(S)R^{c}, —OC(S)R^{c}, —SC(S)R^{c}, —NR C(NR)R^{c}, —OC(NR)R^{c}, or —SC(NR)R^{c}; and

s is 0, 1, 2, 3, or 4.

In some embodiments of the invention, the solution provided in step a) comprises a compound is represented by formula (VII):

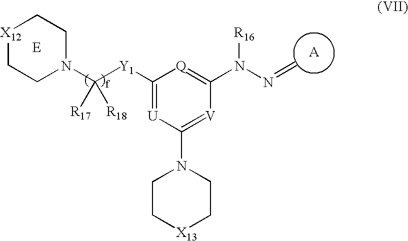

or a pharmaceutically acceptable salt, solvate, clathrate, hydrate, polymorph, or prodrug thereof, wherein:
ring E is optionally substituted with one to four substituents selected from a lower alkyl, a halo, an amino, a lower alkyl amino, a lower dialkyl amino, a cyano, a nitro, a lower haloalkyl, a hydroxyl, and a lower hydroxyalkyl;
X₁₂ is O, S, S(O), S(O)₂, or CR^{g}R^{g};
X₁₃ is O, S, S(O), S(O)₂, or CH₂;
Y₁ is O, S, NR^{k}, or CH₂;
R₁₇ and R₁₈, for each occurrence, are independently, H or a lower alkyl; or R₁₇ and R₁₈ taken together with the carbon to which they are attached form a cycloalkyl; and
f is 0, 1, 2, or 3.

In some embodiments of the invention, the solution provided in step a) comprises a compound is represented by formula (VIII):

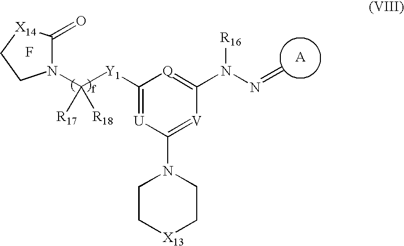

or a pharmaceutically acceptable salt, solvate, clathrate, hydrate, polymorph, or prodrug thereof, wherein:
ring F is optionally substituted with one or two substituents selected from a lower alkyl, a halo, an amino, a lower alkyl amino, a lower dialkyl amino, a cyano, a nitro, a lower haloalkyl, a hydroxyl, and a lower hydroxyalkyl;
X₁₃ is O, S, S(O), S(O)₂, or CH₂;
X₁₄ is O, NR^{k}, or CR^{g}R^{g};
Y, is O, S, NR^{k}, or CH₂;
R₁₇ and R₁₈, for each occurrence, are independently, H or a lower alkyl; or R₁₇ and R₁₈ taken together with the carbon to which they are attached form a cycloalkyl; and
f is 0, 1, 2, or 3.

In some embodiments of the invention, the solution provided in step a) comprises a compound is represented by formula (IX):

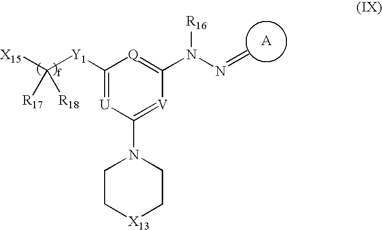

or a pharmaceutically acceptable salt, solvate, clathrate, hydrate, polymorph, or prodrug thereof, wherein:
X₁₃ is O, S, S(O), S(O)₂, or CH₂;
X₁₅ is —OH, —NH₂ or —SH;
Y₁ is O, S, NR^{k}, or CH₂;
R₁₇ and R₁₈, for each occurrence, are independently, H or a lower alkyl; or R₁₇ and R₁₈ taken together with the carbon to which they are attached form a cycloalkyl; and
f is 0, 1, 2, or 3.

In some embodiments of the invention, ring A is a ring system selected from the group consisting of:

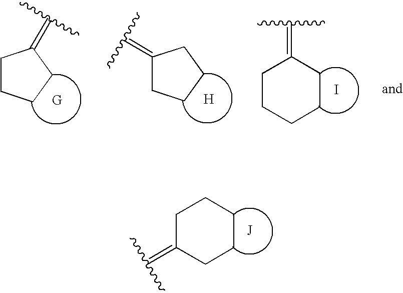

wherein:
[symbol] represents the point of attachment; rings G, H, I, and J are each, independently, an aryl or a heteroaryl; and each ring system is optionally substituted with one or more substituents.

In some embodiments of the invention, ring A is a ring system selected from the group consisting of:

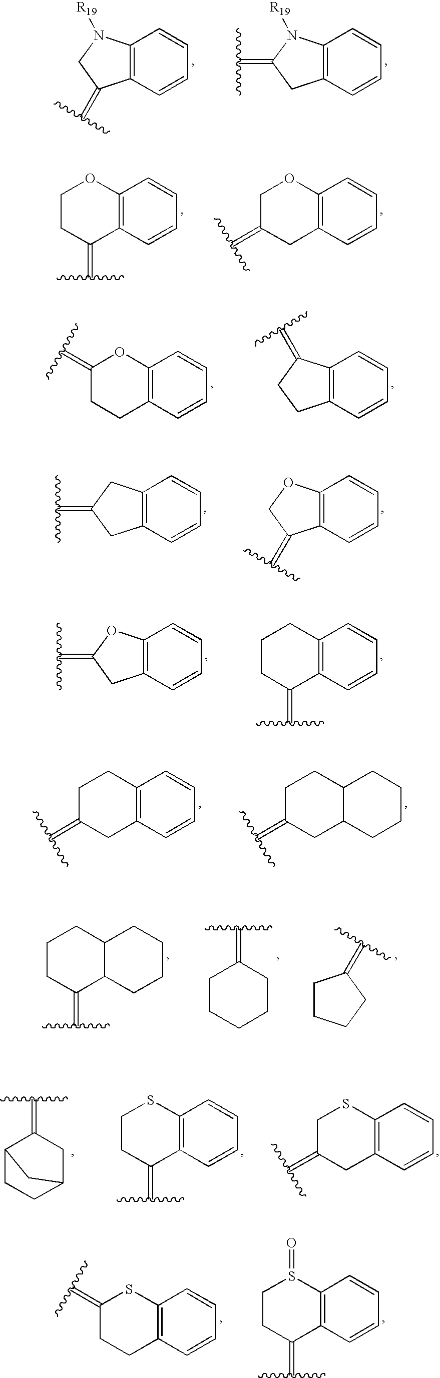

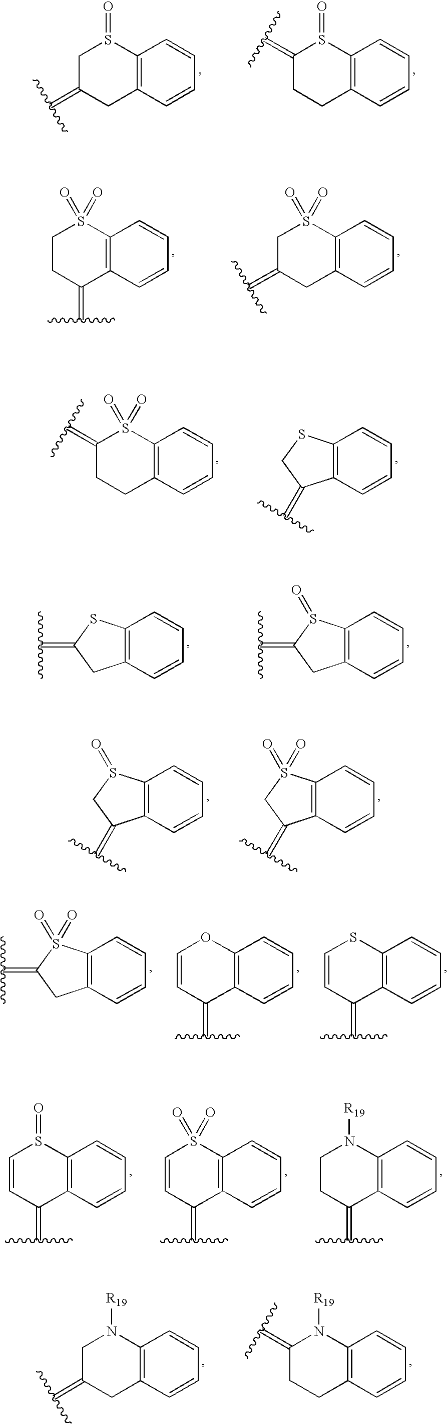

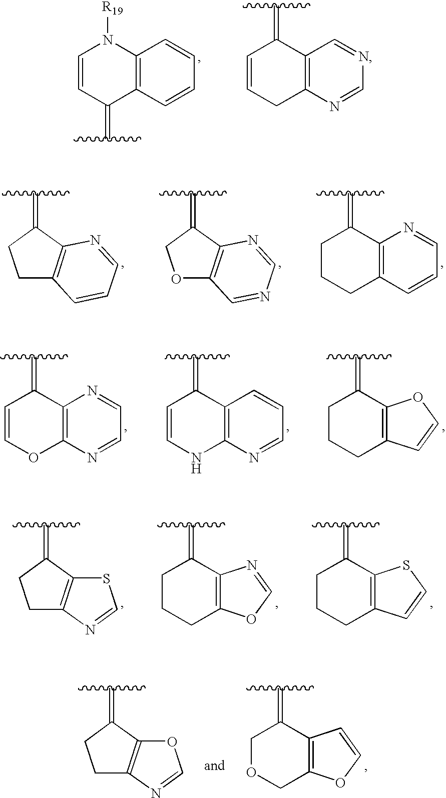

wherein:
each ring system is optionally substituted with one or more substituents;
[symbol] represents the point of attachment; and
R₁₉ is H, an alkyl, an aralkyl, or an alkylcarbonyl.
   In some embodiments of the invention, ring A is a ring system selected from the group consisting of:

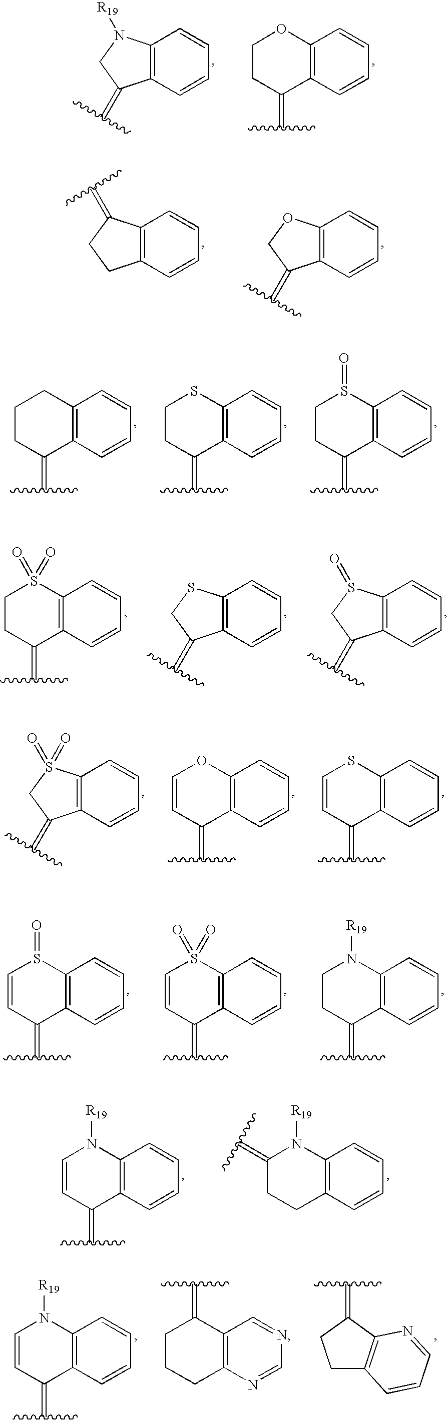

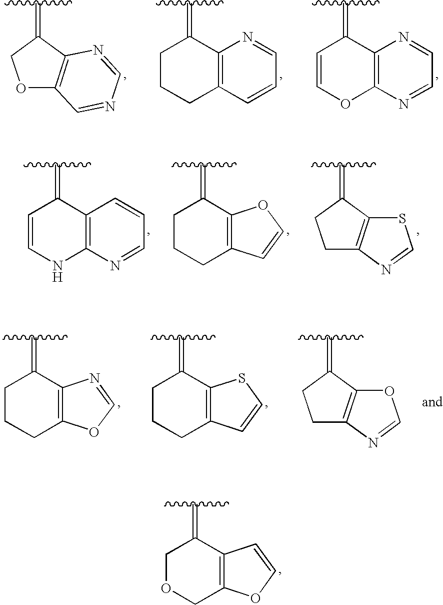

wherein:
each ring system is optionally substituted with one or more substituents.

In some embodiments of the invention, ring A is optionally substituted with one or more substituents selected from the group consisting of an optionally substituted alkyl, an optionally substituted alkoxy, an optionally substituted alkyl sulfanyl, an optionally substituted alkenyl, an optionally substituted alkynyl, an optionally substituted cycloalkyl, an optionally substituted cyclyl, an optionally substituted heterocyclyl, an optionally substituted heterocycloalkyl, an optionally substituted aryl, an optionally substituted heteroaryl, an optionally substituted aralkyl, an optionally substituted heteraralkyl, a haloalkyl, halo, cyano, nitro, haloalkoxy, ═O, ═S, ═NR, —OR^{k}, —NR^{h}R^{j}, —SR^{k}, —C(O)R^{k}, —C(O)NR^{h}R^{j}, —NR^{k}C(O)R^{k}, —C(O)OR^{k}, —OC(O)R^{k}, —NR^{k}C(O)NR^{h}R^{j}, —OC(O)NR^{h}R^{j}, —NR^{k}C(O)OR^{k}, —C(NR)R^{k}, —C(NR)NR^{h}R^{j}, —NR^{k}C(NR)R^{k}, —C(NR)OR^{k}, —OC(NR)R^{k}, —NR^{k}C(NR)NR^{h}R^{j}, —OC(NR)NR^{h}R^{j}, —NR^{k}C(NR)OR^{k}, —C(S)R^{k}, —C(S)NR^{h}R^{j}, —NR^{k}C(S)R^{k}, —C(S)OR^{k}, —OC(S)R^{k}, —NR^{k}C(S)NR^{h}R^{j}, —OC(S)NR^{h}R^{j}, —NR^{k}C(S)OR^{k}, —C(O)SR^{k}, —SC(O)R^{k}, —S(O)ₕR^{k}, —S(O)ₕNR^{h}R^{j}, —OS(O)ₕR^{k}, —S(O)ₕOR^{k}, —OS(O)ₕOR^{k}, —P(O)(OR^{k})₂, —OP(O)(OR^{k})₂, —P(S)(OR^{k})₂, —SP(O)(OR^{k})₂, —P(O)(SR^{k})(OR^{k}), —OP(O)(SR^{k})(OR^{k}), —P(O)(SR^{k})₂, or —OP(O)(SR^{k})₂, wherein h is 1 or 2.

In some embodiments of the invention, ring A is optionally substituted with from one to three substituents selected from the group consisting of a lower alkyl, a lower alkoxy, ═O, nitro, cyano, hydroxy, amino, lower alkyl amino, lower dialkyl amino, mercapto, lower alkyl sulfanyl, halo, or haloalkyl.

In some embodiments of the invention, in the compounds represented by formula (VII), X₁₂, X₁₃, Y₁ is O; and R₁₇ and R₁₈ are each, independently, H or a lower alkyl.

In some embodiments of the invention, in the compounds represented by formula (VIII), X₁₃, X₁₄, and Y₁ are 0; and R₁₇ and R₁₈ are each, independently, H or a lower alkyl.

In some embodiments of the invention, in the compounds represented by formula (IX), X₁₃ and Y₁ are 0; X₁₅ is —OH; and R₁₇ and R₁₈ are each, independently, H or a lower alkyl.

In some embodiments of the invention, X₁ is one of the following formulas:

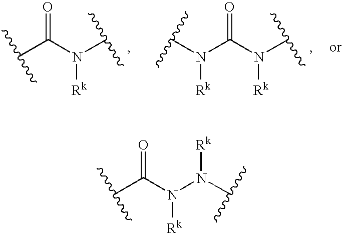

In some embodiments of the invention, X₁ is represented by the following formula:

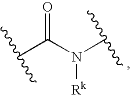

wherein R^{k} is —H or a lower alkyl.

In some embodiments of the invention, X₁ is represented by the following formula:

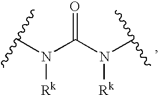

wherein R^{k} is —H or a lower alkyl.

In some embodiments of the invention, X₁ is represented by the following formula:

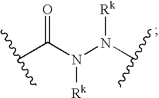

wherein R^{k} is —H or a lower alkyl.

In some embodiments of the first, second, third, and fourth aspects of the invention, z is 2 and the solution of methanesulfonic acid in water contains between about 1.8 to about 2.5 molar equivalents of methanesulfonic acid with respect to the compound of formula (II), (IV), (VI), or (XI) in step a).

In some embodiments of the first, second, third, and fourth aspects of the invention, z is 1 and the solution of methanesulfonic acid in water contains between about 0.9 to about 1.25 molar equivalents of methanesulfonic acid with respect to the compound of formula (II), (IV), (VI), or (XI) in step a).

In some embodiments of the first, second, third, and fourth aspects of the invention, the water miscible organic solvent is selected from the group consisting of acetone or acetonitrile.

In some embodiments of the first, second, third, and fourth aspects of the invention, the solution of the compound of formula (II), (IV), (VI), or (XI) in the water miscible organic solvent in step a) has a molar concentration of between about 20 mM and about 150 mM.

In some embodiments of the first, second, third, and fourth aspects of the invention, the solution of methanesulfonic acid in water has a concentration of between about 1.5 M and about 7 M.

In some embodiments of the first, second, third, and fourth aspects of the invention, the temperature is maintained at about 35° C. or less during the method of producing the methanesulfonic acid salt.

In some embodiments of the first, second, third, and fourth aspects of the invention, the temperature is maintained at about 30° C. or less during the method of producing the methanesulfonic acid salt.

In some embodiments of the first, second, third, and fourth aspects of the invention, the temperature during steps a) and b) is between about 23° C. and about 30° C.

In some embodiments of the fifth, sixth, seventh and eight aspects of the invention, z is 2 and the methanesulfonic acid added to the solution of step a) has about 1.8 to about 2.5 molar equivalents of methanesulfonic acid with respect to the compound of step a).

In some embodiments of the fifth, sixth, seventh and eight aspects of the invention, z is 1 and the solution of methanesulfonic acid added to the solution of step a) has about 0.9 to about 1.25 molar equivalents of methanesulfonic acid with respect to the compound of step a).

In some embodiments of the fifth, sixth, seventh and eight aspects of the invention, the solution of the compound in step a) is heated to between about 35° C. and about 75° C.

In some embodiments of the fifth, sixth, seventh and eight aspects of the invention, the solution is allowed to cool to between about 0° C. and about 25° C. during precipitation of the methanesulfonic acid salt.

In some embodiments of the fifth, sixth, seventh and eight aspects of the invention, the solution of step a) has a molar concentration of the compound of between about 100 mM and about 200 mM.

In some embodiments of the fifth, sixth, seventh and eight aspects of the invention, the organic solvent is ethyl acetate or dichloromethane.

In some embodiments of the fifth, sixth, seventh and eight aspects of the invention, the methanesulfonic acid is added in a solution with an organic solvent. In some embodiments of the fifth, sixth, seventh and eight aspects of the invention, the solution of methanesulfonic acid in an organic solvent has a concentration of between about 1.5 M and about 7 M.

In some embodiments of the first, second, third, fourth, fifth, sixth, seventh and eighth aspects of the invention, the salt is allowed to precipitate out of solution for about 2 hours to about 24 hours.

In some embodiments of the first, second, third, fourth, fifth, sixth, seventh and eighth aspects of the invention, the solution is continuously stirred while the salt is allowed to precipitate out of solution.

The precipitate can be collected by any method known to those skilled in the art. For example, the precipitated may be collected by filtration. Filtration may be carried out by applying a vacuum to the collection flask or by applying pressure to the mixture being filtered to accelerate the filtration. Alternatively, the precipitate may be collected by sedimentation either with or without centrifugation to accelerate the sedimentation.

In some embodiments of the first, second, third, fourth, fifth, sixth, seventh and eighth aspects of the invention, the methanesulfonic acid salt produced by the method may be dried under vacuum for between about 1 hour and about 24 hours. In some embodiments of the first, second, third, fourth, fifth, sixth, seventh and eighth aspects of the invention, the methanesulfonic acid salt is heated to about 40° C. to about 80° C. while it is dried under vacuum.

In some embodiments of the first, second, third, fourth, fifth, sixth, seventh and eighth aspects of the invention, the methanesulfonic acid salt produced by the method may be further purified by recrystallization. The recrystallization process comprises the steps of:
e) dissolving the methanesulfonic acid salt represented collected in step d) in water to form a clear solution having a concentration of between about 1 mM and about 8 mM;
f) adding between about 5 mL and about 15 mL of acetone per gram of the methanesulfonic acid salt;
g) allowing the methanesulfonic acid salt to precipitate out of solution; and
h) collecting the precipitate.

In some embodiments of the first, second, third, fourth, fifth, sixth, seventh and eighth aspects of the invention, the solution is maintained at 30° C. or less, preferably at about 18° C. to about 30° C. during addition of the acetone in the recrystallization process.

In some embodiments of the first, second, third, fourth, fifth, sixth, seventh and eighth aspects of the invention, during the recrystallization process, the methanesulfonic acid salt is allowed to precipitate out of solution for about 0.5 hours to about 24 hours.

In some embodiments of the first, second, third, fourth, fifth, sixth, seventh and eighth aspects of the invention, the recrystallized methanesulfonic acid salt is dried under vacuum for between about 1 hour and about 24 hours. In some embodiments of the first, second, third, fourth, fifth, sixth, seventh and eighth aspects of the invention, the recrystallized methanesulfonic acid salt is heated to about 40° C. to about 80° C. while it is dried under vacuum.

Methanesulfonic acid salts can be isolated, e.g., by filtration of a precipitated disalt. Removal of bulk and/or residual solvents can be carried out, e.g., using one or more of the following techniques. In some embodiments of the invention, solvent removal can be carried out by natural evaporation (e.g., under ambient conditions with substantially no deliberate displacement of solvent vapors from the vicinity of the methanesulfonic acid salt or forced evaporation). In some embodiments of the invention, solvent removal can be carried out by deliberate displacement of solvent vapors from the vicinity of the methanesulfonic acid salt (e.g., by a directed stream of air or an inert gas, such as nitrogen or argon). Solvent removal can be carried out in vacuo, for example, at a pressure of at least about 0.05 mm Hg (e.g., at least about 0.10 mm Hg, at least about 0.50 mm Hg, at least about 1 mm Hg, at least about 5 mm Hg, at least about 10 mm Hg, at least about 30 mm Hg).

The extent of solvent removal can be monitored by gravimetric methods (e.g. drying of the methanesulfonic acid salt until a constant weight of the disalt is achieved) or spectroscopic techniques (e.g., removing a sample of the methanesulfonic acid salt and obtaining a ¹H NMR spectrum of the sample to detect the solvent).

The compounds in Table 1 inhibit the production of IL-12, IL-23 and/or IL-27. Mesylate salts of the compounds in Table 1 can be prepared using the method of the invention disclosed herein.

TABLE 1

| | | |
|---|---|---|
| No. | Structure | Name |

| | | |
|---|---|---|
| 1 | 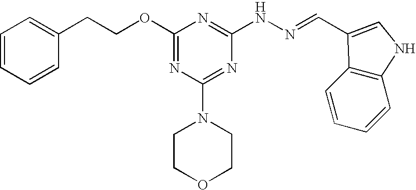 | N-(1H-Indol-3-ylmethylene)- N′-(4-morpholin-4-yl-6- phenethyloxy-[1,3,5]triazin-2- yl)-hydrazine |
| 2 | 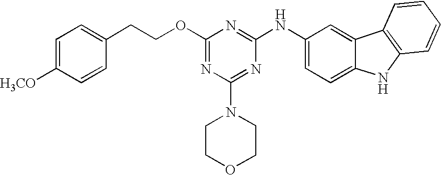 | N-(9H-carbazol-3-yl)-{4-[2-(4- methoxy-phenyl)-ethoxy]-6- morpholin-4-yl-[1,3,5]triazin- 2-yl}-amine |
| 3 | 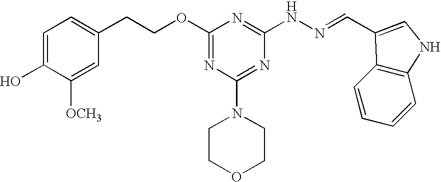 | N-(1H-Indol-3-ylmethylene)- N′-(4-morpholin-4-yl-6-(3- methoxy-4-hydroxy- phenethyloxy-[1,3,5]triazin-2- yl)-hydrazine |
| 4 | 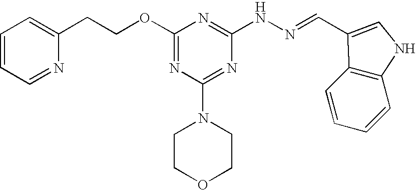 | N-(1H-Indol-3-ylmethylene)- N′-(4-morpholin-4-yl-6-(2- pyridine-2-yl-ethyloxy- [1,3,5]triazin-2-yl)-hydrazine |
| 5 | 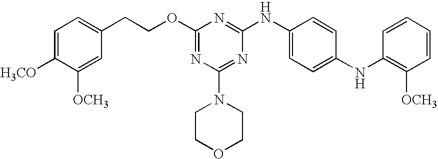 | N-[4-(2-methoxy- phenylamino)-phenyl]-{4-[2- (3,4-dimethoxy-phenethyloxy]- 6-morpholin-4-yl- [1,3,5]triazin-2-yl}-amine |
| 6 | 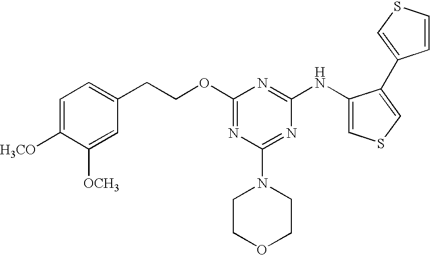 | [3,3′]Bithiophen-4-yl-{4-[2- (3,4-dimethoxy-phenethyloxy]- 6-morpholin-4-yl- [1,3,5]triazin-2-yl}-amine |
| 7 | 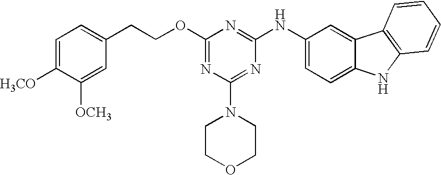 | N-(9H-carbazol-3-yl)-{4-[2- (3,4-dimethoxy-phenyl)- ethoxy]-6-morpholin-4-yl- [1,3,5]triazin-2-yl}-amine |
| 8 | 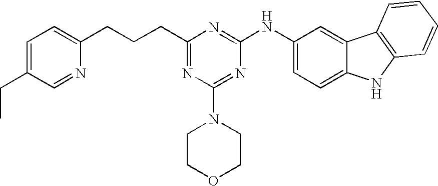 | N-(9H-carbazol-3-yl)-{4-[3-(5- ethyl-phenyl)-propyl]-6- morpholin-4-yl-[1,3,5]triazin- 2-yl}-amine |
| 9 | 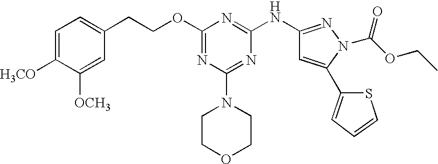 | 3-{4-[2-(3,4-Dimethoxy- phenyl)-ethoxy]-6-morpholin-4-yl- [1,3,5]triazin-2-ylamino}- 5-thiophen-2-yl-pyrazole- 1-carboxylic acid ethyl ester |
| 10 | 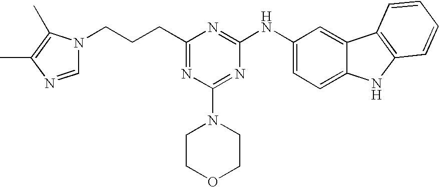 | (9H-Carbazol-3-yl)-{4-[3- (4,5-dimethyl-imidazol- 1-yl)-propyl]-6-morpholin- 4-yl-[1,3,5]triazin-2-yl}- amine |
| 11 | 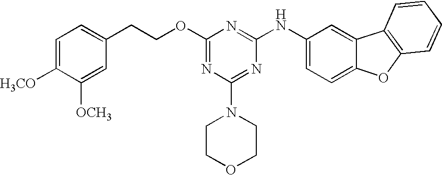 | Dibenzofuran-2-yl-{4-[2- (3,4-dimethoxy-phenyl)- ethoxy]-6-morpholin- 4-yl-[1,3,5]triazin-2-yl}- amine |
| 12 | 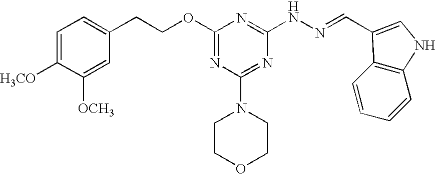 | N-{4-[2-(3,4-Dimethoxy- phenyl)-ethoxy]-6-morpholin- 4-yl-[1,3,5]triazin-2-yl}- N′-(1H-indol-3-ylmethylene)- hydrazine |
| 13 | 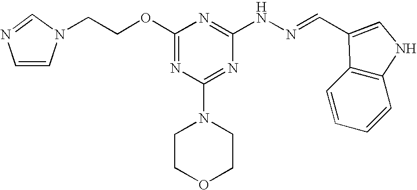 | N-[4-(2-Imidazol-1-yl-ethoxy)- 6-morpholin-4-yl- [1,3,5]triazin-2-yl]-N′-(1H- indol-3-ylmethylene)-hydrazine |
| 14 | 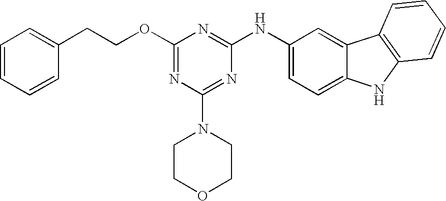 | (9H-Carbazol-3-yl)-(4-morpholin- 4-yl-6-phenethyloxy- [1,3,5]triazin-2-yl)-amine |
| 15 | 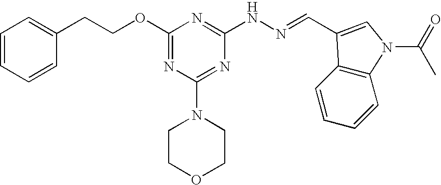 | 1-{3-[(4-Morpholin-4- yl-6-phenethyloxy- [1,3,5]triazin-2-yl)- hydrazonomethyl]-indol- 1-yl}-ethanone |
| 16 | 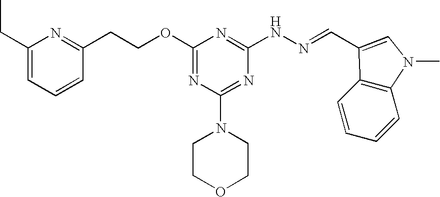 | N-{4-[2-(6-Ethyl-pyridin-2-yl)- ethoxy]-6-morpholin-4-yl- [1,3,5]triazin-2-yl}-N′-(1-methyl- 1H-indol-3-ylmethylene)-hydrazine |
| 17 | 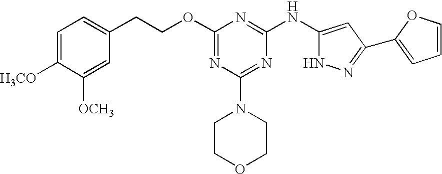 | {4-[2-(3,4-Dimethoxy-phenyl)- ethoxy]-6-morpholin-4-yl- [1,3,5]triazin-2-yl}-(5- furan-2-yl-2H-pyrazol-3- yl)-amine |
| 18 | 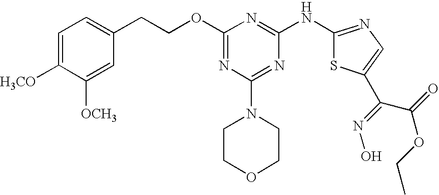 | (2-{4-[2-(3,4-Dimethoxy-phenyl)- ethoxy]-6-morpholin-4-yl- [1,3,5]triazin-2-ylamino}-thiazol- 5-yl)-hydroxyimino-acetic acid ethyl ester |
| 19 | 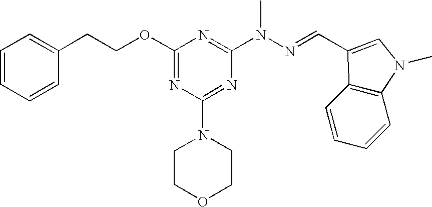 | N-Methyl-N′-(1-methyl-1H- indol-3-ylmethylene)-N- (4-morpholin-4-yl-6- phenethyloxy-[1,3,5]triazin- 2-yl)-hydrazine |
| 20 | 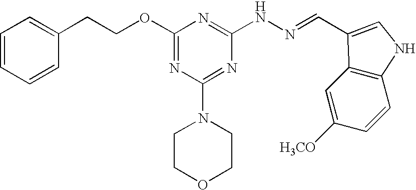 | N-(5-Methoxy-1H-indol-3- ylmethylene)-N′-(4-morpholin- 4-yl-6-phenethyloxy- [1,3,5]triazin-2-yl)-hydrazine |
| 21 | 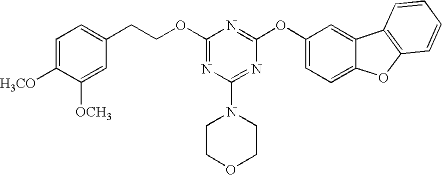 | 2-(Dibenzofuran-2-yloxy)-4- [2-(3,4-dimethoxy-phenyl)- ethoxy]-6-morpholin-4-yl- [1,3,5]triazine |
| 22 | 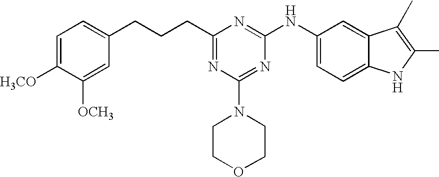 | {4-[3-(3,4-Dimethoxy-phenyl)- propyl]-6-morpholin-4-yl- [1,3,5]triazin-2-yl}-(2,3- dimethyl-1H-indol-5-yl)-amine |
| 23 | 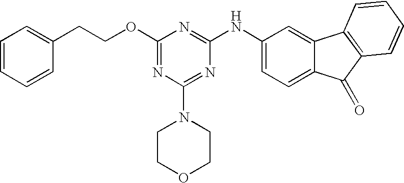 | 3-(4-Morpholin-4-yl-6- phenethyloxy-[1,3,5]triazin- 2-ylamino)-fluoren-9-one |
| 24 | 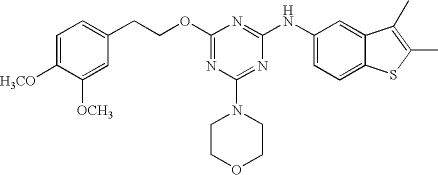 | {4-[2-(3,4-Dimethoxy-phenyl)- ethoxy]-6-morpholin-4-yl- [1,3,5]triazin-2-yl}-(2,3- dimethyl-benzo[b]thiophen-5- yl)-amine |
| 25 | 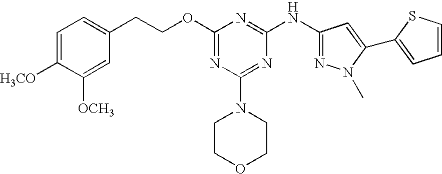 | {4-[2-(3,4-Dimethoxy-phenyl)- ethoxy]-6-morpholin-4-yl- [1,3,5]triazin-2-yl}-(1- methyl-5-thiophen-2-yl- 1H-pyrazol-3-yl)-amine |
| 26 | 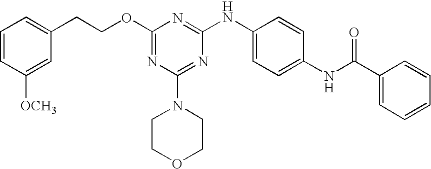 | N-(4-{4-[2-(3-Methoxy-phenyl)- ethoxy]-6-morpholin-4-yl- [1,3,5]triazin-2-ylamino}- phenyl)-benzamide |
| 27 | 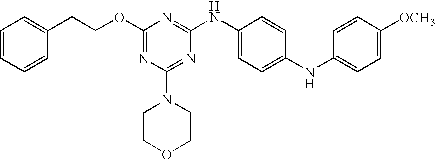 | N-(4-Methoxy-phenyl)-N′-(4- morpholin-4-yl-6-phenethyloxy- [1,3,5]triazin-2-yl)-benzene- 1,4-diamine |
| 28 | 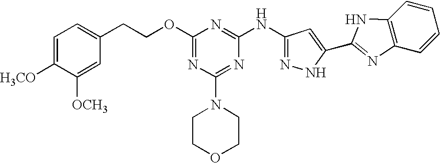 | [5-(1H-Benzoimidazol-2-yl)- 1H-pyrazol-3-yl]-{4-[2-(3,4- dimethoxy-phenyl)-ethoxy]-6- morpholin-4-yl-[1,3,5]triazin- 2-yl}-amine |
| 29 | 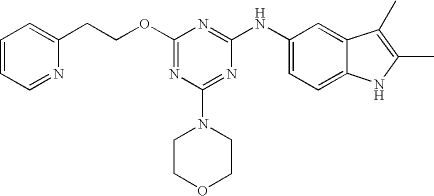 | (2,3-Dimethyl-1H-indol-5-yl)- [4-morpholin-4-yl-6-(2-pyridin- 2-yl-ethoxy)-[1,3,5]triazin-2-yl]- amine |
| 30 | 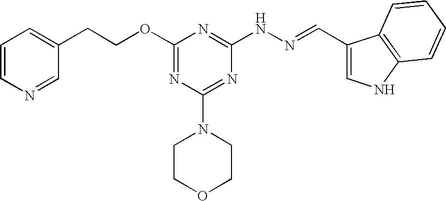 | N-(1H-Indol-3-ylmethylene)-N′- [4-morpholin-4-yl-6-(2-pyridin- 3-yl-ethoxy)-[1,3,5]triazin-2- yl]-hydrazine |
| 31 | 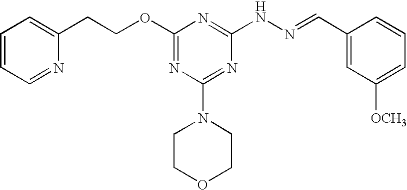 | N-(3-Methoxy-benzylidene)-N′- [4-morpholin-4-yl-6-(2- pyridin-2-yl-ethoxy)- [1,3,5]triazin-2-yl]-hydrazine |
| 32 | 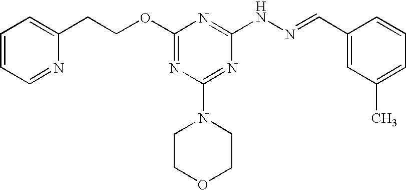 | N-(3-Methyl-benzylidene)-N′- [4-morpholin-4-yl-6-(2- pyridin-2-yl-ethoxy)- [1,3,5]triazin-2-yl]-hydrazine |
| 33 | 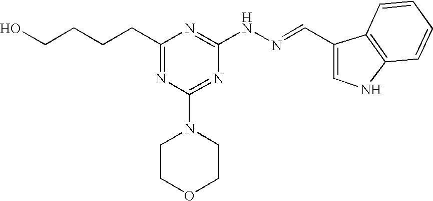 | 4-{4-[N′-(1H-Indol-3- ylmethylene)-hydrazino]-6- morpholin-4-yl-[1,3,5]triazin- 2-yl}-butan-1-ol |
| 34 | 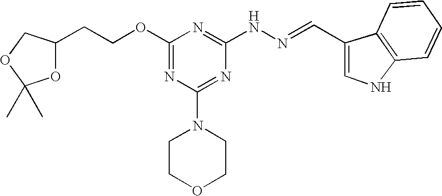 | N-{4-[2-(2,2-Dimethyl- [1,3]dioxolan-4-yl)-ethoxy]- 6-morpholin-4-yl- [1,3,5]triazin-2-yl}-N′-(1H-indol- 3-ylmethylene)-hydrazine |
| 35 | 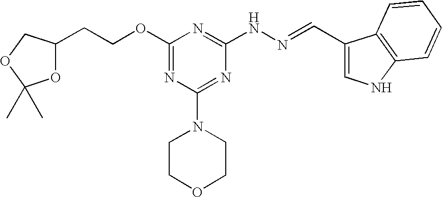 | N-{4-[2-(2,2-Dimethyl- [1,3]dioxolan-4-y1)-ethoxy]- 6-morpholin-4-yl-[1,3,5]triazin- 2-yl}-N′-(1H-indol-3- ylmethylene)-hydrazine |
| 36 | 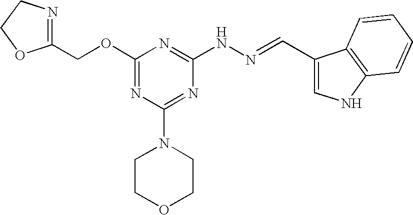 | N-[4-(4,5-Dihydro-oxazol-2- ylmethoxy)-6-morpholin-4-yl- [1,3,5]triazin-2-yl]-N′-(1H-indol- 3-ylmethylene)-hydrazine |
| 37 | 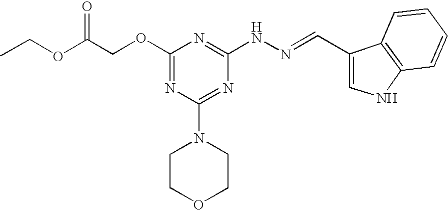 | {4-[N′-(1H-Indol- 3-ylmethylene)-hydrazino]- 6-morpholin-4-yl-[1,3,5]triazin- 2-yloxy}-acetic acid ethyl ester |
| 38 | 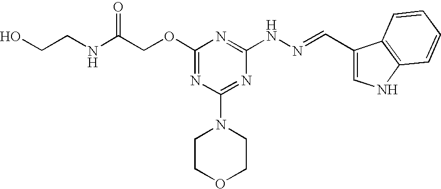 | N-(2-Hydroxy-ethyl)-2-{4-[N′- (1H-indol-3-ylmethylene)- hydrazino]-6-morpholin-4-yl- [1,3,5]triazin-2-yloxy}-acetamide |
| 39 | 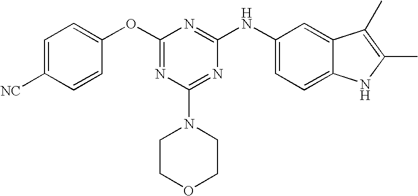 | 4-[4-(2,3-Dimethyl-1H- indol-5-ylamino)-6- morpholin-4-yl-[1,3,5]triazin- 2-yloxy]-benzonitrile |
| 40 | 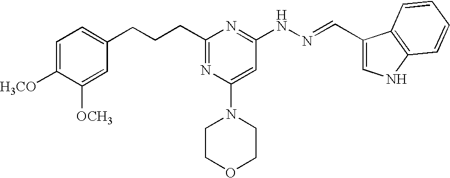 | N-{2-[3-(3,4-Dimethoxy- phenyl)-propyl]-6- morpholin-4-yl-pyrimidin-4-yl}- N′-(1H-indol-3-ylmethylene)- hydrazine |
| 41 | 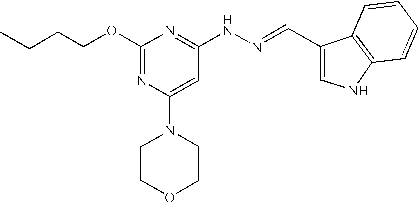 | N-(2-Butoxy-6-morpholin- 4-yl-pyrimidin-4-yl)- N′-(1H-indol-3-ylmethylene)- hydrazine |
| 42 | 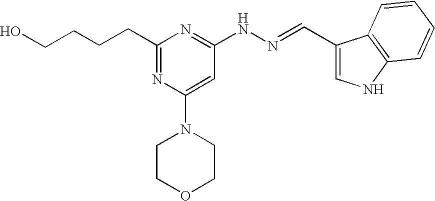 | 4-{4-[N′-(1H-Indol- 3-ylmethylene)-hydrazino]- 6-morpholin-4-yl-pyrimidin- 2-yl}-butan-1-ol |
| 43 | 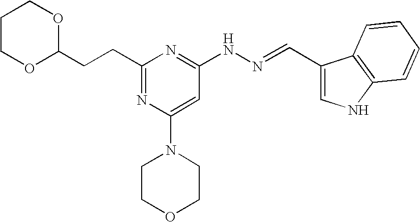 | N-[2-(2-[1,3]Dioxan-2-yl-ethyl)- 6-morpholin-4-yl-pyrimidin-4-yl]- N′-(1H-indol-3-ylmethylene)- hydrazine |
| 44 | 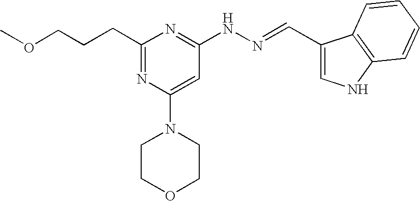 | N-(1H-Indol-3-ylmethylene)- N′-[2-(3-methoxy-propyl)- 6-morpholin-4-yl-pyrimidin- 4-yl]-hydrazine |
| 45 | 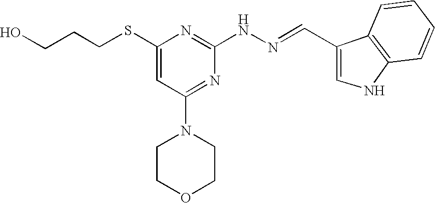 | 3-{2-[N′-(1H-Indol-3- ylmethylene)-hydrazino]-6- morpholin-4-yl-pyrimidin-4- ylsulfanyl}-propan-1-ol |
| 46 | 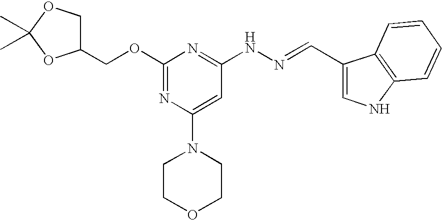 | N-[2-(2,2-Dimethyl- [1,3]dioxolan-4-ylmethoxy)- 6-morpholin-4-yl-pyrimidin- 4-yl]-N′-(1H-indol-3- ylmethylene)-hydrazine |
| 47 | 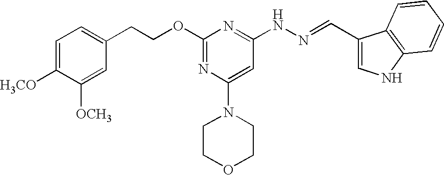 | N-{2-[2-(3,4-Dimethoxy- phenyl)-ethoxy]-6- morpholin-4-yl-pyrimidin-4-yl}- N′-(1H-indol-3-ylmethylene)- hydrazine |
| 48 | 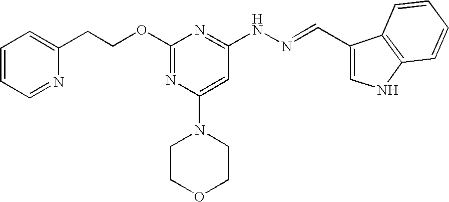 | N-(1H-Indol-3-ylmethylene)- N′-[6-morpholin-4-yl-2-(2- pyridin-2-yl-ethoxy)- pyrimidin-4-yl]-hydrazine |
| 49 | 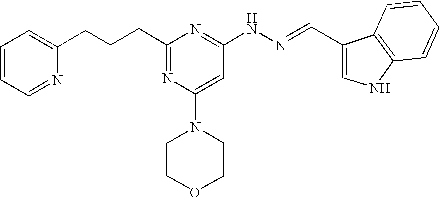 | N-(1H-Indol-3-ylmethylene)- N′-[6-morpholin-4-yl-2-(3- pyridin-2-yl-propyl)- pyrimidin-4-yl]-hydrazine |
| 50 | 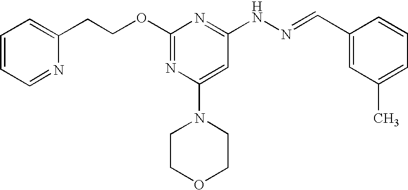 | N-(3-Methyl-benzylidene)- N′-[6-morpholin-4-yl-2-(2- pyridin-2-yl-ethoxy)- pyrimidin-4-yl]-hydrazine |
| 51 | 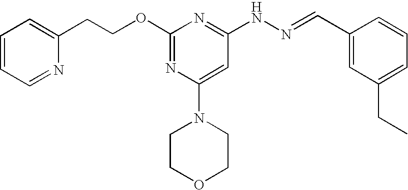 | N-(3-Ethyl-benzylidene)- N′-[6-morpholin-4-yl-2-(2- pyridin-2-yl-ethoxy)- pyrimidin-4-yl]-hydrazine |
| 52 | 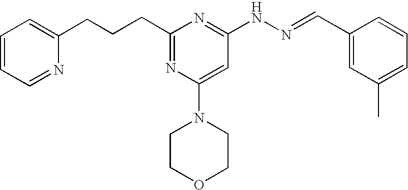 | N-(3-Methyl-benzylidene)- N′-[6-morpholin-4-yl-2-(3- pyridin-2-yl-propyl)- pyrimidin-4-yl]-hydrazine |
| 53 | 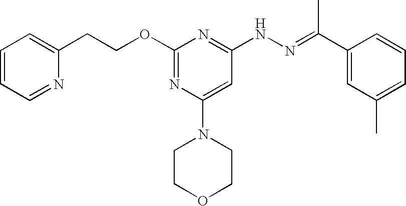 | N-[6-Morpholin-4-yl-2-(2- pyridin-2-yl-ethoxy)- pyrimidin-4-yl]-N′-(1-m- tolyl-ethylidene)-hydrazine |
| 54 | 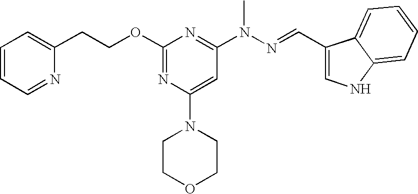 | N′-(1H-Indol-3-ylmethylene)- N-methyl-N-[6-morpholin-4- yl-2-(2-pyridin-2-yl-ethoxy)- pyrimidin-4-yl]-hydrazine |
| 55 | 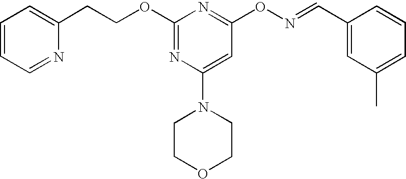 | 3-Methyl-benzaldehyde O-[6-morpholin-4-yl-2-(2- pyridin-2-yl-ethoxy)- pyrimidin-4-yl]-oxime |
| 56 | 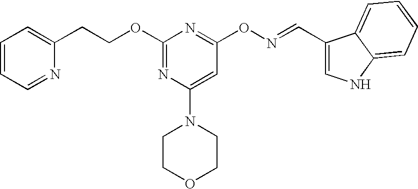 | 1H-Indole-3-carbaldehyde O-[6-morpholin-4-yl-2-(2- pyridin-2-yl-ethoxy)- pyrimidin-4-yl]-oxime |
| 57 | 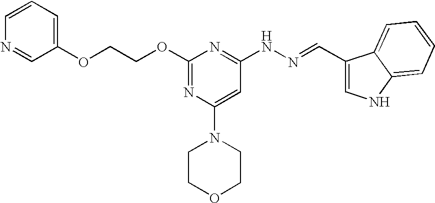 | N-(1H-Indol-3-ylmethylene)- N′-{6-morpholin-4-yl-2-[2- (pyridin-3-yloxy)-ethoxy]- pyrimidin-4-yl}-hydrazine |
| 58 | 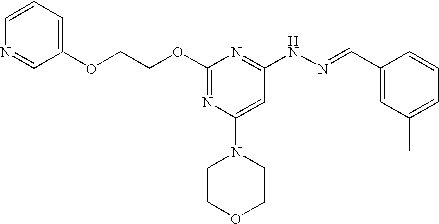 | N-(3-Methyl-benzylidene)- N′-{6-morpholin-4-yl-2-[2- (pyridin-3-yloxy)-ethoxy]- pyrimidin-4-yl}-hydrazine |
| 59 | 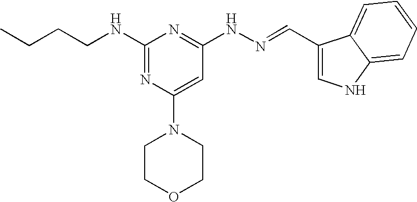 | Butyl-{4-[N′-(1H-indol-3- ylmethylene)-hydrazino]- 6-morpholin-4-yl-pyrimidin- 2-yl}-amine |
| 60 | 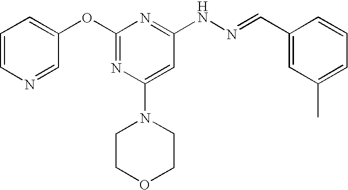 | N-(3-Methyl-benzylidene)- N′-[6-morpholin-4-yl-2- (pyridin-3-yloxy)- pyrimidin-4-yl]-hydrazine |
| 61 | 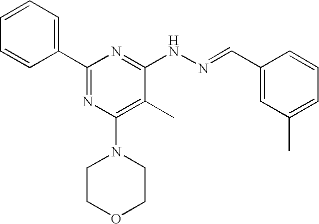 | N-(3-Methyl-benzylidene)- N′-(5-methyl-6-morpholin- 4-yl-2-phenyl-pyrimidin- 4-yl)-hydrazine |
| 62 | 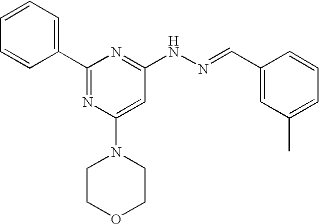 | N-(3-Methyl-benzylidene)- N′-(6-morpholin-4-yl-2- phenyl-pyrimidin-4-yl)- hydrazine |
| 63 | 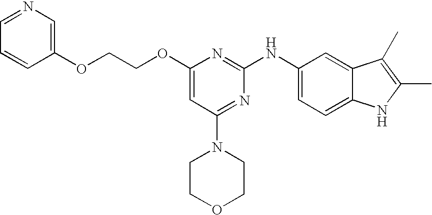 | (2,3-Dimethyl-1H-indol- 5-yl)-{4-morpholin-4-yl-6- [2-(pyridin-3-yloxy)-ethoxy]- pyrimidin-2-yl}-amine |
| 64 | 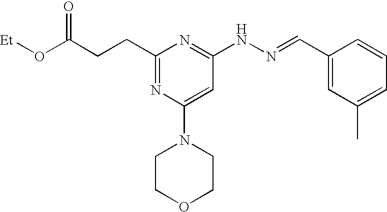 | 3-{4-[N′-(3-Methyl- benzylidene)-hydrazino]-6- morpholin-4-yl-pyrimidin-2- yl}-propionic acid ethyl ester |
| 65 | 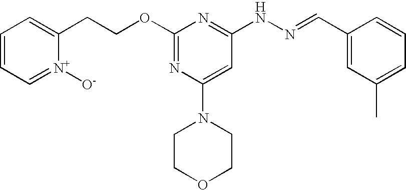 | N-(3-Methyl-benzylidene)- N′-{6-morpholin-4-yl-2-[2- (1-oxy-pyridin-2-yl)-ethoxy]- pyrimidin-4-yl}-hydrazine |
| 66 | 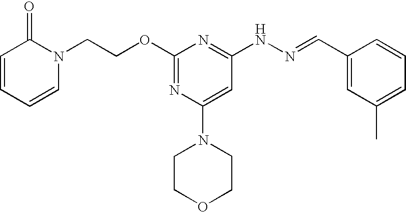 | 1-(2-{4-[N′-(3-Methyl- benzylidene)-hydrazino]-6- morpholin-4-yl-pyrimidin- 2-yloxy}-ethyl)-1H- pyridin-2-one |
| 67 | 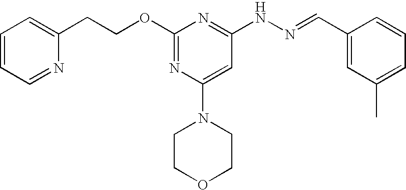 | N-(3-Iodo-benzylidene)- N′-[6-morpholin-4-yl-2- (2-pyridin-2-yl-ethoxy)- pyrimidin-4-yl]-hydrazine |
| 68 | 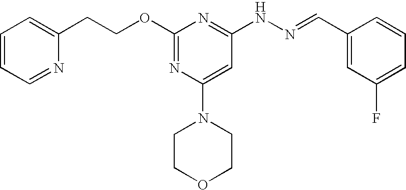 | N-(3-Fluoro-benzylidene)- N′-[6-morpholin-4-yl-2-(2- pyridin-2-yl-ethoxy)- pyrimidin-4-yl]-hydrazine |
| 69 | 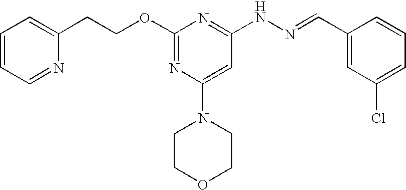 | N-(3-Chloro-benzylidene)- N′-[6-morpholin-4-yl-2-(2- pyridin-2-yl-ethoxy)- pyrimidin-4-yl]-hydrazine |
| 70 | 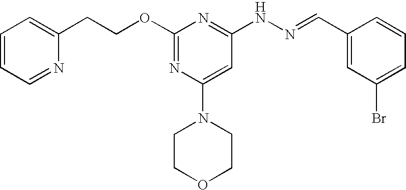 | N-(3-Bromo-benzylidene)- N′-[6-morpholin-4-yl-2-(2- pyridin-2-yl-ethoxy)- pyrimidin-4-yl]-hydrazine |
| 71 | 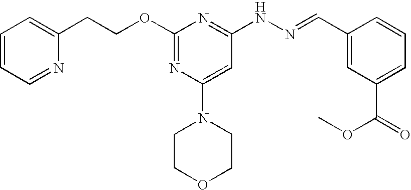 | 3-{[6-Morpholin-4-yl-2- (2-pyridin-2-yl-ethoxy)- pyrimidin-4-yl]- hydrazonomethyl}-benzoic acid methyl ester |
| 72 | 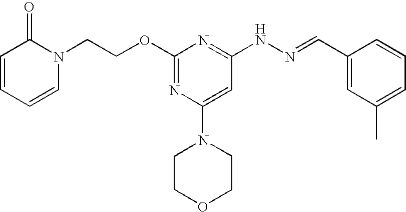 | 1-(2-{4-[N′-(3-Iodo- benzylidene)-hydrazino]- 6-morpholin-4-yl- pyrimidin-2-yloxy}-ethyl)- 1H-pyridin-2-one |
| 73 | 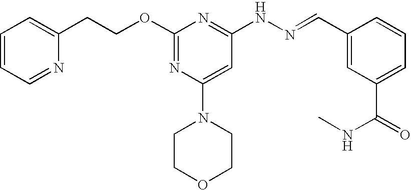 | N-Methyl-3-{[6-morpholin- 4-yl-2-(2-pyridin-2-yl- ethoxy)-pyrimidin-4-yl]- hydrazonomethyl}-benzamide |
| 74 | 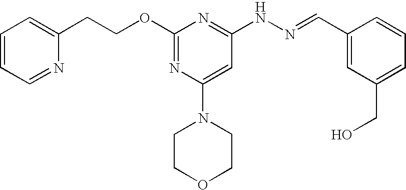 | (3-{[6-Morpholin-4-yl-2- (2-pyridin-2-yl-ethoxy)- pyrimidin-4-yl]- hydrazonomethyl}-phenyl)- methanol |
| 75 | 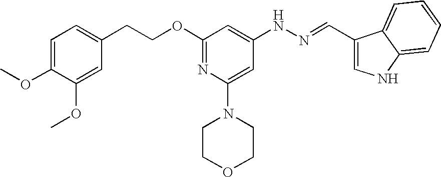 | N-{2-[2-(3,4-Dimethoxy- phenyl)-ethoxy]-6- morpholin-4-yl-pyridin-4- yl}-N′-(1H-indol-3- ylmethylene)-hydrazine |
| 76 | 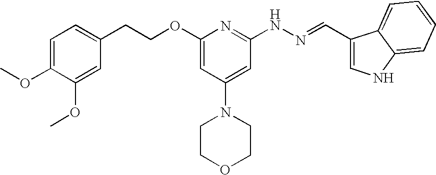 | N-{6-[2-(3,4-Dimethoxy- phenyl)-ethoxy]-4- morpholin-4-yl-pyridin- 2-yl}-N′-(1H-indol-3- ylmethylene)-hydrazine |
| 77 | 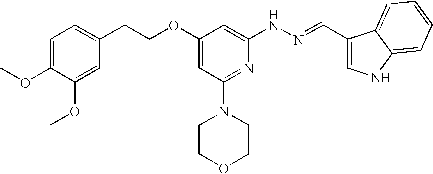 | N-{4-[2-(3,4-Dimethoxy- phenyl)-ethoxy]-6- morpholin-4-yl-pyridin- 2-yl}-N′-(1H-indol-3- ylmethylene)-hydrazine |
| 78 | 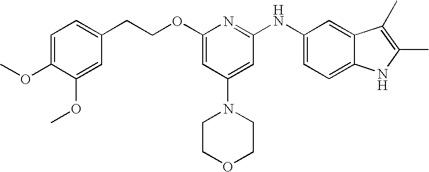 | {6-[2-(3,4-Dimethoxy- phenyl)-ethoxy]-4- morpholin-4-yl-pyridin- 2-yl}-(2,3-dimethyl- 1H-indol-5-yl)-amine |
| 79 | 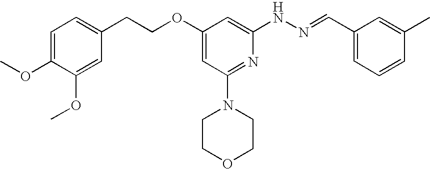 | N-{4-[2-(3,4-Dimethoxy- phenyl)-ethoxy]-6- morpholin-4-yl-pyridin- 2-yl}-N′-(3-methyl- benzylidene)-hydrazine |
| 80 | 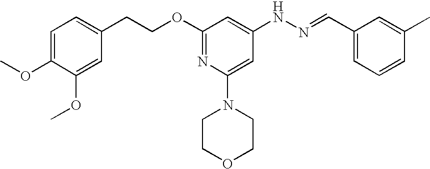 | N-{2-[2-(3,4-Dimethoxy- phenyl)-ethoxy]-6- morpholin-4-yl-pyridin- 4-yl}-N′-(3-methyl- benzylidene)-hydrazine |
| 81 | 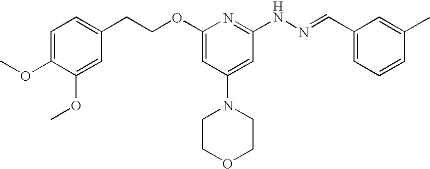 | N-{6-[2-(3,4-Dimethoxy- phenyl)-ethoxy]-4- morpholin-4-yl-pyridin- 2-yl}-N′-(3-methyl- benzylidene)-hydrazine |
| 82 | 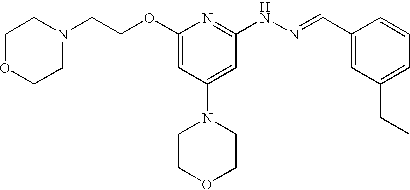 | N-(3-Ethyl-benzylidene)-N′-[4- morpholin-4-yl-6-(2- morpholin-4-yl-ethoxy)- pyridin-2-yl]-hydrazine |
| 83 | 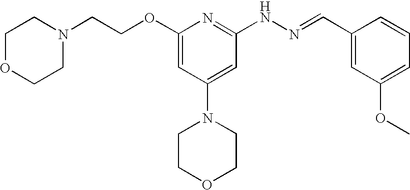 | N-(3-Methoxy-benzylidene)- N′-[4-morpholin-4-yl-6-(2- morpholin-4-yl-ethoxy)- pyridin-2-yl]-hydrazine |
| 84 | 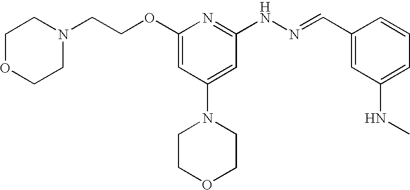 | Methyl-(3-{[4-morpholin-4-yl- 6-(2-morpholin-4-yl-ethoxy)- pyridin-2-yl]- hydrazonomethyl}-phenyl)- amine |
| 85 | 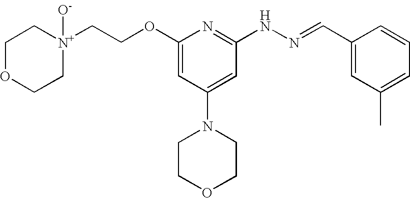 | N-(3-Methyl-benzylidene)-N′- {4-morpholin-4-yl-6-[2-(4-oxy- morpholin-4-yl)-ethoxy]- pyridin-2-yl}-hydrazine |
| 86 | 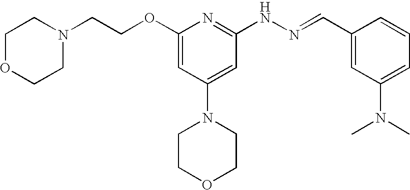 | Dimethyl-(3-{[4-morpholin-4- yl-6-(2-morpholin-4-yl- ethoxy)-pyridin-2-yl]- hydrazonomethyl}-phenyl)- amine |
| 87 | 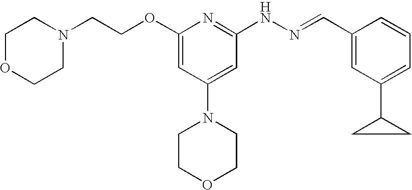 | N-(3-Cyclopropyl- benzylidene)-N′-[4-morpholin- 4-yl-6-(2-morpholin-4-yl- ethoxy)-pyridin-2-yl]- hydrazine |
| 88 | 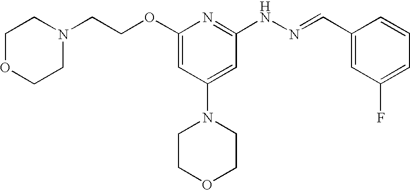 | N-(3-Fluoro-benzylidene)-N′- [4-morpholin-4-yl-6-(2- morpholin-4-yl-ethoxy)- pyridin-2-yl]-hydrazine |
| 89 | 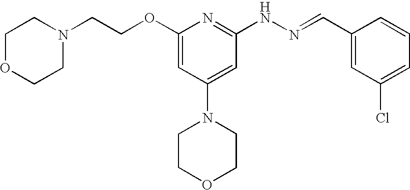 | N-(3-Chloro-benzylidene)-N′- [4-morpholin-4-yl-6-(2- morpholin-4-yl-ethoxy)- pyridin-2-yl]-hydrazine |
| 90 | 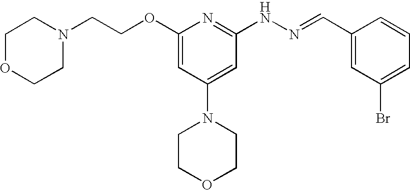 | N-(3-Bromo-benzylidene)-N′- [4-morpholin-4-yl-6-(2- morpholin-4-yl-ethoxy)- pyridin-2-yl]-hydrazine |
| 91 | 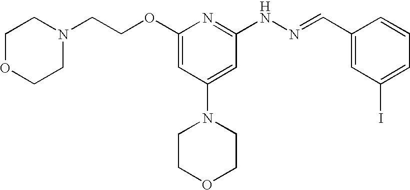 | N-(3-Iodo-benzylidene)-N′-[4- morpholin-4-yl-6-(2- morpholin-4-yl-ethoxy)- pyridin-2-yl]-hydrazine |
| 92 | 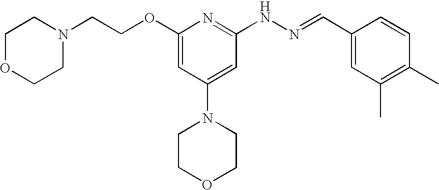 | N-(3,4-Dimethyl-benzylidene)- N′-[4-morpholin-4-yl-6-(2- morpholin-4-yl-ethoxy)- pyridin-2-yl]-hydrazine |
| 93 | 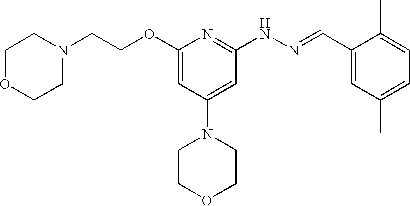 | N-(2,5-Dimethyl-benzylidene)- N′-[4-morpholin-4-yl-6-(2- morpholin-4-yl-ethoxy)- pyridin-2-yl]-hydrazine |
| 94 | 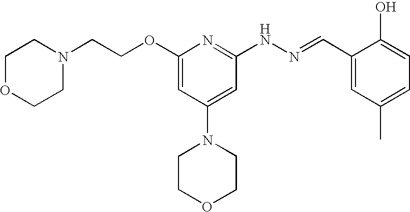 | 4-Methyl-2-{[4-morpholin-4- yl-6-(2-morpholin-4-yl- ethoxy)-pyridin-2-yl]- hydrazonomethyl}-phenol |
| 95 | 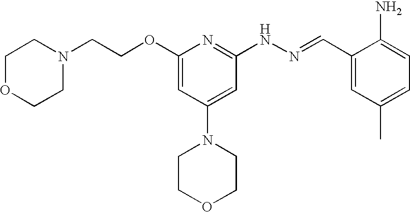 | 4-Methyl-2-{[4-morpholin-4- yl-6-(2-morpholin-4-yl- ethoxy)-pyridin-2-yl]- hydrazonomethyl}- phenylamine |
| 96 | 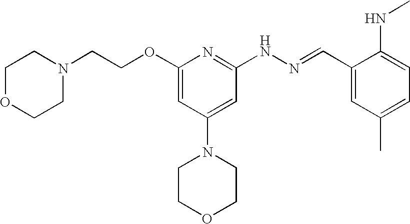 | Methyl-(4-methyl-2-{[4- morpholin-4-yl-6-(2- morpholin-4-yl-ethoxy)- pyridin-2-yl]- hydrazonomethyl}-phenyl)- amine |
| 97 | 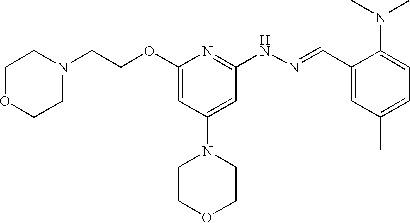 | Dimethyl-(4-methyl-2-{[4- morpholin-4-yl-6-(2- morpholin-4-yl-ethoxy)- pyridin-2-yl]- hydrazonomethyl}-phenyl)- amine |
| 98 | 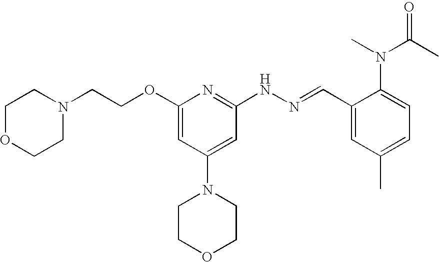 | N-Methyl-N-(4-methyl-2-{[4- morpholin-4-yl-6-(2- morpholin-4-yl-ethoxy)- pyridin-2-yl]- hydrazonomethyl}-phenyl)- acetamide |
| 99 | 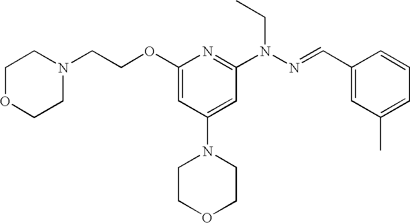 | N-Ethyl-N′-(3-methyl- benzylidene)-N-[4-morpholin- 4-yl-6-(2-morpholin-4-yl- ethoxy)-pyridin-2-yl]- hydrazine |
| 100 | 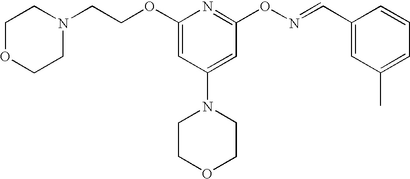 | 3-Methyl-benzaldehyde O-[4- morpholin-4-yl-6-(2- morpholin-4-yl-ethoxy)- pyridin-2-yl]-oxime |
| 101 | 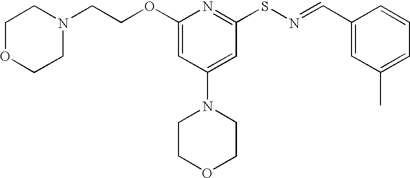 | 3-Methyl-benzaldehyde O-[4- morpholin-4-yl-6-(2- morpholin-4-yl-ethoxy)- pyridin-2-yl]-thiooxime |
| 102 | 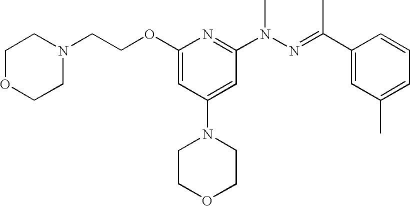 | N-Methyl-N-[4-morpholin-4- yl-6-(2-morpholin-4-yl- ethoxy)-pyridin-2-yl]-N′-(1-m- tolyl-ethylidene)-hydrazine |
| 103 | 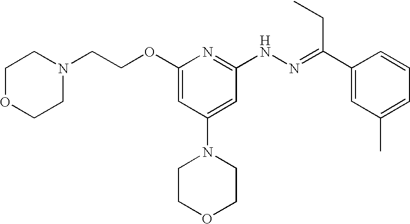 | N-[4-Morpholin-4-yl-6-(2- morpholin-4-yl-ethoxy)- pyridin-2-yl]-N′-( 1-m-tolyl- propylidene)-hydrazine |
| 104 | 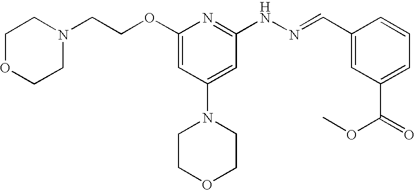 | 3-{[4-Morpholin-4-yl-6-(2- morpholin-4-yl-ethoxy)- pyridin-2-yl]- hydrazonomethyl}-benzoic acid methyl ester |
| 105 | 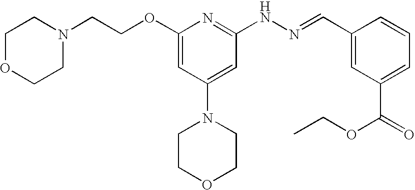 | 3-{[4-Morpholin-4-yl-6-(2- morpholin-4-yl-ethoxy)- pyridin-2-yl]- hydrazonomethyl}-benzoic acid ethyl ester |
| 106 | 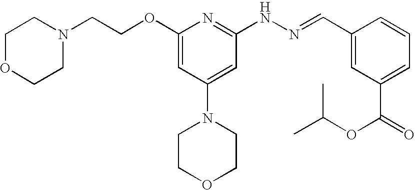 | 3-{[4-Morpholin-4-yl-6-(2- morpholin-4-yl-ethoxy)- pyridin-2-yl]- hydrazonomethyl}-benzoic acid isopropyl ester |
| 107 | 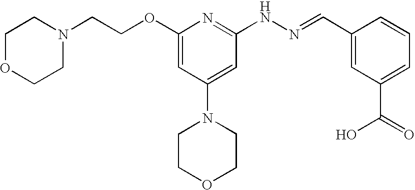 | 3-{[4-Morpholin-4-yl-6-(2- morpholin-4-yl-ethoxy)- pyridin-2-yl]- hydrazonomethyl}-benzoic acid |
| 108 | 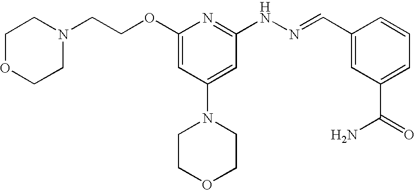 | 3-{[4-Morpholin-4-yl-6-(2- morpholin-4-yl-ethoxy)- pyridin-2-yl]- hydrazonomethyl}-benzamide |
| 109 | 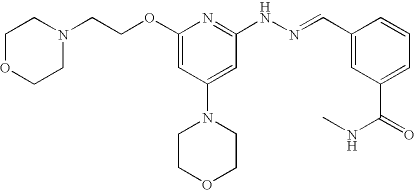 | N-Methyl-3-{[4-morpholin-4- yl-6-(2-morpholin-4-yl- ethoxy)-pyridin-2-yl]- hydrazonomethyl}-benzamide |
| 110 | 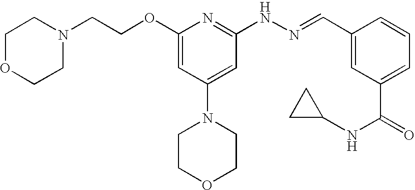 | N-Cyclopropyl-3-{[4- morpholin-4-yl-6-(2- morpholin-4-yl-ethoxy)- pyridin-2-yl]- hydrazonomethyl}-benzamide |
| 111 | 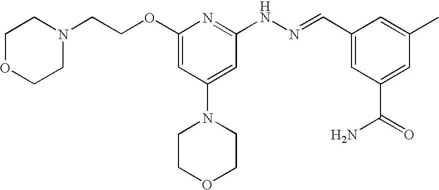 | 3-Methyl-5-{[4-morpholin-4- yl-6-(2-morpholin-4-yl- ethoxy)-pyridin-2-yl]- hydrazonomethyl}-benzamide |
| 112 | 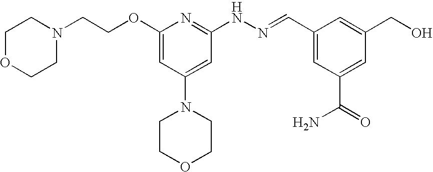 | 3-Hydroxymethyl-5-{[4- morpholin-4-yl-6-(2- morpholin-4-yl-ethoxy)- pyridin-2-yl]- hydrazonomethyl}-benzamide |
| 113 | 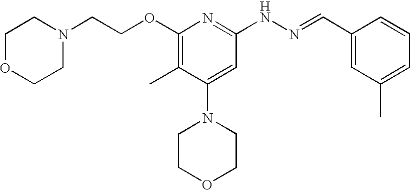 | N-(3-Methyl-benzylidene)-N′- [5-methyl-4-morpholin-4-yl-6- (2-morpholin-4-yl-ethoxy)- pyridin-2-yl]-hydrazine |
| 114 | 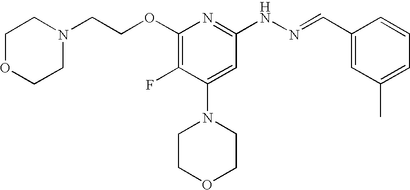 | N-[5-Fluoro-4-morpholin-4-yl- 6-(2-morpholin-4-yl-ethoxy)- pyridin-2-yl]-N′-(3-methyl- benzylidene)-hydrazine |
| 115 | 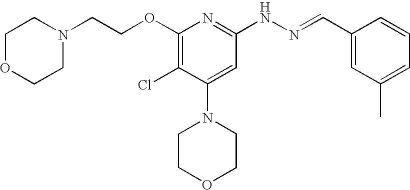 | N-[5-Chloro-4-morpholin-4-yl- 6-(2-morpholin-4-yl-ethoxy)- pyridin-2-yl]-N′-(3-methyl- benzylidene)-hydrazine |
| 116 | 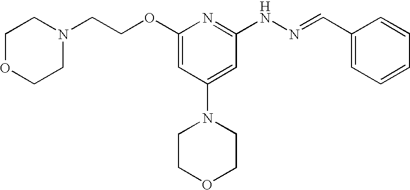 | N-Benzylidene-N′-[4- morpholin-4-yl-6-(2- morpholin-4-yl-ethoxy)- pyridin-2-yl]-hydrazine |
| 117 | 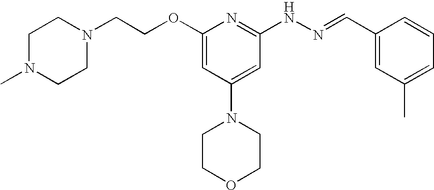 | N-(3-Methyl-benzylidene)-N′- {6-[2-(4-methyl-piperazin-1- yl)-ethoxy]-4-morpholin-4-yl- pyridin-2-yl}-hydrazine |
| 118 | 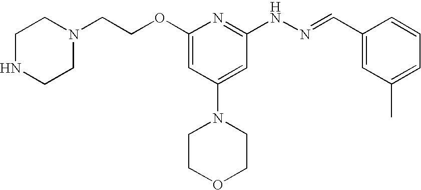 | N-(3-Methyl-benzylidene)-N′- [4-morpholin-4-yl-6-(2- piperazin-1-yl-ethoxy)-pyridin- 2-yl]-hydrazine |
| 119 | 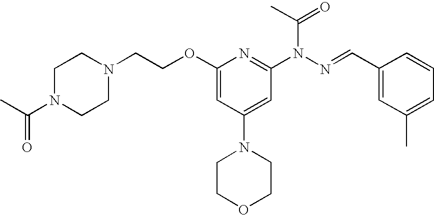 | Acetic acid N-{6-[2-(4-acetyl- piperazin-1-yl)-ethoxy]-4- morpholin-4-yl-pyridin-2-yl}- N′-(3-methyl-benzylidene)- hydrazide |
| 120 | 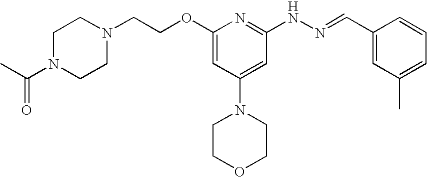 | 1-[4-(2-{6-[N′-(3-Methyl- benzylidene)-hydrazino]-4- morpholin-4-yl-pyridin-2- yloxy}-ethyl)-piperazin-1-yl]- ethanone |
| 121 | 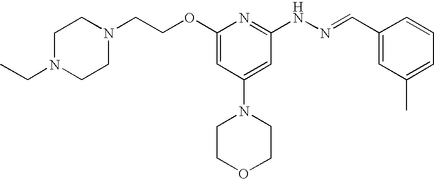 | N-{6-[2-(4-Ethyl-piperazin-1- yl)-ethoxy]-4-morpholin-4-yl- pyridin-2-yl}-N′-(3-methyl- benzylidene)-hydrazine |
| 122 | 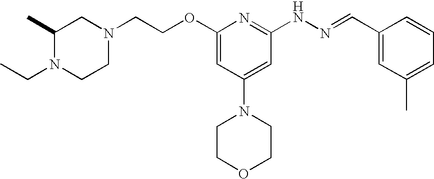 | N-{6-[2-(4-Ethyl-3-methyl- piperazin-1-yl)-ethoxy]-4- morpholin-4-yl-pyridin-2-yl}- N′-(3-methyl-benzylidene)- hydrazine |
| 123 | 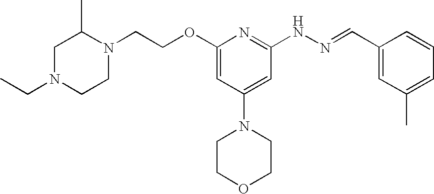 | N-{6-[2-(4-Ethyl-2-methyl- piperazin-1-yl)-ethoxy]-4- morpholin-4-yl-pyridin-2-yl}- N′-(3-methyl-benzylidene)- hydrazine |
| 124 | 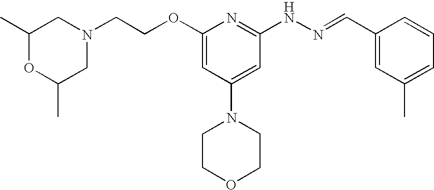 | N-{6-[2-(2,6-Dimethyl- morpholin-4-yl)-ethoxy]-4- morpholin-4-yl-pyridin-2-yl}- N′-(3-methyl-benzylidene)- hydrazine |
| 125 | 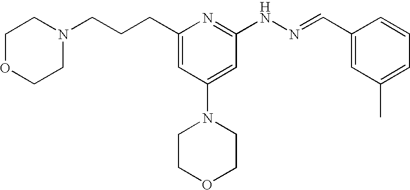 | N-(3-Methyl-benzylidene)-N′- [4-morpholin-4-yl-6-(3- morpholin-4-yl-propyl)- pyridin-2-yl]-hydrazine |
| 126 | 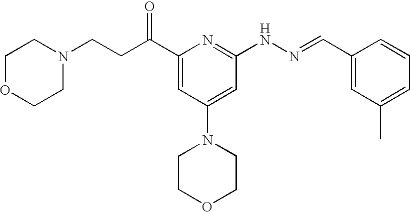 | 1-{6-[N′-(3-Methyl- benzylidene)-hydrazino]-4- morpholin-4-yl-pyridin-2-yl}- 3-morpholin-4-yl-propan-1-one |
| 127 | 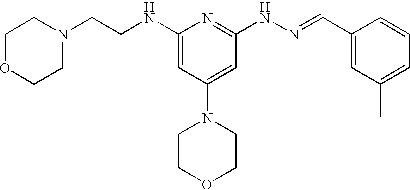 | {6-[N′-(3-Methyl-benzylidene)- hydrazino]-4-morpholin-4-yl- pyridin-2-yl}-(2-morpholin-4- yl-ethyl)-amine |
| 128 | 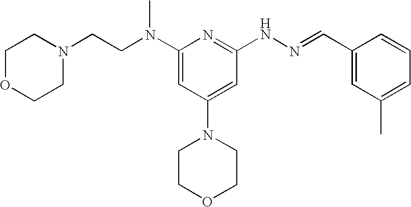 | Methyl-{6-[N′-(3-methyl- benzylidene)-hydrazino]-4- morpholin-4-yl-pyridin-2-yl}- (2-morpholin-4-yl-ethyl)-amine |
| 129 | 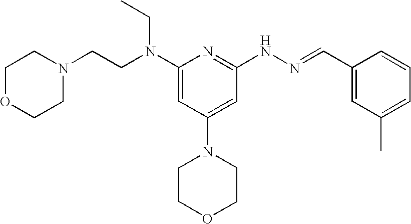 | Ethyl-{6-[N′-(3-methyl- benzylidene)-hydrazino]-4- morpholin-4-yl-pyridin-2-yl}- (2-morpholin-4-yl-ethyl)-amine |
| 130 | 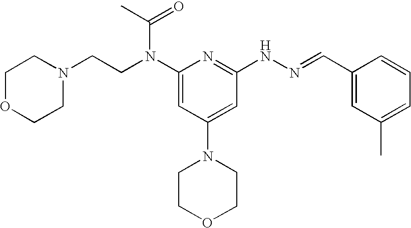 | N-{6-[N′-(3-Methyl- benzylidene)-hydrazino]-4- morpholin-4-yl-pyridin-2-yl}- N-(2-morpholin-4-yl-ethyl)- acetamide |
| 131 | 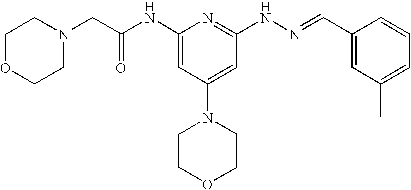 | N-{6-[N′-(3-Methyl- benzylidene)-hydrazino]-4- morpholin-4-yl-pyridin-2-yl}- 2-morpholin-4-yl-acetamide |
| 132 | 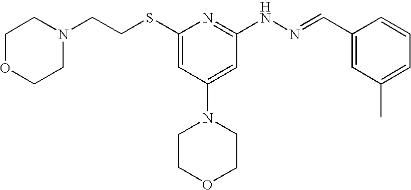 | N-(3-Methyl-benzylidene)-N′- [4-morpholin-4-yl-6-(2- morpholin-4-yl-ethylsulfanyl)- pyridin-2-yl]-hydrazine |
| 133 | 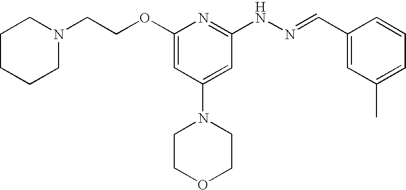 | N-(3-Methyl-benzylidene)-N′- [4-morpholin-4-yl-6-(2- piperidin-1-yl-ethoxy)-pyridin- 2-yl]-hydrazine |
| 134 | 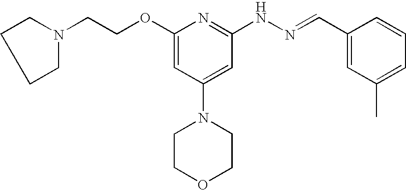 | N-(3-Methyl-benzylidene)-N′- [4-morpholin-4-yl-6-(2- pyrrolidin-1-yl-ethoxy)- pyridin-2-yl]-hydrazine |
| 135 | 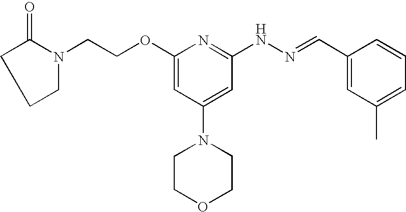 | 1-(2-{6-[N′-(3-Methyl- benzylidene)-hydrazino]-4- morpholin-4-yl-pyridin-2- yloxy}-ethyl)-pyrrolidin-2-one |
| 136 | 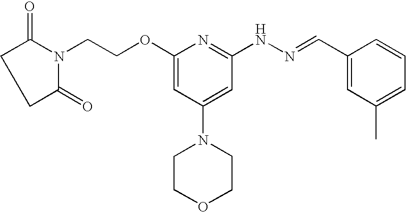 | 1-(2-{6-[N′-(3-Methyl- benzylidene)-hydrazino]-4- morpholin-4-yl-pyridin-2- yloxy}-ethyl)-pyrrolidine-2,5- dione |
| 137 | 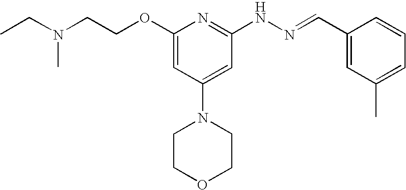 | Ethyl-methyl-(2-{6-[N′-(3- methyl-benzylidene)- hydrazino]-4-morpholin-4-yl- pyridin-2-yloxy}-ethyl)-amine |
| 138 | 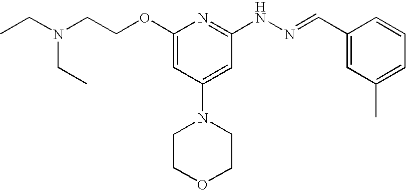 | Diethyl-(2-{6-[N′-(3-methyl- benzylidene)-hydrazino]-4- morpholin-4-yl-pyridin-2- yloxy}-ethyl)-amine |
| 139 | 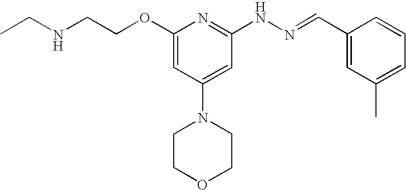 | Ethyl-(2-{6-[N′-(3-methyl- benzylidene)-hydrazino]-4- morpholin-4-yl-pyridin-2- yloxy}-ethyl)-amine |
| 140 | 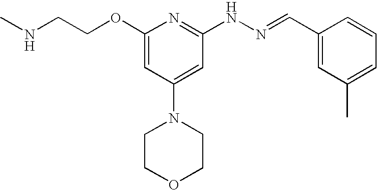 | Methyl-(2-{6-[N′-(3-methyl- benzylidene)-hydrazino]-4- morpholin-4-yl-pyridin-2- yloxy}-ethyl)-amine |
| 141 | 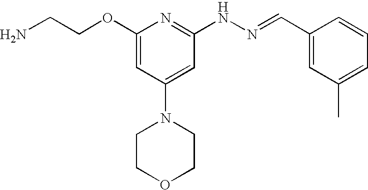 | 2-{6-[N′-(3-Methyl- benzylidene)-hydrazino]-4- morpholin-4-yl-pyridin-2- yloxy}-ethylamine |
| 142 | 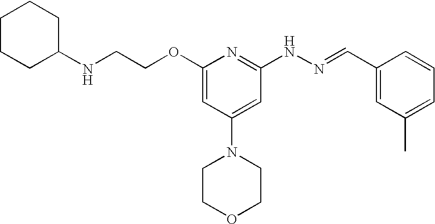 | Cyclohexyl-(2-{6-[N′-(3- methyl-benzylidene)- hydrazino]-4-morpholin-4-yl- pyridin-2-yloxy}-ethyl)-amine |
| 143 | 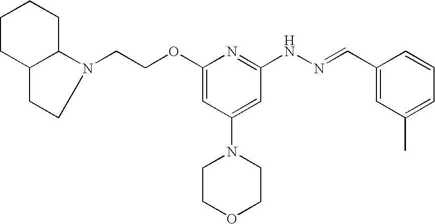 | N-(3-Methyl-benzylidene)-N′- {4-morpholin-4-yl-6-[2- (octahydro-indol-1-yl)-ethoxy]- pyridin-2-yl}-hydrazine |
| 144 | 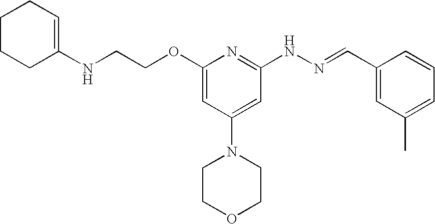 | Cyclohex-1-enyl-(2-{6-[N′-(3- methyl-benzylidene)- hydrazino]-4-morpholin-4-yl- pyridin-2-yloxy}-ethyl)-amine |
| 145 | 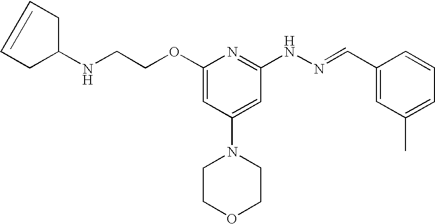 | Cyclopent-3-enyl-(2-{6-[N′-(3- methyl-benzylidene)- hydrazino]-4-morpholin-4-yl- pyridin-2-yloxy}-ethyl)-amine |
| 146 | 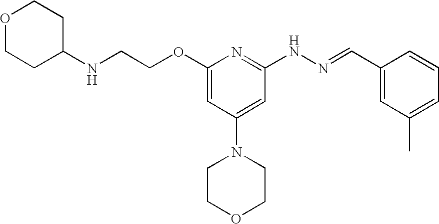 | (2-{6-[N′-(3-Methyl- benzylidene)-hydrazino]-4- morpholin-4-yl-pyridin-2- yloxy}-ethyl)-(tetrahydro- pyran-4-yl)-amine |
| 147 | 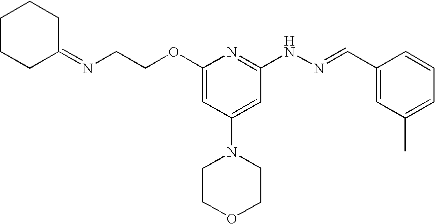 | Cyclohexylidene-(2-{6-[N′-(3- methyl-benzylidene)- hydrazino]-4-morpholin-4-yl- pyridin-2-yloxy}-ethyl)-amine |
| 148 | 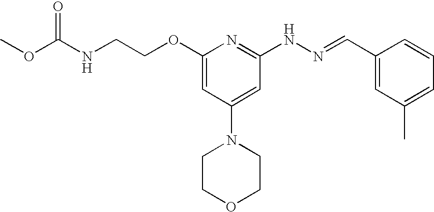 | (2-{6-[N′-(3-Methyl- benzylidene)-hydrazino]-4- morpholin-4-yl-pyridin-2- yloxy}-ethyl)-carbamic acid methyl ester |
| 149 | 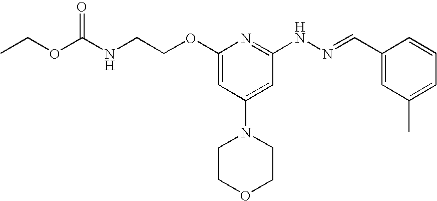 | (2-{6-[N′-(3-Methyl- benzylidene)-hydrazino]-4- morpholin-4-yl-pyridin-2- yloxy}-ethyl)-carbamic acid ethyl ester |
| 150 | 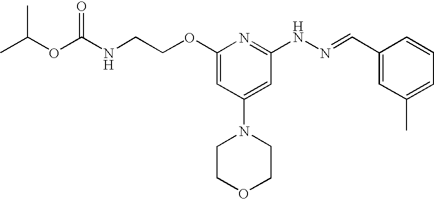 | (2-{6-[N′-(3-Methyl- benzylidene)-hydrazino]-4- morpholin-4-yl-pyridin-2- yloxy}-ethyl)-carbamic acid isopropyl ester |
| 151 | 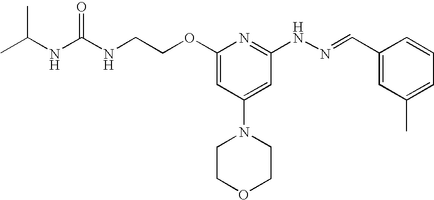 | 1-Isopropyl-3-(2-{6-[N′-(3- methyl-benzylidene)- hydrazino]-4-morpholin-4-yl- pyridin-2-yloxy}-ethyl)-urea |
| 152 | 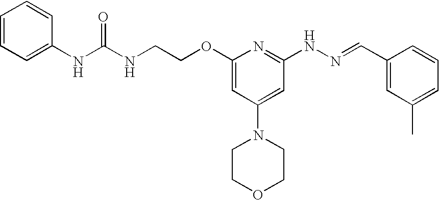 | 1-(2-{6-[N′-(3-Methyl- benzylidene)-hydrazino]-4- morpholin-4-yl-pyridin-2- yloxy}-ethyl)-3-phenyl-urea |
| 153 | 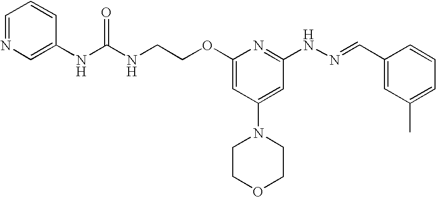 | 1-(2-{6-[N′-(3-Methyl- benzylidene)-hydrazino]-4- morpholin-4-yl-pyridin-2- yloxy}-ethyl)-3-pyridin-3-yl- urea |
| 154 | 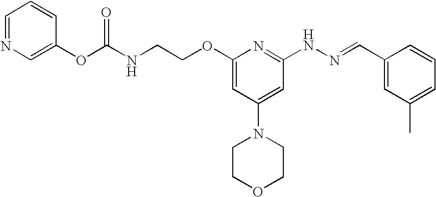 | (2-{6-[N′-(3-Methyl- benzylidene)-hydrazino]-4- morpholin-4-yl-pyridin-2- yloxy}-ethyl)-carbamic acid pyridin-3-yl ester |
| 155 | 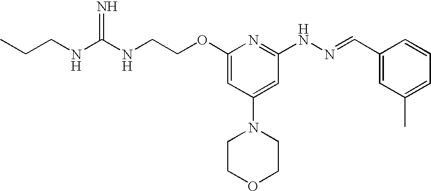 | N-(2-{6-[N′-(3-Methyl- benzylidene)-hydrazino]-4- morpholin-4-yl-pyridin-2- yloxy}-ethyl)-N′-propyl- guanidine |
| 156 | 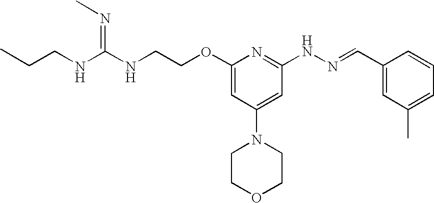 | N-Methyl-N′-(2-{6-[N′-(3- methyl-benzylidene)- hydrazino]-4-morpholin-4-yl- pyridin-2-yloxy}-ethyl)-N″- propyl-guanidine |
| 157 | 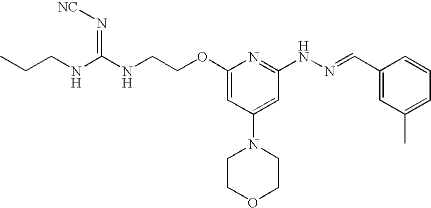 | N-Cyano-N′-(2-{6-[N′-(3- methyl-benzylidene)- hydrazino]-4-morpholin-4-yl- pyridin-2-yloxy}-ethyl)-N″- propyl-guanidine |
| 158 | 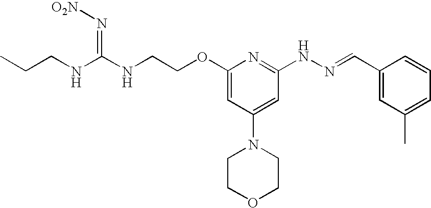 | N-Nitro-N′-(2-{6-[N′-(3- methyl-benzylidene)- hydrazino]-4-morpholin-4-yl- pyridin-2-yloxy}-ethyl)-N″- propyl-guanidine |
| 159 | 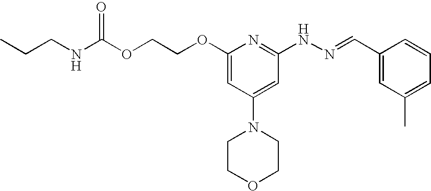 | Propyl-carbamic acid 2-{6-[N′- (3-methyl-benzylidene)- hydrazino]-4-morpholin-4-yl- pyridin-2-yloxy}-ethyl ester |
| 160 | 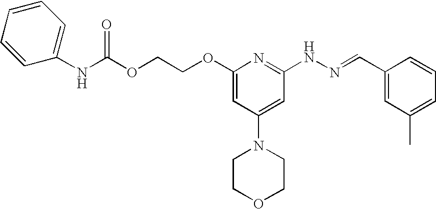 | Phenyl-carbamic acid 2-{6-[N′- (3-methyl-benzylidene)- hydrazino]-4-morpholin-4-yl- pyridin-2-yloxy}-ethyl ester |
| 161 | 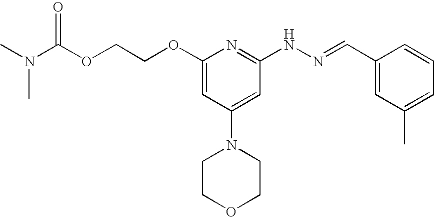 | Dimethyl-carbamic acid 2-{6- [N′-(3-methyl-benzylidene)- hydrazino]-4-morpholin-4-yl- pyridin-2-yloxy}-ethyl ester |
| 162 | 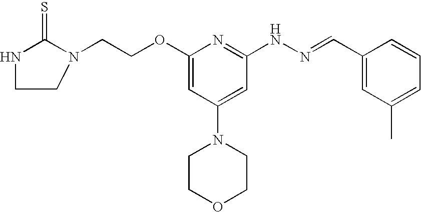 | 1-(2-{6-[N′-(3-Methyl- benzylidene)-hydrazino]-4- morpholin-4-yl-pyridin-2- yloxy}-ethyl)-imidazolidine-2- thione |
| 163 | 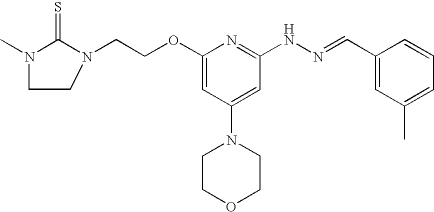 | 1-Methyl-3-(2-{6-[N′-(3- methyl-benzylidene)- hydrazino]-4-morpholin-4-yl- pyridin-2-yloxy}-ethyl)- imidazolidine-2-thione |
| 164 | 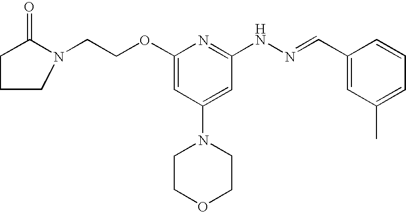 | 1-(2-{6-[N′-(3-Methyl- benzylidene)-hydrazino]-4- morpholin-4-yl-pyridin-2- yloxy}-ethyl)-pyrrolidin-2-one |
| 165 | 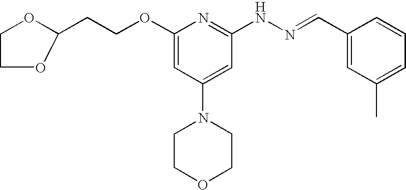 | N-[6-(2-[1,3]Dioxolan-2-yl- ethoxy)-4-morpholin-4-yl- pyridin-2-yl]-N′-(3-methyl- benzylidene)-hydrazine |
| 166 | 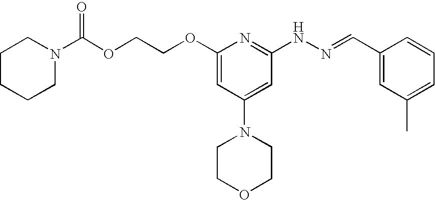 | Piperidine-1-carboxylic acid 2- {6-[N′-(3-methyl-benzylidene)- hydrazino]-4-morpholin-4-yl- pyridin-2-yloxy}-ethyl ester |
| 167 | 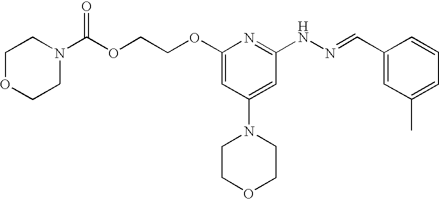 | Morpholine-4-carboxylic acid 2-{6-[N′-(3-methyl- benzylidene)-hydrazino]-4- morpholin-4-yl-pyridin-2- yloxy}-ethyl ester |
| 168 | 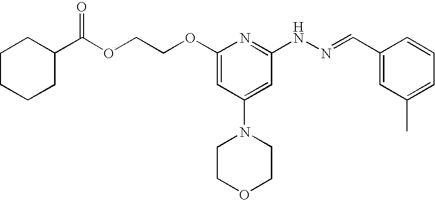 | Cyclohexanecarboxylic acid 2- {6-[N′-(3-methyl-benzylidene)- hydrazino]-4-morpholin-4-yl- pyridin-2-yloxy}-ethyl ester |
| 169 | 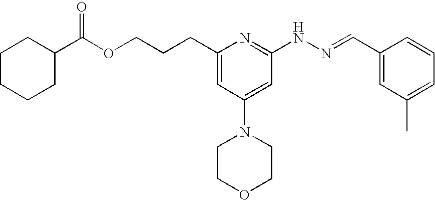 | Cyclohexanecarboxylic acid 3- {6-[N′-(3-methyl-benzylidene)- hydrazino]-4-morpholin-4-yl- pyridin-2-yl}-propyl ester |
| 170 | 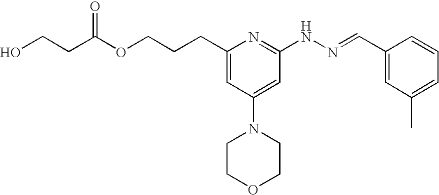 | 3-Hydroxy-propionic acid 3- {6-[N′-(3-methyl-benzylidene)- hydrazino]-4-morpholin-4-yl- pyridin-2-yl}-propyl ester |
| 171 | 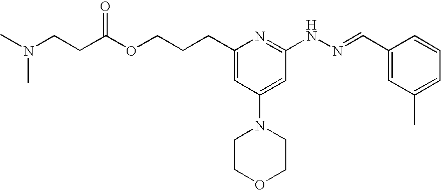 | 3-Dimethylamino-propionic acid 3-{6-[N′-(3-methyl- benzylidene)-hydrazino]-4- morpholin-4-yl-pyridin-2-yl}- propyl ester |
| 172 | 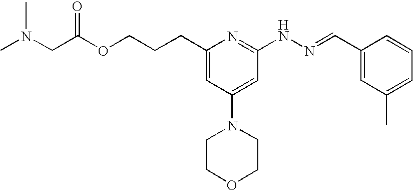 | Dimethylamino-acetic acid 3- {6-[N′-(3-methyl-benzylidene)- hydrazino]-4-morpholin-4-yl- pyridin-2-yl}-propyl ester |
| 173 | 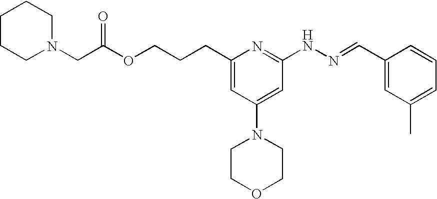 | Piperidin-1-yl-acetic acid 3-{6- [N′-(3-methyl-benzylidene)- hydrazino]-4-morpholin-4-yl- pyridin-2-yl}-propyl ester |
| 174 | 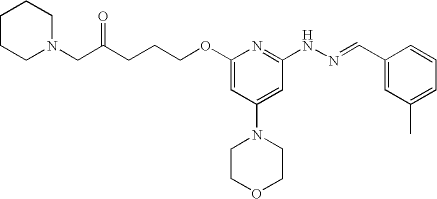 | 5-{6-[N′-(3-Methyl- benzylidene)-hydrazino]-4- morpholin-4-yl-pyridin-2- yloxy}-1-piperidin-1-yl- pentan-2-one |
| 175 | 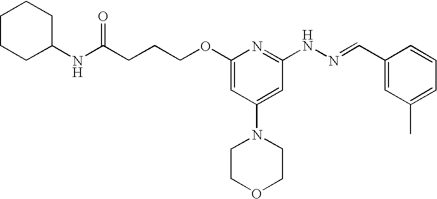 | N-Cyclohexyl-4-{6-[N′-(3- methyl-benzylidene)- hydrazino]-4-morpholin-4-yl- pyridin-2-yloxy}-butyramide |
| 176 | 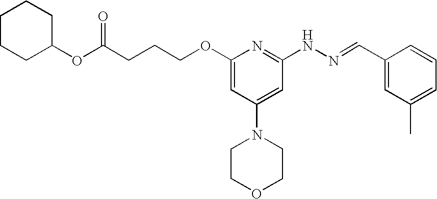 | 4-{6-[N′-(3-Methyl- benzylidene)-hydrazino]-4- morpholin-4-yl-pyridin-2- yloxy}-butyric acid cyclohexyl ester |
| 177 | 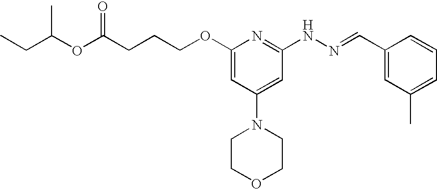 | 4-{6-[N′-(3-Methyl- benzylidene)-hydrazino]-4- morpholin-4-yl-pyridin-2- yloxy}-butyric acid sec-butyl ester |
| 178 | 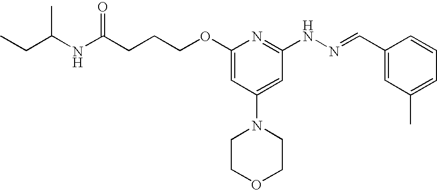 | N-sec-Butyl-4-{6-[N′-(3- methyl-benzylidene)- hydrazino]-4-morpholin-4-yl- pyridin-2-yloxy}-butyramide |
| 179 | 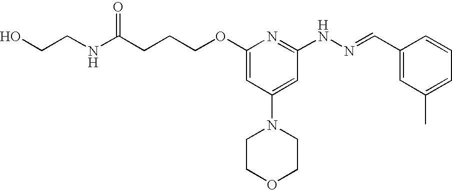 | N-(2-Hydroxy-ethyl)-4-{6-[N′- (3-methyl-benzylidene)- hydrazino]-4-morpholin-4-yl- pyridin-2-yloxy}-butyramide |
| 180 | 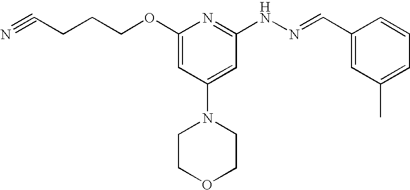 | 4-{6-[N′-(3-Methyl- benzylidene)-hydrazino]-4- morpholin-4-yl-pyridin-2- yloxy}-butyronitrile |
| 181 | 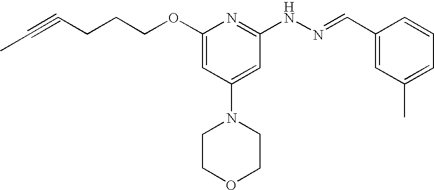 | N-(6-Hex-4-ynyloxy-4- morpholin-4-yl-pyridin-2-yl)- N′-(3-methyl-benzylidene)- hydrazine |
| 182 | 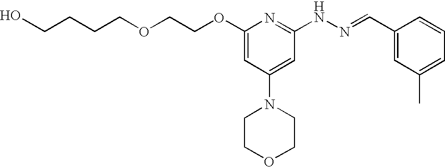 | 4-(2-{6-[N′-(3-Methyl- benzylidene)-hydrazino]-4- morpholin-4-yl-pyridin-2- yloxy}-ethoxy)-butan-1-ol |
| 183 | 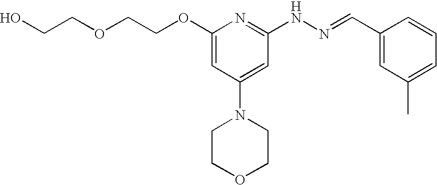 | 2-(2-{6-[N′-(3-Methyl- benzylidene)-hydrazino]-4- morpholin-4-yl-pyridin-2- yloxy}-ethoxy)-ethanol |
| 184 | 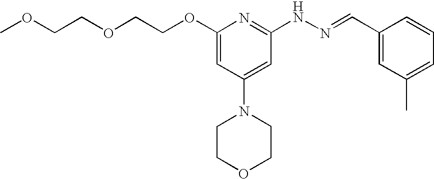 | N-{6-[2-(2-Methoxy-ethoxy)- ethoxy]-4-morpholin-4-yl- pyridin-2-yl}-N′-(3-methyl- benzylidene)-hydrazine |
| 185 | 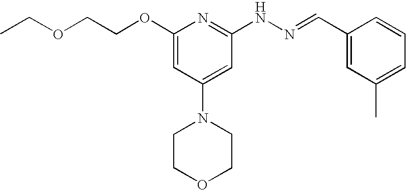 | N-[6-(2-Ethoxy-ethoxy)-4- morpholin-4-yl-pyridin-2-yl]- N′-(3-methyl-benzylidene)- hydrazine |
| 186 | 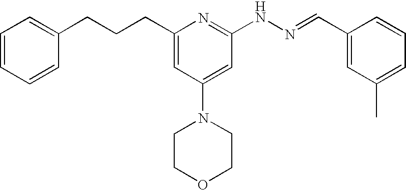 | N-(3-Methyl-benzylidene)-N′- [4-morpholin-4-yl-6-(3-phenyl- propyl)-pyridin-2-yl]-hydrazine |
| 187 | 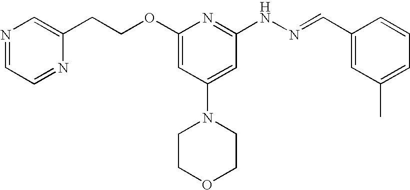 | N-(3-Methyl-benzylidene)-N′- [4-morpholin-4-yl-6-(2- pyrazin-2-yl-ethoxy)-pyridin-2- yl]-hydrazine |
| 188 | 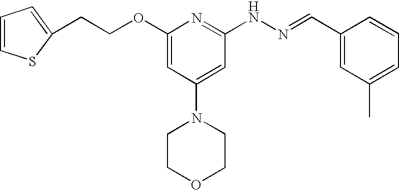 | N-(3-Methyl-benzylidene)-N′- [4-morpholin-4-yl-6-(2- thiophen-2-yl-ethoxy)-pyridin- 2-yl]-hydrazine |
| 189 | 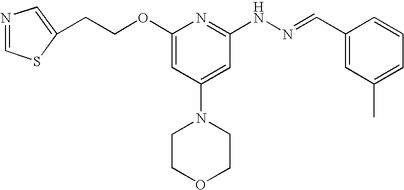 | N-(3-Methyl-benzylidene)-N′- [4-morpholin-4-yl-6-(2-thiazol- 5-yl-ethoxy)-pyridin-2-yl]- hydrazine |
| 190 | 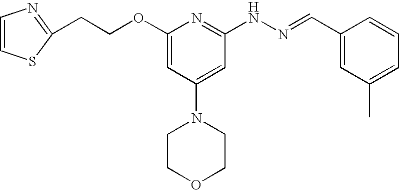 | N-(3-Methyl-benzylidene)-N′- [4-morpholin-4-yl-6-(2-thiazol- 2-yl-ethoxy)-pyridin-2-yl]- hydrazine |
| 191 | 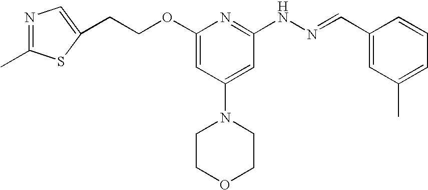 | N-(3-Methyl-benzylidene)-N′- {6-[2-(2-methyl-thiazol-5-yl)- ethoxy]-4-morpholin-4-yl- pyridin-2-yl}-hydrazine |
| 192 | 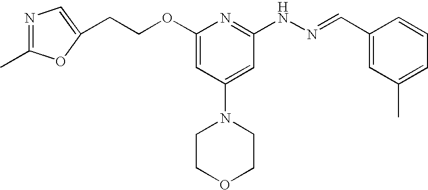 | N-(3-Methyl-benzylidene)-N′- {6-[2-(2-methyl-oxazol-5-yl)- ethoxy]-4-morpholin-4-yl- pyridin-2-yl}-hydrazine |
| 193 | 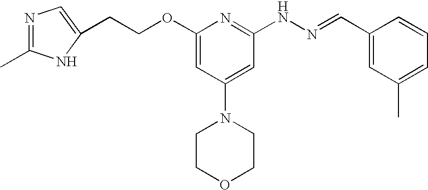 | N-(3-Methyl-benzylidene)-N′- {6-[2-(2-methyl-3H-imidazol- 4-yl)-ethoxy]-4-morpholin-4- yl-pyridin-2-yl}-hydrazine |
| 194 | 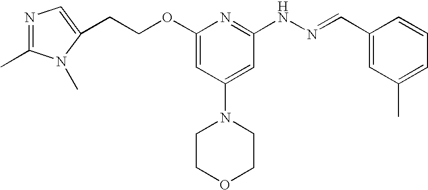 | N-{6-[2-(2,3-Dimethyl-3H- imidazol-4-yl)-ethoxy]-4- morpholin-4-yl-pyridin-2-yl}- N′-(3-methyl-benzylidene)- hydrazine |
| 195 | 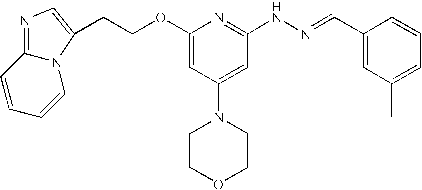 | N-[6-(2-Imidazo[1,2-a]pyridin- 3-yl-ethoxy)-4-morpholin-4-yl- pyridin-2-yl]-N′-(3-methyl- benzylidene)-hydrazine |
| 196 | 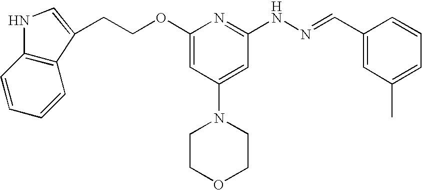 | N-{6-[2-(1H-Indol-3-yl)- ethoxy]-4-morpholin-4-yl- pyridin-2-yl}-N′-(3-methyl- benzylidene)-hydrazine |
| 197 | 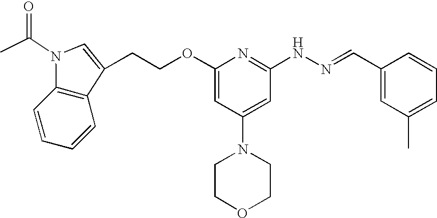 | 1-[3-(2-{6-[N′-(3-Methyl- benzylidene)-hydrazino]-4- morpholin-4-yl-pyridin-2- yloxy}-ethyl)-indol-1-yl]- ethanone |
| 198 | 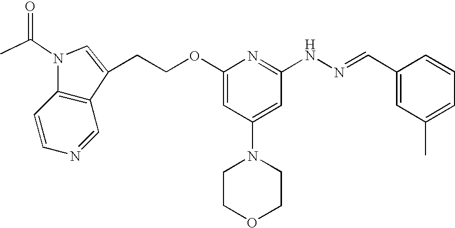 | 1-[3-(2-{6-[N′-(3-Methyl- benzylidene)-hydrazino]-4- morpholin-4-yl-pyridin-2- yloxy}-ethyl)-pyrrolo[3,2- c]pyridin-1-yl]-ethanone |
| 199 | 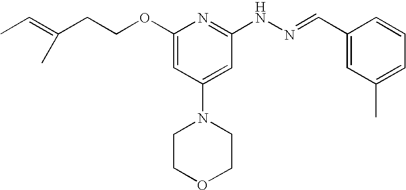 | N-(3-Methyl-benzylidene)-N′- [6-(3-methyl-pent-3-enyloxy)- 4-morpholin-4-yl-pyridin-2-yl]- hydrazine |
| 200 | 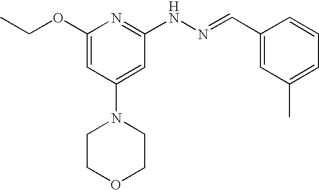 | N-(6-Ethoxy-4-morpholin-4-yl- pyridin-2-yl)-N′-(3-methyl- benzylidene)-hydrazine |
| 202 | 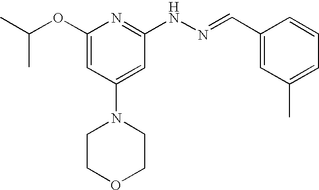 | N-(6-Isopropoxy-4-morpholin- 4-yl-pyridin-2-yl)-N′-(3- methyl-benzylidene)-hydrazine |
| 203 | 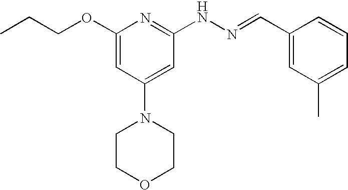 | N-(3-Methyl-benzylidene)-N′- (4-morpholin-4-yl-6-propoxy- pyridin-2-yl)-hydrazine |
| 204 | 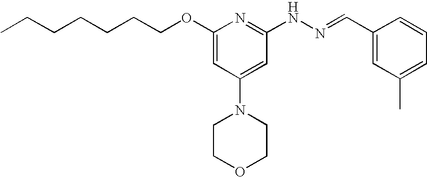 | N-(6-Heptyloxy-4-morpholin- 4-yl-pyridin-2-yl)-N′-(3- methyl-benzylidene)-hydrazine |
| 205 | 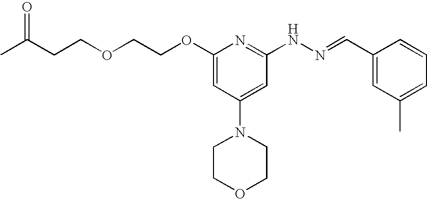 | 4-(2-{6-[N′-(3-Methyl- benzylidene)-hydrazino]-4- morpholin-4-yl-pyridin-2- yloxy}-ethoxy)-butan-2-one |
| 206 | 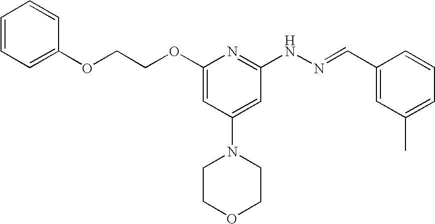 | N-(3-Methyl-benzylidene)-N′- [4-morpholin-4-yl-6-(2- phenoxy-ethoxy)-pyridin-2-yl]- hydrazine |
| 207 | 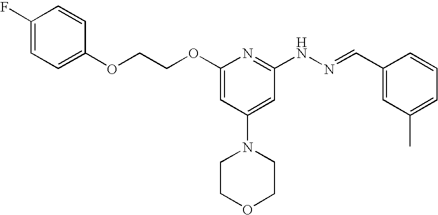 | N-{6-[2-(4-Fluoro-phenoxy)- ethoxy]-4-morpholin-4-yl- pyridin-2-yl}-N′-(3-methyl- benzylidene)-hydrazine |
| 208 | 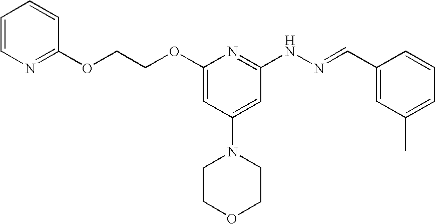 | N-(3-Methyl-benzylidene)-N′- {4-morpholin-4-yl-6-[2- (pyridin-2-yloxy)-ethoxy]- pyridin-2-yl}-hydrazine |
| 209 | 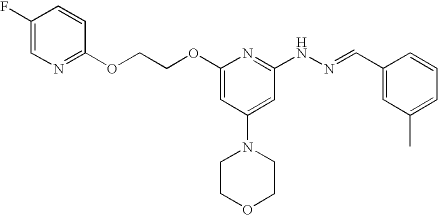 | N-{6-[2-(5-Fluoro-pyridin-2- yloxy)-ethoxy]-4-morpholin-4- yl-pyridin-2-yl}-N′-(3-methyl- benzylidene)-hydrazine |
| 211 | 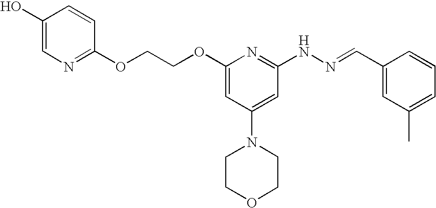 | 6-(2-{6-[N′-(3-Methyl- benzylidene)-hydrazino]-4- morpholin-4-yl-pyridin-2- yloxy}-ethoxy)-pyridin-3-ol |
| 212 | 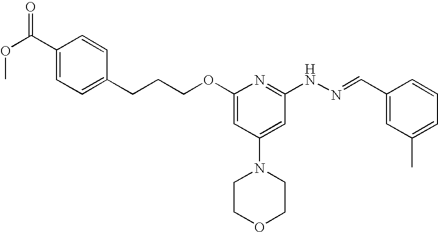 | 4-(3-{6-[N′-(3-Methyl- benzylidene)-hydrazino]-4- morpholin-4-yl-pyridin-2- yloxy}-propyl)-benzoic acid methyl ester |
| 213 | 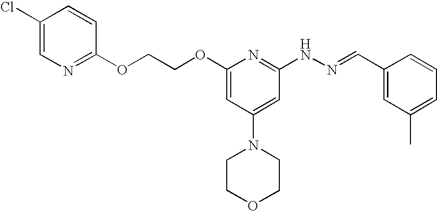 | N-{6-[2-(5-Chloro-pyridin-2- yloxy)-ethoxy]-4-morpholin-4- yl-pyridin-2-yl}-N′-(3-methyl- benzylidene)-hydrazine |
| 214 | 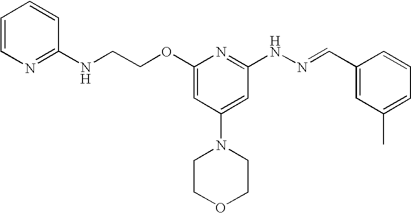 | (2-{6-[N′-(3-Methyl- benzylidene)-hydrazino]-4- morpholin-4-yl-pyridin-2- yloxy}-ethyl)-pyridin-2-yl- amine |
| 215 | 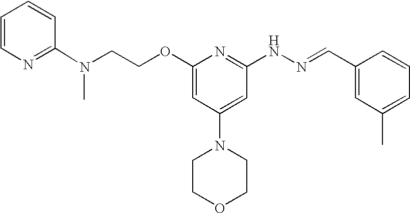 | Methyl-(2-{6-[N′-(3-methyl- benzylidene)-hydrazino]-4- morpholin-4-yl-pyridin-2- yloxy}-ethyl)-pyridin-2-yl- amine |
| 216 | 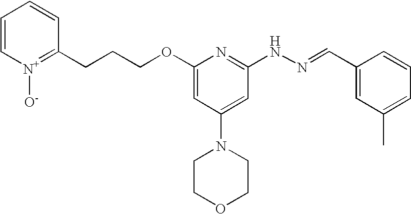 | N-(3-Methyl-benzylidene)-N′- {4-morpholin-4-yl-6-[3-(1-oxy- pyridin-2-yl)-propoxy]-pyridin- 2-yl}-hydrazine |
| 217 | 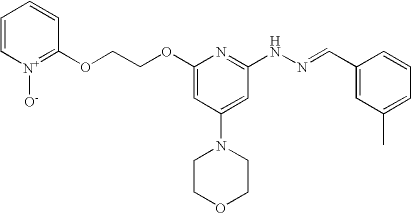 | N-(3-Methyl-benzylidene)-N′- {4-morpholin-4-yl-6-[2-(1-oxy- pyridin-2-yloxy)-ethoxy]- pyridin-2-yl}-hydrazine |
| 218 | 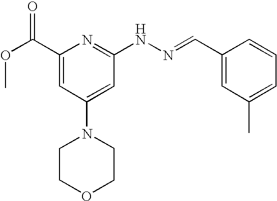 | 6-[N′-(3-Methyl-benzylidene)- hydrazino]-4-morpholin-4-yl- pyridine-2-carboxylic acid methyl ester |
| 219 | 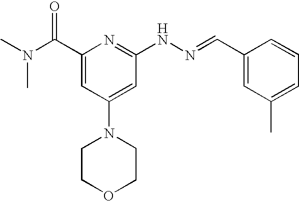 | 6-[N′-(3-Methyl-benzylidene)- hydrazino]-4-morpholin-4-yl- pyridine-2-carboxylic acid dimethylamide |
| 220 | 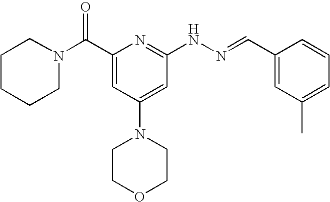 | {6-[N′-(3-Methyl-benzylidene)- hydrazino]-4-morpholin-4-yl- pyridin-2-yl}-piperidin-1-yl- methanone |
| 221 | 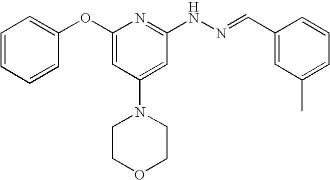 | N-(3-Methyl-benzylidene)-N′- (4-morpholin-4-yl-6-phenoxy- pyridin-2-yl)-hydrazine |
| 222 | 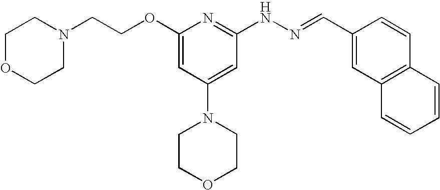 | N-[4-Morpholin-4-yl-6-(2- morpholin-4-yl-ethoxy)- pyridin-2-yl]-N′-naphthalen-2- ylmethylene-hydrazine |
| 223 | 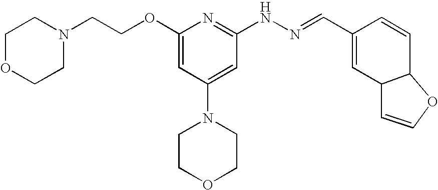 | N-Benzofuran-5-ylmethylene- N′-[4-morpholin-4-yl-6-(2- morpholin-4-yl-ethoxy)- pyridin-2-yl]-hydrazine |
| 224 | 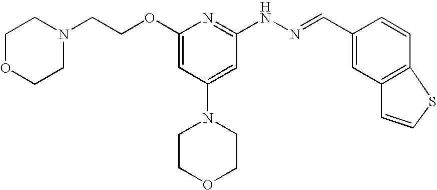 | N-Benzo[b]thiophen-5- ylmethylene-N′-[4-morpholin- 4-yl-6-(2-morpholin-4-yl- ethoxy)-pyridin-2-yl]- hydrazine |
| 225 | 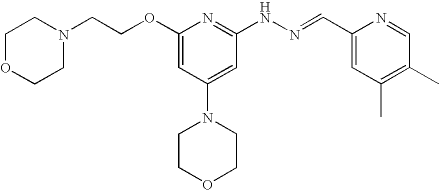 | N-(4,5-Dimethyl-pyridin-2- ylmethylene)-N′-[4-morpholin- 4-yl-6-(2-morpholin-4-yl- ethoxy)-pyridin-2-yl]- hydrazine |
| 226 | 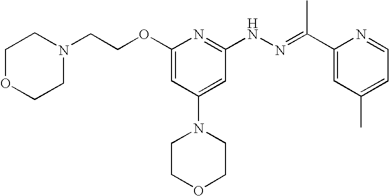 | N-[1-(4-Methyl-pyridin-2-yl)- ethylidene]-N′-[4-morpholin-4- yl-6-(2-morpholin-4-yl- ethoxy)-pyridin-2-yl]- hydrazine |
| 227 | 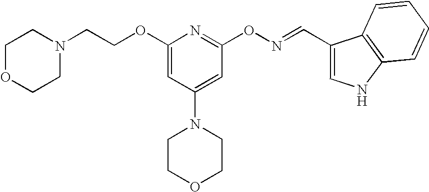 | 1H-Indole-3-carbaldehyde O- [4-morpholin-4-yl-6-(2- morpholin-4-yl-ethoxy)- pyridin-2-yl]-oxime |
| 228 | 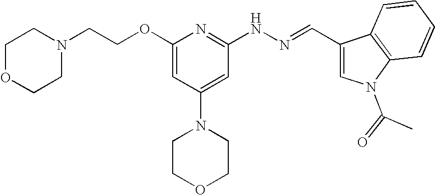 | 1-(3-{[4-Morpholin-4-yl-6-(2- morpholin-4-yl-ethoxy)- pyridin-2-yl]- hydrazonomethyl}-indol-1-yl)- ethanone |
| 229 | 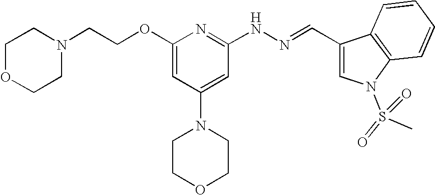 | N-(1-Methanesulfonyl-1H- indol-3-ylmethylene)-N′-[4- morpholin-4-yl-6-(2- morpholin-4-yl-ethoxy)- pyridin-2-yl]-hydrazine |
| 230 | 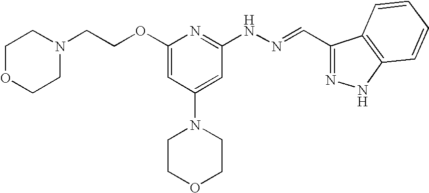 | N-(1H-Indazol-3-ylmethylene)- N′-[4-morpholin-4-yl-6-(2- morpholin-4-yl-ethoxy)- pyridin-2-yl]-hydrazine |
| 231 | 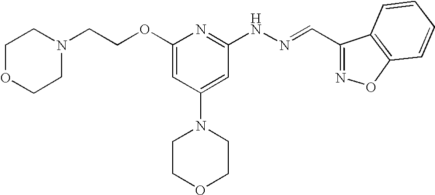 | N-Benzo[d]isoxazol-3- ylmethylene-N′-[4-morpholin- 4-yl-6-(2-morpholin-4-yl- ethoxy)-pyridin-2-yl]- hydrazine |
| 232 | 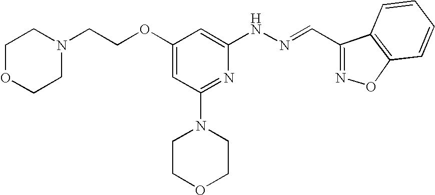 | N-Benzo[d]isoxazol-3- ylmethylene-N′-[6-morpholin- 4-yl-4-(2-morpholin-4-yl- ethoxy)-pyridin-2-yl]- hydrazine |
| 233 | 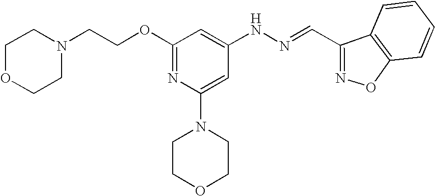 | N-Benzo[d]isoxazol-3- ylmethylene-N′-[2-morpholin- 4-yl-6-(2-morpholin-4-yl- ethoxy)-pyridin-4-yl]- hydrazine |
| 234 | 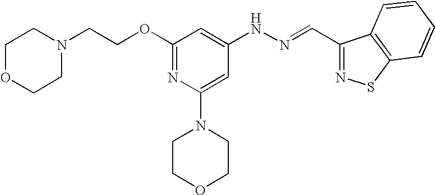 | N-Benzo[d]isothiazol-3- ylmethylene-N′-[2-morpholin- 4-yl-6-(2-morpholin-4-yl- ethoxy)-pyridin-4-yl]- hydrazine |
| 235 | 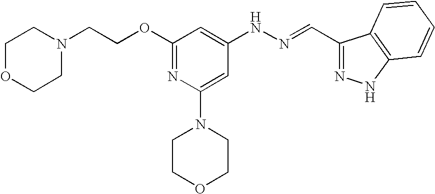 | N-(1H-Indazol-3-ylmethylene)- N′-[2-morpholin-4-yl-6-(2- morpholin-4-yl-ethoxy)- pyridin-4-yl]-hydrazine |
| 236 | 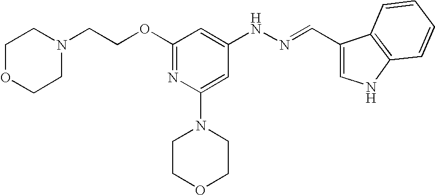 | N-(1H-Indol-3-ylmethylene)- N′-[2-morpholin-4-yl-6-(2- morpholin-4-yl-ethoxy)- pyridin-4-yl]-hydrazine |
| 237 | 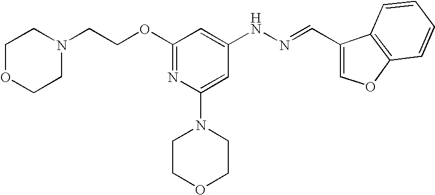 | N-Benzofuran-3-ylmethylene- N′-[2-morpholin-4-yl-6-(2- morpholin-4-yl-ethoxy)- pyridin-4-yl]-hydrazine |
| 238 | 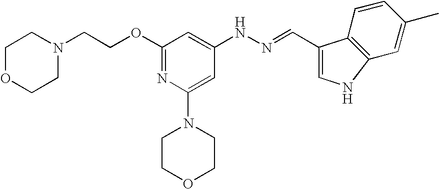 | N-(6-Methyl-1H-indol-3- ylmethylene)-N′-[2-morpholin- 4-yl-6-(2-morpholin-4-yl- ethoxy)-pyridin-4-yl]- hydrazine |
| 239 | 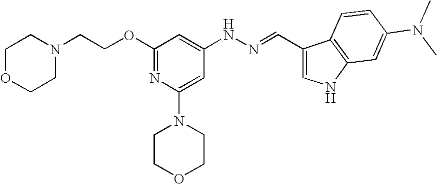 | Dimethyl-(3-{[2-morpholin-4- yl-6-(2-morpholin-4-yl- ethoxy)-pyridin-4-yl]- hydrazonomethyl}-1H-indol-6- yl)-amine |
| 240 | 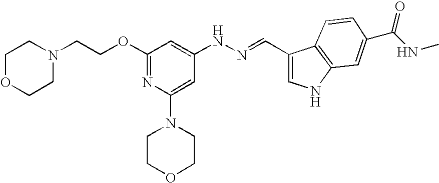 | 3-{[2-Morpholin-4-yl-6-(2- morpholin-4-yl-ethoxy)- pyridin-4-yl]- hydrazonomethyl}-1H-indole- 6-carboxylic acid methylamide |
| 241 | 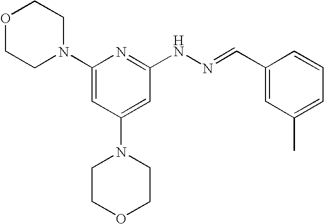 | N-(4,6-Di-morpholin-4-yl- pyridin-2-yl)-N′-(3-methyl- benzylidene)-hydrazine |
| 242 | 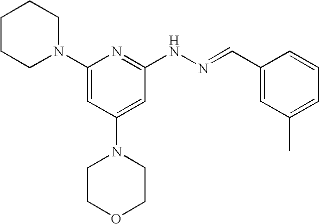 | N-(3-Methyl-benzylidene)-N′- (4′-morpholin-4-yl-3,4,5,6- tetrahydro-2H- [1,2′]bipyridinyl-6′-yl)- hydrazine |
| 243 | 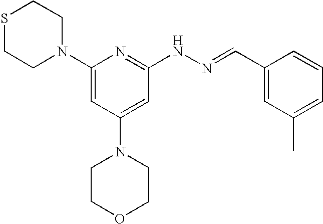 | N-(3-Methyl-benzylidene)-N′- (4-morpholin-4-yl-6- thiomorpholin-4-yl-pyridin-2- yl)-hydrazine |
| 244 | 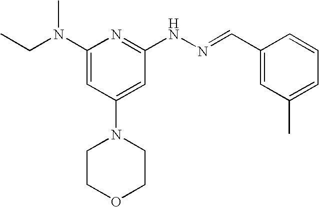 | Ethyl-methyl-{6-[N′-(3-methyl- benzylidene)-hydrazino]-4- morpholin-4-yl-pyridin-2-yl}- amine |
| 245 | 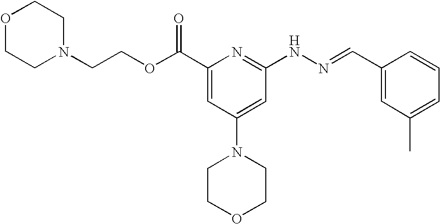 | 6-[N′-(3-Methyl-benzylidene)- hydrazino]-4-morpholin-4-yl- pyridine-2-carboxylic acid 2- morpholin-4-yl-ethyl ester |
| 246 | 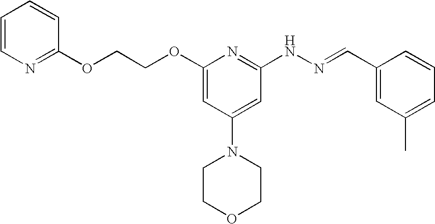 | N-(3-Methyl-benzylidene)-N′- {4-morpholin-4-yl-6-[2- (pyridin-2-yloxy)-ethoxy]- pyridin-2-yl}-hydrazine |
| 247 | 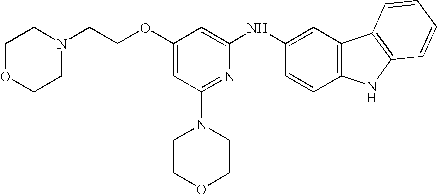 | (9H-Carbazol-3-yl)-[6- morpholin-4-yl-4-(2- morpholin-4-yl-ethoxy)- pyridin-2-yl]-amine |
| 248 | 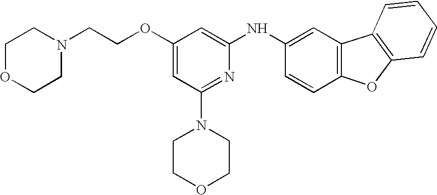 | Dibenzofuran-2-yl-[6- morpholin-4-yl-4-(2- morpholin-4-yl-ethoxy)- pyridin-2-yl]-amine |
| 249 | 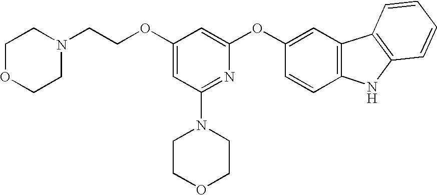 | 3-[6-Morpholin-4-yl-4-(2- morpholin-4-yl-ethoxy)- pyridin-2-yloxy]-9H-carbazole |
| 250 | 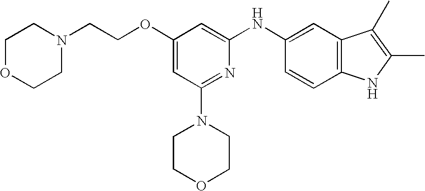 | (2,3-Dimethyl-1H-indol-5-yl)- [6-morpholin-4-yl-4-(2- morpholin-4-yl-ethoxy)- pyridin-2-yl]-amine |
| 251 | 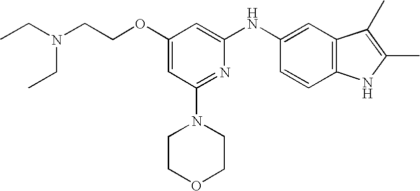 | [4-(2-Diethylamino-ethoxy)-6- morpholin-4-yl-pyridin-2-yl]- (2,3-dimethyl-1H-indol-5-yl)- amine |
| 252 | 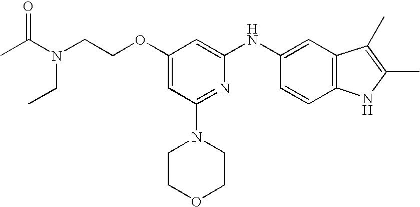 | N-{2-[2-(2,3-Dimethyl-1H- indol-5-ylamino)-6-morpholin- 4-yl-pyridin-4-yloxy]-ethyl}- N-ethyl-acetamide |
| 253 | 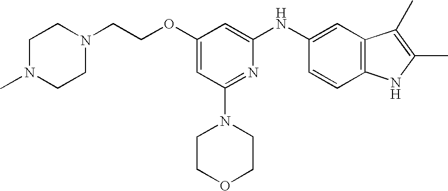 | (2,3-Dimethyl-1H-indol-5-yl)- {4-[2-(4-methyl-piperazin-1- yl)-ethoxy]-6-morpholin-4-yl- pyridin-2-yl}-amine |
| 254 | 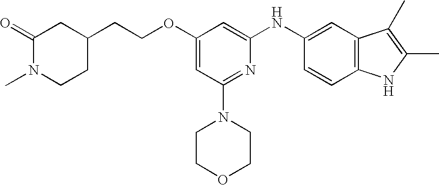 | 4-{2-[2-(2,3-Dimethyl-1H- indol-5-ylamino)-6-morpholin- 4-yl-pyridin-4-yloxy]-ethyl}-1- methyl-piperidin-2-one |
| 255 | 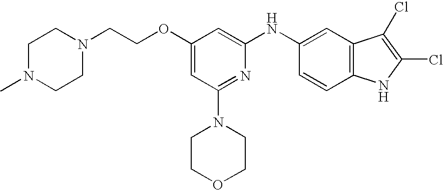 | (2,3-Dichloro-1H-indol-5-yl)- {4-[2-(4-methyl-piperazin-1- yl)-ethoxy]-6-morpholin-4-yl- pyridin-2-yl}-amine |
| 256 | 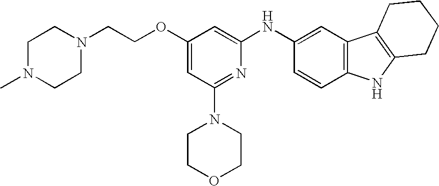 | {4-[2-(4-Methyl-piperazin-1- yl)-ethoxy]-6-morpholin-4-yl- pyridin-2-yl}-(6,7,8,9- tetrahydro-5H-carbazol-3-yl)- amine |
| 257 | 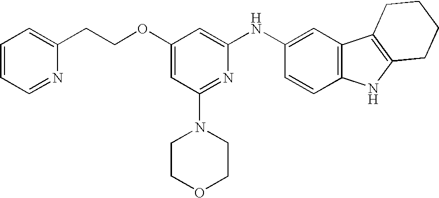 | [6-Morpholin-4-yl-4-(2- pyridin-2-yl-ethoxy)-pyridin-2- yl]-(6,7,8,9-tetrahydro-5H- carbazol-3-yl)-amine |
| 258 | 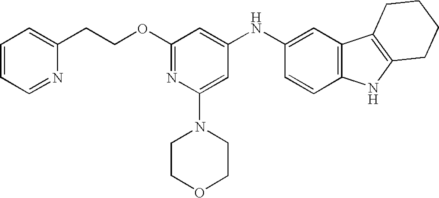 | [2-Morpholin-4-yl-6-(2- pyridin-2-yl-ethoxy)-pyridin-4- yl]-(6,7,8,9-tetrahydro-5H- carbazol-3-yl)-amine |
| 259 | 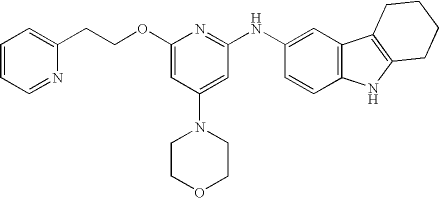 | [4-Morpholin-4-yl-6-(2- pyridin-2-yl-ethoxy)-pyridin-2- yl]-(6,7,8,9-tetrahydro-5H- carbazol-3-yl)-amine |
| 260 | 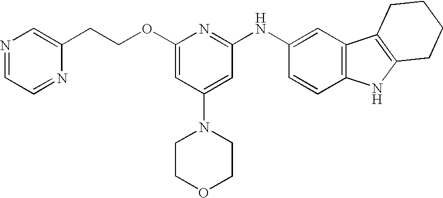 | [4-Morpholin-4-yl-6-(2- pyrazin-2-yl-ethoxy)-pyridin-2- yl]-(6,7,8,9-tetrahydro-5H- carbazol-3-yl)-amine |
| 261 | 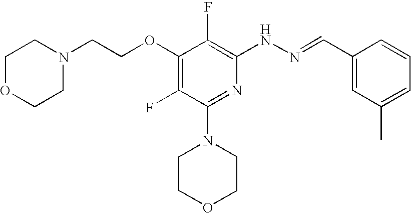 | N-[3,5-Difluoro-6-morpholin- 4-yl-4-(2-morpholin-4-yl- ethoxy)-pyridin-2-yl]-N′-(3- methyl-benzylidene)-hydrazine |
| 262 | 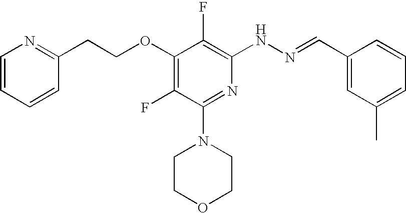 | N-[3,5-Difluoro-6-morpholin- 4-yl-4-(2-pyridin-2-yl-ethoxy)- pyridin-2-yl]-N′-(3-methyl- benzylidene)-hydrazine |
| 263 | 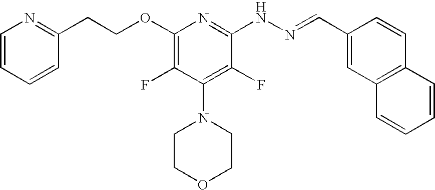 | N-[3,5-Difluoro-4-morpholin- 4-yl-6-(2-pyridin-2-yl-ethoxy)- pyridin-2-yl]-N′-naphthalen-2- ylmethylene-hydrazine |
| 264 | 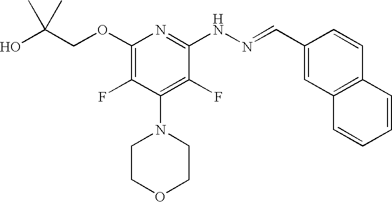 | 1-[3,5-Difluoro-4-morpholin-4- yl-6-(N′-naphthalen-2- ylmethylene-hydrazino)- pyridin-2-yloxy]-2-methyl- propan-2-ol |
| 265 | 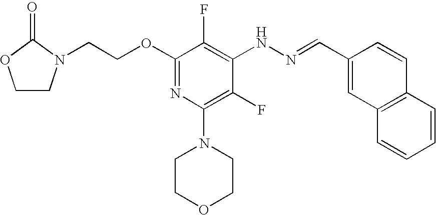 | 3-{2-[3,5-Difluoro-6- morpholin-4-yl-4-(N′- naphthalen-2-ylmethylene- hydrazino)-pyridin-2-yloxy]- ethyl}-oxazolidin-2-one |
| 266 | 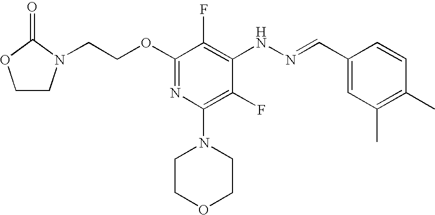 | 3-(2-{4-[N′-(3,4-Dimethyl- benzylidene)-hydrazino]-3,5- difluoro-6-morpholin-4-yl- pyridin-2-yloxy}-ethyl)- oxazolidin-2-one |
| 267 | 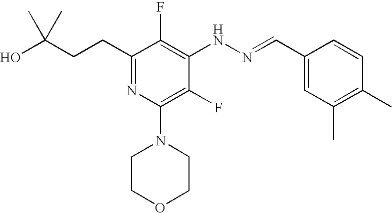 | 4-{4-[N′-(3,4-Dimethyl- benzylidene)-hydrazino]-3,5- difluoro-6-morpholin-4-yl- pyridin-2-yl}-2-methyl-butan- 2-ol |
| 268 | 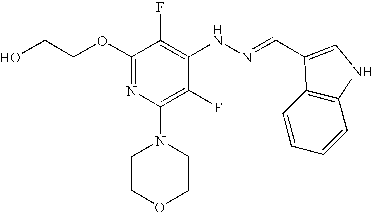 | 2-{3,5-Difluoro-4-[N′-(1H- indol-3-ylmethylene)- hydrazino]-6-morpholin-4-yl- pyridin-2-yloxy}-ethanol |
| 269 | 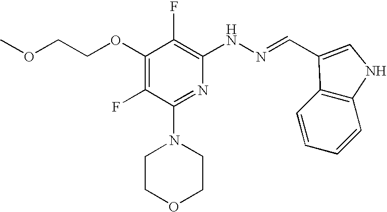 | N-[3,5-Difluoro-4-(2-methoxy- ethoxy)-6-morpholin-4-yl- pyridin-2-yl]-N′-(1H-indol-3- ylmethylene)-hydrazine |
| 270 | 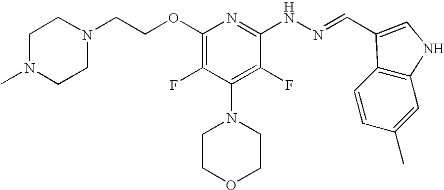 | N-{3,5-Difluoro-6-[2-(4- methyl-piperazin-1-yl)- ethoxy]-4-morpholin-4-yl- pyridin-2-yl}-N′-(6-methyl-1H- indol-3-ylmethylene)-hydrazine |
| 271 | 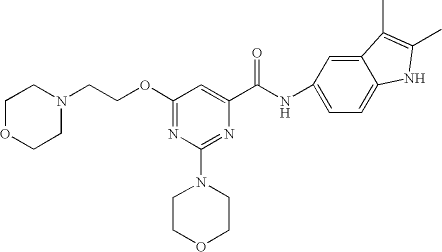 | 2-Morpholin-4-yl-6-(2- morpholin-4-yl-ethoxy)- pyrimidine-4-carboxylic acid (2,3-dimethyl-1H-indol-5-yl)- amide |
| 272 | 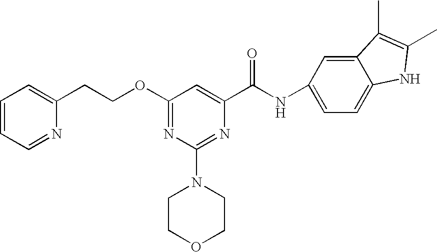 | 2-Morpholin-4-yl-6-(2-pyridin- 2-yl-ethoxy)-pyrimidine-4- carboxylic acid (2,3-dimethyl- 1H-indol-5-yl)-amide |
| 273 | 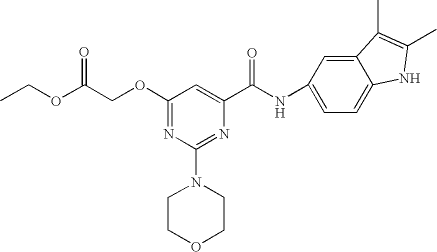 | [6-(2,3-Dimethyl-1H-indol-5- ylcabamoyl)-2-morpholin-4-yl- pyrimidin-4-yloxy]-acetic acid ethyl ester |
| 274 | 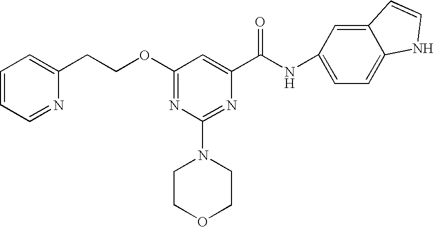 | 2-Morpholin-4-yl-6-(2-pyridin- 2-yl-ethoxy)-pyrimidine-4- carboxylic acid (1H-indol-5- yl)-amide |
| 275 | 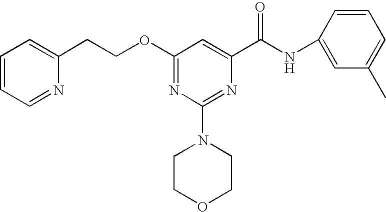 | 2-Morpholin-4-yl-6-(2-pyridin- 2-yl-ethoxy)-pyrimidine-4- carboxylic acid m-tolylamide |
| 276 | 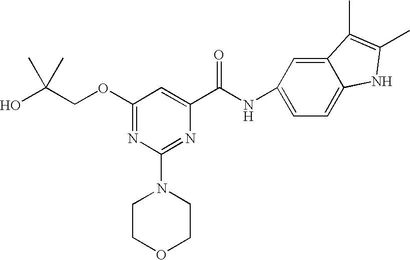 | 6-(2-hydroxy-2-methyl- propoxy)-2-morpholin-4-yl- pyrimidine-4-carboxylic acid (2,3-dimethyl-1H-indol-5-yl)- amide |
| 277 | 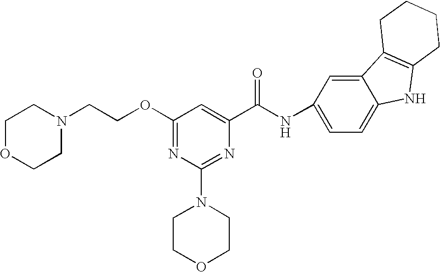 | 2-Morpholin-4-yl-6-(2- morpholin-4-yl-ethoxy)- pyrimidine-4-carboxylic acid (6,7,8,9-tetrahydro-5H- carbazol-3-yl)-amide |
| 278 | 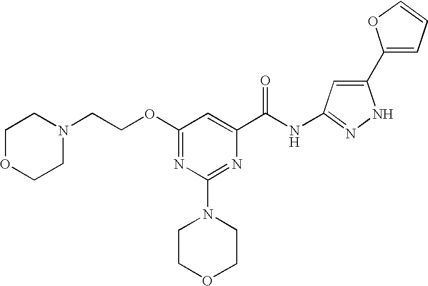 | 2-Morpholin-4-yl-6-(2- morpholin-4-yl-ethoxy)- pyrimidine-4-carboxylic acid (5-furan-2-yl-1H-pyrazol-3-yl)- amide |
| 279 | 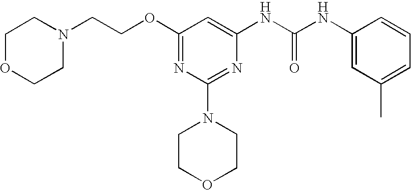 | 1-[2-Morpholin-4-yl-6-(2- morpholin-4-yl-ethoxy)- pyrimidin-4-yl]-3-m-tolyl-urea |
| 280 | 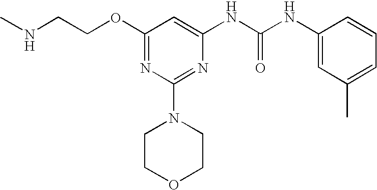 | 1-[6-(2-Methylamino-ethoxy)- 2-morpholin-4-yl-pyrimidin-4- yl]-3-m-tolyl-urea |
| 281 | 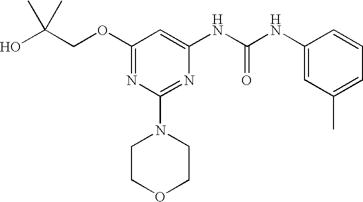 | 1-[6-(2-Hydroxy-2-methyl- propoxy)-2-morpholin-4-yl- pyrimidin-4-yl]-3-m-tolyl-urea |
| 282 | 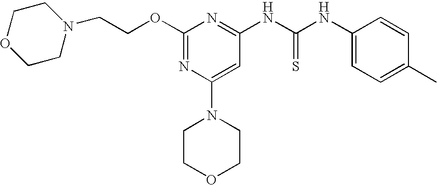 | 1-[6-Morpholin-4-yl-2-(2- morpholin-4-yl-ethoxy)- pyrimidin-4-yl]-3-p-tolyl- thiourea |
| 283 | 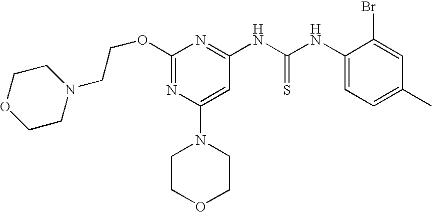 | 1-(2-Bromo-4-methyl-phenyl)- 3-[6-morpholin-4-yl-2-(2- morpholin-4-yl-ethoxy)- pyrimidin-4-yl]-thiourea |
| 284 | 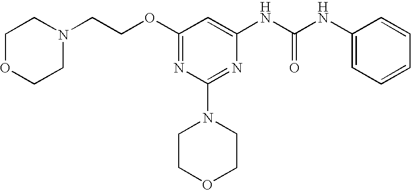 | 1-[2-Morpholin-4-yl-6-(2- morpholin-4-yl-ethoxy)- pyrimidin-4-yl]-3-phenyl-urea |
| 285 | 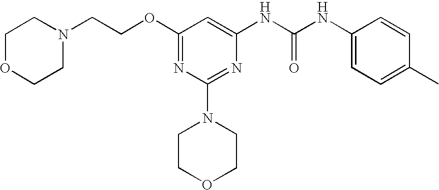 | 1-[2-Morpholin-4-yl-6-(2- morpholin-4-yl-ethoxy)- pyrimidin-4-yl]-3-p-tolyl-urea |
| 286 | 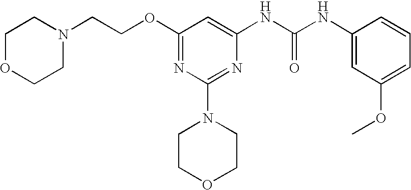 | 1-(3-Methoxy-phenyl)-3-[2- morpholin-4-yl-6-(2- morpholin-4-yl-ethoxy)- pyrimidin-4-yl]-urea |
| 287 | 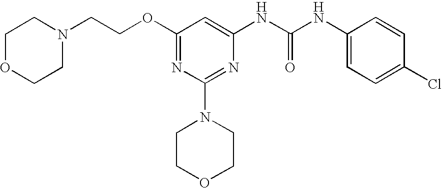 | 1-(4-Chloro-phenyl)-3-[2- morpholin-4-yl-6-(2- morpholin-4-yl-ethoxy)- pyrimidin-4-yl]-urea |
| 288 | 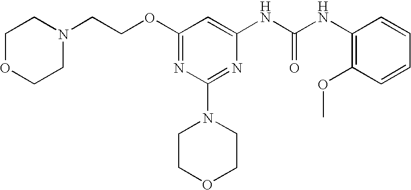 | 1-(2-Methoxy-phenyl)-3-[2- morpholin-4-yl-6-(2- morpholin-4-yl-ethoxy)- pyrimidin-4-yl]-urea |
| 289 | 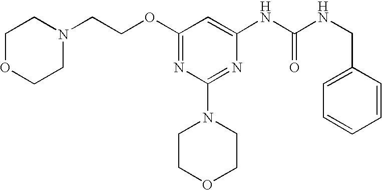 | 1-Benzyl-3-[2-morpholin-4-yl- 6-(2-morpholin-4-yl-ethoxy)- pyrimidin-4-yl]-urea |
| 290 | 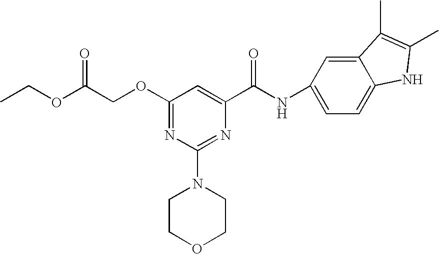 | [6-(2,3-Dimethyl-1H-indol-5- ylcarbamoyl)-2-morpholin-4- yl-pyrimidin-4-yloxy]-acetic acid ethyl ester |
| 291 | 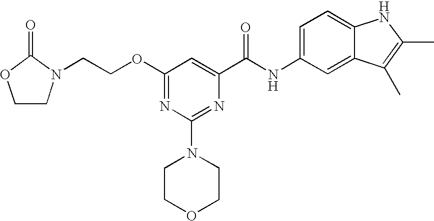 | 2-Morpholin-4-yl-6-[2-(2-oxo- oxazolidin-3-yl)-ethoxy]- pyrimidine-4-carboxylic acid (2,3-dimethyl-1H-indol-5-yl)- amide |
| 292 | 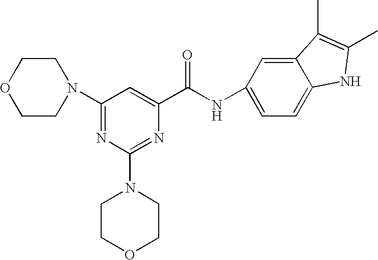 | 2,6-Di-morpholin-4-yl- pyrimidine-4-carboxylic acid (2,3-dimethyl-1H-indol-5-yl)- amide |
| 293 | 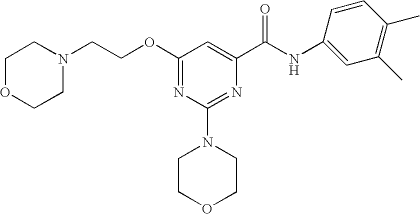 | 2-Morpholin-4-yl-6-(2- morpholin-4-yl-ethoxy)- pyrimidine-4-carboxylic acid (3,4-dimethyl-phenyl)-amide |
| 294 | 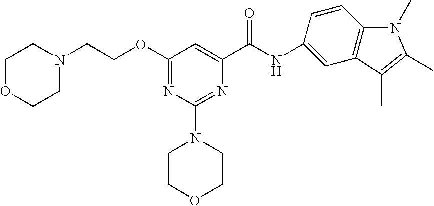 | 2-Morpholin-4-yl-6-(2- morpholin-4-yl-ethoxy)- pyrimidine-4-carboxylic acid (1,2,3-trimethyl-1H-indol-5- yl)-amide |
| 295 | 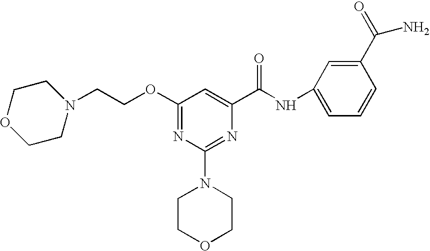 | 2-Morpholin-4-yl-6-(2- morpholin-4-yl-ethoxy)- pyrimidine-4-carboxylic acid (3-carbamoyl-phenyl)-amide |
| 296 | 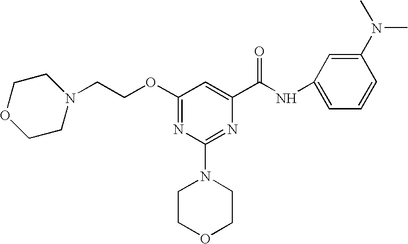 | 2-Morpholin-4-yl-6-(2- morpholin-4-yl-ethoxy)- pyrimidine-4-carboxylic acid (3-dimethylamino-phenyl)- amide |
| 297 | 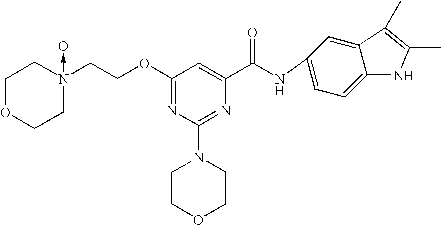 | 2-Morpholin-4-yl-6-[2-(4-oxy- morpholin-4-yl)-ethoxy]-pyrimidine- 4-carboxylic acid (2,3-dimethyl-1H- indol-5-yl)-amide |
| 298 | 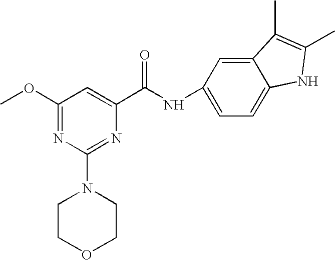 | 6-Methoxy-2-morpholin-4-yl- pyrimidine-4-carboxylic acid (2,3-dimethyl-1H-indol-5-yl)- amide |
| 299 | 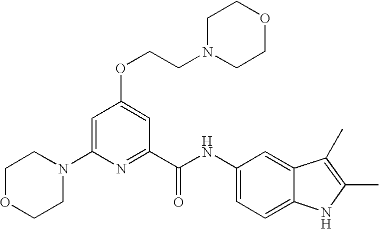 | 6-Morpholin-4-yl-4-(2- morpholin-4-yl-ethoxy)- pyridine-2-carboxylic acid (2,3-dimethyl-1H-indol-5-yl)- amide |
| 300 | 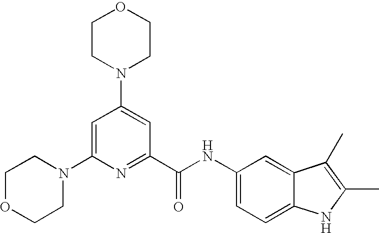 | 4,6-Di-morpholin-4-yl- pyridine-2-carboxylic aci (2,3- dimethyl-1H-indol-5-yl)-amide |
| 301 | 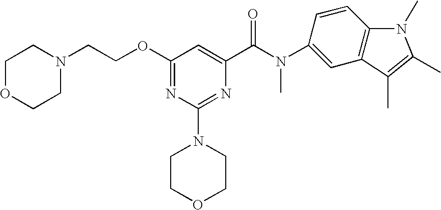 | 2-Morpholin-4-yl-6-(2- morpholin-4-yl-ethoxy)- pyrimidine-4-carboxylic acid methyl-(1,2,3-trimethyl-1H- indol-5-yl)-amide |
| 302 | 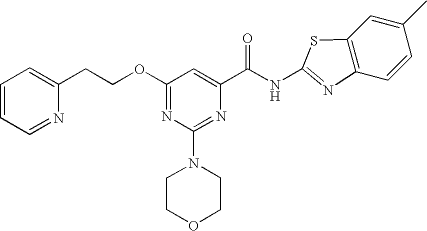 | 2-Morpholin-4-yl-6-(2-pyridin- 2-yl-ethoxy)-pyrimidine-4- carboxylic acid (6-methyl- benzothiazol-2-yl)-amide |
| 303 | 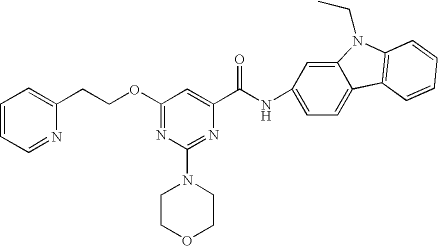 | 2-Morpholin-4-yl-6-(2-pyridin- 2-yl-ethoxy)-pyrimidine-4- carboxylic acid (9-ethyl-9H- carbazol-2-yl)-amide |
| 304 | 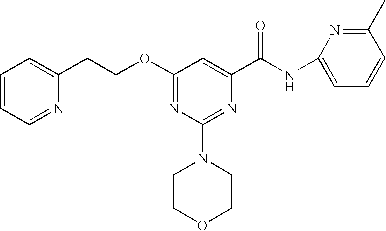 | 2-Morpholin-4-yl-6-(2-pyridin- 2-yl-ethoxy)-pyrimidine-4- carboxylic acid (6-methyl- pyridin-2-yl)-amide |
| 305 | 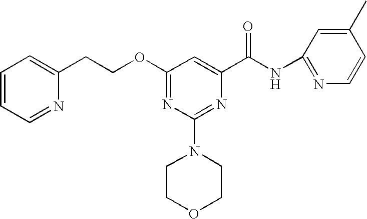 | 2-Morpholin-4-yl-6-(2-pyridin- 2-yl-ethoxy)-pyrimidine-4- carboxylic acid (4-methyl- pyridin-2-yl)-amide |
| 306 | 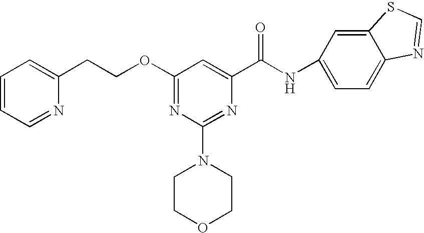 | 2-Morpholin-4-yl-6-(2-pyridin- 2-yl-ethoxy)-pyrimidine-4- carboxylic acid benzothiazol-6- ylamide |
| 307 | 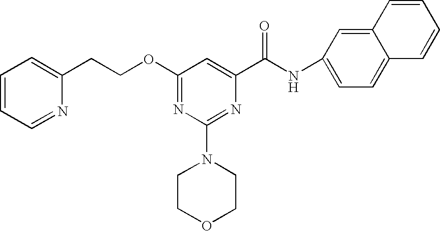 | 2-Morpholin-4-yl-6-(2-pyridin- 2-yl-ethoxy)-pyrimidine-4- carboxylic acid naphthalen-2- ylamide |
| 308 | 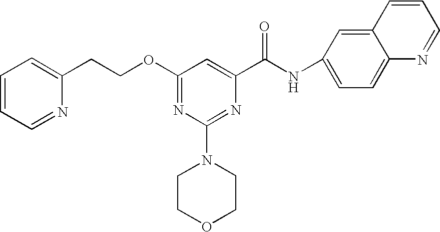 | 2-Morpholin-4-yl-6-(2-pyridin- 2-yl-ethoxy)-pyrimidine-4- carboxylic acid quinolin-6- ylamide |
| 309 | 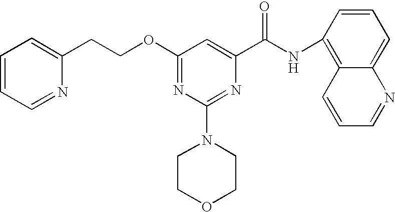 | 2-Morpholin-4-yl-6-(2-pyridin- 2-yl-ethoxy)-pyrimidine-4- carboxylic acid quinolin-5- ylamide |
| 310 | 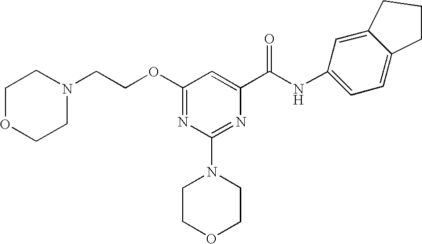 | 2-Morpholin-4-yl-6-(2- morpholin-4-yl-ethoxy)- pyrimidine-4-carboxylic acid indan-5-ylamide |
| 311 | 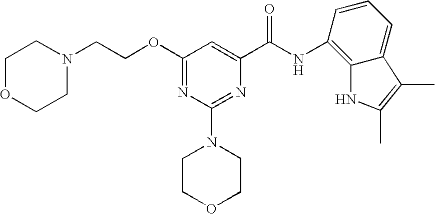 | 2-Morpholin-4-yl-6-(2- morpholin-4-yl-ethoxy)- pyrimidine-4-carboxylic acid (2,3-dimethyl-1H-indol-7-yl)- amide |
| 312 | 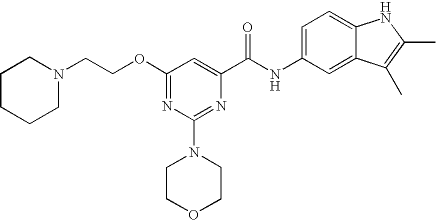 | 2-Morpholin-4-yl-6-(2- piperidin-1-yl-ethoxy)- pyrimidine-4-carboxylic acid (2,3-dimethyl-1H-indol-5-yl)- amide |
| 313 | 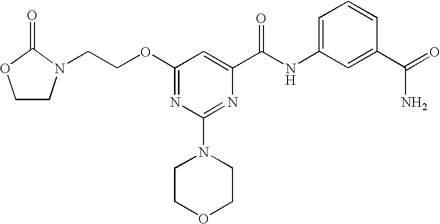 | 2-Morpholin-4-yl-6- [2-(2-oxo- oxazolidin-3-yl)-ethoxy]- pyrimidine-4-carboxylic acid (3-carbamoyl-phenyl)-amide |
| 314 | 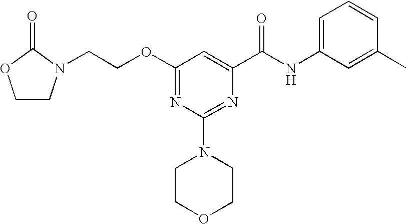 | 2-Morpholin-4-yl-6-[2-(2-oxo- oxazolidin-3-yl)-ethoxy]- pyrimidine-4-carboxylic acid m-tolylamide |
| 315 | 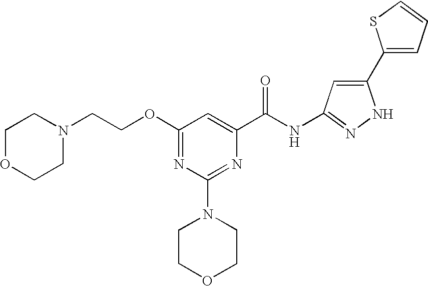 | 2-Morpholin-4-yl-6-(2- morpholin-4-yl-ethoxy)- pyrimidine-4-carboxylic acid (5-thiophen-2-yl-1H-pyrazol-3- yl)-amide |
| 316 | 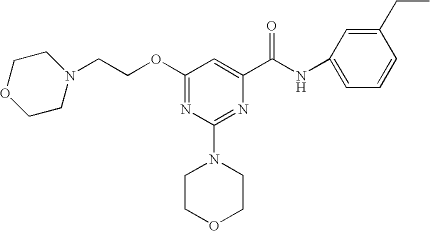 | 2-Morpholin-4-yl-6-(2- morpholin-4-yl-ethoxy)- pyrimidine-4-carboxylic acid (3-ethyl-phenyl)-amide |
| 317 | 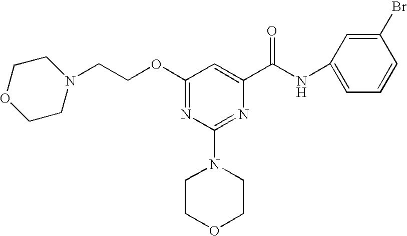 | 2-Morpholin-4-yl-6-(2- morpholin-4-yl-ethoxy)- pyrimidine-4-carboxylic acid (3-bromo-phenyl)-amide |
| 318 | 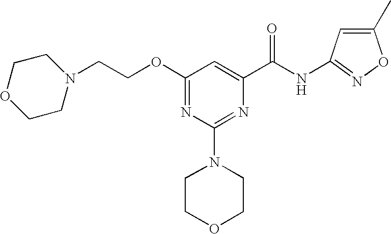 | 2-Morpholin-4-yl-6-(2- morpholin-4-yl-ethoxy)- pyrimidine-4-carboxylic acid (5-methyl-isoxazol-3-yl)-amide |
| 319 | 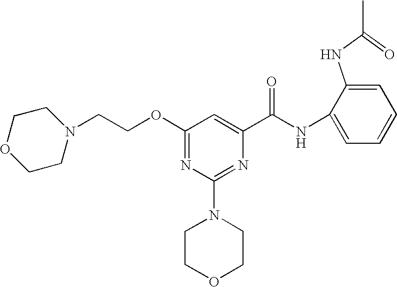 | 2-Morpholin-4-yl-6-(2- morpholin-4-yl-ethoxy)- pyrimidine-4-carboxylic acid (2-acetylamino-phenyl)-amide |
| 320 | 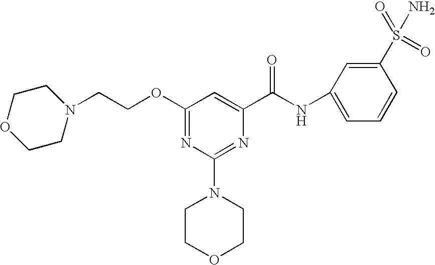 | 2-Morpholin-4-yl-6-(2- morpholin-4-yl-ethoxy)- pyrimidine-4-carboxylic acid (3-sulfamoyl-phenyl)-amide |
| 321 | 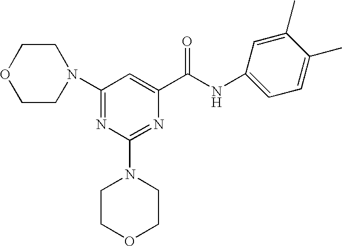 | 2,6-Di-morpholin-4-yl- pyrimidine-4-carboxylic acid (3,4-dimethyl-phenyl)-amide |
| 322 | 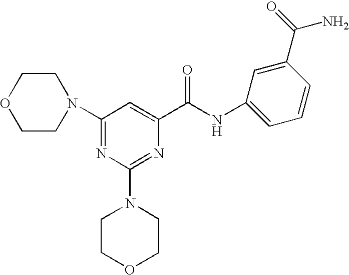 | 2,6-Di-morpholin-4-yl- pyrimidine-4-carboxylic acid (3-carbamoyl-phenyl)-amide |
| 323 | 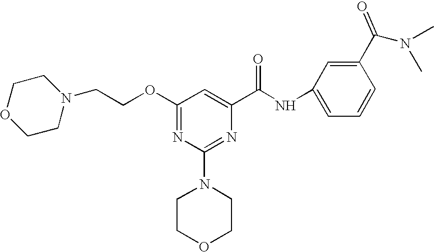 | 2-Morpholin-4-yl-6-(2- morpholin-4-yl-ethoxy)- pyrimidine-4-carboxylic acid (3-dimethylcarbamoyl-phenyl)- amide |
| 324 | 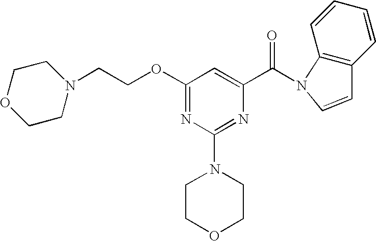 | Indol-1-yl-[2-morpholin-4-yl- 6-(2-morpholin-4-yl-ethoxy)- pyrimidin-4-yl]-methanone |
| 325 | 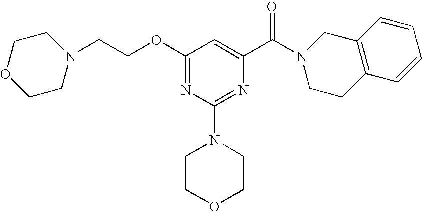 | (3,4-Dihydro-1H-isoquinolin- 2-yl)-[2-morpholin-4-yl-6-(2- morpholin-4-yl-ethoxy)- pyrimidin-4-yl]-methanone |
| 326 | 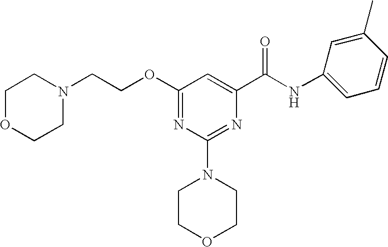 | 2-Morpholin-4-yl-6-(2- morpholin-4-yl-ethoxy)- pyrimidine-4-carboxylic acid m-tolylamide |
| 327 | 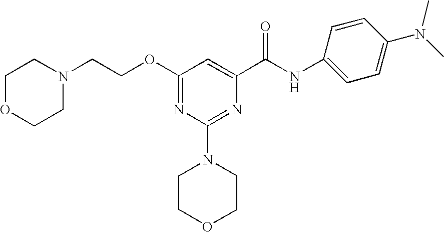 | 2-Morpholin-4-yl-6-(2- morpholin-4-yl-ethoxy)- pyrimidine-4-carboxylic acid (4-dimethylamino-phenyl)- amide |
| 328 | 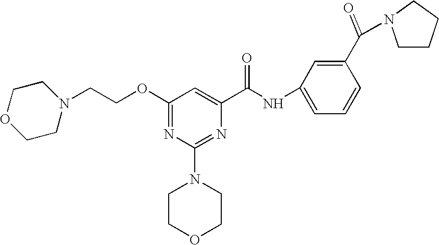 | 2-Morpholin-4-yl-6-(2- morpholin-4-yl-ethoxy)- pyrimidine-4-carboxylic acid [3-(pyrrolidine-1-carbonyl)- phenyl]-amide |
| 329 | 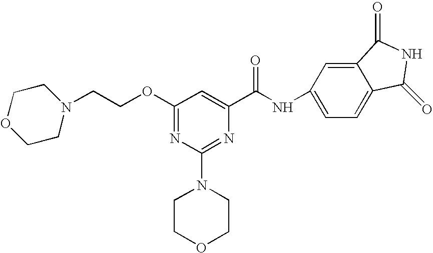 | 2-Morpholin-4-yl-6-(2- morpholin-4-yl-ethoxy)- pyrimidine-4-carboxylic acid (1,3-dioxo-2,3-dihydro-1H- isoindol-5-yl)-amide |
| 330 | 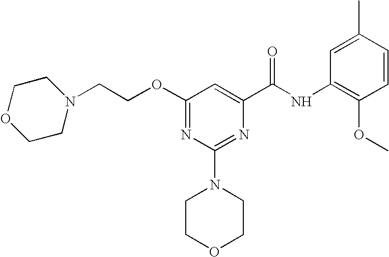 | 2-Morpholin-4-yl-6-(2- morpholin-4-yl-ethoxy)- pyrimidine-4-carboxylic acid (2-methoxy-5-methyl-phenyl)- amide |
| 331 | 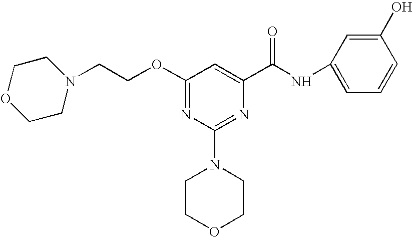 | 2-Morpholin-4-yl-6-(2- morpholin-4-yl-ethoxy)- pyrimidine-4-carboxylic acid (3-hydroxy-phenyl)-amide |
| 332 | 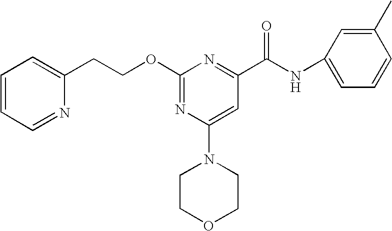 | 6-Morpholin-4-yl-2-(2-pyridin- 2-yl-ethoxy)-pyrimidine-4- carboxylic acid m-tolylamide |
| 333 | 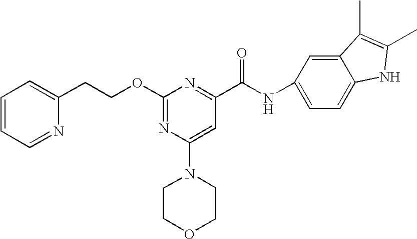 | 6-Morpholin-4-yl-2-(2-pyridin- 2-yl-ethoxy)-pyrimidine-4- carboxylic acid (2,3-dimethyl- 1H-indol-5-yl)-amide |
| 334 | 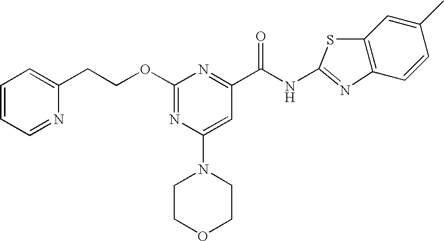 | 6-Morpholin-4-yl-2-(2-pyridin- 2-yl-ethoxy)-pyrimidine-4- carboxylic acid (6-methyl- benzothiazol-2-yl)-amide |
| 335 | 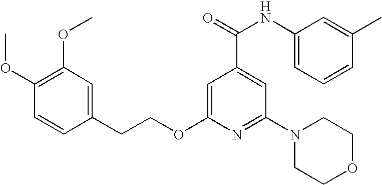 | 2-[2-(3,4-Dimethoxy-phenyl)- ethoxy]-6-morpholin-4-yl-N-m- tolyl-isonicotinamide |
| 336 | 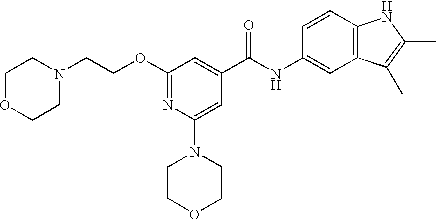 | N-(2,3-Dimethyl-1H-indol-5- yl)-2-morpholin-4-yl-6-(2- morpholin-4-yl-ethoxy)- isonicotinamide |
| 337 | 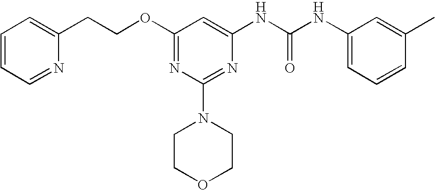 | 1-[2-Morpholin-4-yl-6-(2- pyridin-2-yl-ethoxy)- pyrimidin-4-yl]-3-m-tolyl-urea |
| 338 | 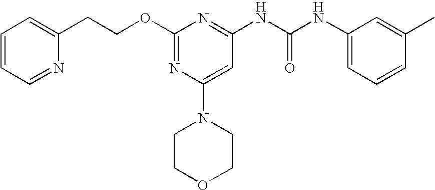 | 1-[6-Morpholin-4-yl-2-(2- pyridin-2-yl-ethoxy)- pyrimidin-4-yl]-3-m-tolyl-urea |
| 339 | 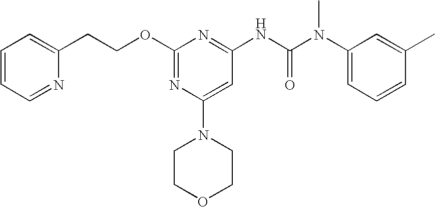 | 1-Methyl-3-[6-morpholin-4-yl- 2-(2-pyridin-2-yl-ethoxy)- pyrimidin-4-yl]-1-m-tolyl-urea |
| 340 | 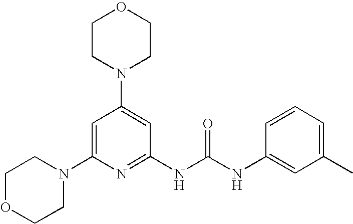 | 1-(4,6-Di-morpholin-4-yl- pyridin-2-yl)-3-m-tolyl-urea |
| 341 | 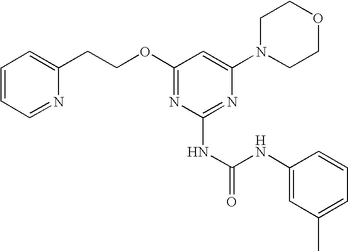 | 1-[(4-Morpholin-4-yl-6-(2- pyridin-2-yl-ethoxy)- pyrimidin-2-yl]-3-m-tolyl-urea |
| 342 | 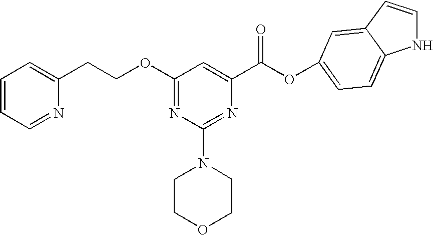 | 2-Morpholin-4-yl-6-(2-pyridin- 2-yl-ethoxy)-pyrimidine-4- carboxylic acid 1H-indol-5-yl ester |
| 343 | 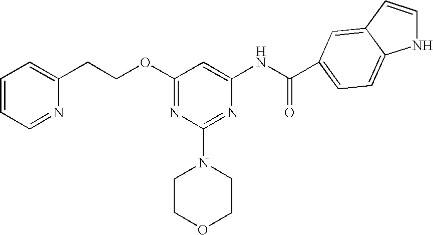 | 1H-Indole-5-carboxylic acid [2-morpholin-4-yl-6-(2- pyridin-2-yl-ethoxy)- pyrimidin-4-yl]-amide |
| 344 | 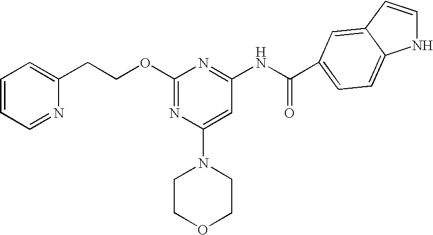 | 1H-Indole-5-carboxylic acid [6-morpholin-4-yl-2-(2- pyridin-2-yl-ethoxy)- pyrimidin-4-yl]-amide |
| 345 | 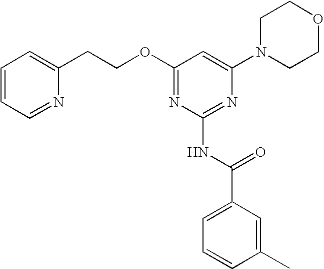 | 3-Methyl-N-[4-morpholin-4-yl- 6-(2-pyridin-2-yl-ethoxy)- pyrimidin-2-yl]-benzamide |
| 346 | 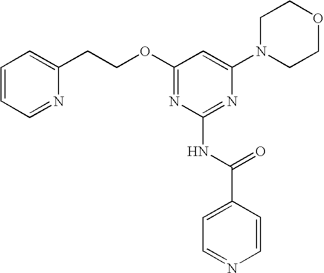 | N-[4-Morpholin-4-yl-6-(2- pyridin-2-yl-ethoxy)- pyrimidin-2-yl]- isonicotinamide |
| 347 | 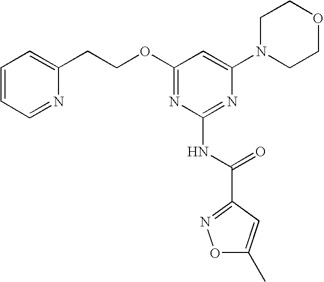 | 5-Methyl-isoxazole-3- carboxylic acid-[4-morpholin- 4-yl-6-(2-pyridin-2-yl-ethoxy)- pyrimidin-2-yl]-amide |
| 348 | 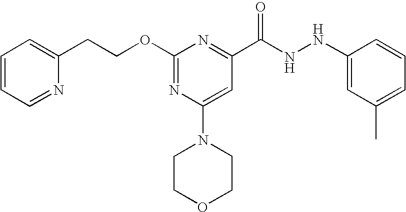 | 6-Morpholin-4-yl-2-(2-pyridin- 2-yl-ethoxy)-pyrimidine-4- carboxylic acid N′-m-tolyl- hydrazide |
| 349 | 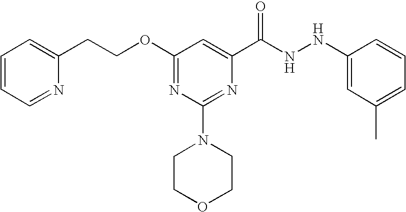 | 2-Morpholin-4-yl-6-(2-pyridin- 2-yl-ethoxy)-pyrimidine-4- carboxylic acid N′-m-tolyl- hydrazide |
| 350 | 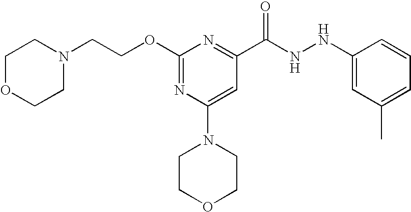 | 6-Morpholin-4-yl-2-(2- morpholin-4-yl-ethoxy)- pyrimidine-4-carboxylic acid N′-m-tolyl-hydrazide |
| 351 | 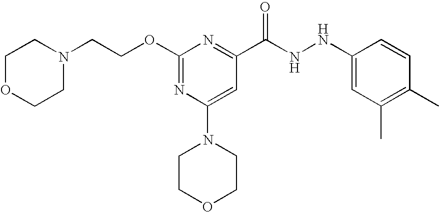 | 6-Morpholin-4-yl-2-(2- morpholin-4-yl-ethoxy)- pyrimidine-4-carboxylic acid N′-(3,4-dimethyl-phenyl)- hydrazide |
| 352 | 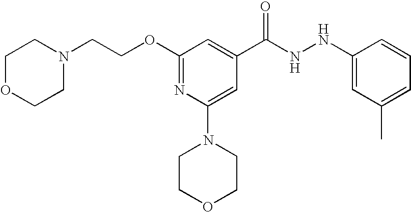 | 2-Morpholin-4-yl-6-(2- morpholin-4-yl-ethoxy)- isonicotinic acid N′-m-tolyl- hydrazide |
| 353 | 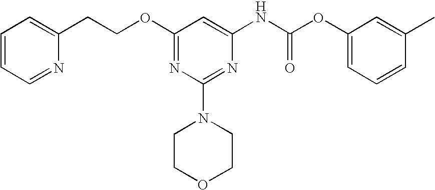 | [2-Morpholin-4-yl-6-(2- pyridin-2-yl-ethoxy)- pyrimidin-4-yl]-carbamic acid m-tolyl ester |
| 354 | 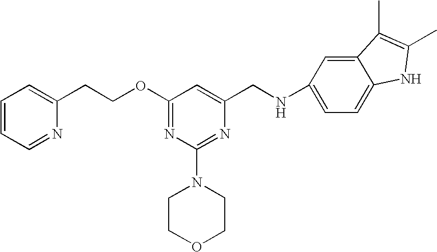 | (2,3-Dimethyl-1H-indol-5-yl)- [2-morpholin-4-yl-6-(2- pyridin-2-yl-ethoxy)- pyrimidin-4-ylmethyl]-amine |
| 355 | 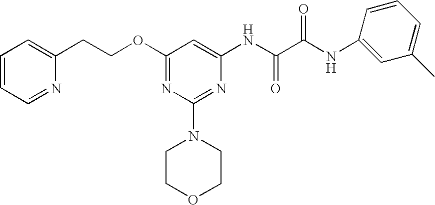 | N-[2-Morpholin-4-yl-6-(2-pyridin-2-yl-ethoxy)- pyrimidin-4-yl]-N′-m-tolyl- oxalamide |
| 356 | 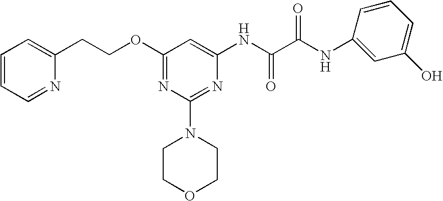 | N-(3-Hydroxy-phenyl)-N′-[2- morpholin-4-yl-6-(2-pyridin-2- yl-ethoxy)-pyrimidin-4-yl]- oxalamide |
| 357 | 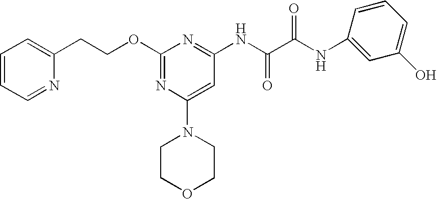 | N-(3-Hydroxy-phenyl)-N′-[6 morpholin-4-yl-2-(2-pyridin-2- yl-ethoxy)-pyrimidin-4-yl]- oxalamide |
| 358 | 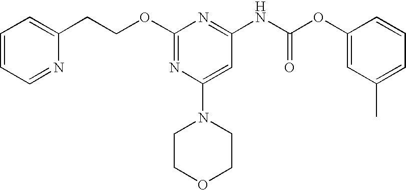 | [6-Morpholin-4-yl-2-(2- pyridin-2-yl-ethoxy)- pyrimidin-4-yl]-carbamic acid m-tolyl ester |
| 359 | 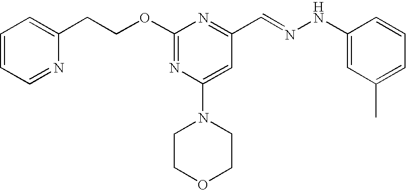 | N-[6-Morpholin-4-yl-2-(2- pyridin-2-yl-ethoxy)- pyrimidin-4-ylmethylene]-N′- m-tolyl-hydrazine |
| 360 | 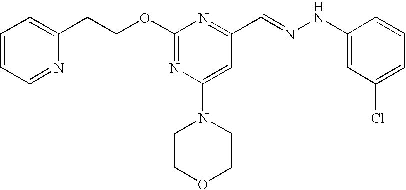 | N-(3-Chloro-phenyl)-N′-[6- morpholin-4-yl-2-(2-pyridin-2- yl-ethoxy)-pyrimidin-4- ylmethylene]-hydrazine |
| 361 | 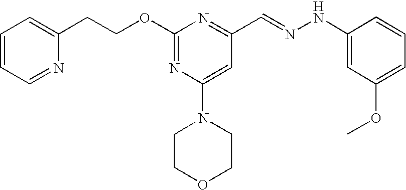 | N-(3-Methoxy-phenyl)-N′-[6- morpholin-4-yl-2-(2-pyridin-2- yl-ethoxy)-pyrimidin-4- ylmethylene]-hydrazine |
| 362 | 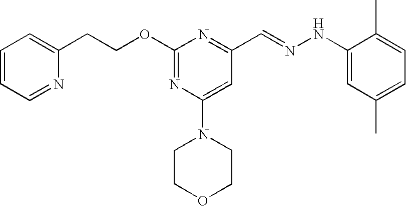 | N-(2,5-Dimethyl-phenyl)-N′- [6-morpholin-4-yl-2-(2- pyridin-2-yl-ethoxy)- pyrimidin-4-ylmethylene]- hydrazine |
| 363 | 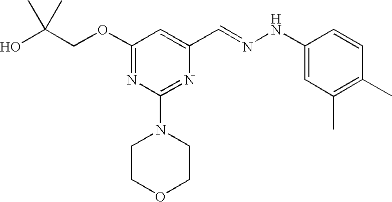 | 1-{6-[(3,4-Dimethyl-phenyl)- hydrazonomethyl]-2- morpholin-4-yl-pyrimidin-4- yloxy}-2-methyl-propan-2-ol |
| 364 | 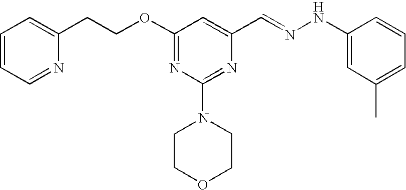 | N-[2-Morpholin-4-yl-6-(2- pyridin-2-yl-ethoxy)- pyrimidin-4-ylmethylene]-N′- m-tolyl-hydrazine |
| 365 | 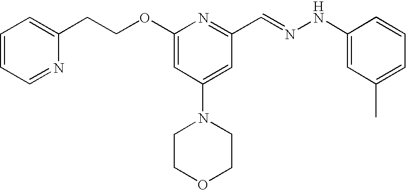 | N-[4-Morpholin-4-yl-6-(2- pyridin-2-yl-ethoxy)-pyridin-2- ylmethylene]-N′-m-tolyl- hydrazine |
| 366 | 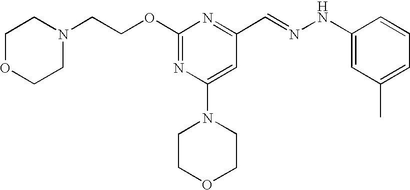 | N-[6-Morpholin-4-yl-2-(2- morpholin-4-yl-ethoxy)- pyrimidin-4-ylmethylene]-N′- m-tolyl-hydrazine |
| 367 | 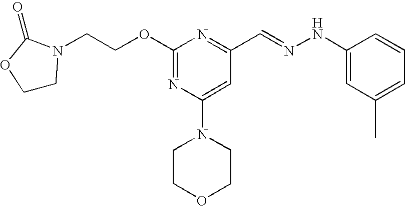 | 3-{2-[4-Morpholin-4-yl-6-(m- tolyl-hydrazonomethyl)- pyrimidin-2-yloxy]-ethyl}- oxazolidin-2-one |
| 368 | 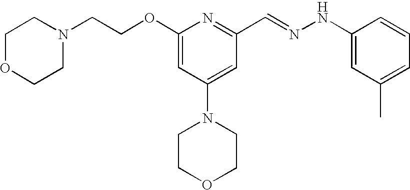 | N-[4-Morpholin-4-yl-6-(2- morpholin-4-yl-ethoxy)- pyridin-2-ylmethylene]-N′-m tolyl-hydrazine |
| 369 | 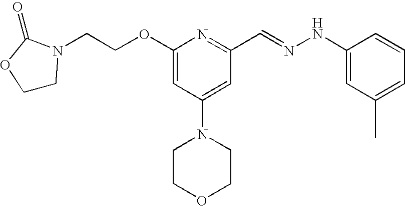 | 3-{2-[4-Morpholin-4-yl-6-(m- tolyl-hydrazonomethyl)- pyridin-2-yloxy]-ethyl}- oxazolidin-2-one |
| 370 | 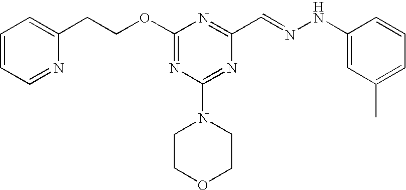 | N-[4-Morpholin-4-yl-6-(2- pyridin-2-yl-ethoxy)- [1,3,5]triazin-2-ylmethylene]- N′-m-tolyl-hydrazine |
| 371 | 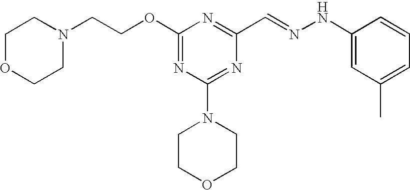 | N-[4-Morpholin-4-yl-6-(2- morpholin-4-yl-ethoxy)- [1,3,5]triazin-2-ylmethylene]- N′-m-tolyl-hydrazine |
| 372 | 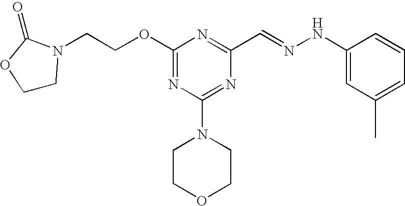 | 3-{2-[4-Morpholin-4-yl-6-(m- tolyl-hydrazonomethyl)- [1,3,5]triazin-2-yloxy]-ethyl}- oxazolidin-2-one |
| 373 | 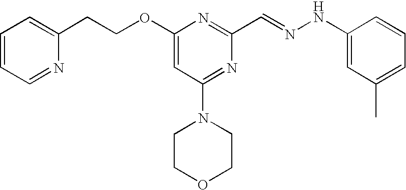 | N-[4-Morpholin-4-yl-6-(2- pyridin-2-yl-ethoxy)- pyrimidin-2-ylmethylene]-N′- m-tolyl-hydrazine |
| 374 | 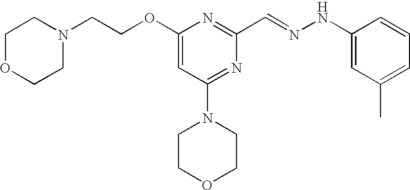 | N-[4-Morpholin-4-yl-6-(2- morpholin-4-yl-ethoxy)- pyrimidin-2-ylmethylene]-N′- m-tolyl-hydrazine |
| 375 | 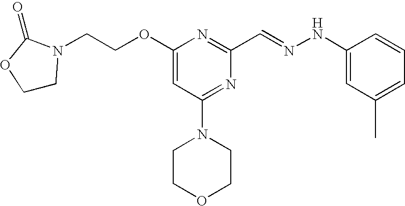 | 3-{2-[6-Morpholin-4-yl-2-(m- tolyl-hydrazonomethyl)- pyrimidin-4-yloxy]-ethyl}- oxazolidin-2-one |
| 376 | 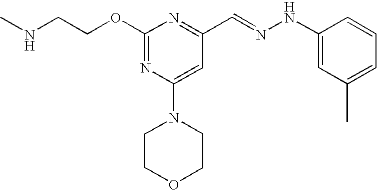 | Methyl-{2-[4-morpholin-4-yl- 6-(m-tolyl-hydrazonomethyl)- pyrimidin-2-yloxy]-ethyl}- amine |
| 377 | 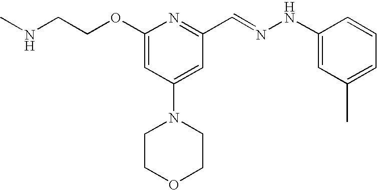 | Methyl-{2-[4-morpholin-4-yl- 6-(m-tolyl-hydrazonomethyl)- pyridin-2-yloxy]-ethyl}-amine |
| 378 | 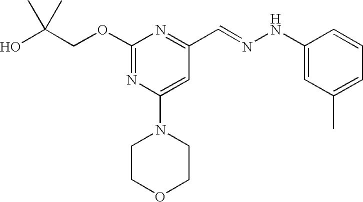 | 2-Methyl-1-[4-morpholin-4-yl- 6-(m-tolyl-hydrazonomethyl)- pyrimidin-2-yloxy]-propan-2-ol |
| 379 | 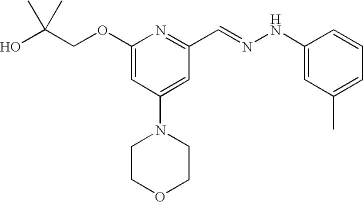 | 2-Methyl-1-[4-morpholin-4-yl- 6-(m-tolyl-hydrazonomethyl)- pyridin-2-yloxy]-propan-2-ol |
| 380 | 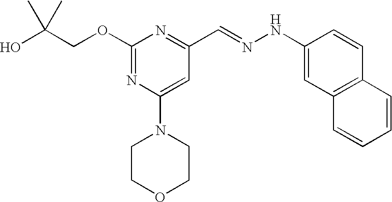 | 2-Methyl-1-[4-morpholin-4-yl- 6-(naphthalen-2-yl- hydrazonomethyl)-pyrimidin-2- yloxy]-propan-2-ol |
| 381 | 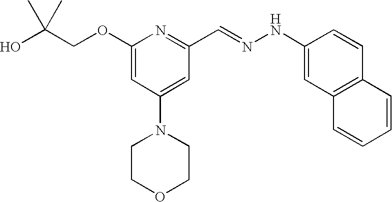 | 2-Methyl-1-[4-morpholin-4-yl- 6-(naphthalen-2-yl- hydrazonomethyl)-pyridin-2- yloxy]-propan-2-ol |
| 382 | 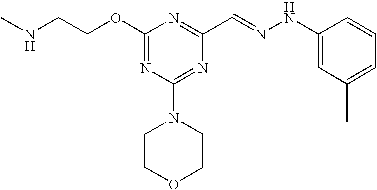 | Methyl-{2-[4-morpholin-4-yl- 6-(m-tolyl-hydrazonomethyl)- [1,3,5]triazin-2-yloxy]-ethyl}- amine |
| 383 | 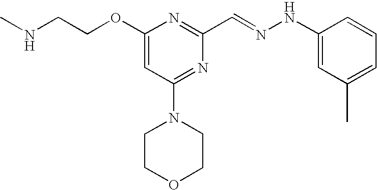 | Methyl-{2-[6-morpholin-4-yl- 2-(m-tolyl-hydrazonomethyl)- pyrimidin-4-yloxy]-ethyl}- amine |
| 384 | 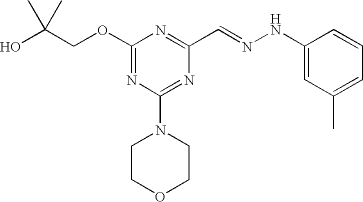 | 2-Methyl-1-[4-morpholin-4-yl- 6-(m-tolyl-hydrazonomethyl)- [1,3,5]triazin-2-yloxy]-propan- 2-ol |
| 385 | 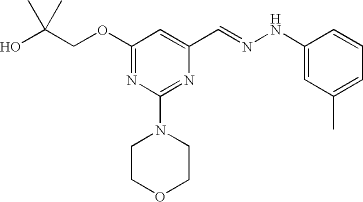 | 2-Methyl-1-[2-morpholin-4-yl- 6-(m-tolyl-hydrazonomethyl)- pyrimidin-4-yloxy]-propan-2-ol |
| 386 | 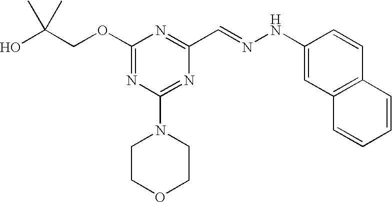 | 2-Methyl-1-[4-morpholin-4-yl- 6-(naphthalen-2-yl- hydrazonomethyl)- [1,3,5]triazin-2-yloxy]-propan- 2-ol |
| 387 | 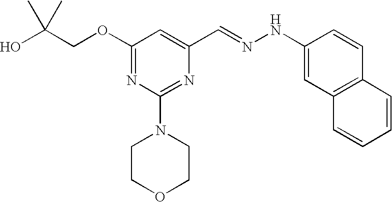 | 2-Methyl-1-[2-morpholin-4-yl- 6-(naphthalen-2-yl- hydrazonomethyl)-pyrimidin-4- yloxy]-propan-2-ol |
| 388 | 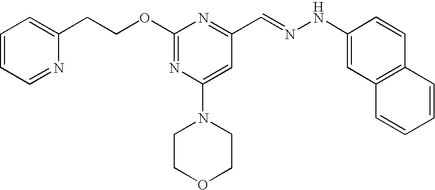 | N-[6-Morpholin-4-yl-2-(2- pyridin-2-yl-ethoxy)- pyrimidin-4-ylmethylene]-N′- naphthalen-2-yl-hydrazine |
| 389 | 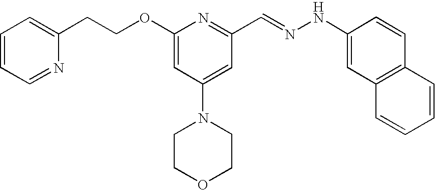 | N-[4-Morpholin-4-yl-6-(2- pyridin-2-yl-ethoxy)-pyridin-2- ylmethylene]-N′-naphthalen-2- yl-hydrazine |
| 390 | 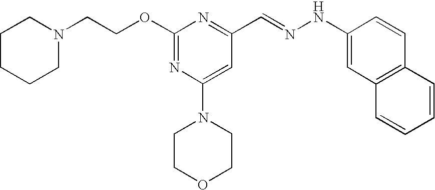 | N-[6-Morpholin-4-yl-2-(2- piperidin-1-yl-ethoxy)- pyrimidin-4-ylmethylene]-N′- naphthalen-2-yl-hydrazine |
| 391 | 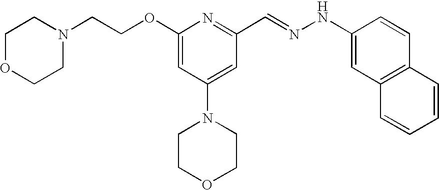 | N-[4-Morpholin-4-yl-6-(2- morpholin-4-yl-ethoxy)- pyridin-2-ylmethylene]-N′- naphthalen-2-yl-hydrazine |
| 392 | 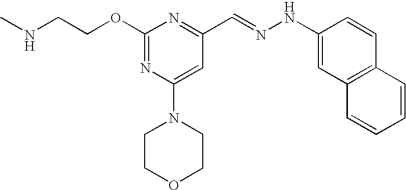 | Methyl-{2-[4-morpholin-4-yl- 6-(naphthalen-2-yl- hydrazonomethyl)-pyrimidin-2- yloxy]-ethyl}-amine |
| 393 | 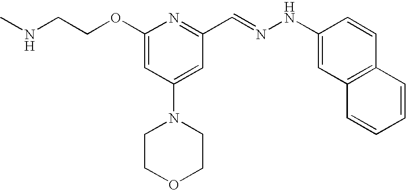 | Methyl-{2-[4-morpholin-4-yl- 6-(naphthalen-2-yl- hydrazonomethyl)-pyridin-2- yloxy]-ethyl}-amine |
| 394 | 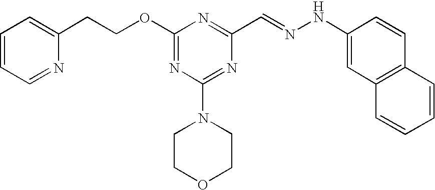 | N-[4-Morpholin-4-yl-6-(2- pyridin-2-yl-ethoxy)- [1,3,5]triazin-2-ylmethylene]- N′-naphthalen-2-yl-hydrazine |
| 395 | 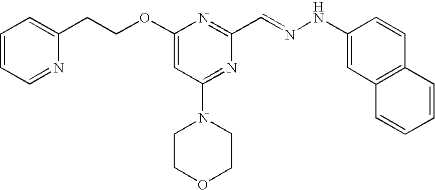 | N-[4-Morpholin-4-yl-6-(2- pyridin-2-yl-ethoxy)- pyrimidin-2-ylmethylene]-N′- naphthalen-2-yl-hydrazine |
| 396 | 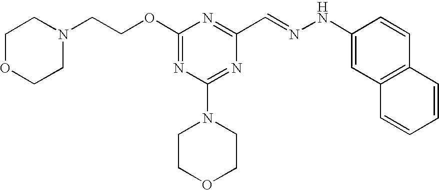 | N-[4-Morpholin-4-yl-6-(2- morpholin-4-yl-ethoxy)- [1,3,5]triazin-2-ylmethylene]- N′-naphthalen-2-yl-hydrazine |
| 397 | 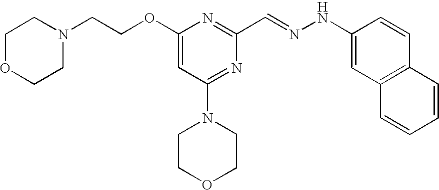 | N-[4-Morpholin-4-yl-6-(2- morpholin-4-yl-ethoxy)- pyrimidin-2-ylmethylene]-N′- naphthalen-2-yl-hydrazine |
| 398 | 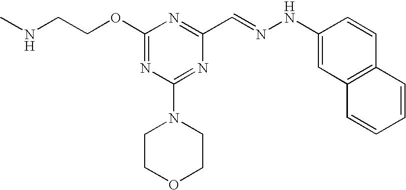 | Methyl-{2-[4-morpholin-4-yl- 6-(naphthalen-2-yl- hydrazonomethyl)- [1,3,5]triazin-2-yloxy]-ethyl}- amine |
| 399 | 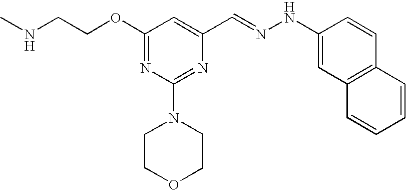 | Methyl-{2-[2-morpholin-4-yl- 6-(naphthalen-2-yl- hydrazonomethyl)-pyrimidin-4- yloxy]-ethyl}-amine |
| 400 | 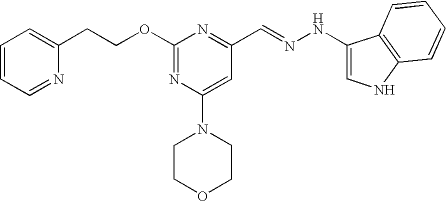 | N-(1H-Indol-3-yl)-N′-[6- morpholin-4-yl-2-(2-pyridin-2- yl-ethoxy)-pyrimidin-4- ylmethylene]-hydrazine |
| 401 | 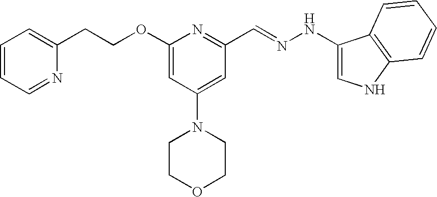 | N-(1H-Indol-3-yl)-N′-[4- morpholin-4-yl-6-(2-pyridin-2- yl-ethoxy)-pyridin-2- ylmethylene]-hydrazine |
| 402 | 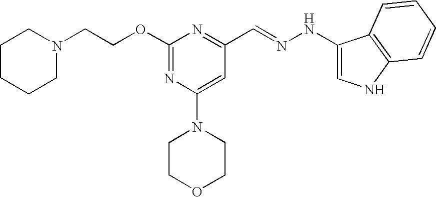 | N-(1H-Indol-3-yl)-N′-[6- morpholin-4-yl-2-(2-piperidin- 1-yl-ethoxy)-pyrimidin-4- ylmethylene]-hydrazine |
| 403 | 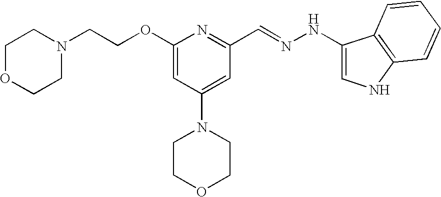 | N-(1H-Indol-3-yl)-N′-[4- morpholin-4-yl-6-(2- morpholin-4-yl-ethoxy)- pyridin-2-ylmethylene]- hydrazine |
| 404 | 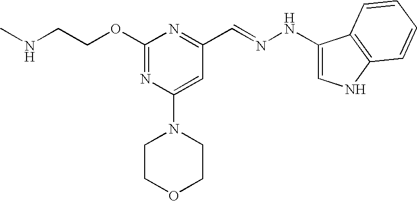 | (2-{4-[(1H-Indol-3-yl)- hydrazonomethyl]-6- morpholin-4-yl-pyrimidin-2- yloxy}-ethyl)-methyl-amine |
| 405 | 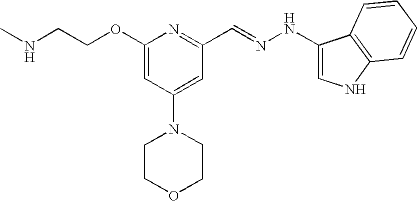 | (2-{6-[(1H-Indol-3-yl)- hydrazonomethyl]-4- morpholin-4-yl-pyridin-2- yloxy}-ethyl)-methyl-amine |
| 406 | 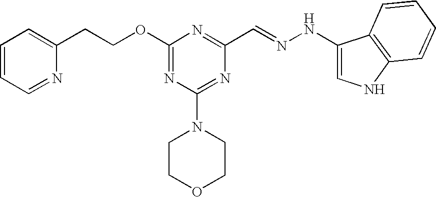 | N-(1H-Indol-3-yl)-N′-[4- morpholin-4-yl-6-(2-pyridin-2- yl-ethoxy)-[1,3,5]triazin-2- ylmethylene]-hydrazine |
| 407 | 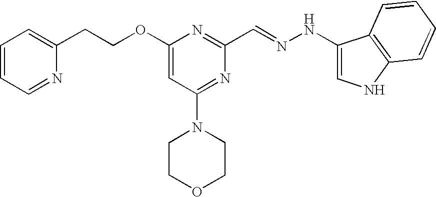 | N-(1H-Indol-3-yl)-N′-[4- morpholin-4-yl-6-(2-pyridin-2- yl-ethoxy)-pyrimidin-2- ylmethylene]-hydrazine |
| 408 | 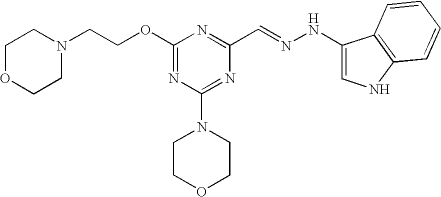 | N-(1H-Indol-3-yl)-N′-[4- morpholin-4-yl-6-(2- morpholin-4-yl-ethoxy)- [1,3,5]triazin-2-ylmethylene]- hydrazine |
| 409 | 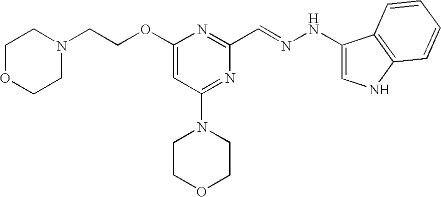 | N-(1H-Indol-3-yl)-N′-[4- morpholin-4-yl-6-(2- morpholin-4-yl-ethoxy)- pyrimidin-2-ylmethylene]- hydrazine |
| 410 | 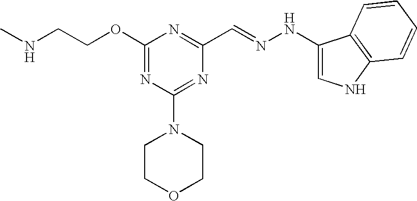 | (2-{4-[(1H-Indol-3-yl)- hydrazonomethyl]-6- morpholin-4-yl-[1,3,5]triazin- 2-yloxy}-ethyl)-methyl-amine |
| 411 | 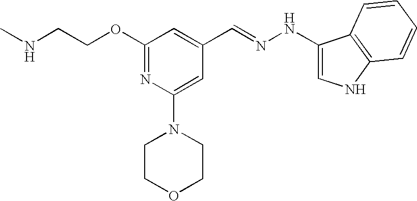 | (2-{6-[(1H-Indol-3-yl)- hydrazonomethyl]-2- morpholin-4-yl-pyrimidin-4- yloxy}-ethyl)-methyl-amine |
| 412 | 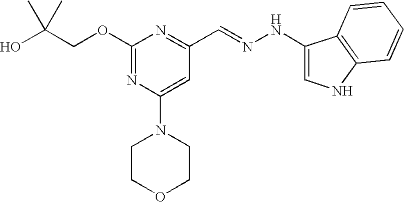 | 1-{4-[(1H-Indol-3-yl)- hydrazonomethyl]-6- morpholin-4-yl-pyrimidin-2- yloxy}-2-methyl-propan-2-ol |
| 413 | 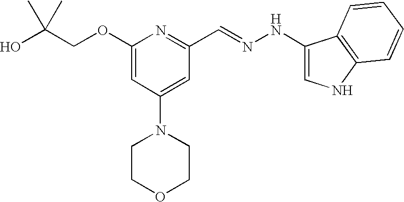 | 1-{6-[(1H-Indol-3-yl)- hydrazonomethyl]-4- morpholin-4-yl-pyridin-2- yloxy}-2-methyl-propan-2-ol |
| 414 | 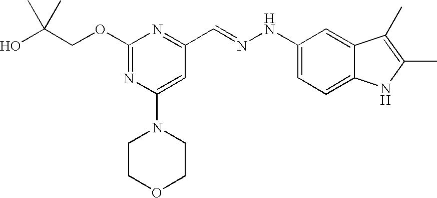 | 1-{4-[(2,3-Dimethyl-1H-indol- 5-yl)-hydrazonomethyl]-6- morpholin-4-yl-pyrimidin-2- yloxy}-2-methyl-propan-2-ol |
| 415 | 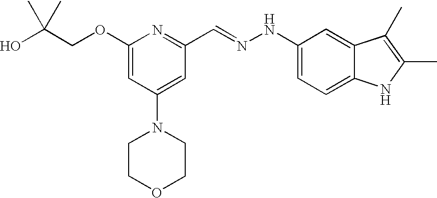 | 1-{6-[(2,3-Dimethyl-1H-indol- 5-yl)-hydrazonomethyl]-4- morpholin-4-yl-pyridin-2- yloxy}-2-methyl-propan-2-ol |
| 416 | 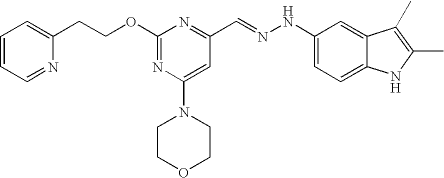 | N-(2,3-Dimethyl-1H-indol-5- yl)-N′-[6-morpholin-4-yl-2-(2- pyridin-2-yl-ethoxy)- pyrimidin-4-ylmethylene]- hydrazine |
| 417 | 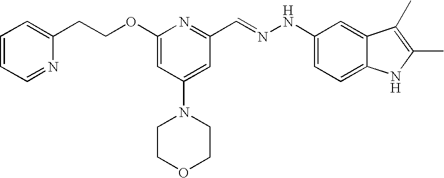 | N-(2,3-Dimethyl-1H-indol-5- yl)-N′-[4-morpholin-4-yl-6-(2- pyridin-2-yl-ethoxy)-pyridin-2- ylmethylene]-hydrazine |
| 418 | 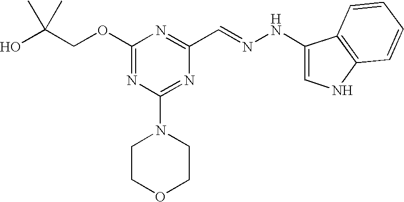 | 1-{4-[(1H-Indol-3-yl)- hydrazonomethyl]-6- morpholin-4-yl-[1,3,5]triazin- 2-yloxy}-2-methyl-propan-2-ol |
| 419 | 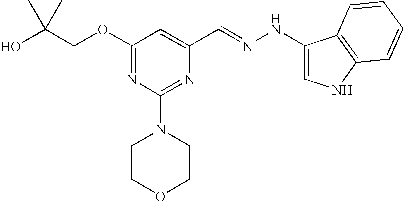 | 1-{6-[(1H-Indol-3-yl)- hydrazonomethyl]-2- morpholin-4-yl-pyrimidin-4- yloxy}-2-methyl-propan-2-ol |
| 420 | 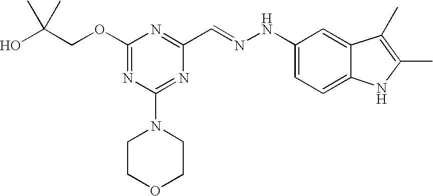 | 1-{4-[(2,3-Dimethyl-1H-indol- 5-yl)-hydrazonomethyl]-6- morpholin-4-yl-[1,3,5]triazin- 2-yloxy}-2-methyl-propan-2-ol |
| 421 | 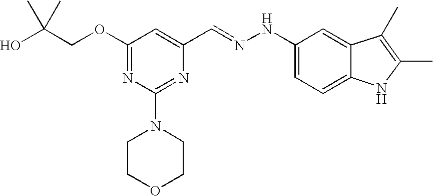 | 1-{6-[(2,3-Dimethyl-1H-indol- 5-yl)-hydrazonomethyl]-2- morpholin-4-yl-pyrimidin-4- yloxy}-2-methyl-propan-2-ol |
| 422 | 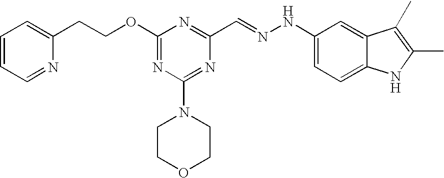 | N-(2,3-Dimethyl-1H-indol-5- yl)-N′-[4-morpholin-4-yl-6-(2- pyridin-2-yl-ethoxy)- [1,3,5]triazin-2-ylmethylene]- hydrazine |
| 423 | 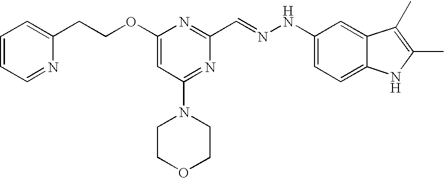 | N-(2,3-Dimethyl-1H-indol-5- yl)-N′-[4-morpholin-4-yl-6-(2- pyridin-2-yl-ethoxy)- pyrimidin-2-ylmethylene]- hydrazine |
| 424 | 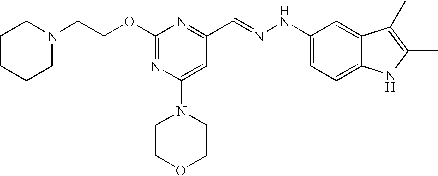 | N-(2,3-Dimethyl-1H-indol-5- yl)-N′-[6-morpholin-4-yl-2-(2- piperidin-1-yl-ethoxy)- pyrimidin-4-ylmethylene]- hydrazine |
| 425 | 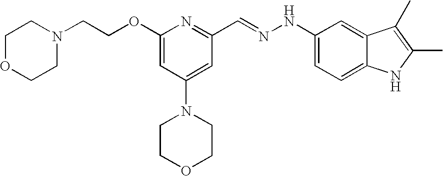 | N-(2,3-Dimethyl-1H-indol-5- yl)-N′-[4-morpholin-4-yl-6-(2- morpholin-4-yl-ethoxy)- pyridin-2-ylmethylene]- hydrazine |
| 426 | 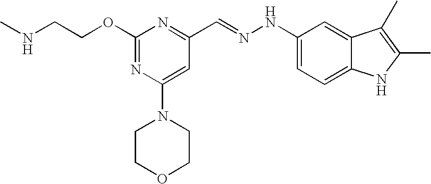 | (2-{4-[(2,3-Dimethyl-1H- indol-5-yl)-hydrazonomethyl]- 6-morpholin-4-yl-pyrimidin-2- yloxy}-ethyl)-methyl-amine |
| 427 | 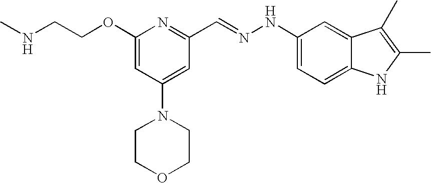 | (2-{6-[(2,3-Dimethyl-1H- indol-5-yl)-hydrazonomethyl]- 4-morpholin-4-yl-pyridin-2- yloxy}-ethyl)-methyl-amine |
| 428 | 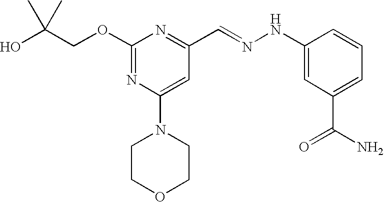 | 3-{N′-[2-(2-Hydroxy-2-methyl- propoxy)-6-morpholin-4-yl- pyrimidin-4-ylmethylene]- hydrazino}-benzamide |
| 429 | 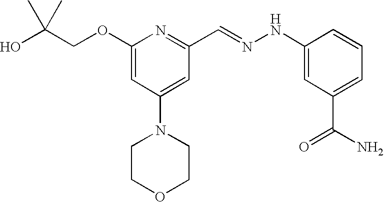 | 3-{N′-[6-(2-Hydroxy-2-methyl- propoxy)-4-morpholin-4-yl- pyridin-2-ylmethylene]- hydrazino}-benzamide |
| 430 | 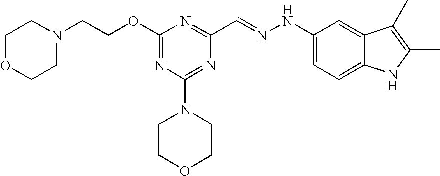 | N-(2,3-Dimethyl-1H-indol-5- yl)-N′-[4-morpholin-4-yl-6-(2- morpholin-4-yl-ethoxy)- [1,3,5]triazin-2-ylmethylene]- hydrazine |
| 431 | 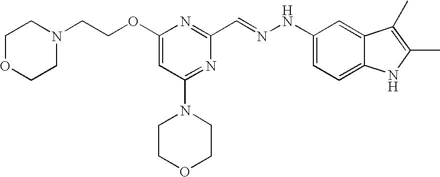 | N-(2,3-Dimethyl-1H-indol-5- yl)-N′-[4-morpholin-4-yl-6-(2- morpholin-4-yl-ethoxy)- pyrimidin-2-ylmethylene]- hydrazine |
| 432 | 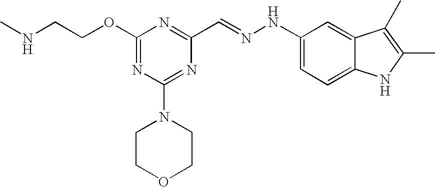 | (2-{4-[(2,3-Dimethyl-1H- indol-5-yl)-hydrazonomethyl]- 6-morpholin-4-yl- [1,3,5]triazin-2-yloxy}-ethyl)- methyl-amine |
| 433 | 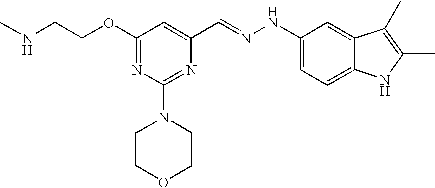 | (2-{6-[(2,3-Dimethyl-1H- indol-5-yl)-hydrazonomethyl]- 2-morpholin-4-yl-pyrimidin-4- yloxy}-ethyl)-methyl-amine |
| 434 | 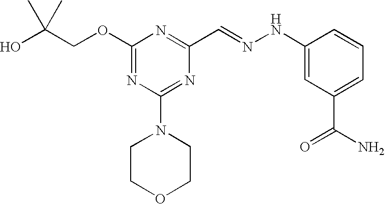 | 3-{N′-[4-(2-Hydroxy-2-methyl- propoxy)-6-morpholin-4-yl- [1,3,5]triazin-2-ylmethylene]- hydrazino}-benzamide |
| 435 | 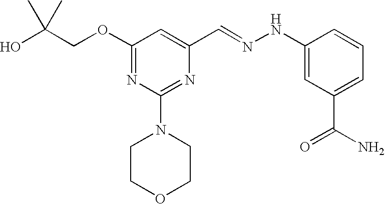 | 3-{N′-[6-(2-Hydroxy-2-methyl- propoxy)-2-morpholin-4-yl- pyrimidin-4-ylmethylene]- hydrazino}-benzamide |
| 436 | 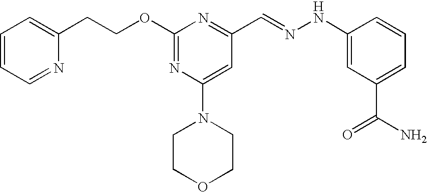 | 3-{N′-[6-Morpholin-4-yl-2-(2- pyridin-2-yl-ethoxy)- pyrimidin-4-ylmethylene]- hydrazino}-benzamide |
| 437 | 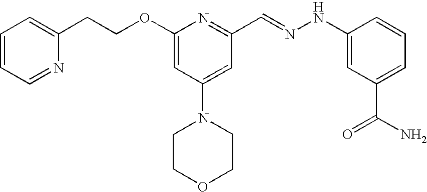 | 3-{N′-[4-Morpholin-4-yl-6-(2- pyridin-2-yl-ethoxy)-pyridin-2- ylmethylene]-hydrazino}- benzamide |
| 438 | 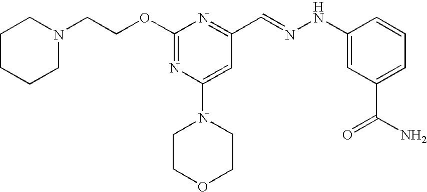 | 3-{N′-[6-Morpholin-4-yl-2-(2- piperidin-1-yl-ethoxy)- pyrimidin-4-ylmethylene]- hydrazino}-benzamide |
| 439 | 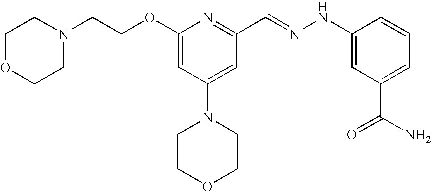 | 3-{N′-[4-Morpholin-4-yl-6-(2- morpholin-4-yl-ethoxy)- pyridin-2-ylmethylene]- hydrazino}-benzamide |
| 440 | 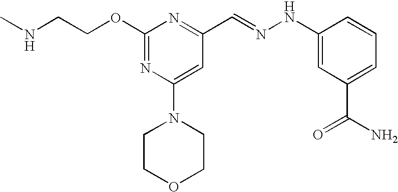 | 3-{N′-[2-(2-Methylamino- ethoxy)-6-morpholin-4-yl- pyrimidin-4-ylmethylene]- hydrazino}-benzamide |
| 441 | 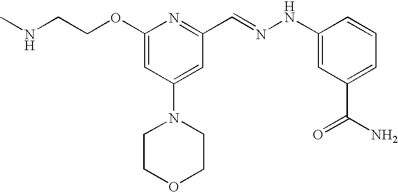 | 3-{N′-[6-(2-Methylamino- ethoxy)-4-morpholin-4-yl- pyridin-2-ylmethylene]- hydrazino}-benzamide |
| 442 | 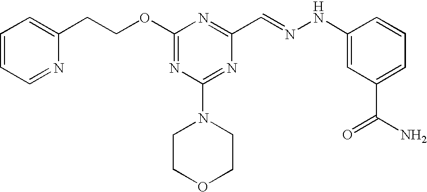 | 3-{N′-[4-Morpholin-4-yl-6-(2- pyridin-2-yl-ethoxy)- [1,3,5]triazin-2-ylmethylene]- hydrazino}-benzamide |
| 443 | 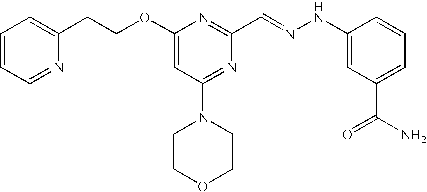 | 3-{N′-[4-Morpholin-4-yl-6-(2- pyridin-2-yl-ethoxy)- pyrimidin-2-ylmethylene]- hydrazino}-benzamide |
| 444 | 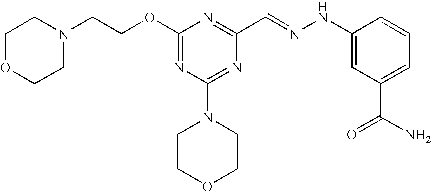 | 3-{N′-[4-Morpholin-4-yl-6-(2- morpholin-4-yl-ethoxy)- [1,3,5]triazin-2-ylmethylene]- hydrazino}-benzamide |
| 445 | 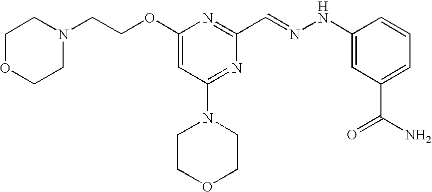 | 3-{N′-[4-Morpholin-4-yl-6-(2- morpholin-4-yl-ethoxy)- pyrimidin-2-ylmethylene]- hydrazino}-benzamide |
| 446 | 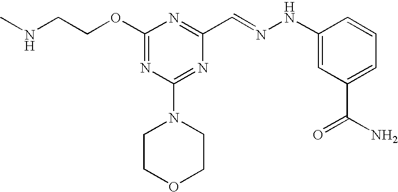 | 3-{N′-[4-(2-Methylamino- ethoxy)-6-morpholin-4-yl- [1,3,5]triazin-2-ylmethylene]- hydrazino}-benzamide |
| 447 | 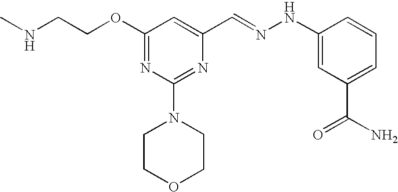 | 3-{N′-[6-(2-Methylamino- ethoxy)-2-morpholin-4-yl- pyrimidin-4-ylmethylene]- hydrazino}-benzamide |
| 448 | 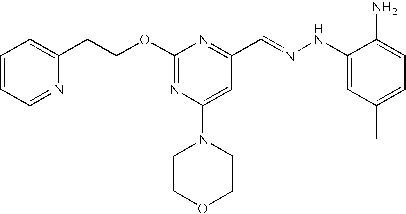 | 4-Methyl-2-{N′-[6-morpholin- 4-yl-2-(2-pyridin-2-yl-ethoxy)- pyrimidin-4-ylmethylene]- hydrazino}-phenylamine |
| 449 | 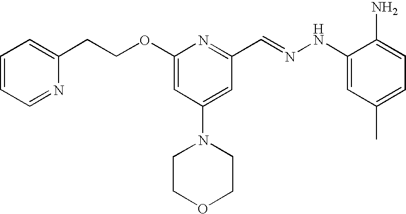 | 4-Methyl-2-{N′-[4-morpholin- 4-yl-6-(2-pyridin-2-yl-ethoxy)- pyridin-2-ylmethylene]- hydrazino}-phenylamine |
| 450 | 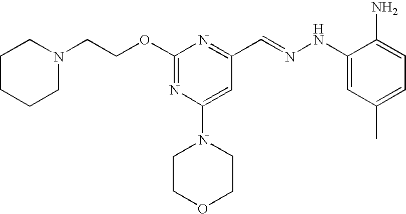 | 4-Methyl-2-{N′-[6-morpholin- 4-yl-2-(2-piperidin-1-yl- ethoxy)-pyrimidin-4- ylmethylene]-hydrazino}- phenylamine |
| 451 | 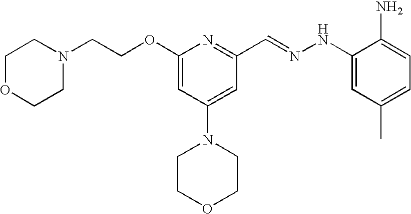 | 4-Methyl-2-{N′-[4-morpholin- 4-yl-6-(2-morpholin-4-yl- ethoxy)-pyridin-2- ylmethylene]-hydrazino}- phenylamine |
| 452 | 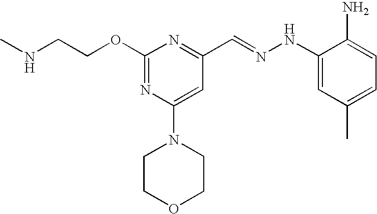 | 4-Methyl-2-{N′-[2-(2- methylamino-ethoxy)-6- morpholin-4-yl-pyrimidin-4- ylmethylene]-hydrazino}- phenylamine |
| 453 | 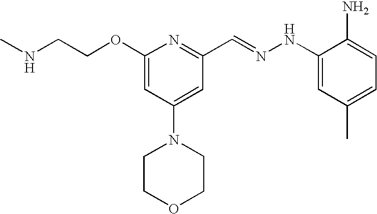 | 4-Methyl-2-{N′-[6-(2- methylamino-ethoxy)-4- morpholin-4-yl-pyridin-2- ylmethylene]-hydrazino}- phenylamine |
| 454 | 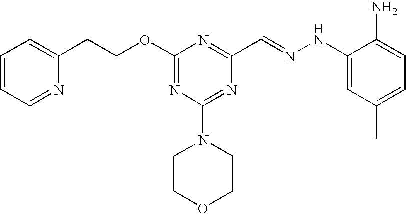 | 4-Methyl-2-{N′-[4-morpholin- 4-yl-6-(2-pyridin-2-yl-ethoxy)- [1,3,5]triazin-2-ylmethylene]- hydrazino}-phenylamine |
| 455 | 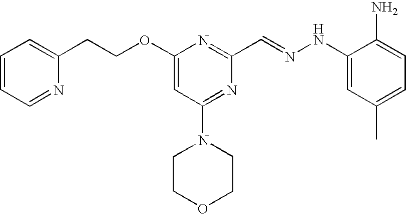 | 4-Methyl-2-{N′-[4-morpholin- 4-yl-6-(2-pyridin-2-yl-ethoxy)- pyrimidin-2-ylmethylene]- hydrazino}-phenylamine |
| 456 | 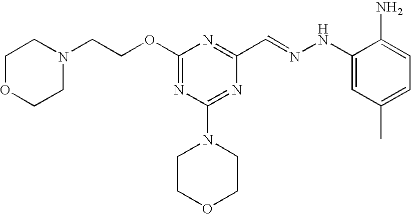 | 4-Methyl-2-{N′-[4-morpholin- 4-yl-6-(2-morpholin-4-yl- ethoxy)-[1,3,5]triazin-2- ylmethylene]-hydrazino}- phenylamine |
| 457 | 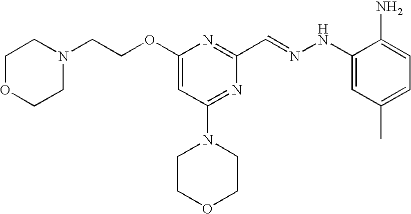 | 4-Methyl-2-{N′-[4-morpholin- 4-yl-6-(2-morpholin-4-yl- ethoxy)-pyrimidin-2- ylmethylene]-hydrazino}- phenylamine |
| 458 | 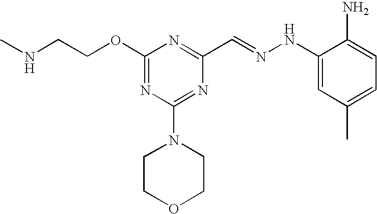 | 4-Methyl-2-{N′-[4-(2- methylamino-ethoxy)-6- morpholin-4-yl-[1,3,5]triazin- 2-ylmethylene]-hydrazino}- phenylamine |
| 459 | 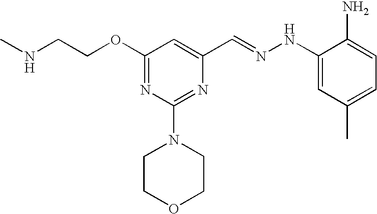 | 4-Methyl-2-{N′-[6-(2- methylamino-ethoxy)-2- morpholin-4-yl-pyrimidin-4- ylmethylene]-hydrazino}- phenylamine |
| 460 | 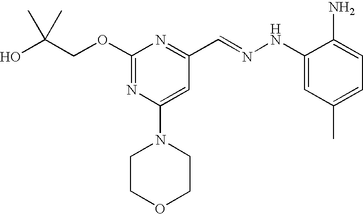 | 1-{4-[(2-Amino-5-methyl- phenyl)-hydrazonomethyl]-6- morpholin-4-yl-pyrimidin-2- yloxy}-2-methyl-propan-2-ol |
| 461 | 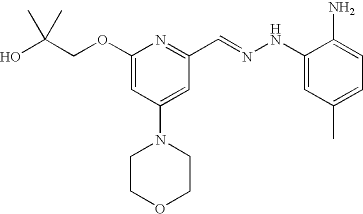 | 1-{6-[(2-Amino-5-methyl- phenyl)-hydrazonomethyl]-4- morpholin-4-yl-pyridin-2- yloxy}-2-methyl-propan-2-ol |
| 462 | 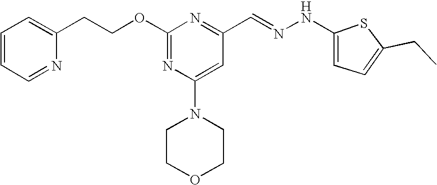 | N-(5-Ethyl-thiophen-2-yl)-N′- [6-morpholin-4-yl-2-(2- pyridin-2-yl-ethoxy)- pyrimidin-4-ylmethylene]- hydrazine |
| 463 | 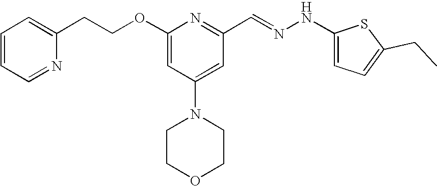 | N-(5-Ethyl-thiophen-2-yl)-N′- [4-morpholin-4-yl-6-(2- pyridin-2-yl-ethoxy)-pyridin-2- ylmethylene]-hydrazine |
| 464 | 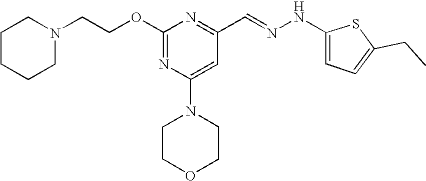 | N-(5-Ethyl-thiophen-2-yl)-N′- [6-morpholin-4-yl-2-(2- piperidin-1-yl-ethoxy)- pyrimidin-4-ylmethylene]- hydrazine |
| 465 | 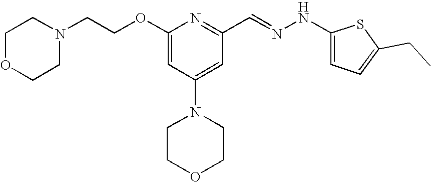 | N-(5-Ethyl-thiophen-2-yl)-N′- [4-morpholin-4-yl-6-(2- morpholin-4-yl-ethoxy)- pyridin-2-ylmethylene]- hydrazine |
| 466 | 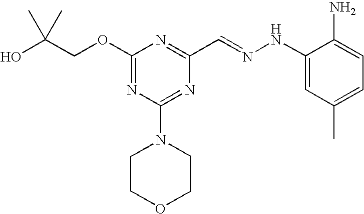 | 1-{4-[(2-Amino-5-methyl- phenyl)-hydrazonomethyl]-6- morpholin-4-yl-[1,3,5]triazin- 2-yloxy}-2-methyl-propan-2-ol |
| 467 | 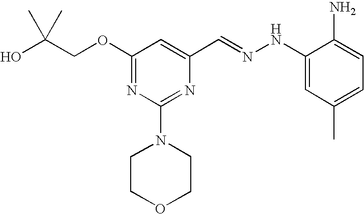 | 1-{6-[(2-Amino-5-methyl- phenyl)-hydrazonomethyl]-2- morpholin-4-yl-pyrimidin-4- yloxy}-2-methyl-propan-2-ol |
| 468 | 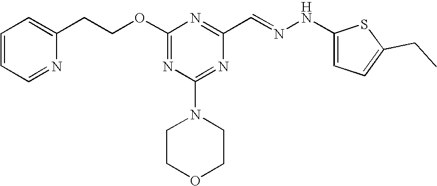 | N-(5-Ethyl-thiophen-2-yl)-N′- [4-morpholin-4-yl-6-(2- pyridin-2-yl-ethoxy)- [1,3,5]triazin-2-ylmethylene]- hydrazine |
| 469 | 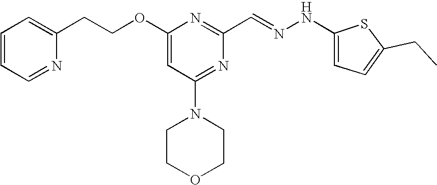 | N-(5-Ethyl-thiophen-2-yl)-N′- [4-morpholin-4-yl-6-(2- pyridin-2-yl-ethoxy)- pyrimidin-2-ylmethylene]- hydrazine |
| 470 | 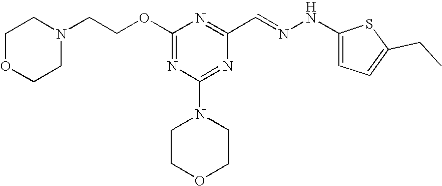 | N-(5-Ethyl-thiophen-2-yl)-N′- [4-morpholin-4-yl-6-(2- morpholin-4-yl-ethoxy)- [1,3,5]triazin-2-ylmethylene]- hydrazine |
| 471 | 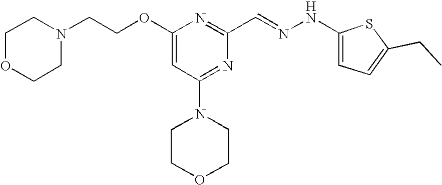 | N-(5-Ethyl-thiophen-2-yl)-N′- [4-morpholin-4-yl-6-(2- morpholin-4-yl-ethoxy)- pyrimidin-2-ylmethylene]- hydrazine |
| 472 | 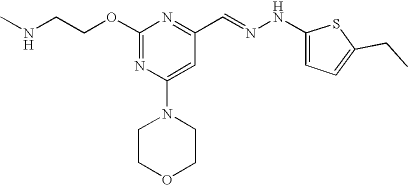 | (2-{4-[(5-Ethyl-thiophen-2-yl)- hydrazonomethyl]-6- morpholin-4-yl-pyrimidin-2- yloxy}-ethyl)-methyl-amine |
| 473 | 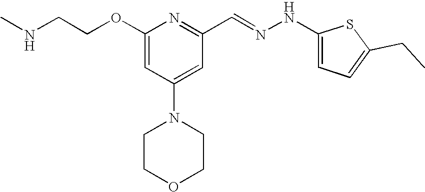 | (2-{6-[(5-Ethyl-thiophen-2-yl)- hydrazonomethyl]-4- morpholin-4-yl-pyridin-2- yloxy}-ethyl)-methyl-amine |
| 474 | 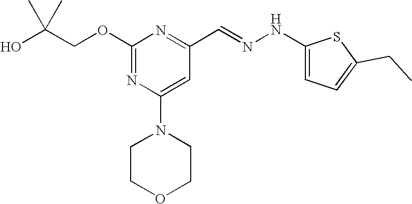 | 1-{4-[(5-Ethyl-thiophen-2-yl)- hydrazonomethyl]-6- morpholin-4-yl-pyrimidin-2- yloxy}-2-methyl-propan-2-ol |
| 475 | 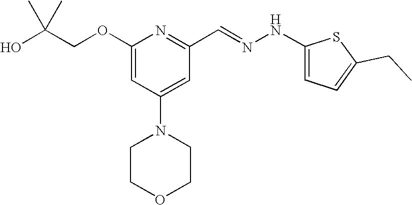 | 1-{6-[(5-Ethyl-thiophen-2-yl)- hydrazonomethyl]-4- morpholin-4-yl-pyridin-2- yloxy}-2-methyl-propan-2-ol |
| 476 | 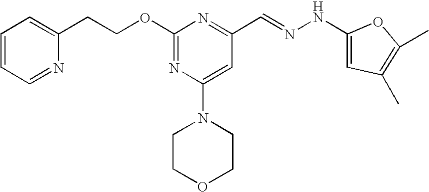 | N-(4,5-Dimethyl-furan-2-yl)- N′-[6-morpholin-4-yl-2-(2- pyridin-2-yl-ethoxy)- pyrimidin-4-ylmethylene]- hydrazine |
| 477 | 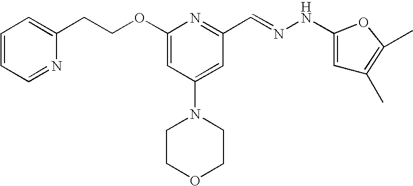 | N-(4,5-Dimethyl-furan-2-yl)- N′-[4-morpholin-4-yl-6-(2- pyridin-2-yl-ethoxy)-pyridin-2- ylmethylene]-hydrazine |
| 478 | 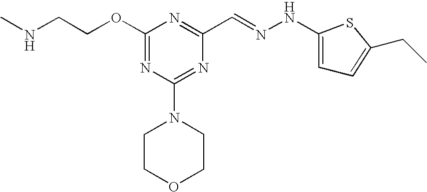 | (2-{4-[(5-Ethyl-thiophen-2-yl)- hydrazonomethyl]-6- morpholin-4-yl-[1,3,5]triazin- 2-yloxy}-ethyl)-methyl-amine |
| 479 | 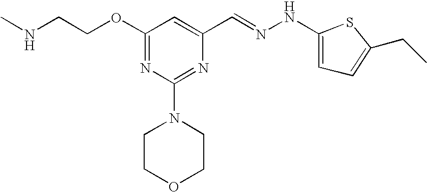 | (2-{6-[(5-Ethyl-thiophen-2-yl)- hydrazonomethyl]-2- morpholin-4-yl-pyrimidin-4- yloxy}-ethyl)-methyl-amine |
| 480 | 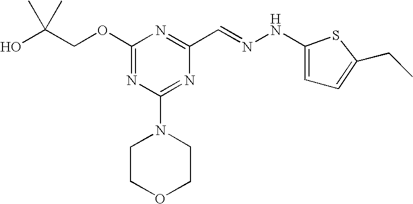 | 1-{4-[(5-Ethyl-thiophen-2-yl)- hydrazonomethyl]-6- morpholin-4-yl-[1,3,5]triazin- 2-yloxy}-2-methyl-propan-2-ol |
| 481 | 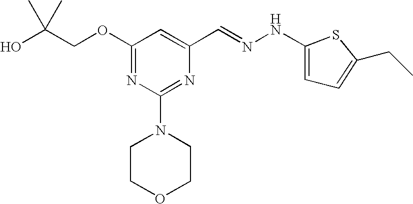 | 1-{6-[(5-Ethyl-thiophen-2-yl)- hydrazonomethyl]-2- morpholin-4-yl-pyrimidin-4- yloxy}-2-methyl-propan-2-ol |
| 482 | 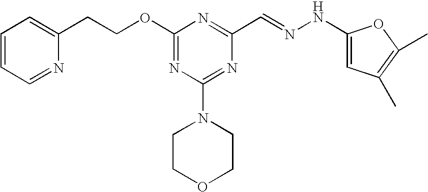 | N-(4,5-Dimethyl-furan-2-yl)- N′-[4-morpholin-4-yl-6-(2- pyridin-2-yl-ethoxy)- [1,3,5]triazin-2-ylmethylene]- hydrazine |
| 483 | 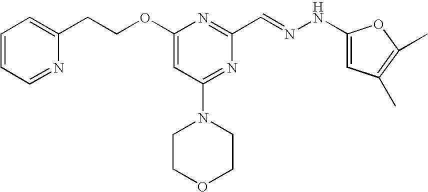 | N-(4,5-Dimethyl-furan-2-yl)- N′-[4-morpholin-4-yl-6-(2- pyridin-2-yl-ethoxy)- pyrimidin-2-ylmethylene]- hydrazine |
| 484 | 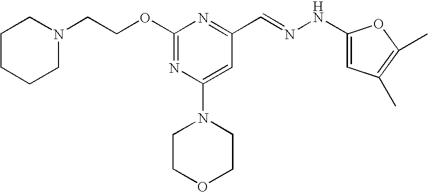 | N-(4,5-Dimethyl-furan-2-yl)- N′-[6-morpholin-4-yl-2-(2- piperidin-1-yl-ethoxy)- pyrimidin-4-ylmethylene]- hydrazine |
| 485 | 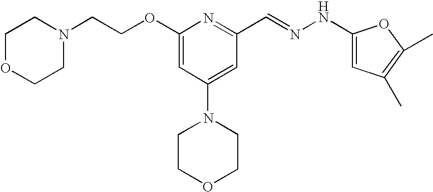 | N-(4,5-Dimethyl-furan-2-yl)- N′-[4-morpholin-4-yl-6-(2- morpholin-4-yl-ethoxy)- pyridin-2-ylmethylene]- hydrazine |
| 486 | 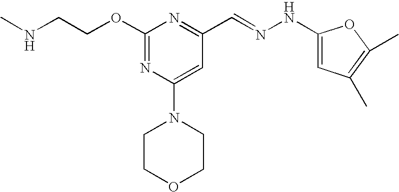 | (2-{4-[(4,5-Dimethyl-furan-2- yl)-hydrazonomethyl]-6- morpholin-4-yl-pyrimidin-2- yloxy}-ethyl)-methyl-amine |
| 487 | 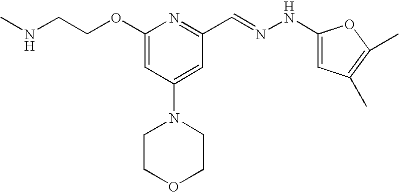 | (2-{6-[(4,5-Dimethyl-furan-2- yl)-hydrazonomethyl]-4- morpholin-4-yl-pyridin-2- yloxy}-ethyl)-methyl-amine |
| 488 | 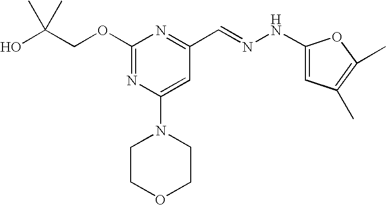 | 1-{4-[(4,5-Dimethyl-furan-2- yl)-hydrazonomethyl]-6- morpholin-4-yl-pyrimidin-2- yloxy}-2-methyl-propan-2-ol |
| 489 | 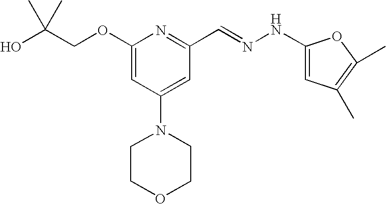 | 1-{6-[(4,5-Dimethyl-furan-2- yl)-hydrazonomethyl]-4- morpholin-4-yl-pyridin-2- yloxy}-2-methyl-propan-2-ol |
| 490 | 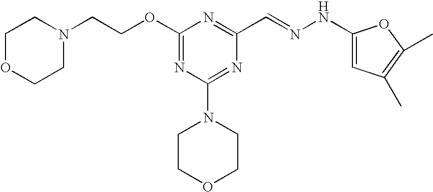 | N-(4,5-Dimethyl-furan-2-yl)- N′-[4-morpholin-4-yl-6-(2- morpholin-4-yl-ethoxy)- [1 ,3,5]triazin-2-ylmethylene]- hydrazine |
| 491 | 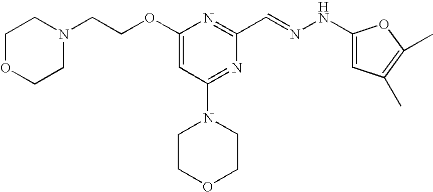 | N-(4,5-Dimethyl-furan-2-yl)- N′-[4-morpholin-4-yl-6-(2- morpholin-4-yl-ethoxy)- pyrimidin-2-ylmethylene]- hydrazine |
| 492 | 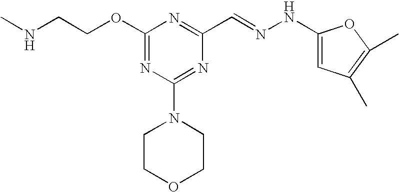 | (2-{4-[(4,5-Dimethyl-furan-2- yl)-hydrazonomethyl]-6- morpholin-4-yl-[1,3,5]triazin- 2-yloxy}-ethyl)-methyl-amine |
| 493 | 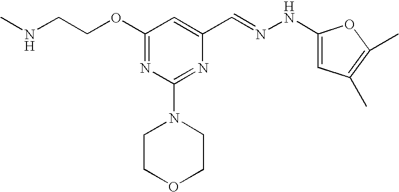 | (2-{6-[(4,5-Dimethyl-furan-2- yl)-hydrazonomethyl]-2- morpholin-4-yl-pyridin-4- yloxy}-ethyl)-methyl-amine |
| 494 | 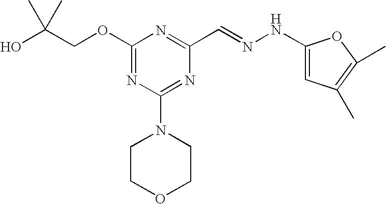 | {4-[(4,5-Dimethyl-furan-2-yl)- hydrazonomethyl]-6- morpholin-4-yl-[1,3,5]triazin- 2-yloxy}-2-methyl-propan-2-ol |
| 495 | 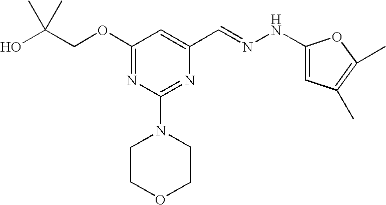 | 1-{6-[(4,5-Dimethyl-furan-2- yl)-hydrazonomethyl]-2- morpholin-4-yl-pyrimidin-4- yloxy}-2-methyl-propan-2-ol |
| 496 | 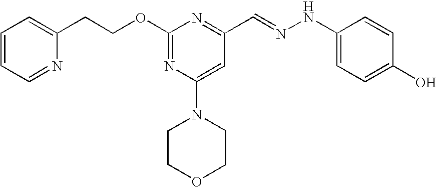 | 4-{N′-[6-Morpholin-4-yl-2-(2- pyridin-2-yl-ethoxy)- pyrimidin-4-ylmethylene]- hydrazino}-phenol |
| 497 | 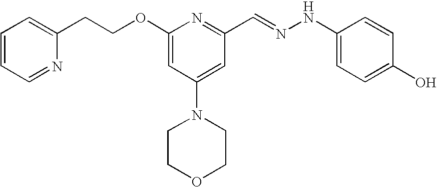 | 4-{N′-[4-Morpholin-4-yl-6-(2- pyridin-2-yl-ethoxy)-pyridin-2- ylmethylene]-hydrazino}- phenol |
| 498 | 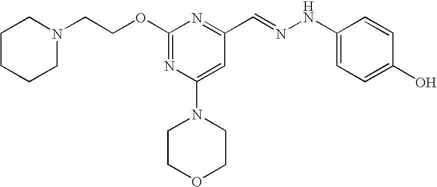 | 4-{N′-[6-Morpholin-4-yl-2-(2- piperidin-1-yl-ethoxy)- pyrimidin-4-ylmethylene]- hydrazino}-phenol |
| 499 | 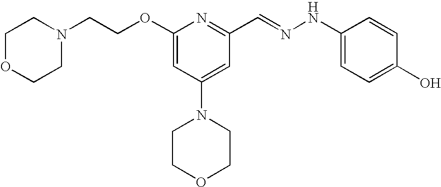 | 4-{N′-[4-Morpholin-4-yl-6-(2- morpholin-4-yl-ethoxy)- pyridin-2-ylmethylene]- hydrazino}-phenol |
| 500 | 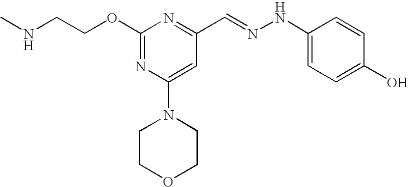 | 4-{N′-[2-(2-Methylamino- ethoxy)-6-morpholin-4-yl- pyrimidin-4-ylmethylene]- hydrazino}-phenol |
| 501 | 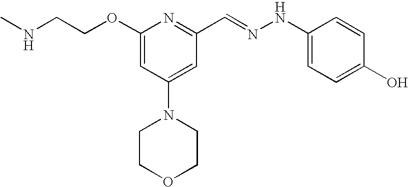 | 4-{N′-[6-(2-Methylamino- ethoxy)-4-morpholin-4-yl- pyridin-2-ylmethylene]- hydrazino}-phenol |
| 502 | 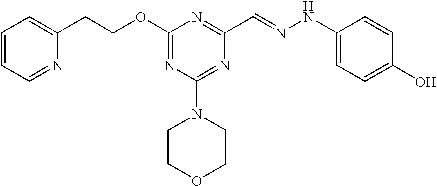 | 4-{N′-[4-Morpholin-4-yl-6-(2- pyridin-2-yl-ethoxy)- [1,3,5]triazin-2-ylmethylene]- hydrazino}-phenol |
| 503 | 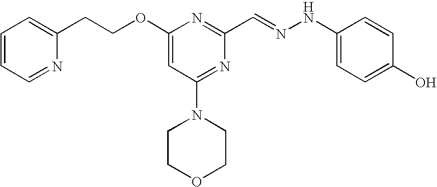 | 4-{N′-[4-Morpholin-4-yl-6-(2- pyridin-2-yl-ethoxy)- pyrimidin-2-ylmethylene]- hydrazino}-phenol |
| 504 | 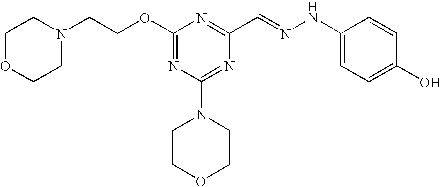 | 4-{N′-[4-Morpholin-4-yl-6-(2- morpholin-4-yl-ethoxy)- [1,3,5]triazin-2-ylmethylene]- hydrazino}-phenol |
| 505 | 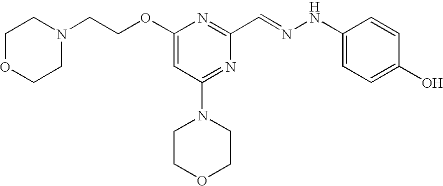 | 4-{N′-[4-Morpholin-4-yl-6-(2- morpholin-4-yl-ethoxy)- pyrimidin-2-ylmethylene]- hydrazino}-phenol |
| 506 | 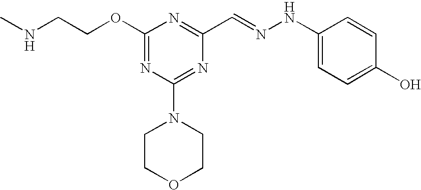 | 4-{N′-[4-(2-Methylamino- ethoxy)-6-morpholin-4-yl- [1,3,5]triazin-2-ylmethylene]- hydrazino}-phenol |
| 507 | 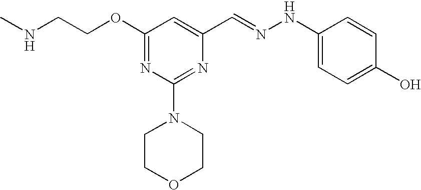 | 4-{N′-[6-(2-Methylamino- ethoxy)-2-morpholin-4-yl- pyrimidin-4-ylmethylene]- hydrazino}-phenol |
| 508 | 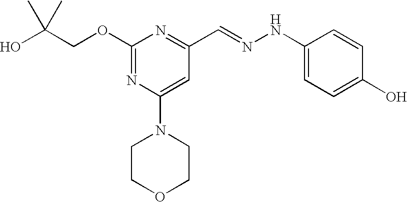 | 4-{N′-[2-(2-Hydroxy-2-methyl- propoxy)-6-morpholin-4-yl- pyrimidin-4-ylmethylene]- hydrazino}-phenol |
| 509 | 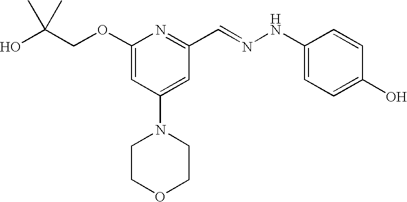 | 4-{N′-[6-(2-Hydroxy-2-methyl- propoxy)-4-morpholin-4-yl- pyridin-2-ylmethylene]- hydrazino}-phenol |
| 510 | 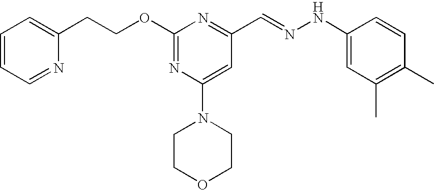 | N-(3,4-Dimethyl-phenyl)-N′- [6-morpholin-4-yl-2-(2- pyridin-2-yl-ethoxy)- pyrimidin-4-ylmethylene]- hydrazine |
| 511 | 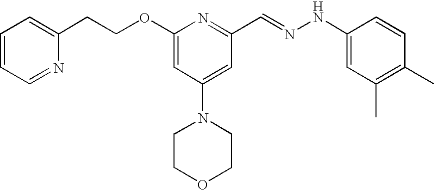 | N-(3,4-Dimethyl-phenyl)-N′- [4-morpholin-4-yl-6-(2- pyridin-2-yl-ethoxy)-pyridin-2- ylmethylene]-hydrazine |
| 512 | 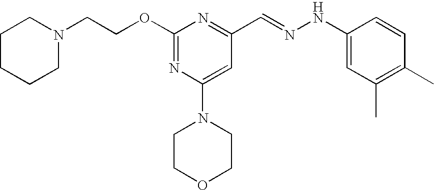 | N-(3,4-Dimethyl-phenyl)-N′- [6-morpholin-4-yl-2-(2- piperidin-1-yl-ethoxy)- pyrimidin-4-ylmethylene]- hydrazine |
| 513 | 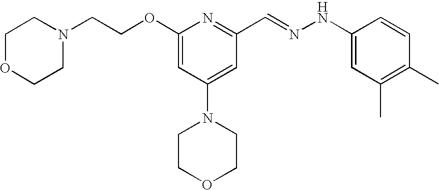 | N-(3,4-Dimethyl-phenyl)-N′- [4-morpholin-4-yl-6-(2- morpholin-4-yl-ethoxy)- pyridin-2-ylmethylene]- hydrazine |
| 514 | 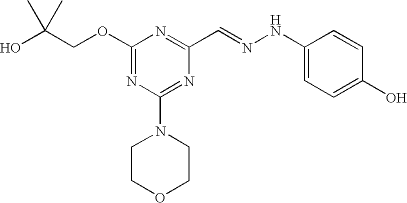 | 4-{N′-[4-(2-Hydroxy-2-methyl- propoxy)-6-morpholin-4-yl- [1,3,5]triazin-2-ylmethylene]- hydrazino}-phenol |
| 515 | 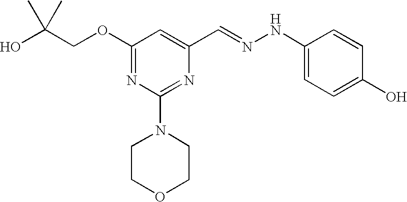 | 4-{N′-[6-(2-Hydroxy-2-methyl- propoxy)-2-morpholin-4-yl- pyrimidin-4-ylmethylene]- hydrazino}-phenol |
| 516 | 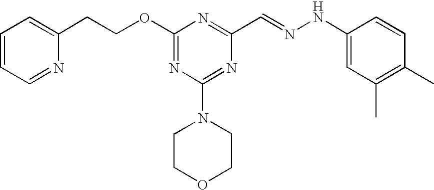 | N-(3,4-Dimethyl-phenyl)-N′- [4-morpholin-4-yl-6-(2- pyridin-2-yl-ethoxy)- [1,3,5]triazin-2-ylmethylene]- hydrazine |
| 517 | 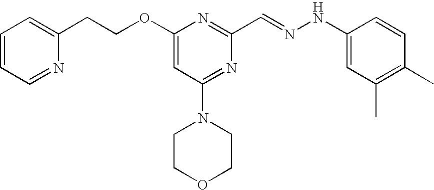 | N-(3,4-Dimethyl-phenyl)-N′- [4-morpholin-4-yl-6-(2- pyridin-2-yl-ethoxy)- pyrimidin-2-ylmethylene]- hydrazine |
| 518 | 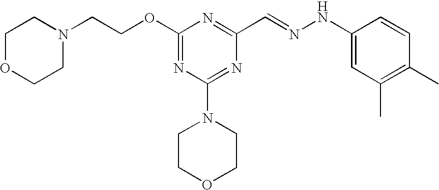 | N-(3,4-Dimethyl-phenyl)-N′- [4-morpholin-4-yl-6-(2- morpholin-4-yl-ethoxy)- [1,3,5]triazin-2-ylmethylene]- hydrazine |
| 519 | 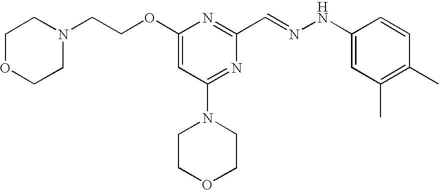 | N-(3,4-Dimethyl-phenyl)-N′- [4-morpholin-4-yl-6-(2- morpholin-4-yl-ethoxy)- pyrimidin-2-ylmethylene]- hydrazine |
| 520 | 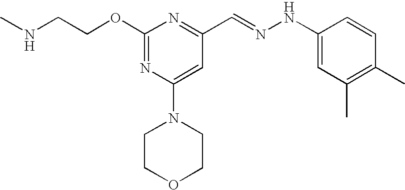 | (2-{4-[(3,4-Dimethyl-phenyl)- hydrazonomethyl]-6- morpholin-4-yl-pyrimidin-2- yloxy}-ethyl)-methyl-amine |
| 521 | 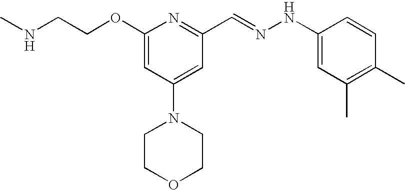 | (2-{6-[(3,4-Dimethyl-phenyl)- hydrazonomethyl]-4- morpholin-4-yl-pyridin-2- yloxy}-ethyl)-methyl-amine |
| 522 | 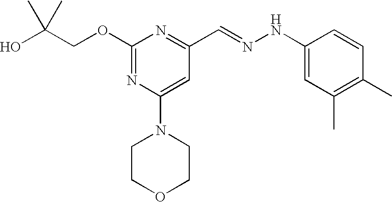 | 1-{4-[(3,4-Dimethyl-phenyl)- hydrazonomethyl]-6- morpholin-4-yl-pyrimidin-2- yloxy}-2-methyl-propan-2-ol |
| 523 | 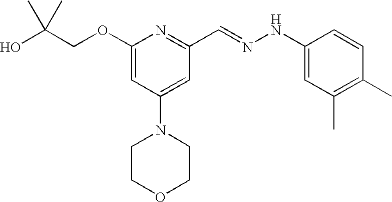 | 1-{6-[(3,4-Dimethyl-phenyl)- hydrazonomethyl]-4- morpholin-4-yl-pyridin-2- yloxy}-2-methyl-propan-2-ol |
| 524 | 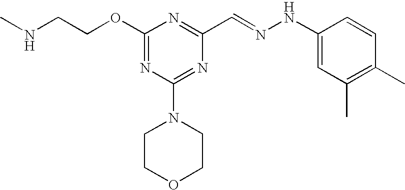 | (2-{4-[(3,4-Dimethyl-phenyl)- hydrazonomethyl]-6- morpholin-4-yl-[1,3,5]triazin- 2-yloxy}-ethyl)-methyl-amine |
| 525 | 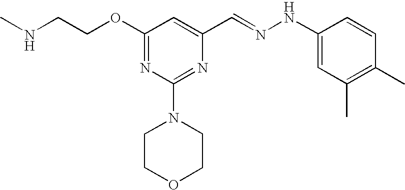 | (2-{6-[(3,4-Dimethyl-phenyl)- hydrazonomethyl]-2- morpholin-4-yl-pyrimidin-4- yloxy}-ethyl)-methyl-amine |
| 526 | 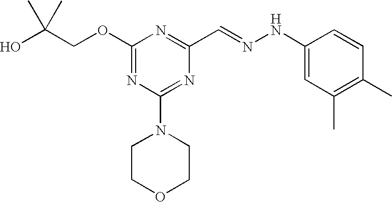 | 1-{4-[(3,4-Dimethyl-phenyl)- hydrazonomethyl]-6- morpholin-4-yl-[1,3,5]triazin- 2-yloxy}-2-methyl-propan-2-ol |
| 527 | 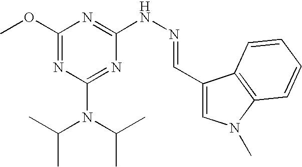 | Diisopropyl-{4-methoxy-6-[N′- (1-methyl-1H-indol-3- ylmethylene)-hydrazino]- [1,3,5]triazin-2-yl}-amine |
| 528 | 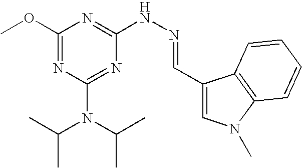 | {4-[N′-(1H-Indol-3- ylmethylene)-hydrazino]-6- methoxy-[1,3,5]triazin-2-yl}- diisopropyl-amine |
| 529 | 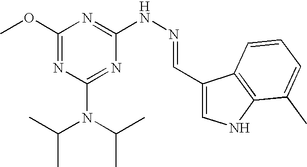 | Diisopropyl-{4-methoxy-6-[N′- (7-methyl-1H-indol-3- ylmethylene)-hydrazino]- [1,3,5]triazin-2-yl}-amine |
| 530 | 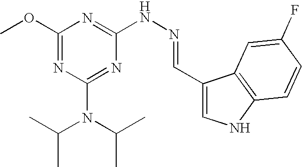 | {4-[N′-(5-Fluoro-1H-indol-3- ylmethylene)-hydrazino]-6- methoxy-[1,3,5]triazin-2-yl}- diisopropyl-amine |
| 531 | 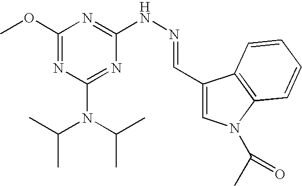 | 1-{3-[(4-Diisopropylamino-6- methoxy-[1,3,5]triazin-2-yl)- hydrazonomethyl]-indol-1-yl}- ethanone |
| 532 | 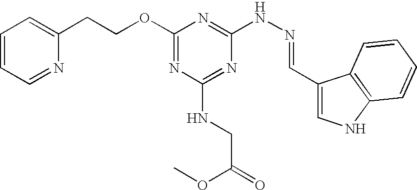 | [4-[N′-(1H-Indol-3- ylmethylene)-hydrazino]-6-(2- pyridin-2-yl-ethoxy)- [1,3,5]triazin-2-ylamino]-acetic acid methyl ester |
| 533 | 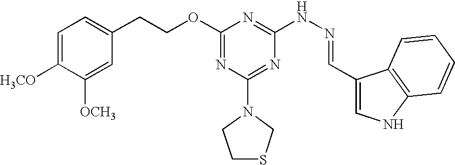 | N-{4-[2-(3,4-Dimethoxy- phenyl)-ethoxy]-6-thiazolidin- 3-yl-[1,3,5]triazin-2-yl}-N′- (1H-indol-3-ylmethylene)- hydrazine |
| 534 | 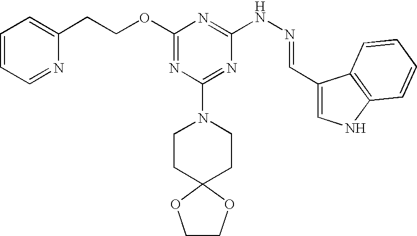 | N-[4-(1,4-Dioxa-8-aza- spiro[4.5]dec-8-yl)-6-(2- pyridin-2-yl-ethoxy)- [1,3,5]triazin-2-yl]-N′-(1H- indol-3-ylmethylene)-hydrazine |
| 535 | 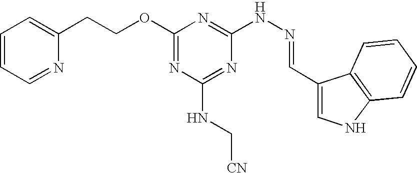 | [4-[N′-(1H-Indol-3- ylmethylene)-hydrazino]-6-(2- pyridin-2-yl-ethoxy)- [1,3,5]triazin-2-ylamino]- acetonitrile |
| 536 | 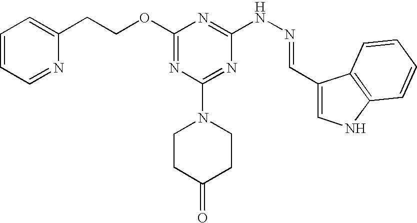 | 1-[4-[N′-(1H-Indol-3- ylmethylene)-hydrazino]-6-(2- pyridin-2-yl-ethoxy)- [1,3,5]triazin-2-yl]-piperidin-4- one |
| 537 | 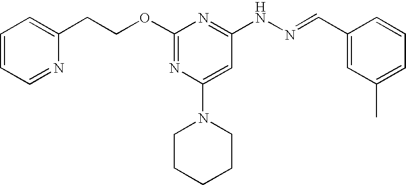 | N-(3-Methyl-benzylidene)-N′- [6-piperidin-1-yl-2-(2-pyridin- 2-yl-ethoxy)-pyrimidin-4-yl]- hydrazine |
| 538 | 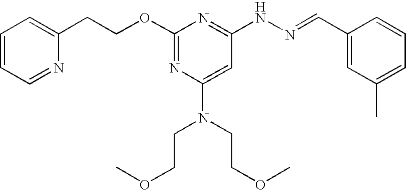 | Bis-(2-methoxy-ethyl)-[6-[N′- (3 -methyl-benzylidene)- hydrazino]-2-(2-pyridin-2-yl- ethoxy)-pyrimidin-4-yl]-amine |
| 539 | 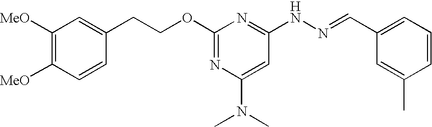 | {2-[2-(3,4-Dimethoxy-phenyl)- ethoxy]-6-[N′-(3-methyl- benzylidene)-hydrazino]- pyrimidin-4-yl}-dimethyl- amine |
| 540 | 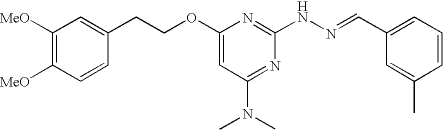 | {6-[2-(3,4-Dimethoxy-phenyl)- ethoxy]-2-[N′-(3-methyl- benzylidene)-hydrazino]- pyrimidin-4-yl}-dimethyl- amine |
| 541 | 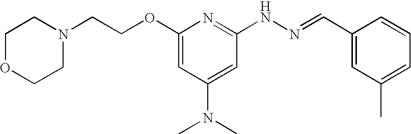 | Dimethyl-[2-[N′-(3-methyl- benzylidene)-hydrazino]-6-(2- morpholin-4-yl-ethoxy)- pyridin-4-yl]-amine |
| 542 | 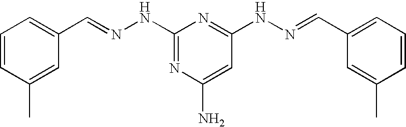 | 2,6-Bis-[N′-(3-methyl- benzylidene)-hydrazino]- pyrimidin-4-ylamine |
| 543 | 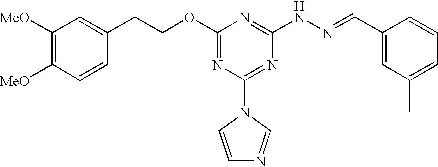 | N-{4-[2-(3,4-Dimethoxy- phenyl)-ethoxy]-6-imidazol-1- yl-[1,3,5]triazin-2-yl}-N′-(3- methyl-benzylidene)- hydrazine |
| 544 | 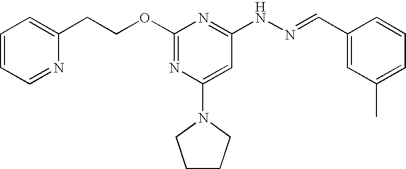 | N-(3-Methyl-benzylidene)-N′- [2-(2-pyridin-2-yl-ethoxy)-6- pyrrolidin-1-yl-pyrimidin-4- yl]-hydrazine |
| 545 | 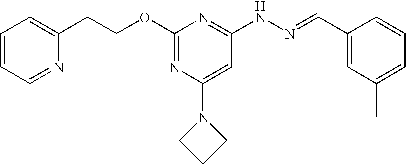 | N-[6-Azetidin-1-yl-2-(2- pyridin-2-yl-ethoxy)- pyrimidin-4-yl]-N′-(3-methyl- benzylidene)-hydrazine |
| 546 | 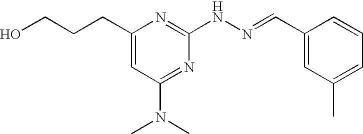 | 3-{6-Dimethylamino-2-[N′-(3- methyl-benzylidene)- hydrazino]-pyrimidin-4-yl}- propan-1-ol |
| 547 | 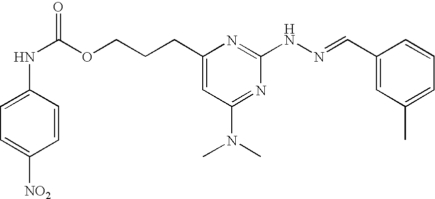 | (4-Nitro-phenyl)-carbamic acid 3-{6-dimethylamino-2-[N′-(3- methyl-benzylidene)- hydrazino]-pyrimidin-4-yl}- propyl ester |
| 548 | 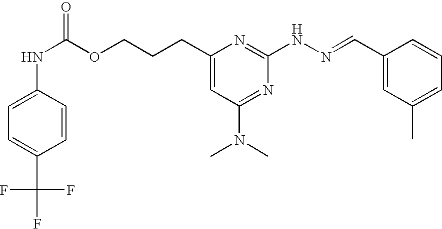 | (4-Trifluoromethyl-phenyl)- carbamic acid 3-{6- dimethylamino-2-[N′-(3- methyl-benzylidene)- hydrazino]-pyrimidin-4-yl}- propyl ester |
| 549 | 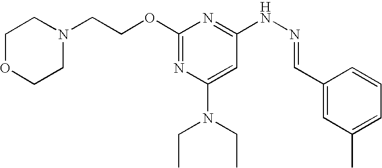 | Diethyl-[6-[N′-(3-methyl- benzylidene)-hydrazino]-2-(2- morpholin-4-yl-ethoxy)- pyrimidin-4-yl]-amine |
| 550 | 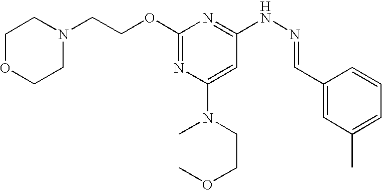 | (2-Methoxy-ethyl)-methyl-[6- [N′-(3-methyl-benzylidene)- hydrazino]-2-(2-morpholin-4- yl)-ethoxy)-pyrimidin-4-yl]- amine |
| 551 | 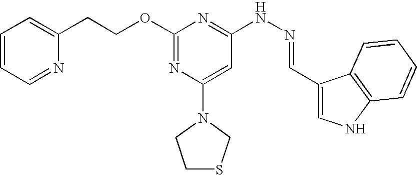 | N-(1H-Indol-3-ylmethylene)- N′-[2-(2-pyridin-2-yl-ethoxy)- 6-thiazolidin-3-yl- pyrimidin-4-yl]-hydrazine |
| 552 | 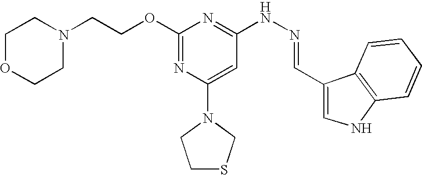 | N-(1H-Indol-3-ylmethylene)- N′-[2-(2-morpholin-4-yl- ethoxy)-6-thiazolidin-3- yl-pyrimidin-4-yl]-hydrazine |
| 553 | 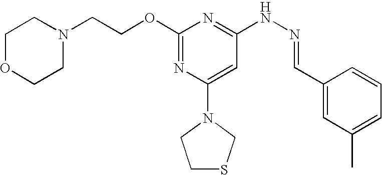 | N-(3-Methyl-benzylidene)-N′- [2-(2-morpholin-4-yl-ethoxy)- 6-thiazolidin- 3-yl-pyrimidin-4-yl]-hydrazine |
| 554 | 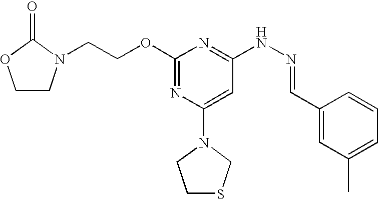 | 3-(2-{4-[N′-(3-Methyl- benzylidene)-hydrazino]-6- thiazolidin-3-yl-pyrimidin- 2-yloxy}-ethyl)-oxazolidin-2- one |
| 555 | 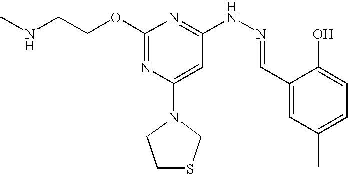 | 4-Methyl-2-{[2-(2- methylamino-ethoxy)-6- thiazolidin-3-yl-pyrimidin-4- yl]-hydrazonomethyl}-phenol |
| 556 | 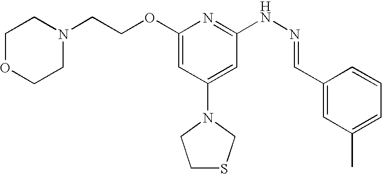 | N-(3-Methyl-benzylidene)-N′- [6-(2-morpholin-4-yl-ethoxy)- 4-thiazolidin-3-yl-pyridin-2- yl]-hydrazine |
| 557 | 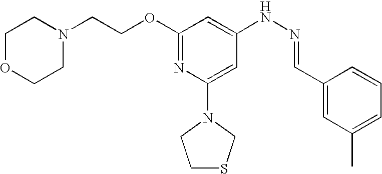 | N-(3-Methyl-benzylidene)-N′- [2-(2-morpholin-4-yl-ethoxy)- 6-thiazolidin-3-yl-pyridin-4- yl]-hydrazine |
| 558 | 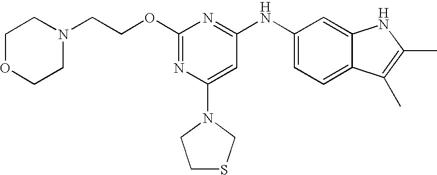 | (2,3-Dimethyl-1H-indol-6-yl)- [2-(2-morpholin-4-yl-ethoxy)- 6-thiazolidin-3-yl-pyrimidin-4- yl]-amine |
| 559 | 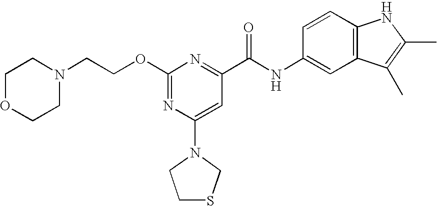 | 2-(2-Morpholin-4-yl-ethoxy)-6- thiazolidin-3-yl-pyrimidine-4- carboxylic acid (2,3-dimethyl- 1H-indol-5-yl)-amide |
| 560 | 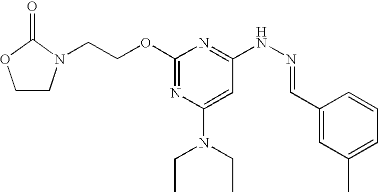 | 3-(2-{4-Diethylamino-6-[N′-(3- methyl-benzylidene)- hydrazino]-pyrimidin-2- yloxy}-ethyl)-oxazolidin-2-one |
| 561 | 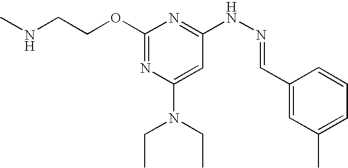 | Diethyl-{2-(2-methylamino- ethoxy)-6-[N′-(3-methyl- benzylidene)-hydrazino]- pyrimidin-4-yl}-amine |
| 562 | 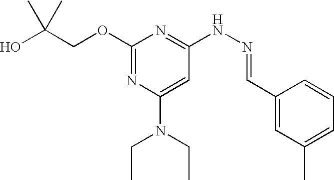 | 1-{4-Diethylamino-6-[N′-(3- methyl-benzylidene)- hydrazino]-pyrimidin-2- yloxy}-2-methyl-propan-2-ol |
| 563 | 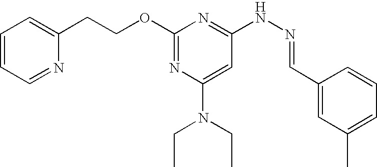 | Diethyl-[6-[N′-(3-methyl- benzylidene)-hydrazino]-2-(2- pyridin-2-yl-ethoxy)- pyrimidin-4-yl]-amine |
| 564 | 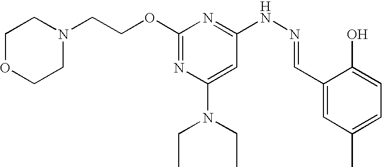 | 2-{[6-Diethylamino-2-(2- morpholin-4-yl-ethoxy)- pyrimidin-4-yl]- hydrazonomethyl}-4-methyl- phenol |
| 565 | 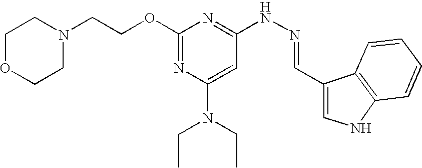 | Diethyl-[6-[N′-(1H-indol-3- ylmethylene)-hydrazino]-2-(2- morpholin-4-yl-ethoxy)- pyrimidin-4-yl]-amine |
| 566 | 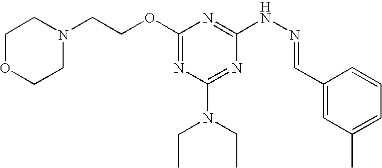 | Diethyl-[4-[N′-(3-methyl- benzylidene)-hydrazino]-6-(2- morpholin-4-yl-ethoxy)- [1,3,5]triazin-2-yl]-amine |
| 567 | 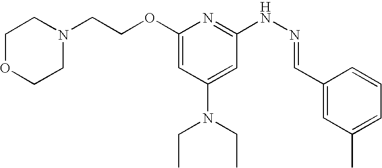 | Diethyl-[2-[N′-(3-methyl- benzylidene)-hydrazino]-6-(2- morpholin-4-yl-ethoxy)- pyridin-4-yl]-amine |
| 568 | 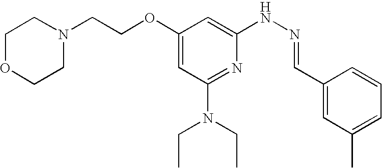 | Diethyl-[6-[N′-(3-methyl- benzylidene)-hydrazino]-4-(2- morpholin-4-yl-ethoxy)- pyridin-2-yl]-amine |
| 569 | 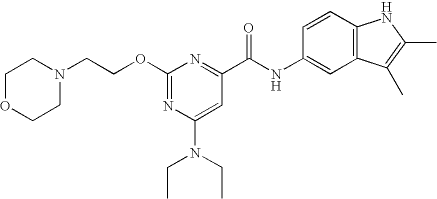 | 6-Diethylamino-2-(2- morpholin-4-yl-ethoxy)- pyrimidine-4-carboxylic acid (2,3-dimethyl-1H-indol-5-yl)- amide |
| 570 | 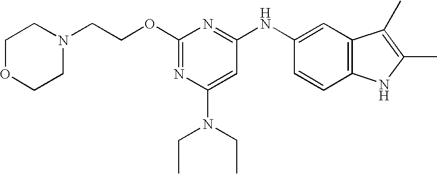 | 6-Diethylamino-2-(2- morpholin-4-yl-ethoxy)-4- [(2,3-dimethy-1H-indol-5-yl)- amino]-pyrimidine |
| 571 | 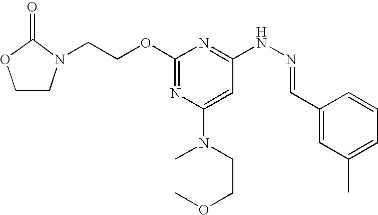 | 3-(2-{4-[(2-Methoxy-ethyl)- methyl-amino]-6-[N′-(3- methyl-benzylidene)- hydrazino]-pyrimidin-2- yloxy}-ethyl)-oxazolidin-2-one |
| 572 | 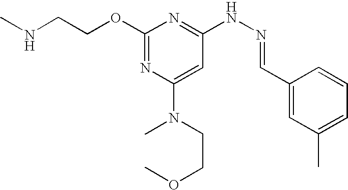 | (2-Methoxy-ethyl)-methyl-{2- (2-methylamino-ethoxy)-6-[N′- (3-methyl-benzylidene)- hydrazino]-pyrimidin-4-yl}- amine |
| 573 | 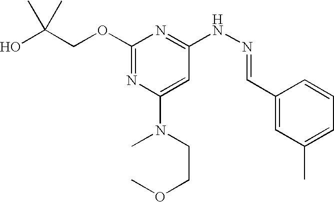 | 1-{4-[(2-Methoxy-ethyl)- methyl-amino]-6-[N′-(3- methyl-benzylidene)- hydrazino]-pyrimidin-2- yloxy}-2-methyl-propan-2-ol |
| 574 | 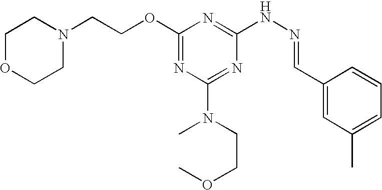 | (2-Methoxy-ethyl)-methyl-[4- [N′-(3-methyl-benzylidene)- hydrazino]-6-(2-morpholin-4- yl-ethoxy)-[1,3,5]triazin-2-yl]- amine |
| 575 | 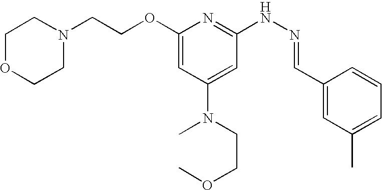 | (2-Methoxy-ethyl)-methyl-[2- [N′-(3-methyl-benzylidene)- hydrazino]-6-(2-morpholin-4- yl-ethoxy)-pyridin-4-yl]-amine |
| 576 | 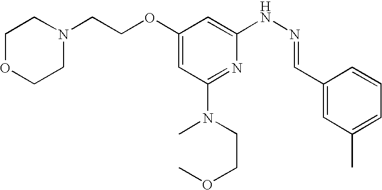 | (2-Methoxy-ethyl)-methyl-[6- [N′-(3-methyl-benzylidene)- hydrazino]-4-(2-morpholin-4- yl-ethoxy)-pyridin-2-yl]-amine |
| 577 | 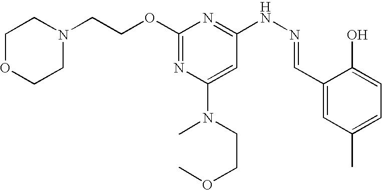 | 2-{[6-[(2-Methoxy-ethyl)- methyl-amino]-2-(2-morpholin- 4-yl-ethoxy)-pyrimidin-4-yl]- hydrazonomethyl}-4-methyl- phenol |
| 578 | 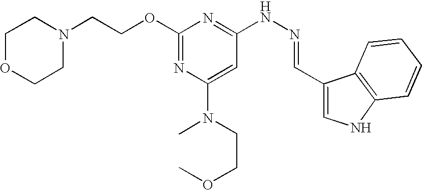 | [6-[N′-(1H-Indol-3- ylmethylene)-hydrazino]-2-(2- morpholin-4-yl-ethoxy)- pyrimidin-4-yl]-(2-methoxy- ethyl)-methyl-amine |
| 579 | 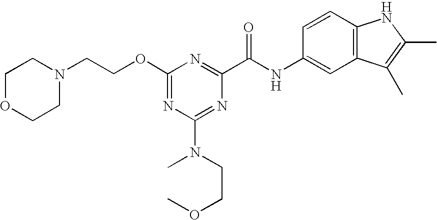 | 4-[(2-Methoxy-ethyl)-methyl- amino]-6-(2-morpholin-4-yl- ethoxy)-[1,3,5]triazine-2- carboxylic acid (2,3-dimethyl- 1H-indol-5-yl)-amide |
| 580 | 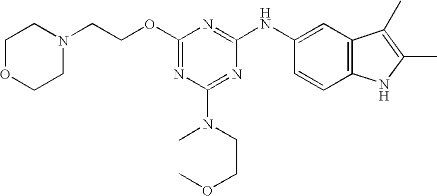 | N-(2,3-Dimethyl-1H-indol-5- yl)-N′-(2-methoxy-ethyl)-N′- methyl-6-(2-morpholin-4-yl- ethoxy)-[1,3,5]triazine-2,4- diamine |
| 581 | 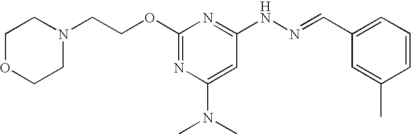 | Dimethyl-[6-[N′-(3-methyl- benzylidene)-hydrazino]-2-(2- morpholin-4-yl-ethoxy)- pyrimidin-4-yl]-amine |
| 582 | 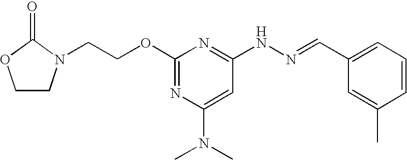 | 3-(2-{4-Dimethylamino-6-[N′- (3-methyl-benzylidene)- hydrazino]-pyrimidin-2- yloxy}-ethyl)-oxazolidin-2-one |
| 583 | 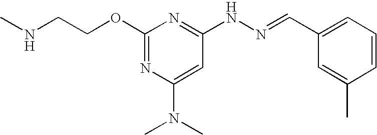 | Dimethyl-{2-(2-methylamino- ethoxy)-6-[N′-(3-methyl- benzylidene)-hydrazino]- pyrimidin-4-yl}-amine |
| 584 | 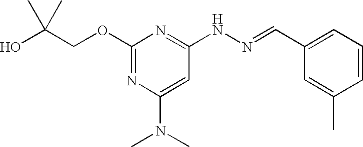 | 1-{4-Dimethylamino-6-[N′-(3- methyl-benzylidene)- hydrazino]-pyrimidin-2- yloxy}-2-methyl-propan-2-ol |
| 585 | 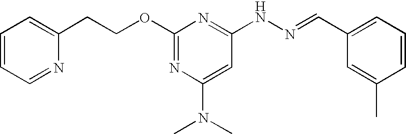 | Dimethyl-[6-[N′-(3-methyl- benzylidene)-hydrazino]-2-(2- pyridin-2-yl-ethoxy)- pyrimidin-4-yl]-amine |
| 586 | 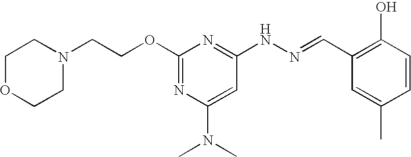 | 2-{[6-Dimethylamino-2-(2- morpholin-4-yl-ethoxy)- pyrimidin-4-yl]- hydrazonomethyl}-4-methyl- phenol |
| 587 | 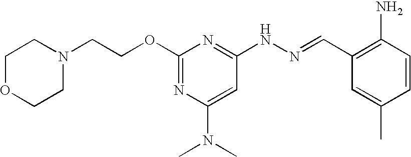 | [6-[N′-(2-Amino-5-methyl- benzylidene)-hydrazino]-2-(2- morpholin-4-yl-ethoxy)- pyrimidin-4-yl]-dimethyl- amine |
| 588 | 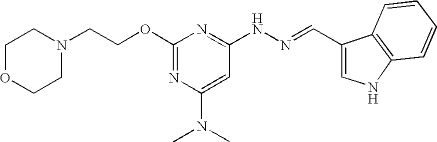 | [6-[N′-(1H-Indol-3- ylmethylene)-hydrazino]-2-(2- morpholin-4-yl-ethoxy)- pyrimidin-4-yl]-dimethyl- amine |
| 589 | 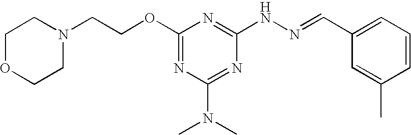 | Dimethyl-[4-[N′-(3-methyl- benzylidene)-hydrazino]-6-(2- morpholin-4-yl-ethoxy)- [1,3,5]triazin-2-yl]-amine |
| 590 | 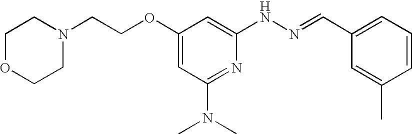 | Dimethyl-[6-[N′-(3-methyl- benzylidene)-hydrazino]-4-(2- morpholin-4-yl-ethoxy)- pyridin-2-yl]-amine |
| 591 | 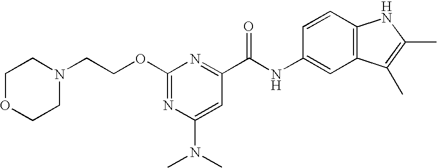 | 6-Dimethylamino-2-(2- morpholin-4-yl-ethoxy)- pyrimidine-4-carboxylic acid (2,3-dimethyl-1H-indol-5-yl)- amide |
| 592 | 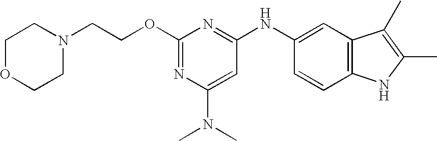 | 6-Dimethylamino-2-(2- morpholin-4-yl-ethoxy)-4- [(2,3-dimethyl-1H-indol-5-yl)- amino]pyrimidine |
| 593 | 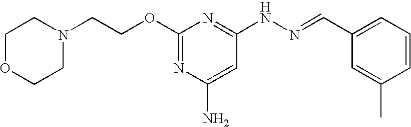 | 6-[N′-(3-Methyl-benzylidene)- hydrazino]-2-(2-morpholin-4- yl-ethoxy)-pyrimidin-4- ylamine |
| 594 | 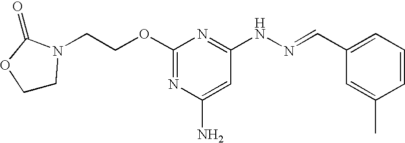 | 3-(2-{4-Amino-6-[N′-(3- methyl-benzylidene)- hydrazino]-pyrimidin-2- yloxy}-ethyl)-oxazolidin-2-one |
| 595 | 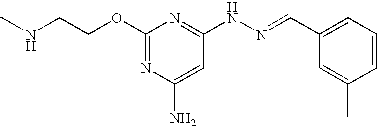 | 2-(2-Methylamino-ethoxy)-6- [N′-(3-methyl-benzylidene)- hydrazino]-pyrimidin-4- ylamine |
| 596 | 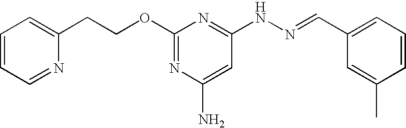 | 6-[N′-(3-Methyl-benzylidene)- hydrazino]-2-(2-pyridin-2-yl- ethoxy)-pyrimidin-4-ylamine |
| 597 | 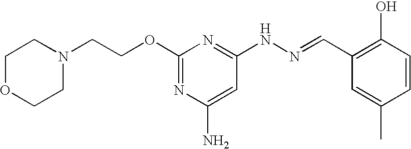 | 2-{[6-Amino-2-(2-morpholin- 4-yl-ethoxy)-pyrimidin-4-yl]- hydrazonomethyl}-4-methyl- phenol |
| 598 | 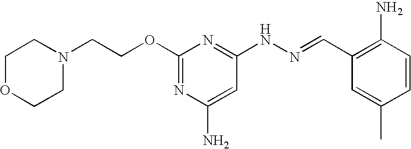 | 6-[N′-(2-Amino-5-methyl- benzylidene)-hydrazino]-2-(2- morpholin-4-yl-ethoxy)- pyrimidin-4-ylamine |
| 599 | 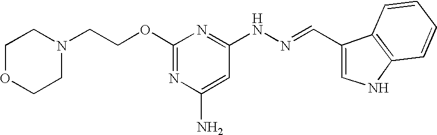 | 6-[N′-(1H-Indol-3- ylmethylene)-hydrazino]-2-(2- morpholin-4-yl-ethoxy)- pyrimidin-4-ylamine |
| 600 | 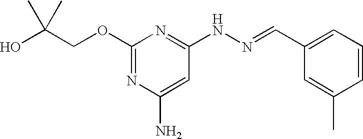 | 1-{4-Amino-6-[N′-(3-methyl- benzylidene)-hydrazino]- pyrimidin-2-yloxy}-2-methyl- propan-2- |
| 601 | 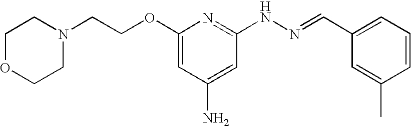 | 2-[N′-(3-Methyl-benzylidene)- hydrazino]-6-(2-morpholin-4- yl-ethoxy)-pyridin-4-ylamine |
| 602 | 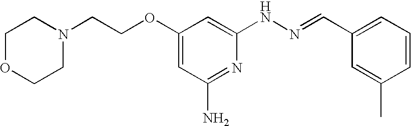 | 6-[N′-(3-Methyl-benzylidene)- hydrazino]-4-(2-morpholin-4- yl-ethoxy)-pyridin-2-ylamine |
| 603 | 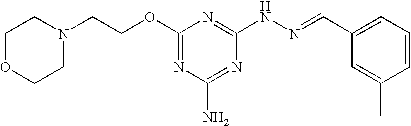 | 4-[N′-(3-Methyl-benzylidene)- hydrazino]-6-(2-morpholin-4- yl-ethoxy)-[1,3,5]triazin-2- ylamine |
| 604 | 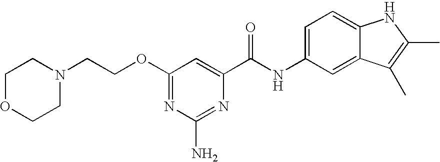 | 2-Amino-6-(2-morpholin-4-yl- ethoxy)-pyrimidine-4- carboxylic acid (2,3-dimethyl- 1H-indol-5-yl)-amide |
| 605 | 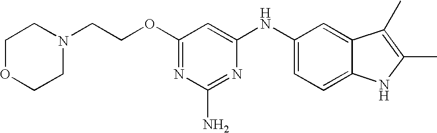 | N4-(2,3-Dimethyl-1H-indol-5- yl)-6-(2-morpholin-4-yl- ethoxy)-pyrimidine-2,4- diamine |
| 606 | 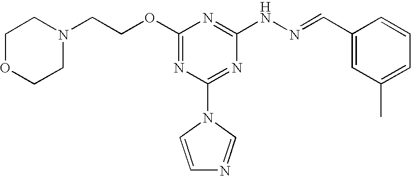 | N-[4-Imidazol-1-yl-6-(2- morpholin-4-yl-ethoxy)- [1,3,5]triazin-2-yl]-N′-(3- methyl-benzylidene)-hydrazine |
| 607 | 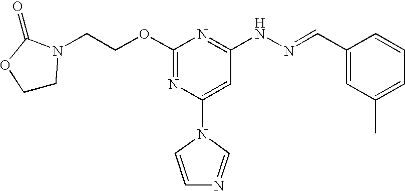 | 3-(2-{4-Imidazol-1-yl-6-[N′-(3- methyl-benzylidene)- hydrazino]-pyrimidin-2- yloxy}-ethyl)-oxazolidin-2-one |
| 608 | 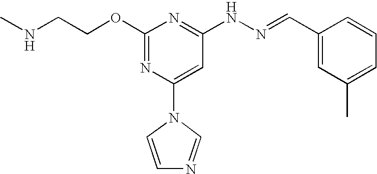 | (2-{4-Imidazol-1-yl-6-[N′-(3- methyl-benzylidene)- hydrazino]-pyrimidin-2- yloxy}-ethyl)-methyl-amine |
| 609 | 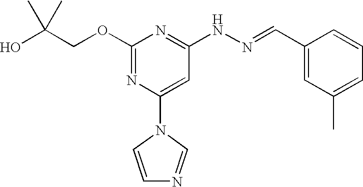 | 1-{4-Imidazol-1-yl-6-[N′-(3- methyl-benzylidene)- hydrazino]-pyrimidin-2- yloxy}-2-methyl-propan-2-ol |
| 610 | 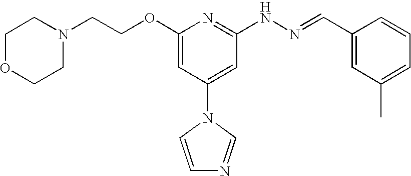 | N-[4-Imidazol-1-yl-6-(2- morpholin-4-yl-ethoxy)- pyridin-2-yl]-N′-(3-methyl- benzylidene)-hydrazine |
| 611 | 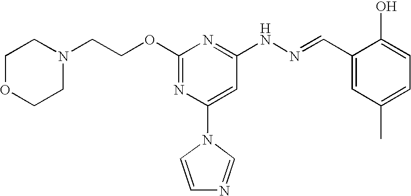 | 2-{[6-Imidazol-1-yl-2-(2- morpholin-4-yl-ethoxy)- pyrimidin-4-yl]- hydrazonomethyl}-4-methyl- phenol |
| 612 | 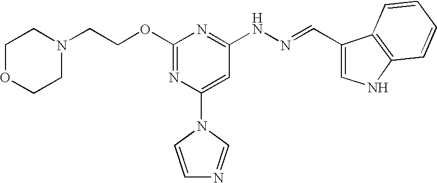 | N-[6-Imidazol-1-yl-2-(2- morpholin-4-yl-ethoxy)- pyrimidin-4-yl]-N′-(1H-indol- 3-ylmethylene)-hydrazine |
| 613 | 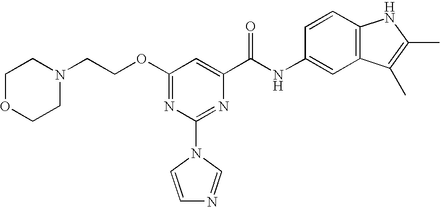 | 2-Imidazol-1-yl-6-(2- morpholin-4-yl-ethoxy)- pyrimidine-4-carboxylic acid (2,3-dimethyl-1H-indol-5-yl)- amide |
| 614 | 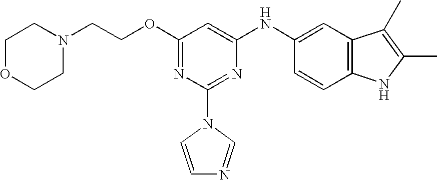 | (2,3-Dimethyl-1H-indol-5-yl)- [2-imidazol-1-yl-6-(2- morpholin-4-yl-ethoxy)- pyrimidin-4-yl]-amine |
| 615 | 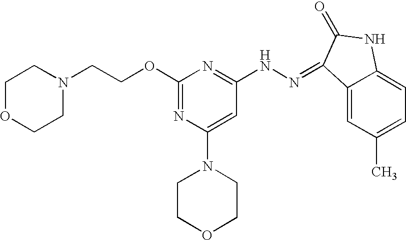 | 5-Methyl-3-{[6-morpholin-4- yl-2-(2-morpholin-4-yl- ethoxy)-pyrimidin-4-yl]- hydrazono}-1,3-dihydro-indol- 2-one |
| 616 | 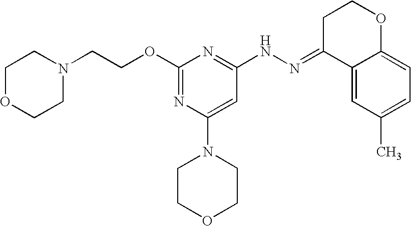 | N-(6-Methyl-chroman-4- ylidene)-N′-[6-morpholin-4-yl- 2-(2-morpholin-4-yl-ethoxy)- pyrimidin-4-yl]-hydrazine |
| 617 | 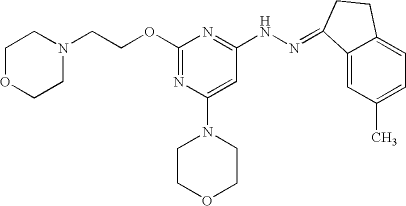 | N-(6-Methyl-indan-1-ylidene)- N′-[6-morpholin-4-yl-2-(2- morpholin-4-yl-ethoxy)- pyrimidin-4-yl]-hydrazine |
| 618 | 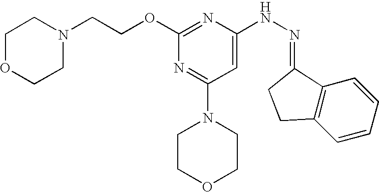 | N-(Indan-1-ylidene)-N′-[6- morpholin-4-yl-2-(2- morpholin-4-yl-ethoxy)- pyrimidin-4-yl]-hydrazine |
| 619 | 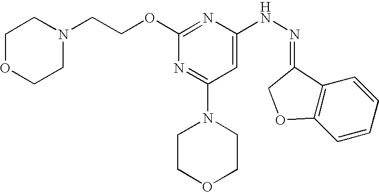 | N-(Benzofuran-3-ylidene)-N′- [6-morpholin-4-yl-2-(2- morpholin-4-yl-ethoxy)- pyrimidin-4-yl]-hydrazine |
| 620 | 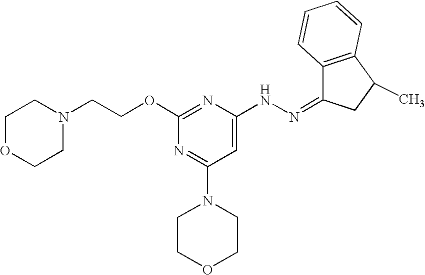 | N-(3-Methyl-indan-1-ylidene)- N′-[6-morpholin-4-yl-2-(2- morpholin-4-yl-ethoxy)- pyrimidin-4-yl]-hydrazine |
| 621 | 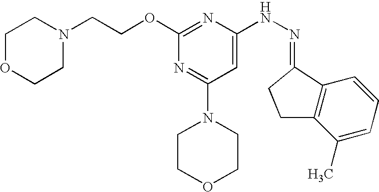 | N-(4-Methyl-indan-1-ylidene)- N′-[6-morpholin-4-yl-2-(2- morpholin-4-yl-ethoxy)- pyrimidin-4-yl]-hydrazine |
| 622 | 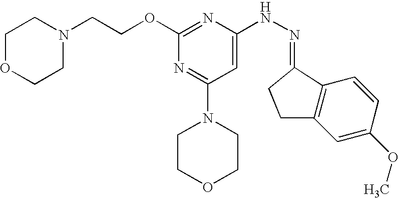 | N-(5-Methoxy-indan-1- ylidene)-N′-[6-morpholin-4-yl- 2-(2-morpholin-4-yl-ethoxy)- pyrimidin-4-yl]-hydrazine |
| 623 | 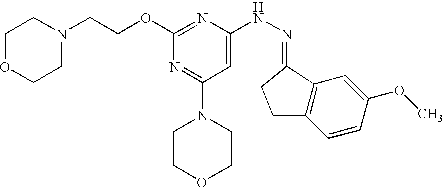 | N-(6-Methoxy-indan-1- ylidene)-N′-[6-morpholin-4-yl- 2-(2-morpholin-4-yl-ethoxy)- pyrimidin-4-yl]-hydrazine |
| 624 | 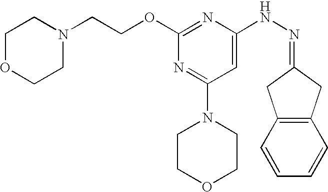 | N-(Indan-2-ylidene)-N′-[6- morpholin-4-yl-2-(2- morpholin-4-yl-ethoxy)- pyrimidin-4-yl]-hydrazine |
| 625 | 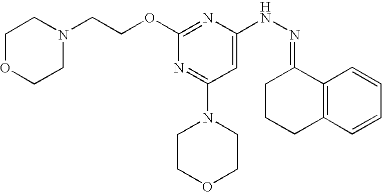 | N-(3,4-Dihydro-2H- naphthalen-1-ylidene)-N′-[6- morpholin-4-yl-2-(2- morpholin-4-yl-ethoxy)- pyrimidin-4-yl]-hydrazine |
| 626 | 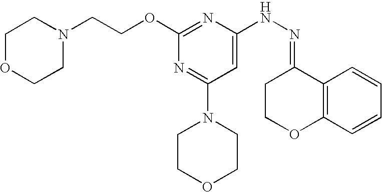 | N-(Chroman-4-ylidene)-N′-[6- morpholin-4-yl-2-(2- morpholin-4-yl-ethoxy)- pyrimidin-4-yl]-hydrazine |
| 627 | 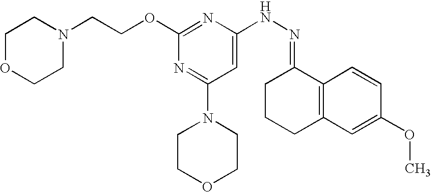 | N-(6-Methoxy-3,4-dihydro-2H- naphthalen-1-ylidene)-N′-[6- morpholin-4-yl-2-(2- morpholin-4-yl-ethoxy)- pyrimidin-4-yl]-hydrazine |
| 628 | 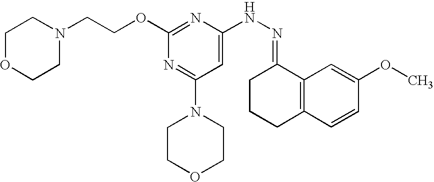 | N-(7-Methoxy-3,4-dihydro-2H- naphthalen-1-ylidene)-N′-[6- morpholin-4-yl-2-(2- morpholin-4-yl-ethoxy)- pyrimidin-4-yl]-hydrazine |
| 629 | 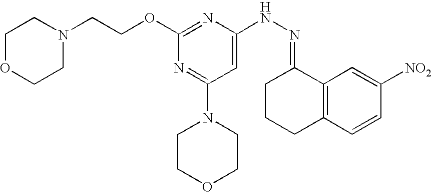 | N-(7-Nitro-3,4-dihydro-2H- naphthalen-1-ylidene)-N′-[6- morpholin-4-yl-2-(2- morpholin-4-yl-ethoxy)- pyrimidin-4-yl]-hydrazine |
| 630 | 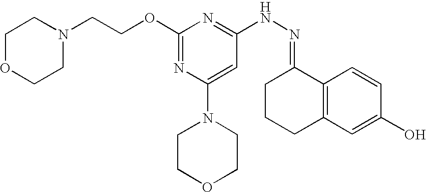 | N-(6-Hydroxy-3,4-dihydro-2H- naphthalen-1-ylidene)-N′-[6- morpholin-4-yl-2-(2- morpholin-4-yl-ethoxy)- pyrimidin-4-yl]-hydrazine |
| 631 | 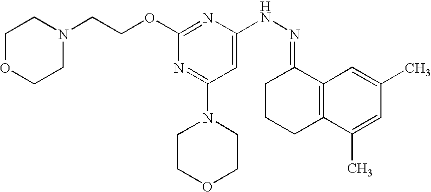 | N-(5,7-Dimethyl-3,4-dihydro- 2H-naphthalen-1-ylidene)-N′- [6-morpholin-4-yl-2-(2- morpholin-4-yl-ethoxy)- pyrimidin-4-yl]-hydrazine |
| 632 | 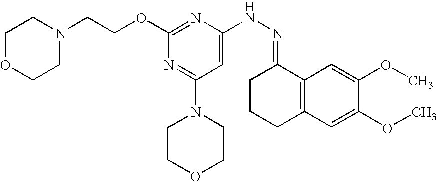 | N-(6,7-Dimethoxy-3,4- dihydro-2H-naphthalen-1- ylidene)-N′-[6-morpholin-4-yl- 2-(2-morpholin-4-yl-ethoxy)- pyrimidin-4-yl]-hydrazine |
| 633 | 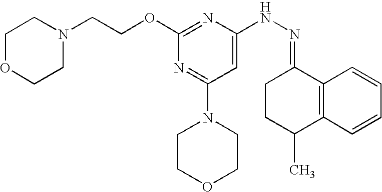 | N-(4-Methyl-3,4-dihydro-2H- naphthalen-1-ylidene)-N′-[6- morpholin-4-yl-2-(2- morpholin-4-yl-ethoxy)- pyrimidin-4-yl]-hydrazine |
| 634 | 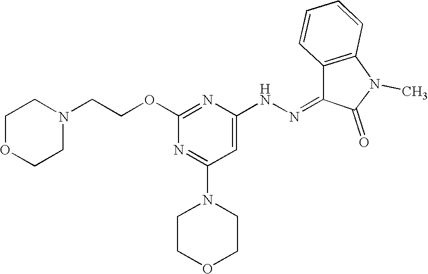 | 1-Methyl-3-{[6-morpholin-4- yl-2-(2-morpholin-4-yl- ethoxy)-pyrimidin-4-yl]- hydrazono}-1,3-dihydro-indol- 2-one |
| 635 | 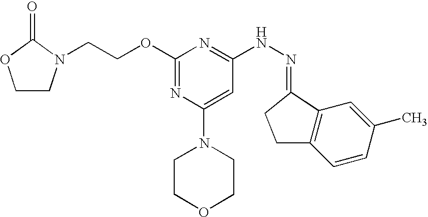 | 3-(2-{4-[N′-(6-Methyl-indan- 1-ylidene)-hydrazino]-6- morpholin-4-yl-pyrimidin-2- yloxy}-ethyl)-oxazolidin-2-one |
| 636 | 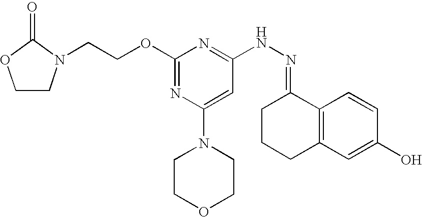 | 3-(2-{4-[N′-(6-Hydroxy-3,4- dihydro-2H-naphthalen-1- ylidene)-hydrazino]-6- morpholin-4-yl-pyrimidin-2- yloxy}-ethyl)-oxazolidin-2-one |
| 637 | 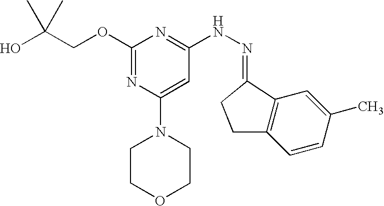 | 2-Methyl-1-{4-[N′-(6-methyl- indan-1-ylidene)-hydrazino]-6- morpholin-4-yl-pyrimidin-2- yloxy}-propan-2-ol |
| 638 | 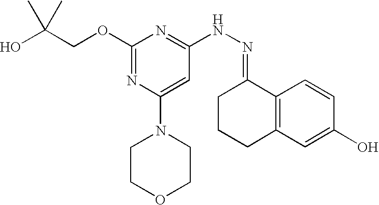 | 5-{[2-(2-Hydroxy-2-methyl- propoxy)-6-morpholin-4-yl- pyrimidin-4-yl]-hydrazono}- 5,6,7,8-tetrahydro-naphthalen- 2-ol |
| 639 | 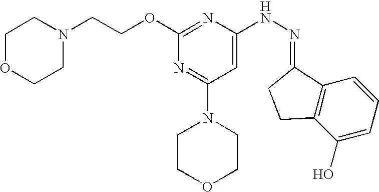 | N-(4-Hydroxy-indan-1- ylidene)-N′-[6-morpholin-4-yl- 2-(2-morpholin-4-yl-ethoxy)- pyrimidin-4-yl]-hydrazine |
| 640 | 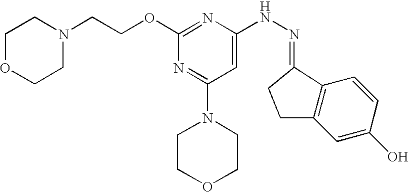 | N-(5-Hydroxy-indan-1- ylidene)-N′-[6-morpholin-4-yl- 2-(2-morpholin-4-yl-ethoxy)- pyrimidin-4-yl]-hydrazine |
| 641 | 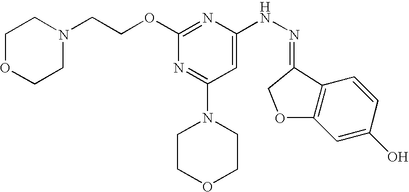 | 3-{[6-Morpholin-4-yl-2-(2- morpholin-4-yl-ethoxy)- pyrimidin-4-yl]-hydrazono}- 2,3-dihydro-benzofuran-6-ol |
| 642 | 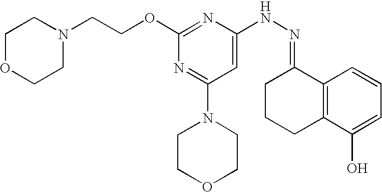 | N-(5-Hydroxy-3,4-dihydro- naphthalen-1-ylidene)-N′-[6- morpholin-4-yl-2-(2- morpholin-4-yl-ethoxy)- pyrimidin-4-yl]-hydrazine |
| 643 | 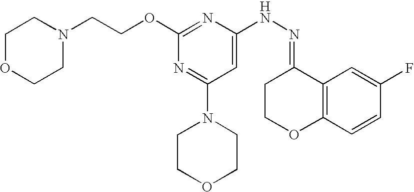 | N-(6-Fluoro-chroman-4- ylidene)-N′-[6-morpholin-4-yl- 2-(2-morpholin-4-yl-ethoxy)- pyrimidin-4-yl]-hydrazine |
| 644 | 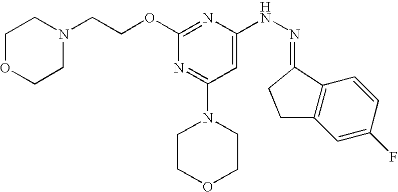 | N-(5-Fluoro-indan-1-ylidene)- N′-[6-morpholin-4-yl-2-(2- morpholin-4-yl-ethoxy)- pyrimidin-4-yl]-hydrazine |
| 645 | 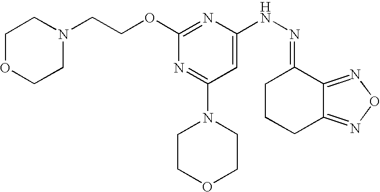 | N-(6,7-Dihydro-5H- benzo[1,2,5]oxadiazol-4- ylidene)-N′-[6-morpholin-4-yl- 2-(2-morpholin-4-yl-ethoxy)- pyrimidin-4-yl]-hydrazine |
| 646 | 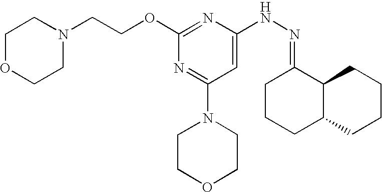 | N-[6-Morpholin-4-yl-2-(2- morpholin-4-yl-ethoxy)- pyrimidin-4-yl]-N′-(octahydro- naphthalen-1-ylidene)- hydrazine |
| 647 | 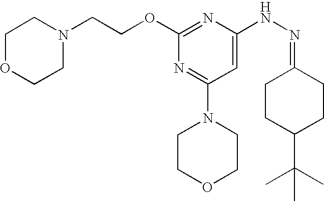 | N-(4-tert-Butyl- cyclohexylidene)-N′-[6- morpholin-4-yl-2-(2- morpholin-4-yl-ethoxy)- pyrimidin-4-yl]-hydrazine |
| 648 | 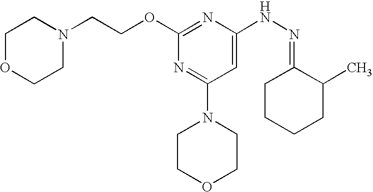 | N-(2-Methyl-cyclohexylidene)- N′-[6-morpholin-4-yl-2-(2- morpholin-4-yl-ethoxy)- pyrimidin-4-yl]-hydrazine |
| 649 | 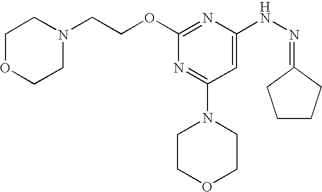 | N-Cyclopentylidene-N′-[6- morpholin-4-yl-2-(2- morpholin-4-yl-ethoxy)- pyrimidin-4-yl]-hydrazine |
| 650 | 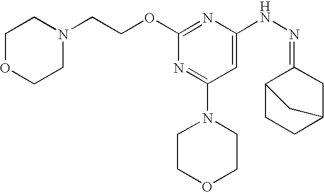 | N-Bicyclo[2.2.1]hept-2- ylidene-N′-[6-morpholin-4-yl- 2-(2-morpholin-4-yl-ethoxy)- pyrimidin-4-yl]-hydrazine |
| 651 | 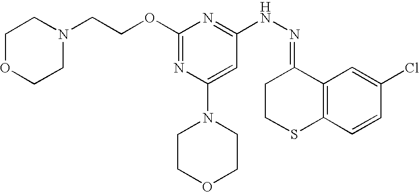 | N-(6-Chloro-thiochroman-4- ylidene)-N′-[6-morpholin-4-yl- 2-(2-morpholin-4-yl-ethoxy)- pyrimidin-4-yl]-hydrazine |
| 652 | 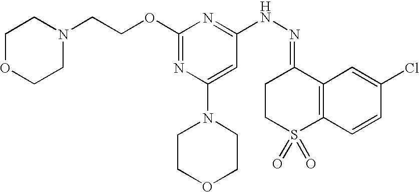 | N-(6-Chloro-1,1-dioxo-1λ⁶- thiochroman-4-ylidene)-N′-[6- morpholin-4-yl-2-(2- morpholin-4-yl-ethoxy)- pyrimidin-4-yl]-hydrazine |
| 653 | 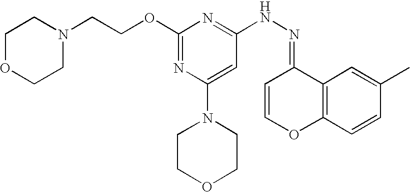 | N-(6-Methyl-chromen-4- ylidene)-N′-[6-morpholin-4-yl- 2-(2-morpholin-4-yl-ethoxy)- pyrimidin-4-yl]-hydrazine |
| 654 | 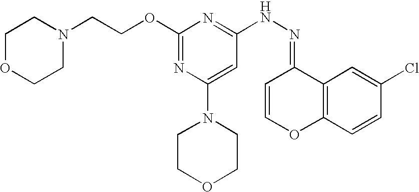 | N-(6-Chloro-chromen-4- ylidene)-N′-[6-morpholin-4-yl- 2-(2-morpholin-4-yl-ethoxy)- pyrimidin-4-yl]-hydrazine |

Method of Preparing Compounds that Inhibit IL-12, IL-23 and/or IL-27

Methods for making compounds that inhibit IL-12, IL-23 and/or IL-27 that can be used to form the mesylate salts of the invention have been disclosed in the U.S. patents and patent applications listed in Table 2. The entire teachings of these patents and patent applications are incorporated herein by reference.

TABLE 2

| | | | Publication |
|---|---|---|---|
| Serial No. | Filing Date | Publication No. | Date |
| U.S. Pat. No. | Jun. 15, 2000 | | |
| 6,384,032 | | | |
| U.S. Pat. No. | Nov. 30, 2001 | | |
| 6,680,315 | | | |
| U.S. Pat. No. | Nov. 30, 2001 | | |
| 6,693,097 | | | |
| U.S. Pat. No. | Jul. 10, 2002 | | |
| 6,660,733 | | | |
| U.S. Pat. No. | Nov. 26, 2002 | | |
| 6,858,606 | | | |
| U.S. Application | Sep. 5, 2003 | 2004-0053926 | Mar. 18, 2004 |
| No. 10/656,360 | | | |
| U.S. Application | Sep. 5, 2003 | 2004-0048873 | Mar. 11, 2004 |
| No. 10/656,671 | | | |
| U.S. Application | Sep. 5, 2003 | 2004-0053937 | Mar. 18, 2004 |
| No. 10/655,672 | | | |
| U.S. Application | Oct. 14, 2003 | 2004-0198725 | Oct. 7, 2004 |
| No. 10/686,505 | | | |
| PCT Application No. | May 28, 2004 | WO 2005/ | Jan. 6, 2005 |
| PCT/US2004/017064 | | 000404 | |
| U.S. Provisional | Jul. 1, 2004 | | |
| Application | | | |
| No. 60/585,124 | | | |
| U.S. Application | Nov. 10, 2004 | | |
| No. 10/985,696 | | | |
| U.S. Application | Nov. 10, 2004 | | |
| No. 10/985,716 | | | |
| U.S. Application | Nov. 10, 2004 | | |
| No. 10/985,627 | | | |
| U.S. Provisional | Nov. 19, 2004 | | |
| Application No. | | | |
| 60/629,505 | | | |
| U.S. Provisional | Nov. 10, 2004 | | |
| Application No. | | | |
| 60/626,609 | | | |
| U.S. Provisional | Nov. 10, 2004 | | |
| Application No. | | | |
| 60/627,001 | | | |
| U.S. Provisional | Nov. 10, 2004 | | |
| Application No. | | | |
| 60/626,761 | | | |
| U.S. Application No. | Nov. 10, 2004 | | |
| 10/986,553 | | | |
| U.S. Application No. | Jan. 21, 2005 | | |
| 11/041,537 | | | |
| U.S. Provisional | Jan. 28, 2005 | | |
| Application No. | | | |
| 60/648,645 | | | |
| PCT Application No. | Apr. 13, 2005 | | |
| PCT/US05/12578 | | | |
| U.S. Provisional | May 13, 2005 | | |
| Application No. <not | | | |
| yet assigned> | | | |
| Title: “IL-12 | | | |
| Modulatory | | | |
| Compounds” | | | |
| Attorney Docket No. | | | |
| ILI-015-01PR-00 | | | |

### Methods for Using the Mesylate Salts Prepared by the Method of the Invention

This invention relates to a method of preparing mesylate salts of nitrogen-heteroaryl inhibitors of IL-12, IL-23 and/or IL-27 production. Mesylate salts produced by the method of the invention are useful for treating T_{H}1 dominant autoimmune diseases such as multiple sclerosis, sepsis, myasthenia gravis, autoimmune neuropathies, Guillain-Barré syndrome, autoimmune uveitis, autoimmune hemolytic anemia, pernicious anemia, autoimmune thrombocytopenia, temporal arteritis, anti-phospholipid syndrome, vasculitides, Wegener's granulomatosis, Behcet's disease, psoriasis, psoriatic arthritis, dermatitis herpetiformis, pemphigus vulgaris, vitiligo, Crohn's disease, ulcerative colitis, interstitial pulmonary fibrosis, myelofibrosis, hepatic fibrosis, myocarditis, thyroditis, primary biliary cirrhosis, autoimmune hepatitis, Type 1 or immune-mediated diabetes mellitus, Grave's disease, Hashimoto's thyroiditis, autoimmune oophoritis and orchitis, autoimmune disease of the adrenal gland; rheumatoid arthritis, juvenile rheumatoid arthritis, systemic lupus erythematosus, scleroderma, polymyositis, dermatomyositis, spondyloarthropathies, ankylosing spondylitis, Sjogren's syndrome, and graft-versus-host disease. See, for example, Gately et al. (1998) Annu Rev Immunol. 16: 495; and Abbas et al. (1996) Nature 383: 787.

Mesylate salts formed by the method of the invention have been shown to inhibit the formation of osteoclasts (see International Patent Application Number PCT/US2004/017064, filed on May 28, 2005, the entire teachings of which are incorporated herein by reference). The regulation of osteoclastic formation and activity is only partly understood but it is known that excessive bone resorption by osteoclasts contributes to the pathology of many human diseases associated with excessive bone loss, excessive bone loss, including periodontal disease, non-malignant bone disorders (such as osteoporosis, Paget's disease of bone, osteogenesis imperfecta, fibrous dysplasia, and primary hyperparathyroidism) estrogen deficiency, inflammatory bone loss, bone malignancy, arthritis, osteopetrosis, and certain cancer-related disorders (such as hypercalcemia of malignancy (HCM), osteolytic bone lesions of multiple myeloma and osteolytic bone metastases of breast cancer and other metastatic cancers).

Thus, mesylate salts formed by the method of the invention useful in treating disease characterized by excessive bone loss.

### EXAMPLES

### Example 1

### Formation of the Mesylate Salt of Compound 50 in Water/Acetone at Room Temperature

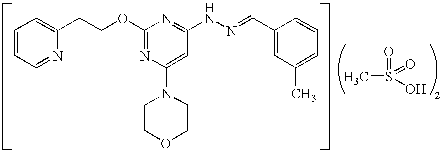

A 22-L round-bottom flask, equipped with overhead stirrer, thermometer, addition funnel and inlet gas adapter, was charged with Compound 50 (415 g, 0.99 mol) and acetone (11.8 L). The mixture was warmed to 24° C. with stirring until a clear solution was obtained. An addition funnel was charged with methanesulfonic acid (190.3 g, 1.98 mol) and deionized water (623 mL) and the resulted solution was allowed to cool down to room temperature after exothermic mixing of the methanesulfonic acid and water. The solution of methanesulfonic acid was added over 17 minutes while maintaining the temperature at ˜24° C. to the solution of Compound 50 in acetone. The methanesulfonic acid salt of Compound 50 began precipitating out of solution upon addition of the acid. The temperature of the reaction mixture rose about 4.2° C. during the addition. The reaction mixture was left overnight at room temperature with stirring. Next morning the precipitate was filtered out, the reaction flask rinsed twice with acetone (1.6 L) and rinses were transferred to the filter so that all of the precipitate was transferred to the filter. The wet cake was dried on the filter for 3 hours followed by vacuum-drying at about 1-5 mmHg at a temperature of 50° C. for 48 hours.

Yield: 95%. HPLC: 100% purity.

### Example 2

### Formation of the Mesylate Salt of Compound 50 in Water/Acetone with Heating

A 1 L three-necked flask, equipped with overhead stirrer, thermometer and addition funnel, was charged with Compound 50, 10 g (0.024 mol) and acetone, 475 mL. The mixture was stirred and heated. A clear solution formed before the temperature reached 50° C. An addition funnel was charged with methanesulfonic acid, 4.593 g (2 eq), and deionized water, 24 mL. After the temperature of the solution of Compound 50 reached 55° C., the solution of methanesulfonic acid in water was added rapidly (in 35 seconds). The temperature dropped to 51.5° C., heating was turned off (without removal of the heating mantle), allowing the solution to cool slowly while continuing stirring. In 5-6 minutes solution turned cloudy, and the methanesulfonic acid salt of Compound 50 (shown in Example 1) began to precipitate out. After 14 hours, the methanesulfonic acid salt of Compound 50 was filtered out, the flask rinsed twice with acetone (35-40 mL) and rinses were transferred to the filter so that all of the precipitate was transferred to the filter. Solid was dried on filter for a short period of time, transferred to the round-bottom flask while still wet, and dried in vacuo at room temperature for 1.5 hours followed by vacuum-oven drying (60° C., p ˜1 mmHg) for ˜24 hours. Yield: 90%; residual acetone: 1150 ppm.

### Example 3

### Formation of the Mesylate Salt of Compound 50 in Water/Acetonitrile

A 1 L three-necked flask, equipped with overhead stirrer, thermometer and addition funnel, was charged with Compound 50, 10 g (0.024 mol) and acetonitrile, 222 mL, and the mixture was stirred and heated. A clear solution formed before the temperature reached 50° C. An addition funnel was charged with methanesulfonic acid, 4.593 g (2 eq), and deionized water, 8.07 mL. After the temperature of the solution of Compound 50 reached 65° C., the solution of methanesulfonic acid in water was added rapidly (in 20 seconds) with stirring. The temperature dropped during the addition to 63° C., heating was turned off and the solution was allowed to cool slowly with stirring. After 14 minutes, when the temperature reached 52° C., the solution slowly turned cloudy, and the methanesulfonic acid salt of Compound 50 (shown in Example 1) began to precipitate out. After 8 hours, the methanesulfonic acid salt of Compound 50 was filtered out, the flask was rinsed twice with ethyl acetate (35-40 mL) and rinses were transferred to the filter so that all the precipitate was transferred to the filter. The methanesulfonic acid salt of Compound 50 was dried on the filter for a short period of time, transferred to the round-bottom flask while still wet, and dried in vacuo at room temperature for 1.5 hours followed by vacuum-oven drying (60° C., p ˜1 mmHg) for ˜23 hours. Yield: 84.6%; residual acetonitrile: 290 ppm.

### Example 4

### Formation of the Mesylate Salt of Compound 50 in Ethyl Acetate

A 1 L three-necked flask, equipped with overhead stirrer, thermometer and addition funnel, was charged with Compound 50, 10 g (0.024 mol) and ethyl acetate, 212 mL, and the mixture was stirred and heated. A clear solution formed before the temperature reached 50° C. An addition funnel was charged with methanesulfonic acid, 4.593 g (2 eq), and ethyl acetate, 10 mL. After the temperature of the solution of Compound 50 reached 65.5° C. a solution of acid was added (in 2 minutes). The precipitate of the methanesulfonic acid salt of Compound 50 (shown in Example 1) began to form when first drops of acid reached the solution of Compound 50. The temperature increased to 70.5° C. by the time addition was completed, and kept rising until 72° C. Heating was turned off and the suspension was allowed to cool slowly. After 3 hours, the methanesulfonic acid salt of Compound 50 was filtered out, the flask rinsed twice with ethyl acetate (35-40 mL) and rinses were transferred to the filter so that all of the precipitate was transferred to the filter. Solid was dried on the filter for a short period of time, transferred to the round-bottom flask while still wet, and dried in vacuo at room temperature for 4 hours followed by vacuum-oven drying (60° C., p ˜1 mmHg) for ˜23 hours. Yield 97%

### Example 5

### Formation of the Mesylate Salt of Compound 50 in Dichloromethane

Compound 50, 5 g, was dissolved in 60 mL of dichloromethane and heated to 40° C. 1.55 mL methanesulfonic acid (2 eq. with respect to Compound 50) was added drop-wise to the stirred solution (exothermic, reflux). Heating was turned off and the mixture was allowed to cool with stirring. The methanesulfonic acid salt of Compound 50 (shown in Example 1) started to precipitate out after 10 minutes when the temperature had dropped to about 38° C. The suspension was allowed to cool to room temperature. After 2 hours a solid was filtered out, the reaction flask was rinsed twice with 1:1 mixture of dichloromethane:heptane (20 mL) to transfer all of the precipitate to the filter. The precipitate was dried for 30 min on the filter followed by 8 hours at 75° C. Yield: 5.95 g (81.5%) of the mesylate salt of Compound 50.

### Example 6

### Recrystallization of the Mesylate Salt of Compound 50

### A. 95 v/v % Aqueous Ethanol

1. Method 1

A flask was charged with 10 g of the mesylate salt of Compound 50 and 100 mL of aqueous ethanol (5 mL of water and 95 mL of absolute ethanol) and heated in oil bath (60-65° C.) with stirring until clear solution was formed after about 20-25 min. The heat was turned off and the solution was allowed to cool to room temperature. After 4 hours, the precipitate of the mesylate salt of Compound 50 was filtered out, washed once with 30 mL of absolute ethanol, dried on the filter for 15-20 min, followed by vacuum-drying (˜16 hours; ˜1 mmHg). Drying continued using vacuum-oven (˜1 mmHg), at 55° C., for 5 hours. Yield: 8.06 g (80.6%) of the mesylate salt of Compound 50, m.p. 191-192° C., residual ethanol: 3826 p.p.m.

2. Method 2

A flask was charged with 10 g of the mesylate salt of Compound 50 and 5.5 mL of purified water. The mixture was heated to 36-37° C. and stirred for 0.5 hour to achieve a clear solution. Ethanol, 104.5 mL, was added causing reaction temperature drop of 1° C. The heat was turned off and precipitation of the mesylate salt of Compound 50 started when the temperature reached 32.5° C. After 3 hours, the precipitate was collected by filtration, and the flask was rinsed twice with 20 ml of absolute ethanol to transfer all of the precipitate to the filter. The precipitate was dried on filter followed by vacuum-drying (55° C., 5 hours). Yield: 8.7 g (87%) of the mesylate salt of Compound 50, m.p. 189.5-190° C., residual ethanol: 4749 p.p.m.

### B. 98 v/v % Aqueous Ethanol

A flask was charged with 10 g of the mesylate salt of Compound 50 and 175 mL of aqueous ethanol (3.5 mL of water and 171.5 mL of absolute ethanol) and heated in oil bath (75-78° C.) with stirring until clear solution was formed after about 20-25 min. The heat was turned off and solution was allowed to cool to room temperature. After 3 hours, the precipitate of the mesylate salt of Compound 50 was collected by filtration, then the flask was rinsed twice with 30 ml of absolute ethanol to help to transfer the solid into filter. The precipitate was dried on filter for 30 min, followed by vacuum-drying (1 hour at room temperature, then ˜9 hours at 60° C., vacuum pressure ˜1 mmHg). Yield: 8.9 g (89%) of the mesylate salt of Compound 50, m.p. 191.5-192° C., residual ethanol: 3442 p.p.m.

### C. 95 v/v % Aqueous Acetone

1. Method 1

A flask was charged with 10 g of the mesylate salt of Compound 50 and 5.5 mL of purified water. A mixture was heated to 55° C. with stirring for 0.5 hour to achieve a clear solution. Acetone, 104.5 mL, was added causing solution to turn cloudy. The heat was turned off and precipitation of the mesylate salt of Compound 50 started in about 1-2 minutes. After 3 hours, the precipitate was collected by filtration, and the flask was rinsed twice with 20 ml of acetone to transfer all the precipitate to the filter. The precipitated was dried on the filter for 30 min. followed by vacuum-drying (55° C., 5 hours). Yield: 8.7 g (94.5%) of the mesylate salt of Compound 50, m.p. 190.5-191.5° C., residual acetone: 1261 p.p.m. Purity: 99.3% (AUC), by-products: 0.23%.

2. Method 2

A flask is charged with 10 g of the mesylate salt of Compound 50 and 5.5 mL of purified water. The mixture is heated to 34° C. with stirring for 0.5 hour to achieve a clear solution. Heating mantle is removed, and acetone, 104.5 mL, is added slowly causing precipitation of the mesylate salt of Compound 50. A mixture is stirred for 2 hours. The precipitate is collected by filtration, and the flask is washed with acetone (2×20 mL) to transfer the precipitate to the filter. The precipitate is dried on filter for about 30 min. followed by vacuum-drying (55° C., 5 hours). Expected yield: >94%; expected by-product amount: 0-0.1.%

1. A method of preparing a methanesulfonic acid salt represented by formula (I): 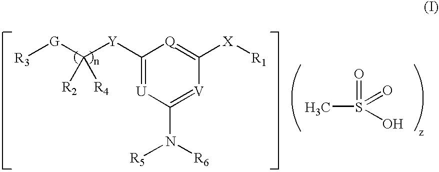
or a pharmaceutically acceptable solvate, clathrate, hydrate, polymorph or prodrug thereof, wherein:
R₁ is optionally substituted aryl, optionally substituted heteroaryl, or a group represented by the following formula: 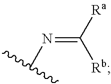
R₂ and R₄, for each occurrence, are independently, H, an optionally substituted alkyl, an optionally substituted alkylcarbonyl, —OR^{k}, —SR^{k}, —NR^{h}R^{j}, hydroxylalkyl, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, haloalkyl, aminoalkyl, mercaptoalkyl, cyano, nitro, nitroso, azide, an optionally substituted alkylcarbonylalkyl, an optionally substituted cyclyl, an optionally substituted cycloalkyl, an optionally substituted heterocyclyl, an optionally substituted heterocycloalkyl, an optionally substituted aryl, an optionally substituted aralkyl, an optionally substituted heteroaryl, an optionally substituted heteroaralkyl, or isothionitro; or R₂ and R₄ taken together are ═O, ═S, or ═NR;
R₃ is R^{g};
R₅ and R₆ are each, independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R₅ and R₆ taken together with the N to which they are attached is an optionally substituted heterocyclyl, an optionally substituted heterocycloalkyl, or an optionally substituted heteroaryl;
X is O, S, S(O), S(O)₂, or NR^{k};
Y is (CH(R^{g}))ₘ, C(O), C(NR), O, S, S(O), S(O)₂, N(R^{k}), or absent;
G is a bond, —C(O)NR^{k}NR^{k}—, —NR^{k}NR^{k}C(O)—, —NR^{k}N═CR^{k}—, —CR^{k}═NNR^{k}—, —NR^{k}NR^{k}—, —N(OH)—, —NR^{k}O—, —ONR^{k}—, —C(O)—, —C(NR)—, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k}—C(NR)—NR^{k}—, —NR^{k}—C(O)—NR^{k}—, NR^{k}—C(S)—NR^{k}, —NR^{k}S(O)₂—NR^{k}—, —P(O)(R^{c})—, —P(O)(R^{c})O—, —OP(O)(R^{c})—, —OP(O)(R^{c})O—, an optionally substituted cycloalkylene, an optionally substituted cyclylene, an optionally substituted heterocycloalkylene, an optionally substituted heterocyclylene, an optionally substituted arylene, an optionally substituted aralkylene, an optionally substituted heteroarylene, an optionally substituted heteroaralkylene, an optionally substituted heteroarylene-NR^{k}—, an optionally substituted heteroarylene-S—, an optionally substituted heteroaralkylene-O—, —Si(OR^{k})₂—, —B(OR^{k})—, —C(NR)—NR^{k}—, —NR^{k}—CR^{g}R^{g}—C(O)—, —C(O)—ONR^{k}—, —C(O)—NR^{k}O—, —C(S)—ONR^{k}—, —C(S)—NR^{k}O—, —C(NR)—ONR^{k}—, —C(NR)—NR^{k}O—, —OS(O)₂—NR^{k}NR^{k}—, —OC(O)—NR^{k}NR^{k}—, —OC(S)—NR^{k}NR^{k}—, —OC(NR)—NR^{k}NR^{k}—, —NR^{k}NR^{k}S(O)₂O—, —NR^{k}NR^{k}C(S)O—, —NR^{k}NR^{k}C(NR)O—, —OP(O)(R^{c})O—, —NR^{k}P(O)(R^{c})O—, —OP(O)(R^{c})NR^{k}—, —NR^{k}P(O)(R^{c})NR^{k}—, —P(O)(R^{c})NR^{k}—, —NR^{k}P(O)(R^{c})—, —O-alkylene-heterocycloalkylene-NR^{k}—, —NR^{k}—CHR^{g}—C(O)—NR^{k}—CHR^{g}—C(O)—, —NR^{k}—CHR^{g}—C(O)—, —NR^{k}—C(O)—CHR^{g}—, or —C(O)—NR^{k}—CHR^{g}—C(O)—; and
each of Q, U, and V are independently N or CR^{g}, wherein at least one of Q, U, or V is N; and each CR^{g} may be the same or different;
R, for each occurrence, is independently H, an optionally substituted alkyl, an optionally substituted cycloalkyl, an optionally substituted cyclyl, an optionally substituted heterocycloalkyl, an optionally substituted heterocyclyl, an optionally substituted heteroaryl, an optionally substituted aralkyl, an optionally substituted heteroaralkyl, —C(O)R^{c}, —OR^{k}, —SR^{k}, NR^{h}R^{j}, hydroxylalkyl, nitro, cyano, haloalkyl, aminoalkyl, or —S(O)₂R^{c};
each of R^{a} and R^{b}, independently, is H, optionally substituted alkyl, an optionally substituted cycloalkyl, an optionally substituted cyclyl, an optionally substituted heterocycloalkyl, an optionally substituted heterocyclyl, optionally substituted aryl, or optionally substituted heteroaryl;
R^{c}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, or thioalkoxy;
R^{g}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, thioalkoxy, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, aminoalkyl, mercaptoalkyl, cyano, nitro, nitroso, or azide;
R^{h} and R^{j}, for each occurrence, are independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R^{h} and R^{j} taken together with the N to which they are attached is an optionally substituted heterocyclyl, an optionally substituted heterocycloalkyl, or an optionally substituted heteroaryl;
R^{k}, for each occurrence, is independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, or an optionally substituted heteroaryl;
n is 0, 1, 2, 3, 4, 5, 6 or 7;
m is 0, 1, 2, 3, or 4; and
z is 1 or 2;
said method comprising the steps of:
a) providing a solution of a compound represented by formula II: 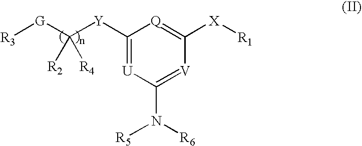
in a water miscible organic solvent, provided that the water miscible solvent is not an alcohol; and
b) adding to the solution provided in step a) a solution of methanesulfonic acid in water;
c) allowing the salt represented by formula (I) to precipitate out of solution; and
d) collecting the precipitate formed in step c), thereby preparing a methanesulfonic acid salt represented by formula (I).

2. The method of claim 1, wherein z is 2 and wherein the solution of methanesulfonic acid in water contains between about 1.8 to about 2.5 molar equivalents of methanesulfonic acid with respect to the compound in step a).

3. The method of claim 1, wherein z is 1 and wherein the solution of methanesulfonic acid in water contains between about 0.9 to about 1.25 molar equivalents of methanesulfonic acid with respect to the compound in step a).

4. The method of claim 1, wherein the water miscible organic solvent is selected from the group consisting of acetone or acetonitrile.

5. The method of claim 4, wherein the solution of the compound in the water miscible organic solvent in step a) has a molar concentration of between about 20 mM and about 150 mM.

6. The method of claim 1, wherein the solution of methanesulfonic acid has a concentration of between about 1.5 M and about 7 M.

7. The method of claim 1, wherein the temperature is maintained at about 35° C. or less during the process.

8.-13. (canceled)

14. The method of claim 1, further comprising the steps of:
e) dissolving the methanesulfonic acid salt collected in step d) in water to form a clear solution having a concentration of between about 1 mM and about 8 mM;
f) adding between about 5 mL and about 15 mL of acetone per gram of the methanesulfonic acid salt;
g) allowing the methanesulfonic acid salt to precipitate out of solution; and
h) collecting the precipitate.

15. The method of claim 14, wherein the solution is maintained at about 18° C. to about 30° C. during addition of the acetone.

16.-18. (canceled)

19. A method of preparing a methanesulfonic acid salt represented by formula (III): 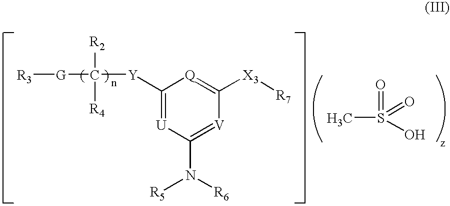
or a pharmaceutically acceptable solvate, clathrate, hydrate, prodrug or polymorph thereof, wherein:
X₃ is —C(R)═N-A-;
A is O, S, S(O), S(O)₂, C(CR^{g})₂, or NR^{k};
R₂ and R₄, for each occurrence, are independently, H, an optionally substituted alkyl, an optionally substituted alkylcarbonyl, —OR^{k}, —SR^{k}, —NR^{h}R^{j}, hydroxylalkyl, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, haloalkyl, aminoalkyl, mercaptoalkyl, cyano, nitro, nitroso, azide, an optionally substituted alkylcarbonylalkyl, an optionally substituted cyclyl, an optionally substituted cycloalkyl, an optionally substituted heterocyclyl, an optionally substituted heterocycloalkyl, an optionally substituted aryl, an optionally substituted aralkyl, an optionally substituted heteroaryl, an optionally substituted heteroaralkyl, or isothionitro; or R₂ and R₄ taken together are ═O, ═S, or ═NR;
R₃ is R^{g};
R₅ and R₆ are each, independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R₅ and R₆ taken together with the N to which they are attached is an optionally substituted heterocyclyl, an optionally substituted heterocycloalkyl, or an optionally substituted heteroaryl;
R₇ is an optionally substituted aryl or an optionally substituted heteroaryl;
Y is (CH(R^{g}))ₘ, C(O), C(NR), O, S, S(O), S(O)₂, N(R^{k}), or absent;
G is a bond, —C(O)NR^{k}NR^{k}—, —NR^{k}NR^{k}C(O)—, —NR^{k}N═CR^{k}—, —CR^{k}═NNR^{k}—, —NR^{k}NR^{k}—, —N(OH)—, —NR^{k}O—, —ONR^{k}—, —C(O)—, —C(NR)—, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k}—C(NR)—NR^{k}—, —NR^{k}—C(O)—NR^{k}—, NR^{k}C(S)—NR^{k}—, —NR^{k}—S(O)₂—NR^{k}—, —P(O)(R^{c})—, —P(O)(R^{c})O—, —OP(O)(R^{c})—, —OP(O)(R^{c})O—, an optionally substituted cycloalkylene, an optionally substituted cyclylene, an optionally substituted heterocycloalkylene, an optionally substituted heterocyclylene, an optionally substituted arylene, an optionally substituted aralkylene, an optionally substituted heteroarylene, an optionally substituted heteroaralkylene, an optionally substituted heteroarylene-NR^{k}—, an optionally substituted heteroarylene-S—, an optionally substituted heteroaralkylene-O—, —Si(OR^{k})₂—, —B(OR^{k})—, —C(NR)—NR^{k}—, —NR^{k}CR^{g}R^{g}—C(O)—, —OC(O)—ONR^{k}, —C(O)—NR^{k}O—, —C(S)—ONR^{k}—, —C(S)—NR^{k}O—, —C(NR)—ONR^{k}—, —C(NR)—NR^{k}O—, —OS(O)₂—NR^{k}NR^{k}—, —OC(O)—NR^{k}NR^{k}—, —OC(S)—NR^{k}NR^{k}, —OC(NR)—NR^{k}NR^{k}—, —NR^{k}NR^{k}S(O)₂O—, —NR^{k}NR^{k}C(S)O—, —NR^{k}NR^{k}C(NR)O—, —OP(O)(R^{c})O—, —NR^{k}P(O)(R^{c})O—, —OP(O)(R^{c})NR^{k}—, —NR^{k}P(O)(R^{c})NR^{k}, —P(O)(R^{c})NR^{k}, —NR^{k}P(O)(R^{c})—, —O-alkylene-heterocycloalkylene-NR^{k}—, —NR^{k}—CHR^{g}—C(O)—NR^{k}—CHR^{g}—C(O)—, —NR^{k}CHR^{g}—C(O)—, —NR^{k}C(O)—CHR^{g}—, or —C(O)—NR^{k}CHR^{g}—C(O)—; and
each of Q, U, and V are independently N or CR^{g}, wherein at least one of Q, U, or V is N; and each CR^{g} may be the same or different;
R, for each occurrence, is independently H, an optionally substituted alkyl, an optionally substituted cycloalkyl, an optionally substituted cyclyl, an optionally substituted heterocycloalkyl, an optionally substituted heterocyclyl, an optionally substituted heteroaryl, an optionally substituted aralkyl, an optionally substituted heteroaralkyl, —C(O)R^{c}, —OR^{k}, —SR^{k}, —NR^{h}R^{j}, hydroxylalkyl, nitro, cyano, haloalkyl, aminoalkyl, or —S(O)₂R^{c};
R^{c}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, or thioalkoxy;
R^{g}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, thioalkoxy, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, aminoalkyl, mercaptoalkyl, cyano, nitro, nitroso, or azide;
R^{h} and R^{j}, for each occurrence, are independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R^{h} and R^{j} taken together with the N to which they are attached is an optionally substituted heterocyclyl, an optionally substituted heterocycloalkyl, or an optionally substituted heteroaryl;
R^{k}, for each occurrence, is independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, or an optionally substituted heteroaryl;
n is 0, 1, 2, 3, 4, 5, 6 or 7;
m is 0, 1, 2, 3, or 4; and
z is 1 or 2;
said method comprising the steps of:
a) providing a solution of a compound represented by formula IV: 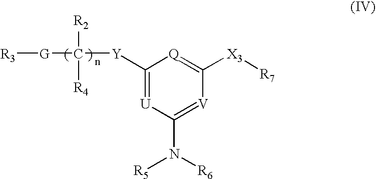
in a water miscible organic solvent, provided that the water miscible solvent is not an alcohol; and
b) adding to the solution provided in step a) a solution of methanesulfonic acid in water;
c) allowing the salt represented by formula (III) to precipitate out of solution; and
d) collecting the precipitate formed in step c), thereby preparing a methanesulfonic acid salt represented by formula (III).

20. (canceled)

21. A method of preparing a methanesulfonic acid salt represented by formula (X): 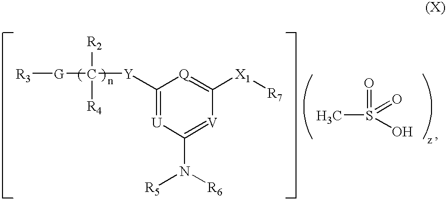
or a pharmaceutically acceptable solvate, clathrate, hydrate or polymorph thereof, wherein:
X₁ is represented by a formula selected from the group consisting of: 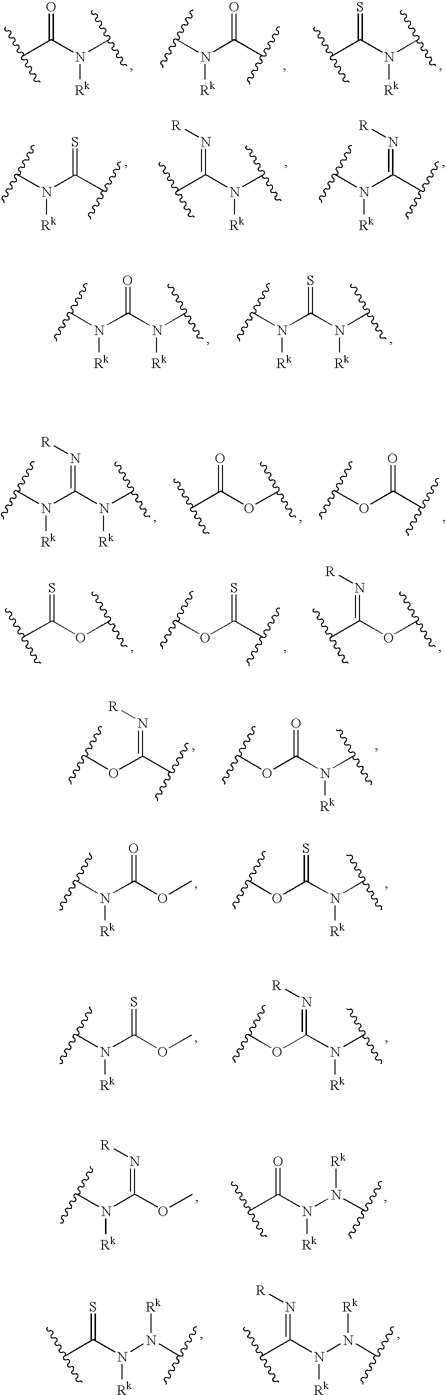 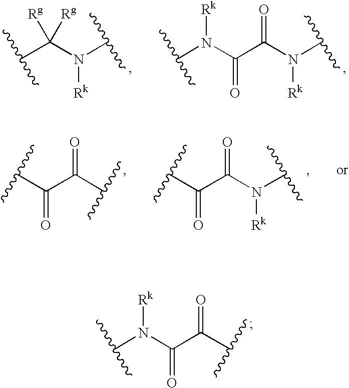
R₂ and R₄, for each occurrence, are independently, H, an optionally substituted alkyl, an optionally substituted alkylcarbonyl, —OR^{k}, —SR^{k}, —NR^{h}R^{j}, hydroxylalkyl, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, haloalkyl, aminoalkyl, mercaptoalkyl, cyano, nitro, nitroso, azide, an optionally substituted alkylcarbonylalkyl, an optionally substituted cyclyl, an optionally substituted cycloalkyl, an optionally substituted heterocyclyl, an optionally substituted heterocycloalkyl, an optionally substituted aryl, an optionally substituted aralkyl, an optionally substituted heteroaryl, an optionally substituted heteroaralkyl, or isothionitro; or R₂ and R₄ taken together are ═O, ═S, or ═NR;
R₃ is R^{g};
R₅ and R₆ are each, independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R₅ and R₆ taken together with the N to which they are attached is an optionally substituted heterocyclyl, an optionally substituted heterocycloalkyl, or an optionally substituted heteroaryl;
R₇ is an optionally substituted aryl or an optionally substituted heteroaryl;
Y is (CH(R^{g}))ₘ, C(O), C(NR), O, S, S(O), S(O)₂, N(R^{k}), or absent;
G is a bond, —C(O)NR^{k}NR^{k}—, —NR^{k}NR^{k}C(O)—, —NR^{k}N═CR^{k}—, —CR^{k}═NNR^{k}—, —NR^{k}NR^{k}—, —N(OH)—, —NR^{k}O—, —ONR^{k}, —C(O)—, —C(NR)—, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k}—C(NR)—NR^{k}—, —NR^{k}—C(O)—NR^{k}—NR^{k}C(S)—NR^{k}, —NR^{k}S(O)₂—NR^{k}—, —P(O)(R^{c})—, —P(O)(R^{c})O—, —OP(O)(R^{c})—, —OP(O)(R^{c})O—, an optionally substituted cycloalkylene, an optionally substituted cyclylene, an optionally substituted heterocycloalkylene, an optionally substituted heterocyclylene, an optionally substituted arylene, an optionally substituted aralkylene, an optionally substituted heteroarylene, an optionally substituted heteroaralkylene, an optionally substituted heteroarylene-NR^{k}—, an optionally substituted heteroarylene-S—, an optionally substituted heteroaralkylene-O—, —Si(OR^{k})₂—, —B(OR^{k})—, —C(NR)—NR^{k}—, —NR^{k}CR^{g}R^{g}—C(O)—, —C(O)—ONR^{k}, —C(O)—NR^{k}O—, —C(S)—ONR^{k}—, —C(S)—NR^{k}O—, —C(NR)—ONR^{k}—, —C(NR)—NR^{k}O—, —OS(O)₂—NR^{k}NR^{k}—, —OC(O)—NR^{k}NR^{k}—, —OC(S)—NR^{k}NR^{k}, —OC(NR)—NR^{k}NR^{k}—, —NR^{k}NR^{k}S(O)₂O—, —NR^{k}NR^{k}C(S)O—, —NR^{k}NR^{k}C(NR)O—, —OP(O)(R^{c})O—, —NR^{k}P(O)(R^{c})O—, —OP(O)(R^{c})NR^{k}—, —NR^{k}P(O)(R^{c})NR^{k}—, —P(O)R^{c})NR^{k}—, —NR^{k}P(O)(R^{c})—, —O-alkylene-heterocycloalkylene-NR^{k}—, —NR^{k}—CHR^{g}—C(O)—NR^{k}—CHR^{g}—C(O)—, —NR^{k}CHR^{g}—C(O)—, —NR^{k}C(O)—CHR^{g}—, or —C(O)—NR^{k}CHR^{g}—C(O)—; and
each of Q, U, and V are independently N or CR^{g}, wherein at least one of Q, U, or V is N; and each CR^{g} may be the same or different;
R, for each occurrence, is independently H, an optionally substituted alkyl, an optionally substituted cycloalkyl, an optionally substituted cyclyl, an optionally substituted heterocycloalkyl, an optionally substituted heterocyclyl, an optionally substituted heteroaryl, an optionally substituted aralkyl, an optionally substituted heteroaralkyl, —C(O)R^{c}, —OR^{k}, —SR^{k}, —NR^{h}R^{j}, hydroxylalkyl, nitro, cyano, haloalkyl, aminoalkyl, or —S(O)₂R^{c};
R^{c}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, or thioalkoxy;
R^{g}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, thioalkoxy, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, aminoalkyl, mercaptoalkyl, cyano, nitro, nitroso, or azide;
R^{h} and R^{j}, for each occurrence, are independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R^{h} and R^{j} taken together with the N to which they are attached is an optionally substituted heterocyclyl, an optionally substituted heterocycloalkyl, or an optionally substituted heteroaryl;
R^{k}, for each occurrence, is independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, or an optionally substituted heteroaryl;
n is 0, 1, 2, 3, 4, 5, 6 or 7;
m is 0, 1, 2, 3, or 4; and
z is 1 or 2;
said method comprising the steps of:
a) providing a solution of a compound represented by formula (XI): 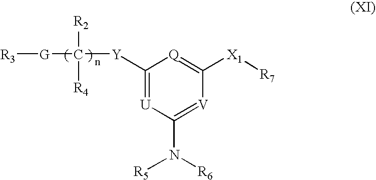
in a water miscible organic solvent, provided that the water miscible solvent is not an alcohol; and
b) adding to the solution provided in step a) a solution of methanesulfonic acid in water;
c) allowing the salt represented by formula (XI) to precipitate out of solution; and
d) collecting the precipitate formed in step c), thereby preparing a methanesulfonic acid salt represented by formula (XI).

22. A method of preparing a methanesulfonic acid salt represented by formula (I): 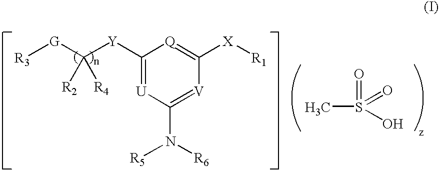
or a pharmaceutically acceptable solvate, clathrate, hydrate, polymorph or prodrug thereof, wherein:
R₁ is optionally substituted aryl, optionally substituted heteroaryl, or a group represented by the following formula: 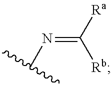
R₂ and R₄, for each occurrence, are independently, H, an optionally substituted alkyl, an optionally substituted alkylcarbonyl, —OR^{k}, —SR^{k}, —NR^{h}R^{j}, hydroxylalkyl, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, haloalkyl, aminoalkyl, mercaptoalkyl, cyano, nitro, nitroso, azide, an optionally substituted alkylcarbonylalkyl, an optionally substituted cyclyl, an optionally substituted cycloalkyl, an optionally substituted heterocyclyl, an optionally substituted heterocycloalkyl, an optionally substituted aryl, an optionally substituted aralkyl, an optionally substituted heteroaryl, an optionally substituted heteroaralkyl, or isothionitro; or R₂ and R₄ taken together are ═O, ═S, or ═NR;
R₃ is R^{g};
R₅ and R₆ are each, independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R₅ and R₆ taken together with the N to which they are attached is an optionally substituted heterocyclyl, an optionally substituted heterocycloalkyl, or an optionally substituted heteroaryl;
X is O, S, S(O), S(O)₂, or NR^{k};
Y is (CH(R^{g}))ₘ, C(O), C(NR), O, S, S(O), S(O)₂, N(R^{k}), or absent;
G is a bond, —C(O)NR^{k}NR^{k}—, —NR^{k}NR^{k}C(O)—, —NR^{k}N═CR^{k}—, —CR^{k}═NNR^{k}—, —NR^{k}NR^{k}—, —N(OH)—, —NR^{k}O—, —ONR^{k}, —C(O)—, —C(NR)—, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k}—C(NR)—NR^{k}—, —NR^{k}—C(O)—NR^{k}—NR^{k}C(S)—NR^{k}, —NR^{k}S(O)₂—NR^{k}—, —P(O)(R^{c})—, —P(O)(R^{c})O—, —OP(O)(R^{c})—, —OP(O)(R^{c})O—, an optionally substituted cycloalkylene, an optionally substituted cyclylene, an optionally substituted heterocycloalkylene, an optionally substituted heterocyclylene, an optionally substituted arylene, an optionally substituted aralkylene, an optionally substituted heteroarylene, an optionally substituted heteroaralkylene, an optionally substituted heteroarylene-NR^{k}—, an optionally substituted heteroarylene-S—, an optionally substituted heteroaralkylene-O—, —Si(OR^{k})₂—, —B(OR^{k})—, —C(NR)—NR^{k}—, —NR^{k}CR^{g}R^{g}—C(O)—, —C(O)—ONR^{k}, —C(O)—NR^{k}O—, —C(S)—ONR^{k}—, —C(S)—NR^{k}O—, —C(NR)—ONR^{k}—, —C(NR)—NR^{k}O—, —OS(O)₂—NR^{k}NR^{k}—, —OC(O)—NR^{k}NR^{k}—, —OC(S)—NR^{k}NR^{k}, —OC(NR)—NR^{k}NR^{k}—, —NR^{k}NR^{k}S(O)₂O—, —NR^{k}NR^{k}C(S)O—, —NR^{k}NR^{k}C(NR)O—, —OP(O)(R^{c})O—, —NR^{k}P(O)(R^{c})O—, —OP(O)(R^{c})NR^{k}—, —NR^{k}P(O)(R^{c})NR^{k}—, —P(O)(R^{c})NR^{k}—, —NR^{k}P(O)(R^{c})—, —O-alkylene-heterocycloalkylene-NR^{k}—, —NR^{k}—CHR^{g}—C(O)—NR^{k}—CHR^{g}—C(O)—, —NR^{k}CHR^{g}—C(O)—, —NR^{k}C(O)—CHR^{g}—, or —C(O)—NR^{k}CHR^{g}—C(O)—; and
each of Q, U, and V are independently N or CR^{g}, wherein at least one of Q, U, or V is N; and each CR^{g} may be the same or different;
R, for each occurrence, is independently H, an optionally substituted alkyl, an optionally substituted cycloalkyl, an optionally substituted cyclyl, an optionally substituted heterocycloalkyl, an optionally substituted heterocyclyl, an optionally substituted heteroaryl, an optionally substituted aralkyl, an optionally substituted heteroaralkyl, —C(O)R^{c}, —OR^{k}, —SR^{k}, —NR^{h}R^{j}, hydroxylalkyl, nitro, cyano, haloalkyl, aminoalkyl, or —S(O)₂R^{c};
each of R^{a} and R^{b}, independently, is H, optionally substituted alkyl, an optionally substituted cycloalkyl, an optionally substituted cyclyl, an optionally substituted heterocycloalkyl, an optionally substituted heterocyclyl, optionally substituted aryl, or optionally substituted heteroaryl;
R^{c}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, or thioalkoxy;
R^{g}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, thioalkoxy, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, aminoalkyl, mercaptoalkyl, cyano, nitro, nitroso, or azide;
R^{h} and R^{j}, for each occurrence, are independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R^{h} and R^{j} taken together with the N to which they are attached is an optionally substituted heterocyclyl, an optionally substituted heterocycloalkyl, or an optionally substituted heteroaryl;
R^{k}, for each occurrence, is independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, or an optionally substituted heteroaryl;
n is 0, 1, 2, 3, 4, 5, 6 or 7;
m is 0, 1, 2, 3, or 4; and
z is 1 or 2;
said method comprising the steps of:
a) providing a solution of a compound represented by formula II: 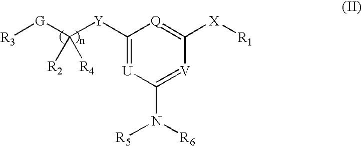
in an organic solvent, provided that the organic solvent is not an alcohol; and
b) adding to the solution provided in step a) methanesulfonic acid;
c) allowing the methanesulfonic acid salt represented by formula (I) to precipitate out of solution; and
d) collecting the precipitate formed in step c), thereby preparing a methanesulfonic acid salt represented by formula (I).

23. The method of claim 22, wherein z is 2 and wherein the methanesulfonic acid added to the solution of step a) has about 1.8 to about 2.5 molar equivalents of methanesulfonic acid with respect to the compound of step a).

24. The method of claim 22, wherein z is 1 and wherein the solution of methanesulfonic acid added to the solution of step a) has about 0.9 to about 1.25 molar equivalents of methanesulfonic acid with respect to the compound of step a).

25. The method of claim 22, wherein the solution of the compound in step a) is heated to between about 35° C. and about 75° C.

26.-28. (canceled)

29. The method of claim 22, wherein the solution of step a) has a molar concentration of the compound of between about 100 mM and about 200 mM.

30. The method of claim 29, wherein the organic solvent is ethyl acetate or dichloromethane.

31. The method of claim 29, wherein the methanesulfonic acid is added in a solution with an organic solvent.

32. The method of claim 31, wherein the solution of methanesulfonic acid in an organic solvent has a concentration of between about 1.5 M and about 7 M.

33.-34. (canceled)

35. The method of claim 22, further comprising the steps of:
e) dissolving the methanesulfonic acid salt represented collected in step
d) in water to form a clear solution having a concentration of between about 1 mM and about 8 mM;
f) adding between about 5 mL and about 15 mL of acetone per gram of the methanesulfonic acid salt;
g) allowing the methanesulfonic acid salt to precipitate out of solution; and
h) collecting the precipitate.

36. The method of claim 35, wherein the solution is maintained at about 18° C. to about 30° C. during addition of the acetone.

37.-39. (canceled)

40. A method of preparing a methanesulfonic acid salt represented by formula (III): 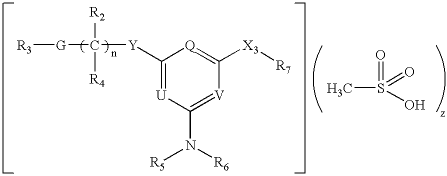
or a pharmaceutically acceptable solvate, clathrate, hydrate, prodrug or polymorph thereof, wherein:
X₃ is —C(R)═N-A-;
A is O, S, S(O), S(O)₂, C(CR^{g})₂, or NR^{k};
R₂ and R₄, for each occurrence, are independently, H, an optionally substituted alkyl, an optionally substituted alkylcarbonyl, —OR^{k}, —SR^{k}, —NR^{h}R^{j}, hydroxylalkyl, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, haloalkyl, aminoalkyl, mercaptoalkyl, cyano, nitro, nitroso, azide, an optionally substituted alkylcarbonylalkyl, an optionally substituted cyclyl, an optionally substituted cycloalkyl, an optionally substituted heterocyclyl, an optionally substituted heterocycloalkyl, an optionally substituted aryl, an optionally substituted aralkyl, an optionally substituted heteroaryl, an optionally substituted heteroaralkyl, or isothionitro; or R₂ and R₄ taken together are ═O, ═S, or ═NR;
R₃ is R^{g};
R₅ and R₆ are each, independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R₅ and R₆ taken together with the N to which they are attached is an optionally substituted heterocyclyl, an optionally substituted heterocycloalkyl, or an optionally substituted heteroaryl;
R₇ is an optionally substituted aryl or an optionally substituted heteroaryl;
Y is (CH(R^{g}))ₘ, C(O), C(NR), O, S, S(O), S(O)₂, N(R^{k}), or absent;
G is a bond, —C(O)NR^{k}NR^{k}—, —NR^{k}NR^{k}C(O)—, —NR^{k}N═CR^{k}—, —CR^{k}═NNR^{k}—, —NR^{k}NR^{k}—, —N(OH)—, —NR^{k}O—, —ONR^{k}—, —C(O)—, —C(NR)—, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k}—C(NR)—NR^{k}—, —NR^{k}—C(O)—NR^{k}— NR^{k}C(S)—NR^{k}—, —NR^{k}—S(O)₂—NR^{k}—, —P(O)(R^{c})—, —P(O)(R^{c})O—, —OP(O)(R^{c})—, —OP(O)(R^{c})O—, an optionally substituted cycloalkylene, an optionally substituted cyclylene, an optionally substituted heterocycloalkylene, an optionally substituted heterocyclylene, an optionally substituted arylene, an optionally substituted aralkylene, an optionally substituted heteroarylene, an optionally substituted heteroaralkylene, an optionally substituted heteroarylene-NR^{k}—, an optionally substituted heteroarylene-S—, an optionally substituted heteroaralkylene-O—, —Si(OR^{k})₂—, —B(OR^{k})—, —C(NR)—NR^{k}—, —NR^{k}CR^{g}R^{g}— C(O)—, —C(O)—ONR^{k}—, —C(O)—NR^{k}O—, —C(S)—ONR^{k}—, —C(S)—NR^{k}O—, —C(NR)—ONR^{k}—, —C(NR)—NR^{k}O—, —OS(O)₂—NR^{k}NR^{k}—, —OC(O)—NR^{k}NR^{k}—, —OC(S)—NR^{k}NR^{k}, —OC(NR)—NR^{k}NR^{k}—, —NR^{k}NR^{k}S(O)₂O—, —NR^{k}NR^{k}C(S)O—, —NR^{k}NR^{k}C(NR)O—, —OP(O)(R^{c})O—, —NR^{k}P(O)(R^{c})O—, —OP(O)(R^{c})NR^{k}—, —NR^{k}P(O)(R^{c})NR^{k}—, —P(O)(R^{c})NR^{k}—, —NR^{k}P(O)(R^{c})—, —O-alkylene-heterocycloalkylene-NR^{k}—, —NR^{k}—CHR^{g}—C(O)—NR^{k}—CHR^{g}—C(O)—, —NR^{k}CHR^{g}—C(O)—, —NR^{k}C(O)—CHR^{g}—, or —C(O)—NR^{k}CHR^{g}—C(O)—; and
each of Q, U, and V are independently N or CR^{g}, wherein at least one of Q, U, or V is N; and each CR^{g} may be the same or different;
R, for each occurrence, is independently H, an optionally substituted alkyl, an optionally substituted cycloalkyl, an optionally substituted cyclyl, an optionally substituted heterocycloalkyl, an optionally substituted heterocyclyl, an optionally substituted heteroaryl, an optionally substituted aralkyl, an optionally substituted heteroaralkyl, —C(O)R^{c}, —OR^{k}, —SR^{k}, NR^{h}R^{j}, hydroxylalkyl, nitro, cyano, haloalkyl, aminoalkyl, or —S(O)₂R^{c};
R^{c}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, or thioalkoxy;
R^{g}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, thioalkoxy, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, aminoalkyl, mercaptoalkyl, cyano, nitro, nitroso, or azide;
R^{h} and R^{j}, for each occurrence, are independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R^{h} and R^{j} taken together with the N to which they are attached is an optionally substituted heterocyclyl, an optionally substituted heterocycloalkyl, or an optionally substituted heteroaryl;
R^{k}, for each occurrence, is independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, or an optionally substituted heteroaryl;
n is 0, 1, 2, 3, 4, 5, 6 or 7;
m is 0, 1, 2, 3, or 4; and
z is 1 or 2;
said method comprising the steps of:
a) providing a solution of a compound represented by formula IV: 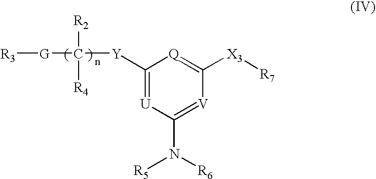
in an organic solvent, provided that the organic solvent is not an alcohol; and
b) adding to the solution provided in step a) methanesulfonic acid;
c) allowing the methanesulfonic acid salt represented by formula (III) to precipitate out of solution; and
d) collecting the precipitate formed in step c), thereby preparing a methanesulfonic acid salt represented by formula (III).

41. (canceled)

42. A method of preparing a methanesulfonic acid salt represented by formula (X): 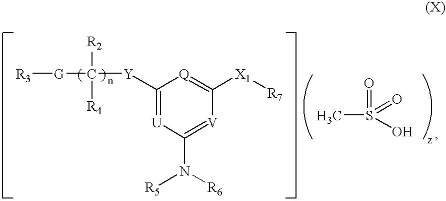
or a pharmaceutically acceptable solvate, clathrate, hydrate or polymorph thereof, wherein:
X₁ is represented by a formula selected from the group consisting of: 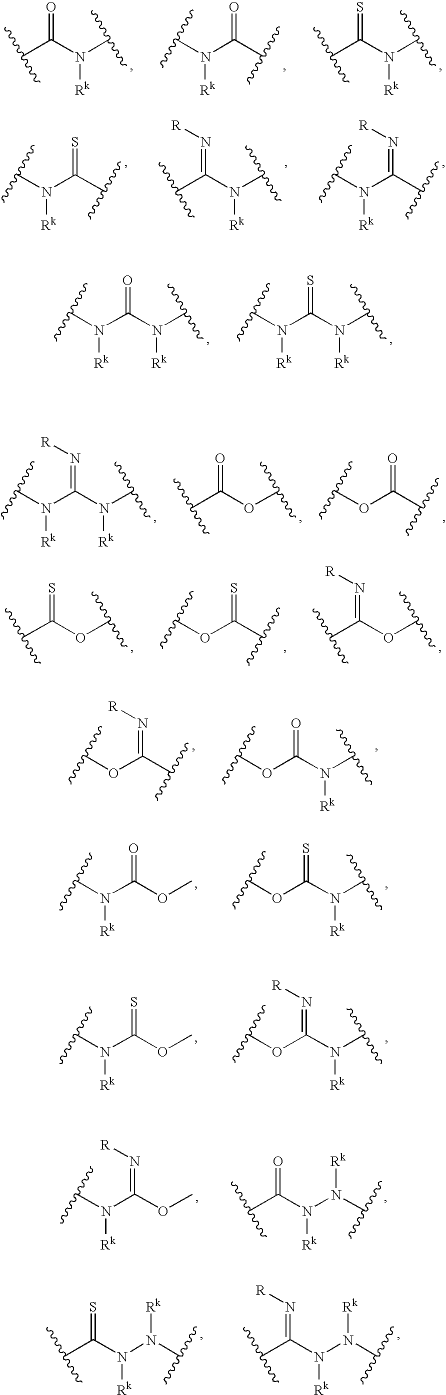 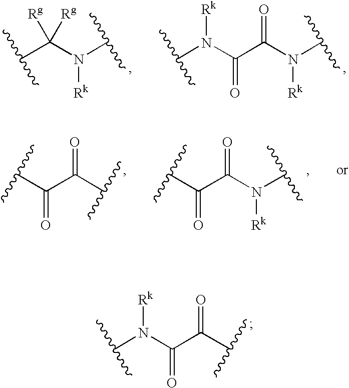
R₂ and R₄, for each occurrence, are independently, H, an optionally substituted alkyl, an optionally substituted alkylcarbonyl, —OR^{k}, —SR^{k}, —NR^{h}R^{j}, hydroxylalkyl, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, haloalkyl, aminoalkyl, mercaptoalkyl, cyano, nitro, nitroso, azide, an optionally substituted alkylcarbonylalkyl, an optionally substituted cyclyl, an optionally substituted cycloalkyl, an optionally substituted heterocyclyl, an optionally substituted heterocycloalkyl, an optionally substituted aryl, an optionally substituted aralkyl, an optionally substituted heteroaryl, an optionally substituted heteroaralkyl, or isothionitro; or R₂ and R₄ taken together are ═O, ═S, or ═NR;
R₃ is R^{g};
R₅ and R₆ are each, independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R₅ and R₆ taken together with the N to which they are attached is an optionally substituted heterocyclyl, an optionally substituted heterocycloalkyl, or an optionally substituted heteroaryl;
R₇ is an optionally substituted aryl or an optionally substituted heteroaryl;
Y is (CH(R^{g}))ₘ, C(O), C(NR), O, S, S(O), S(O)₂, N(R^{k}), or absent;
G is a bond, —C(O)NR^{k}NR^{k}—, —NR^{k}NR^{k}C(O)—, —NR^{k}N═CR^{k}—, —CR^{k}═NNR^{k}—, —NR^{k}NR^{k}—, —N(OH)—, —NR^{k}O—, —ONR^{k}, —C(O)—, —C(NR)—, —NR^{k}C(O)—, —C(O)NR^{k}—, —OC(O)—, —C(O)O—, —OC(O)O—, —NR^{k}C(O)O—, —OC(O)NR^{k}—, —NR^{k}C(S)O—, —OC(S)NR^{k}—, —NR^{k}—C(NR)—NR^{k}—, —NR^{k}—C(O)—NR^{k}—NR^{k}C(S)—NR^{k}, —NR^{k}S(O)₂—NR^{k}—, —P(O)(R^{c})—, —P(O)(R^{c})O—, —OP(O)(R^{c})—, —OP(O)(R^{c})O—, an optionally substituted cycloalkylene, an optionally substituted cyclylene, an optionally substituted heterocycloalkylene, an optionally substituted heterocyclylene, an optionally substituted arylene, an optionally substituted aralkylene, an optionally substituted heteroarylene, an optionally substituted heteroaralkylene, an optionally substituted heteroarylene-NR^{k}—, an optionally substituted heteroarylene-S—, an optionally substituted heteroaralkylene-O—, —Si(OR^{k})₂—, —B(OR^{k})—, —C(NR)—NR^{k}—, —NR^{k}CR^{g}R^{g}—C(O)—, —C(O)—ONR^{k}, —C(O)—NR^{k}O—, —C(S)—ONR^{k}—, —C(S)—NR^{k}O—, —C(NR)—ONR^{k}—, —C(NR)—NR^{k}O—, —OS(O)₂—NR^{k}NR^{k}—, —OC(O)—NR^{k}NR^{k}—, —OC(S)—NR^{k}NR^{k}, —OC(NR)—NR^{k}NR^{k}—, —NR^{k}NR^{k}S(O)₂O—, —NR^{k}NR^{k}C(S)O—, —NR^{k}NR^{k}C(NR)O—, —OP(O)(R^{c})O—, —NR^{k}P(O)(R^{c})O—, —OP(O)(R^{c})NR^{k}—, —NR^{k}P(O)(R^{c})NR^{k}—, —P(O)(R^{c})NR^{k}—, —NR^{k}P(O)(R^{c})—, —O-alkylene-heterocycloalkylene-NR^{k}—, —NR^{k}—CHR^{g}—C(O)—NR^{k}—CHR^{g}—C(O)—, —NR^{k}CHR^{g}—C(O)—, —NR^{k}C(O)—CHR^{g}—, or —C(O)—NR^{k}CHR^{g}—C(O)—; and
each of Q, U, and V are independently N or CR^{g}, wherein at least one of Q, U, or V is N; and each CR^{g} may be the same or different;
R, for each occurrence, is independently H, an optionally substituted alkyl, an optionally substituted cycloalkyl, an optionally substituted cyclyl, an optionally substituted heterocycloalkyl, an optionally substituted heterocyclyl, an optionally substituted heteroaryl, an optionally substituted aralkyl, an optionally substituted heteroaralkyl, —C(O)R^{c}, —OR^{k}, —SR^{k}, —NR^{h}R^{j}, hydroxylalkyl, nitro, cyano, haloalkyl, aminoalkyl, or —S(O)₂R^{c};
R^{c}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, or thioalkoxy;
R^{g}, for each occurrence, is independently, H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl, haloalkyl, —OR^{k}, —SR^{k}, —NR^{h}R^{j}, hydroxylalkyl, alkylcarbonylalkyl, mercaptoalkyl, aminoalkyl, sulfonylalkyl, sulfonylaryl, thioalkoxy, —C(O)R^{c}, —OC(O)R^{c}, —SC(O)R^{c}, —NR^{k}C(O)R^{c}, —C(S)R^{c}, —OC(S)R^{c}, —SC(S)R^{c}, —NR^{k}C(S)R^{c}, —C(NR)R^{c}, —OC(NR)R^{c}, —SC(NR)R^{c}, —NR^{k}C(NR)R^{c}, —SO₂R^{c}, —S(O)R^{c}, —NR^{k}SO₂R^{c}, —OS(O)₂R^{c}, —OP(O)R^{c}R^{c}, —P(O)R^{c}R^{c}, halo, aminoalkyl, mercaptoalkyl, cyano, nitro, nitroso, or azide;
R^{h} and R^{j}, for each occurrence, are independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, an optionally substituted heteroaryl; or R^{h} and R^{j} taken together with the N to which they are attached is an optionally substituted heterocyclyl, an optionally substituted heterocycloalkyl, or an optionally substituted heteroaryl;
R^{k}, for each occurrence, is independently H, an optionally substituted alkyl, an optionally substituted alkenyl, an optionally substituted alkynyl, an optionally substituted cyclyl, an optionally substituted cycloalkyl, an optionally substituted heterocyclyl, an optionally substituted heterocycloalkyl, an optionally substituted aralkyl, an optionally substituted heteroaralkyl, an optionally substituted aryl, or an optionally substituted heteroaryl;
n is 0, 1, 2, 3, 4, 5, 6 or 7;
m is 0, 1, 2, 3, or 4; and
z is 1 or 2;
said method comprising the steps of:
a) providing a solution of a compound represented by formula (XI): 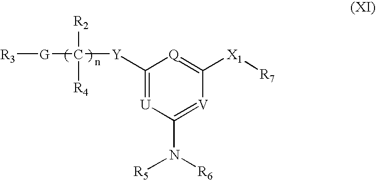
in an organic solvent, provided that the organic solvent is not an alcohol; and
b) adding to the solution provided in step a) methanesulfonic acid;
c) allowing the methanesulfonic acid salt represented by formula (X) to precipitate out of solution; and
d) collecting the precipitate formed in step c), thereby preparing a methanesulfonic acid salt represented by formula (X).
